(19) United States
(12) Patent Application Publication
(10) Pub. No.: US 2023/0414725 A1
(43) Pub. Date: Dec. 28, 2023

(54) COMPOSITIONS AND METHODS FOR TREATING POMPE DISEASE

(71) Applicant: AvroBio, Inc., Cambridge, MA (US)

(72) Inventors: Chris MASON, Nashua, NH (US); Nico Peter VAN TIL, Leiden (NL)

(21) Appl. No.: 18/037,030

(22) PCT Filed: Nov. 16, 2021
(86) PCT No.: PCT/US2021/059519

§ 371 Date: May 15, 2023

Related U.S. Application Data
(60) Provisional application No. 63/114,306, filed on Nov. 16, 2020.

(51) Int. Cl. A61K 38/47; C07K 14/65; C12N 9/26; C07K 14/775; A61K 48/00; A61P 3/00; C12N 15/86

(52) U.S. Cl. CPC A61K 38/47; C07K 14/65; C12N 9/2411; C12Y 302/0102; C07K 14/775; A61K 48/0058; A61P 3/00; C12N 15/86; C07K 2319/06; C07K 2319/02; C12N 2740/16043

(57) ABSTRACT

Described herein are compositions and methods for treating a subject having or at risk of developing Pompe disease. For example, using the compositions and methods of the disclosure, a subject having or at risk of developing Pompe disease may be administered one or more cells that contain a transgene encoding acid-alpha glucosidase (GAA) fused to a glycosylation independent lysosomal targeting (GILT) tag (GILT. GAA), wherein the GILT tag is a human insulin-like growth factor II (IGF-II) mutein containing an Ala amino acid substitution at a position corresponding to Arg37 of SEQ ID NO: 15, such as a population of CD34+ hematopoietic stem or progenitor cells that express GILT. GAA, thereby treating or preventing Pompe disease.

### SEQUENCE LISTING

The instant application contains a Sequence Listing which has been submitted electronically in ASCII format and is hereby incorporated by reference in its entirety. Said ASCII copy, created on Nov. 16, 2021, is named “51182-032WO2_Sequence_Listing_11_16_21_ST25” and is 57,146 bytes in size.

### FIELD OF THE INVENTION

The disclosure relates to compositions and methods for treating Pompe disease.

### BACKGROUND

Mutations in the lysosomal enzyme acid α-glucosidase (GAA) alter lysosomal glycogen catabolism and lead to Pompe disease, also referred to as glycogen storage disease type II. Mutations in the GAA gene result in a deficiency or absence of GAA activity, which leads to an accumulation of glycogen, which is thought to lead to progressive muscle myopathy throughout the body, affecting various body tissues, particularly the heart, skeletal muscles, liver, and nervous system. It is now currently accepted that the disease is a spectrum of phenotypes, ranging from the more severe early-onset form to the less severe late-onset form. The disorder is clinically heterogeneous in age of onset, extent of organ involvement, and rate of progression.

Current treatment of Pompe disease involves symptomatic treatment of the cardiac and respiratory symptoms. There is no approved treatment for the underlying genetic defect. Use of enzyme replacement therapy (ERT) for GAA, is approved by the F.D.A. in the United States. However, clinical evaluations using ERT to replace defective GAA in infantile Pompe patients was only moderately successful in improving cardiac and skeletal function (Klinge et al., Neuropediatrics. 2005; 36: 6-11). Recombinant GAA was shown to be more effective in resolving the cardiomyopathy than the skeletal muscle myopathy (Raben et al., Mol Ther. 2005; 11: 48-56), largely because recombinant enzyme cannot penetrate connective tissue. One of the main challenges to obtaining high therapeutic efficacy with ERT is the attainment and maintenance of therapeutically-effective amounts of exogenously delivered GAA while minimizing immunogenicity. Therefore, stable and long-lasting expression of therapeutic GAA proteins has not been achieved. Accordingly, there remains a need for compositions and methods for the treatment of Pompe disease.

### SUMMARY OF THE INVENTION

The present disclosure provides methods for treating Pompe disease by administering cells, such as pluripotent cells (e.g., embryonic stem cells (ESCs) or induced pluripotent stem cells (ISPCs)), multipotent cells (e.g., CD34+ cells such as, e.g., hematopoietic stem cells (HSCs) or myeloid precursor cells (MPCs)), blood lineage progenitor cells (BLPCS; e.g., monocytes), macrophages, microglial progenitor cells, or microglia containing a transgene encoding acid-alpha glucosidase (GAA) fused to a glycosylation-independent lysosomal targeting (GILT) tag (GILT.GAA protein), such as, e.g., a tag that includes a human insulin-like growth factor (IGF-II) mutein. The cells may be administered to a subject (e.g., mammalian subject, such as, e.g., a human) having Pompe disease by one or more of a variety of routes, including directly to the central nervous system of the subject (e.g., by intracerebroventricular administration) or systemically (e.g., by intravenous administration), among others. The disclosure also features compositions containing such cells, as well as kits containing these cells for the treatment of Pompe disease.

In a first aspect, the disclosure provides a method of treating a subject diagnosed as having Pompe disease by administering to the subject a composition containing a population of cells (e.g., pluripotent cells, ESCs, iPSCs, multipotent cells, CD34+ cells, HSCs, MPCs, BLPCs, monocytes, macrophages, microglial progenitor cells, or microglia) that contain a transgene encoding a GILT.GAA protein, wherein the GILT tag includes a human IGF-II mutein that includes an Ala amino acid substitution at a position corresponding to Arg 37 of SEQ ID NO: 15 (i.e., an R37A substitution/mutation).

In another aspect, the disclosure provides a method of improving muscle function in a subject diagnosed as having Pompe disease, the method including administering to the subject a composition containing a population of cells (e.g., pluripotent cells, ESCs, iPSCs, multipotent cells, CD34+ cells, HSCs, MPCs, BLPCs, monocytes, macrophages, microglial progenitor cells, or microglia) that contain a transgene encoding a GILT.GAA protein, wherein the GILT tag includes a human IGF-II mutein that includes an Ala amino acid substitution at a position corresponding to Arg 37 of SEQ ID NO: 15.

In another aspect, the disclosure provides a method of reducing glycogen accumulation in a subject diagnosed as having Pompe disease, the method including administering to the subject a composition containing a population of cells (e.g., pluripotent cells, ESCs, iPSCs, multipotent cells, CD34+ cells, HSCs, MPCs, BLPCs, monocytes, macrophages, microglial progenitor cells, or microglia) that contain a transgene encoding a GILT.GAA protein, wherein the GILT tag includes a human IGF-II mutein that includes an Ala amino acid substitution at a position corresponding to Arg 37 of SEQ ID NO: 15.

In another aspect, the disclosure provides a method of improving pulmonary function in a subject diagnosed as having Pompe disease, the method including administering to the subject a composition containing a population of cells (e.g., pluripotent cells, ESCs, iPSCs, multipotent cells, CD34+ cells, HSCs, MPCs, BLPCs, monocytes, macrophages, microglial progenitor cells, or microglia) that contain a transgene encoding a GILT.GAA protein, wherein the GILT tag includes a human IGF-II mutein that includes an Ala amino acid substitution at a position corresponding to Arg 37 of SEQ ID NO: 15.

In another aspect, the disclosure provides a method of increasing GAA expression in a subject diagnosed as having Pompe disease, the method including administering to the subject a composition containing a population of cells (e.g., pluripotent cells, ESCs, iPSCs, multipotent cells, CD34+ cells, HSCs, MPCs, BLPCs, monocytes, macrophages, microglial progenitor cells, or microglia) that contain a transgene encoding a GILT.GAA protein, wherein the GILT tag includes a human IGF-II mutein that includes an Ala amino acid substitution at a position corresponding to Arg 37 of SEQ ID NO: 15.

In some embodiments of any of the foregoing aspects, the human IGF-II mutein has an amino acid sequence that is at least 70% (e.g., at least 75%, 80%, 85%, 90%, 95%, 96%, 97%, 98%, 99%, or more) identical to the amino acid sequence of mature human IGF-II (SEQ ID NO: 15). In some embodiments, the human IGF-II mutein has an amino acid sequence that is at least 80% (e.g., at least 85%, 90%, 91%, 92%, 93%, 94%, 95%, 96%, 97%, 98%, 99%, or more) identical to the amino acid sequence of mature human IGF-II (SEQ ID NO: 15). In some embodiments, the human IGF-II mutein has an amino acid sequence that is at least 90% (e.g., at least 91%, 92%, 93%, 94%, 95%, 96%, 97%, 98%, 99%, or more) identical to the amino acid sequence of mature human IGF-II (SEQ ID NO: 15).

In some embodiments of any of the foregoing aspects, the GILT tag has an amino acid sequence that is at least 70% (e.g., at least 75%, 80%, 85%, 90%, 95%, 96%, 97%, 98%, 99%, or more) identical to the amino acid sequence of SEQ ID NO: 16. In some embodiments of any of the foregoing aspects, the GILT tag has an amino acid sequence that is at least 80% (e.g., at least 85%, 90%, 95%, 96%, 97%, 98%, 99%, or more) identical to the amino acid sequence of SEQ ID NO: 16. In some embodiments of any of the foregoing aspects, the GILT tag has an amino acid sequence that is at least 90% (e.g., at least 95%, 96%, 97%, 98%, 99%, or more) identical to the amino acid sequence of SEQ ID NO: 16.

In some embodiments of any of the foregoing aspects, the GILT tag has an amino acid sequence that is at least 70% (e.g., at least 75%, 80%, 85%, 90%, 95%, 96%, 97%, 98%, 99%, or more) identical to the amino acid sequence of SEQ ID NO: 17. In some embodiments of any of the foregoing aspects, the GILT tag has an amino acid sequence that is at least 80% (e.g., at least 85%, 90%, 95%, 96%, 97%, 98%, 99%, or more) identical to the amino acid sequence of SEQ ID NO: 17. In some embodiments of any of the foregoing aspects, the GILT tag has an amino acid sequence that is at least 90% (e.g., at least 95%, 96%, 97%, 98%, 99%, or more) identical to the amino acid sequence of SEQ ID NO: 17.

In some embodiments of any of the foregoing aspects, the GILT tag has an amino acid sequence that is at least 70% (e.g., at least 75%, 80%, 85%, 90%, 95%, 96%, 97%, 98%, 99%, or more) identical to the amino acid sequence of SEQ ID NO: 18. In some embodiments of any of the foregoing aspects, the GILT tag has an amino acid sequence that is at least 80% (e.g., at least 85%, 90%, 95%, 96%, 97%, 98%, 99%, or more) identical to the amino acid sequence of SEQ ID NO: 18. In some embodiments of any of the foregoing aspects, the GILT tag has an amino acid sequence that is at least 90% (e.g., at least 95%, 96%, 97%, 98%, 99%, or more) identical to the amino acid sequence of SEQ ID NO: 18.

In some embodiments of any of the foregoing aspects, the GILT tag has an amino acid sequence that is at least 70% (e.g., at least 75%, 80%, 85%, 90%, 95%, 96%, 97%, 98%, 99%, or more) identical to the amino acid sequence of SEQ ID NO: 19. In some embodiments of any of the foregoing aspects, the GILT tag has an amino acid sequence that is at least 80% (e.g., at least 85%, 90%, 95%, 96%, 97%, 98%, 99%, or more) identical to the amino acid sequence of SEQ ID NO: 19. In some embodiments of any of the foregoing aspects, the GILT tag has an amino acid sequence that is at least 90% (e.g., at least 95%, 96%, 97%, 98%, 99%, or more) identical to the amino acid sequence of SEQ ID NO: 19.

In some embodiments of any of the foregoing aspects, the GILT tag has an amino acid sequence that is at least 70% (e.g., at least 75%, 80%, 85%, 90%, 95%, 96%, 97%, 98%, 99%, or more) identical to the amino acid sequence of SEQ ID NO: 20. In some embodiments of any of the foregoing aspects, the GILT tag has an amino acid sequence that is at least 80% (e.g., at least 85%, 90%, 95%, 96%, 97%, 98%, 99%, or more) identical to the amino acid sequence of SEQ ID NO: 20. In some embodiments of any of the foregoing aspects, the GILT tag has an amino acid sequence that is at least 90% (e.g., at least 95%, 96%, 97%, 98%, 99%, or more) identical to the amino acid sequence of SEQ ID NO: 20.

In some embodiments of any of the foregoing aspects, the GILT tag is encoded by a polynucleotide having a nucleic acid sequence that is at least 85% (e.g., at least 90%, 91%, 92%, 93%, 94%, 95%, 96%, 97%, 98%, 99%, or more) identical to the nucleic acid sequence of SEQ ID NO: 21. In some embodiments of any of the foregoing aspects, the GILT tag is encoded by a polynucleotide having a nucleic acid sequence that is at least 90% (e.g., at least 91%, 92%, 93%, 94%, 95%, 96%, 97%, 98%, 99%, or more) identical to the nucleic acid sequence of SEQ ID NO: 21. In some embodiments of any of the foregoing aspects, the GILT tag is encoded by a polynucleotide having a nucleic acid sequence that is at least 95% (e.g., at least 96%, 97%, 98%, 99%, or more) identical to the nucleic acid sequence of SEQ ID NO: 21. In some embodiments, the GILT tag is encoded by a polynucleotide having the nucleic acid sequence of SEQ ID NO: 21.

In some embodiments of any of the foregoing aspects, the GILT tag is encoded by a polynucleotide having a nucleic acid sequence that is at least 85% (e.g., at least 90%, 91%, 92%, 93%, 94%, 95%, 96%, 97%, 98%, 99%, or more) identical to the nucleic acid sequence of SEQ ID NO: 22. In some embodiments of any of the foregoing aspects, the GILT tag is encoded by a polynucleotide having a nucleic acid sequence that is at least 90% (e.g., at least 91%, 92%, 93%, 94%, 95%, 96%, 97%, 98%, 99%, or more) identical to the nucleic acid sequence of SEQ ID NO: 22. In some embodiments of any of the foregoing aspects, the GILT tag is encoded by a polynucleotide having a nucleic acid sequence that is at least 95% (e.g., at least 96%, 97%, 98%, 99%, or more) identical to the nucleic acid sequence of SEQ ID NO: 22. In some embodiments, the GILT tag is encoded by a polynucleotide having the nucleic acid sequence of SEQ ID NO: 22.

In some embodiments of any of the foregoing aspects, the GILT tag is encoded by a polynucleotide having a nucleic acid sequence that is at least 85% (e.g., at least 90%, 91%, 92%, 93%, 94%, 95%, 96%, 97%, 98%, 99%, or more) identical to the nucleic acid sequence of SEQ ID NO: 23. In some embodiments of any of the foregoing aspects, the GILT tag is encoded by a polynucleotide having a nucleic acid sequence that is at least 90% (e.g., at least 91%, 92%, 93%, 94%, 95%, 96%, 97%, 98%, 99%, or more) identical to the nucleic acid sequence of SEQ ID NO: 23. In some embodiments of any of the foregoing aspects, the GILT tag is encoded by a polynucleotide having a nucleic acid sequence that is at least 95% (e.g., at least 96%, 97%, 98%, 99%, or more) identical to the nucleic acid sequence of SEQ ID NO: 23. In some embodiments, the GILT tag is encoded by a polynucleotide having the nucleic acid sequence of SEQ ID NO: 23.

In some embodiments of any of the foregoing aspects, the human IGF-II mutein has diminished binding affinity for the insulin receptor relative to the affinity of naturally-occurring human IGF-II for the insulin receptor, wherein the IGF-II mutein is resistant to furin cleavage, wherein the IGF-II mutein binds to the human cation-independent mannose-6-phosphate receptor in a mannose-6-phosphate-independent manner.

In some embodiments, the transgene is operably linked to a promoter. In some embodiments, the promoter is a ubiquitous promoter. In some embodiments, the ubiquitous promoter is an elongation factor 1-alpha (EF1α) promoter, phosphoglycerate kinase 1 (PGK) promoter, or an EF1a promoter containing elements of locus control region of the β-globin gene containing regions of erythroid-specific DNase I hypersensitivity (HS) regions 2, 3, and 4 (β-LCR(HS4,3,2)-EFS) promoter. In some embodiments, the promoter is a cell lineage-specific promoter. In some embodiments, the cell lineage-specific promoter is a CD68 molecule (CD68) promoter, the CD11 b molecule (CD11 b) promoter, C-X3-C motif chemokine receptor 1 (CX3CR1) promoter, allograft inflammatory factor 1 promoter (AIF1), purinergic receptor P2Y12 (P2Y12) promoter, transmembrane protein 119 promoter (TMEM119), or colony stimulating factor 1 receptor (CSF1R) promoter. In some embodiments, promoter is a synthetic promoter. In some embodiments, the promoter is a viral promoter. In some embodiments, the viral promoter is an adenovirus late promoter, vaccinia virus 7.5K promoter, simian virus 40 (SV40) promoter, cytomegalovirus (CMV) promoter, tk promoter of herpes simplex virus (HSV), mouse mammary tumor virus (MMTV) promoter, long terminal repeat (LTR) promoter of human immunodeficiency virus (HIV), Moloney virus promoter, Epstein-Barr virus promoter (EBV), or Rous sarcoma virus (RSV) promoter. In some embodiments, the synthetic promoter is a “Myeloproliferative Sarcoma Virus Enhancer, Negative Control Region Deleted, dl587rev Primer-Binding Site Substituted” (MND) promoter. In some embodiments, the MND promoter includes a polynucleotide having at least 85% (e.g., at least 90%, 91%, 92%, 93%, 94%, 95%, 96%, 97%, 98%, 99%, or more) sequence identity to the nucleic acid sequence of SEQ ID NO: 10. In some embodiments, the MND promoter includes a polynucleotide having at least 90% (e.g., at least 91%, 92%, 93%, 94%, 95%, 96%, 97%, 98%, 99%, or more) sequence identity to the nucleic acid sequence of SEQ ID NO: 10. In some embodiments, the MND promoter includes a polynucleotide having at least 95% (e.g., at least 96%, 97%, 98%, 99%, or more) sequence identity to the nucleic acid sequence of SEQ ID NO: 10. In some embodiments, the MND promoter includes a polynucleotide having at least 98% (e.g., at least 99%, or more) sequence identity to the nucleic acid sequence of SEQ ID NO: 10. In some embodiments, the MND promoter includes a polynucleotide having at least 99% sequence identity to the nucleic acid sequence of SEQ ID NO: 10. In some embodiments, the MND promoter includes a polynucleotide having the nucleic acid sequence of SEQ ID NO: 10. In some embodiments, the MND promoter includes a polynucleotide having at least 85% (e.g., at least 90%, 91%, 92%, 93%, 94%, 95%, 96%, 97%, 98%, 99%, or more) sequence identity to the nucleic acid sequence of SEQ ID NO: 11. In some embodiments, the MND promoter includes a polynucleotide having at least 90% (e.g., at least 91%, 92%, 93%, 94%, 95%, 96%, 97%, 98%, 99%, or more) sequence identity to the nucleic acid sequence of SEQ ID NO: 11. In some embodiments, the MND promoter includes a polynucleotide having at least 95% (e.g., at least 96%, 97%, 98%, 99%, or more) sequence identity to the nucleic acid sequence of SEQ ID NO: 11. In some embodiments, the MND promoter includes a polynucleotide having at least 98% (e.g., at least 99%, or more) sequence identity to the nucleic acid sequence of SEQ ID NO: 11. In some embodiments, the MND promoter includes a polynucleotide having at least 99% sequence identity to the nucleic acid sequence of SEQ ID NO: 11. In some embodiments, the MND promoter includes a polynucleotide having the nucleic acid sequence of SEQ ID NO: 11.

In some embodiments, the GAA is full-length GAA, such as GAA having an amino acid sequence of any one of SEQ ID NOs: 1-4 or a variant thereof having at least 85% (e.g., 90%, 95%, 96%, 97%, 98%, 99%, or more) sequence identity thereto (e.g. at least 85%, 90%, 95%, 96%, 97%, 98%, 99%, or more) sequence identity to the amino acid sequence of any one of SEQ ID NOs: 1-4.

In some embodiments, the transgene encodes a GAA protein having an amino acid sequence of SEQ ID NO: 1 or a variant thereof having at least 85% (e.g., at least 90%, 91%, 92%, 93%, 94%, 95%, 96%, 97%, 98%, 99%, or more) sequence identity to the amino acid sequence of SEQ ID NO: 1. In some embodiments, the transgene encodes a GAA protein having an amino acid sequence having at least 90% (e.g., at least 91%, 92%, 93%, 94%, 95%, 96%, 97%, 98%, 99%, or more) sequence identity to the amino acid sequence of SEQ ID NO: 1. In some embodiments the transgene encodes a GAA protein having an amino acid sequence having at least 95% (e.g., at least 96%, 97%, 98%, 99%, or more) sequence identity to the amino acid sequence of SEQ ID NO: 1. In some embodiments, the transgene encodes a GAA protein having an amino acid sequence having at least 98% (e.g., at least 99%, or more) sequence identity to the amino acid sequence of SEQ ID NO: 1. In some embodiments, the transgene encodes a GAA protein having an amino acid sequence having at least 99% sequence identity to the amino acid sequence of SEQ ID NO: 1. In some embodiments, the transgene encodes a GAA protein having the amino acid sequence of SEQ ID NO: 1.

In some embodiments, the transgene encodes a GAA protein having an amino acid sequence of SEQ ID NO: 2 or a variant thereof having at least 85% (e.g., at least 90%, 91%, 92%, 93%, 94%, 95%, 96%, 97%, 98%, 99%, or more) sequence identity to the amino acid sequence of SEQ ID NO: 2. In some embodiments, the transgene encodes a GAA protein having an amino acid sequence having at least 90% (e.g., at least 91%, 92%, 93%, 94%, 95%, 96%, 97%, 98%, 99%, or more) sequence identity to the amino acid sequence of SEQ ID NO: 2. In some embodiments the transgene encodes a GAA protein having an amino acid sequence having at least 95% (e.g., at least 96%, 97%, 98%, 99%, or more) sequence identity to the amino acid sequence of SEQ ID NO: 2. In some embodiments, the transgene encodes a GAA protein having an amino acid sequence having at least 98% (e.g., at least 99%, or more) sequence identity to the amino acid sequence of SEQ ID NO: 2. In some embodiments, the transgene encodes a GAA protein having an amino acid sequence having at least 99% sequence identity to the amino acid sequence of SEQ ID NO: 2. In some embodiments, the transgene encodes a GAA protein having the amino acid sequence of SEQ ID NO: 2.

In some embodiments, the transgene encodes a GAA protein having an amino acid sequence of SEQ ID NO: 3 or a variant thereof having at least 85% (e.g., at least 90%, 91%, 92%, 93%, 94%, 95%, 96%, 97%, 98%, 99%, or more) sequence identity to the amino acid sequence of SEQ ID NO: 3. In some embodiments, the transgene encodes a GAA protein having an amino acid sequence having at least 90% (e.g., at least 91%, 92%, 93%, 94%, 95%, 96%, 97%, 98%, 99%, or more) sequence identity to the amino acid sequence of SEQ ID NO: 3. In some embodiments the transgene encodes a GAA protein having an amino acid sequence having at least 95% (e.g., at least 96%, 97%, 98%, 99%, or more) sequence identity to the amino acid sequence of SEQ ID NO: 3. In some embodiments, the transgene encodes a GAA protein having an amino acid sequence having at least 98% (e.g., at least 99%, or more) sequence identity to the amino acid sequence of SEQ ID NO: 3. In some embodiments, the transgene encodes a GAA protein having an amino acid sequence having at least 99% sequence identity to the amino acid sequence of SEQ ID NO: 3. In some embodiments, the transgene encodes a GAA protein having the amino acid sequence of SEQ ID NO: 3.

In some embodiments, the transgene encodes a GAA protein having an amino acid sequence of SEQ ID NO: 4 or a variant thereof having at least 85% (e.g., at least 90%, 91%, 92%, 93%, 94%, 95%, 96%, 97%, 98%, 99%, or more) sequence identity to the amino acid sequence of SEQ ID NO: 4. In some embodiments, the transgene encodes a GAA protein having an amino acid sequence having at least 90% (e.g., at least 91%, 92%, 93%, 94%, 95%, 96%, 97%, 98%, 99%, or more) sequence identity to the amino acid sequence of SEQ ID NO: 4. In some embodiments the transgene encodes a GAA protein having an amino acid sequence having at least 95% (e.g., at least 96%, 97%, 98%, 99%, or more) sequence identity to the amino acid sequence of SEQ ID NO: 4. In some embodiments, the transgene encodes a GAA protein having an amino acid sequence having at least 98% (e.g., at least 99%, or more) sequence identity to the amino acid sequence of SEQ ID NO: 4. In some embodiments, the transgene encodes a GAA protein having an amino acid sequence having at least 99% sequence identity to the amino acid sequence of SEQ ID NO: 4. In some embodiments, the transgene encodes a GAA protein having the amino acid sequence of SEQ ID NO: 4.

In some embodiments, the GAA protein is encoded by a polynucleotide having the nucleic acid sequence of SEQ ID NO: 5 or a variant thereof having at least 85% (e.g., at least 90%, 91%, 92%, 93%, 94%, 95%, 96%, 97%, 98%, 99%, or more) sequence identity to the nucleic acid sequence of SEQ ID NO: 5. In some embodiments, the GAA protein is encoded by a polynucleotide having at least 90% (e.g., at least 91%, 92%, 93%, 94%, 95%, 96%, 97%, 98%, 99%, or more) sequence identity to the nucleic acid sequence of SEQ ID NO: 5. In some embodiments, the GAA protein is encoded by a polynucleotide having at least 95% (e.g., at least 96%, 97%, 98%, 99%, or more) sequence identity to the nucleic acid sequence of SEQ ID NO: 5. In some embodiments, the GAA protein is encoded by a polynucleotide having at least 98% (e.g., at least 99%, or more) sequence identity to the nucleic acid sequence of SEQ ID NO: 5. In some embodiments, the GAA protein is encoded by a polynucleotide having at least 99% sequence identity to the nucleic acid sequence of SEQ ID NO: 5. In some embodiments, the GAA protein is encoded by a polynucleotide having the nucleic acid sequence of SEQ ID NO: 5.

In some embodiments, the GAA protein is encoded by a polynucleotide having the nucleic acid sequence of SEQ ID NO: 6 or a variant thereof having at least 85% (e.g., at least 90%, 91%, 92%, 93%, 94%, 95%, 96%, 97%, 98%, 99%, or more) sequence identity to the nucleic acid sequence of SEQ ID NO: 6. In some embodiments, the GAA protein is encoded by a polynucleotide having at least 90% (e.g., at least 91%, 92%, 93%, 94%, 95%, 96%, 97%, 98%, 99%, or more) sequence identity to the nucleic acid sequence of SEQ ID NO: 6. In some embodiments, the GAA protein is encoded by a polynucleotide having at least 95% (e.g., at least 96%, 97%, 98%, 99%, or more) sequence identity to the nucleic acid sequence of SEQ ID NO: 6. In some embodiments, the GAA protein is encoded by a polynucleotide having at least 98% (e.g., at least 99%, or more) sequence identity to the nucleic acid sequence of SEQ ID NO: 6. In some embodiments, the GAA protein is encoded by a polynucleotide having at least 99% sequence identity to the nucleic acid sequence of SEQ ID NO: 6. In some embodiments, the GAA protein is encoded by a polynucleotide having the nucleic acid sequence of SEQ ID NO: 6.

In some embodiments, the GAA protein is encoded by a polynucleotide having the nucleic acid sequence of SEQ ID NO: 7 or a variant thereof having at least 85% (e.g., at least 90%, 91%, 92%, 93%, 94%, 95%, 96%, 97%, 98%, 99%, or more) sequence identity to the nucleic acid sequence of SEQ ID NO: 7. In some embodiments, the GAA protein is encoded by a polynucleotide having at least 90% (e.g., at least 91%, 92%, 93%, 94%, 95%, 96%, 97%, 98%, 99%, or more) sequence identity to the nucleic acid sequence of SEQ ID NO: 7. In some embodiments, the GAA protein is encoded by a polynucleotide having at least 95% (e.g., at least 96%, 97%, 98%, 99%, or more) sequence identity to the nucleic acid sequence of SEQ ID NO: 7. In some embodiments, the GAA protein is encoded by a polynucleotide having at least 98% (e.g., at least 99%, or more) sequence identity to the nucleic acid sequence of SEQ ID NO: 7. In some embodiments, the GAA protein is encoded by a polynucleotide having at least 99% sequence identity to the nucleic acid sequence of SEQ ID NO: 7. In some embodiments, the GAA protein is encoded by a polynucleotide having the nucleic acid sequence of SEQ ID NO: 7.

In some embodiments, the GAA protein is encoded by a polynucleotide having the nucleic acid sequence of SEQ ID NO: 8 or a variant thereof having at least 85% (e.g., at least 90%, 91%, 92%, 93%, 94%, 95%, 96%, 97%, 98%, 99%, or more) sequence identity to the nucleic acid sequence of SEQ ID NO: 8. In some embodiments, the GAA protein is encoded by a polynucleotide having at least 90% (e.g., at least 91%, 92%, 93%, 94%, 95%, 96%, 97%, 98%, 99%, or more) sequence identity to the nucleic acid sequence of SEQ ID NO: 8. In some embodiments, the GAA protein is encoded by a polynucleotide having at least 95% (e.g., at least 96%, 97%, 98%, 99%, or more) sequence identity to the nucleic acid sequence of SEQ ID NO: 8. In some embodiments, the GAA protein is encoded by a polynucleotide having at least 98% (e.g., at least 99%, or more) sequence identity to the nucleic acid sequence of SEQ ID NO: 8. In some embodiments, the GAA protein is encoded by a polynucleotide having at least 99% sequence identity to the nucleic acid sequence of SEQ ID NO: 8. In some embodiments, the GAA protein is encoded by a polynucleotide having the nucleic acid sequence of SEQ ID NO: 8.

In some embodiments, the GAA protein is encoded by a polynucleotide having the nucleic acid sequence of SEQ ID NO: 9 or a variant thereof having at least 85% (e.g., at least 90%, 91%, 92%, 93%, 94%, 95%, 96%, 97%, 98%, 99%, or more) sequence identity to the nucleic acid sequence of SEQ ID NO: 9. In some embodiments, the GAA protein is encoded by a polynucleotide having at least 90% (e.g., at least 91%, 92%, 93%, 94%, 95%, 96%, 97%, 98%, 99%, or more) sequence identity to the nucleic acid sequence of SEQ ID NO: 9. In some embodiments, the GAA protein is encoded by a polynucleotide having at least 95% (e.g., at least 96%, 97%, 98%, 99%, or more) sequence identity to the nucleic acid sequence of SEQ ID NO: 9. In some embodiments, the GAA protein is encoded by a polynucleotide having at least 98% (e.g., at least 99%, or more) sequence identity to the nucleic acid sequence of SEQ ID NO: 9. In some embodiments, the GAA protein is encoded by a polynucleotide having at least 99% sequence identity to the nucleic acid sequence of SEQ ID NO: 9. In some embodiments, the GAA protein is encoded by a polynucleotide having the nucleic acid sequence of SEQ ID NO: 9.

In some embodiments of any of the foregoing aspects, the GAA is full-length GAA.

In some embodiments of any of the foregoing aspects, the GAA includes a signal peptide.

In some embodiments of any of the foregoing aspects, the signal peptide is a GAA signal peptide.

In some embodiments of any of the foregoing aspects, the signal peptide is an IGF-II signal peptide. In some embodiments, the IGF-II signal peptide includes an amino acid sequence of SEQ ID NO: 12.

In some embodiments of any of the foregoing aspects, the transgene encodes two or more GAA (or GILT.GAA) proteins (e.g., at least 2, 3, 4, 5, 6, 7, 8, 9, 10 or more GAA (or GILT.GAA) proteins). In some embodiments, the transgene encodes from two to ten GAA (or GILT.GAA) proteins (e.g., 2, 3, 4, 5, 6, 7, 8, 9, or 10 GAA (or GILT.GAA) proteins). In some embodiments, the transgene encodes from two to five GAA (or GILT.GAA) proteins (e.g., 2, 3, 4, or 5 GAA (or GILT.GAA) proteins). In some embodiments, the transgene encodes two GAA (or GILT.GAA) proteins. In some embodiments, the GAA transgenes are expressed from a single, polycistronic expression cassette. In some embodiments, the GAA transgenes are separated from one another by way of one or more (e.g., 1, 2, 3, 4, 5, 6, 7, 8, 9, or more) internal ribosome entry sites (IRES). In some embodiments, the GAA transgenes are expressed from one or more (e.g., 1, 2, 3, 4, 5, 6, 7, 8, 9, 10, or more) monocistronic expression cassettes.

In some embodiments of any of the foregoing aspects, the GILT.GAA protein includes a low-density lipoprotein receptor family (LDLRf) binding (Rb) domain of apolipoprotein E (ApoE), or a fragment, variant, or oligomer thereof. In some embodiments, the Rb domain of ApoE, or a fragment, variant, or oligomer thereof, is operably linked to the N-terminus of the GILT.GAA. In some embodiments, the Rb domain of ApoE, or a fragment, variant, or oligomer thereof is operably linked to the C-terminus of the GILT.GAA. In some embodiments, the GILT.GAA fusion protein contains 1 or more (e.g., 1, 2, 3, 4, 5, 6, 7, 8, 9, 10, or more) oligomers of the Rb domain of ApoE. In some embodiments, the Rb domain contains a region of ApoE having at least 70% sequence identity (e.g., at least 70%, 75%, 80%, 85%, 90%, 95%, 96%, 97%, 98%, 99%, or greater, sequence identity) to residues 25-185 of SEQ ID NO: 24. In some embodiments, the Rb domain contains a region of ApoE having at least 70% sequence identity (e.g., at least 70%, 75%, 80%, 85%, 90%, 95%, 96%, 97%, 98%, 99%, or greater, sequence identity) to residues 50-180 of SEQ ID NO: 24. In some embodiments, the Rb domain contains a region of ApoE having at least 70% sequence identity (e.g., at least 70%, 75%, 80%, 85%, 90%, 95%, 96%, 97%, 98%, 99%, or greater, sequence identity) to residues 75-175 of SEQ ID NO: 24. In some embodiments, the Rb domain contains a region of ApoE having at least 70% sequence identity (e.g., at least 70%, 75%, 80%, 85%, 90%, 95%, 96%, 97%, 98%, 99%, or greater, sequence identity) to residues 100-170 of SEQ ID NO: 24. In some embodiments, the Rb domain contains a region of ApoE having at least 70% sequence identity (e.g., at least 70%, 75%, 80%, 85%, 90%, 95%, 96%, 97%, 98%, 99%, or greater, sequence identity) to residues 125-160 of SEQ ID NO: 24. In some embodiments, the Rb domain contains a region of ApoE having at least 70% sequence identity (e.g., at least 70%, 75%, 80%, 85%, 90%, 95%, 96%, 97%, 98%, 99%, or greater, sequence identity) to residues 130-150 of SEQ ID NO: 24. In some embodiments, the Rb domain contains a region of ApoE having at least 70% sequence identity (e.g., at least 70%, 75%, 80%, 85%, 90%, 95%, 96%, 97%, 98%, 99%, or greater, sequence identity) to residues 148-173 or a portion thereof containing residues 159-167 of SEQ ID NO: 24, or a variant having at least 70% sequence identity (e.g., at least 70%, 75%, 80%, 85%, 90%, 95%, 96%, 97%, 98%, 99%, or greater, sequence identity) to residues 159-167 of SEQ ID NO: 24. In some embodiments, the Rb domain contains a region having at least 70% sequence identity (e.g., at least 70%, 75%, 80%, 85%, 90%, 95%, 96%, 97%, 98%, 99%, or greater, sequence identity) to the amino acid sequence of residues 159-167 of SEQ ID NO: 24.

In some embodiments of any of the foregoing aspects, the GILT.GAA protein does not include a low-density lipoprotein receptor family (LDLRf) binding (Rb) domain of apolipoprotein E (ApoE), or a fragment, variant, or oligomer thereof.

In some embodiments of any of the foregoing aspects, the transgene encoding GILT.GAA further contains a micro RNA (miRNA) targeting sequence (e.g., a miR-126 targeting sequence). In some embodiments, the miRNA targeting sequence (e.g., a miR-126 targeting sequence) is located within the 3′-untranslated region (UTR) of the transgene.

In some embodiments of any of the foregoing aspects, the GILT.GAA protein penetrates the blood brain barrier (BBB) in the subject.

In some embodiments of any of the foregoing aspects, the transgene encoding GAA includes a polynucleotide encoding polypeptide that contains one or more amino acid substitutions, such as one or more conservative amino acid substitutions (e.g., 1, 2, 3, 4, 5, 6, 7, 8, 9, or 10 or more amino acid substitutions, such as 1, 2, 3, 4, 5, 6, 7, 8, 9, or 10 or more conservative amino acid substitutions), relative to a polypeptide having the sequence of any one of SEQ ID NOs: 1-4.

In some embodiments of any of the foregoing aspects, the cells are pluripotent cells or multipotent cells. In some embodiments, the pluripotent cells are ESCs. In some embodiments, the pluripotent cells are iPSCs. In some embodiments, the cells are CD34+ cells. In some embodiments, the cells are multipotent cells. In some embodiments, the multipotent cells are CD34+ cells. In some embodiments, the CD34+ cells are hematopoietic stem cells. In some embodiments, the CD34+ cells are myeloid cells. In some embodiments, the myeloid cells are myeloid progenitor cells. In some embodiments, the myeloid cells are erythrocytes, mast cells, megakaryocytes, thrombocytes, myeloblasts, basophils, neutrophils, eosinophils, monocytes, or macrophages. In some embodiments, the cells are blood line progenitor cells (BLPCs). In some embodiments, the BLPCs are monocytes. In some embodiments the cells are macrophages. In some embodiments, the cells are microglial progenitor cells. In some embodiments, the cells are microglia.

In some embodiments of any of the foregoing aspects, expression of the GAA transgene is measured in one or more organs, tissues, or body fluids of the subject. In some embodiments, the one or more body fluids is peripheral blood. In some embodiments, the one or more tissues is muscle tissue or nervous system tissue. In some embodiments, the one or more organs is the liver and/or the heart.

In some embodiments, a population of endogenous cells in the subject has been ablated prior to administration of the composition to the subject. In some embodiments, the method includes ablating a population of endogenous cells in the subject prior to administering the composition to the subject. In some embodiments, the microglia are ablated using an agent selected from the group consisting of busulfan, PLX3397, PLX647, PLX5622, treosulfan, and clodronate liposomes, by radiation therapy, or a combination thereof. In some embodiments, the endogenous cells are ablated using busulfan.

In some embodiments, the endogenous cells are pluripotent cells or multipotent cells. In some embodiments, the pluripotent cells are ESCs. In some embodiments, the pluripotent cells are iPSCs. In some embodiments, the cells are CD34+ cells. In some embodiments, the endogenous cells are multipotent cells. In some embodiments, the multipotent cells are CD34+ cells. In some embodiments, the CD34+ cells are hematopoietic stem cells. In some embodiments, the CD34+ cells are myeloid cells. In some embodiments, the myeloid cells are myeloid progenitor cells. In some embodiments, the myeloid cells are erythrocytes, mast cells, megakaryocytes, thrombocytes, myeloblasts, basophils, neutrophils, eosinophils, monocytes, or macrophages. In some embodiments, the endogenous cells are blood line progenitor cells (BLPCs). In some embodiments, the BLPCs are monocytes. In some embodiments the endogenous cells are macrophages. In some embodiments, the endogenous cells are microglial progenitor cells. In some embodiments, the endogenous cells are microglia.

In some embodiments of any of the foregoing aspects, the composition is administered systemically to the subject. In some embodiments, the composition is administered to the subject by way of intravenous injection. In some embodiments, the composition is administered directly to the central nervous system of the subject. In some embodiments, the composition is administered to the cerebrospinal fluid of the subject. For example, the composition may be administered to the subject by way of intracerebroventricular injection, intrathecal injection, stereotactic injection, or a combination thereof. In some embodiments, the composition is administered to the subject by way of intraparenchymal injection.

In some embodiments, the composition is administered to the subject by way of a bone marrow transplant. In some embodiments, the composition is administered directly to the bone marrow of the subject, such as by way of intraosseous injection.

In some embodiments, the composition is administered to the subject by way of intracerebroventricular injection. In some embodiments, the composition is administered to the subject by way of intravenous injection.

In some embodiments, the composition is administered to the subject by direct administration to the central nervous system of the subject and by systemic administration. In some embodiments, the composition is administered to the subject by way of intracerebroventricular injection and intravenous injection. In some embodiments, the composition is administered to the subject by way of intrathecal injection and intravenous injection. In some embodiments, the composition is administered to the subject by way of intraparenchymal injection and intravenous injection.

In some embodiments, the cells are autologous cells. In some embodiments, the cells are allogeneic cells.

In some embodiments, the cells are transduced ex vivo to express the GILT.GAA.

In some embodiments, the cells are transduced with a viral vector selected from the group including an adeno-associated virus (AAV), an adenovirus, a parvovirus, a coronavirus, a rhabdovirus, a paramyxovirus, a picornavirus, an alphavirus, a herpes virus, a poxvirus, and a Retroviridae family virus.

In some embodiments, the viral vector is a Retroviridae family viral vector. In some embodiments, the Retroviridae family viral vector is a lentiviral vector. In some embodiments, the Retroviridae family viral vector is an alpharetroviral vector. In some embodiments, the Retroviridae family viral vector is a gammaretroviral vector. In some embodiments, the Retroviridae family viral vector includes a central polypurine tract, a woodchuck hepatitis virus post-transcriptional regulatory element, a 5′-LTR, HIV signal sequence, HIV Psi signal 5′-splice site, delta-GAG element, 3′-splice site, and a 3′-self inactivating LTR.

In some embodiments, the viral vector is an AAV selected from the group including AAV1, AAV2, AAV3, AAV4, AAV5, AAV6, AAV7, AAVS, AAV9, AAV10, and AAVrh74.

In some embodiments, the viral vector is a pseudotyped viral vector. In some embodiments, the viral vector is a pseudotyped AAV, a pseudotyped adenovirus, a pseudotyped parvovirus, a pseudotyped coronavirus, a pseudotyped rhabdovirus, a pseudotyped paramyxovirus, a pseudotyped picornavirus, a pseudotyped alphavirus, a pseudotyped herpes virus, a pseudotyped poxvirus, and a pseudotyped Retroviridae family virus.

In some embodiments, the cells are transfected ex vivo to express the GILT.GAA.

In some embodiments, the cells are transfected using an agent selected from the group including a cationic polymer, diethylaminoethyl-dextran, polyethylenimine, a cationic lipid, a liposome, calcium phosphate, an activated dendrimer, and a magnetic bead; or a technique selected from the group including electroporation, Nucleofection, squeeze-poration, sonoporation, optical transfection, Magnetofection, and impalefection.

In some embodiments of any of the foregoing aspects, the subject with Pompe disease is a cross-reactive immunological material (CRIM)-negative subject. In some embodiments, the method further includes administering an immune tolerance induction (ITI) agent to the CRIM-negative subject prior to, concurrently with, or after the administration of the composition. In some embodiments, the ITI agent includes rituximab, methotrexate, and intravenous immunoglobulin (IVIG). In some embodiments, the subject with Pompe disease is a CRIM-positive subject.

In some embodiments of any of the foregoing aspects, the Pompe disease is an infantile-onset Pompe disease. In some embodiments, the subject is from about one month to about one year (e.g., about 1, 2, 3, 4, 5, 6, 7, 8, 9, 10, 11, or 12 months) of age. In some embodiments, the subject is from about six months to about one year (e.g., about 6, 7, 8, 9, 10, 11, or 12 months) of age. In some embodiments, prior to administration of the composition to the subject, the subject exhibits one or more symptoms selected from feeding difficulties, failure to thrive, hypotonia, progressive weakness, respiratory distress, macroglossia, and cardiac hypertrophy.

In some embodiments of any of the foregoing aspects, the Pompe disease is a late-onset Pompe disease. In some embodiments, the subject exhibits endogenous GAA activity from about 1% to about 40% (e.g., about 1%, 5%, 10%, 15%, 20%, 25%, 30%, 35%, or 40%) of the endogenous GAA activity of a human of the same gender and similar body mass index that does not have Pompe disease.

In some embodiments of any of the foregoing aspects, following the administration of the composition to the subject, the subject exhibits endogenous GAA activity of from about 10% to about 2000% (e.g., about 10%, 20%, 30%, 40%, 50%, 60%, 70%, 80%, 90%, 100%, 200%, 300%, 400%, 500%, 600%, 700%, 800%, 900%, 1,000%, 1,500%, or 2000%) of the endogenous GAA activity of a human of the same gender and similar body mass index that does not have Pompe disease.

In some embodiments of any of the foregoing aspects, the composition is administered to the subject in a dosage of 1×10⁵ as cells/kg of recipient to about 30×10⁷ cells/kg (e.g., from about 2×10⁵ as cells/kg to about 29×10⁷ cells/kg, from about 3×10⁵ as cells/kg to about 28×10⁷ cells/kg, from about 4×10⁵ as cells/kg to about 27×10⁷ cells/kg, from about 5×10⁵ as cells/kg to about 26×10⁷ cells/kg, from about 5×10⁵ as cells/kg to about 25×10⁷ cells/kg, from about 6×10⁵ as cells/kg to about 24×10⁷ cells/kg, from about 7×10⁵ as cells/kg to about 23×10⁷ cells/kg, from about 8×10⁵ as cells/kg to about 22×10⁷ cells/kg, from about 9×10⁵ as cells/kg to about 21×10⁷ cells/kg, from about 1×10⁶ cells/kg to about 20×10⁷ cells/kg, from about 2×10⁶ cells/kg to about 19×10⁷ cells/kg, from about 3×10⁶ cells/kg to about 19×10⁷ cells/kg, from about 4×10⁶ cells/kg to about 18×10⁷ cells/kg, from about 5×10⁶ cells/kg to about 17×10⁷ cells/kg, from about 6×10⁶ cells/kg to about 16×10⁷ cells/kg, from about 7×10⁶ cells/kg to about 15×10⁷ cells/kg, from about 8×10⁶ cells/kg to about 10×10⁷ cells/kg, and from about 9×10⁶ cells/kg to about 5×10⁷ cells/kg). In some embodiments, the composition is administered in dosages that are from about 1×10¹⁰ cells/kg of recipient to about 1×10¹² cells/kg (e.g., from about 2×10¹⁰ cells/kg to about 9×10¹¹ cells/kg, from about 3×10¹⁰ cells/kg to about 8×10¹¹ cells/kg, from about 4×10¹⁰ cells/kg to about 7×10¹¹ cells/kg, from about 5×10¹⁰ cells/kg to about 6×10¹¹ cells/kg, from about 5×10¹⁰ cells/kg to about 1×10¹² cells/kg, from about 6×10¹⁰ cells/kg to about 1×10¹² cells/kg, from about 7×10¹⁰ cells/kg to about 1×10¹² cells/kg, from about 8×10¹⁰ cells/kg to about 1×10¹² cells/kg, from about 9×10¹⁰ cells/kg to about 1×10¹² cells/kg, and from about 1×10¹¹ cells/kg to about 1×10¹² cells/kg).

In some embodiments of any of the foregoing aspects, the subject is female. In some embodiments of any of the foregoing aspects, the subject is male.

In some embodiments of any of the foregoing aspects, the composition is administered in an amount sufficient to reduce one or more of cardiomegaly, hypotonia, cardiomyopathy, respiratory distress, muscle weakness, feeding difficulties, failure to thrive, floppy baby appearance, delay in motor development, hepatomegaly, macroglossia, wide open mouth, wide open eyes, nasal flaring, respiratory rate, engagement of accessory muscles for breathing, frequency of chest infections, arrhythmia, heart failure, impaired cough, muscle weakness, difficulty masticating and swallowing, or the composition is administered in an amount sufficient to increase one or more of facial muscle tone, air flow in the left lower zone, and vital capacity.

In some embodiments of any of the foregoing aspects, the composition is administered in an amount sufficient to reduce glycogen accumulation in muscle cells, neural cells, and/or liver cells. In some embodiments, the composition is administered in an amount sufficient to increase GAA expression level and/or enzymatic activity in muscle cells, neural cells, and/or liver cells of the subject. In some embodiments, the neural cells are neurons or glial cells. In some embodiments, the muscle cells are skeletal muscle cells and/or cardiac muscle cells.

In some embodiments of any of the foregoing aspects, the composition is administered in an amount sufficient to reduce glycogen accumulation in muscle tissue and/or nervous tissue. In some embodiments, the composition is administered in an amount sufficient to increase GAA expression level and/or enzymatic activity in muscle tissue or nervous tissue. In some embodiments, the muscle tissue is of the heart, diaphragm, gastrocnemius muscle, quadriceps femoris muscle, and/or tibialis anterior muscle. In some embodiments, the nervous tissue is of the cerebellum, cerebrum, thoracic or cervical spinal cord, and/or hippocampus.

In some embodiments of any of the foregoing aspects, the subject has not previously received GAA enzyme replacement therapy (ERT). In some embodiments, the subject has previously received GAA ERT.

In some embodiments, the subject has atrophy in one or more tissues selected from heart, diaphragm, gastrocnemius muscle, quadriceps femoris muscle, tibialis anterior muscle, cerebellum, cerebrum, thoracic spinal cord, cervical spinal cord, and hippocampus tissue.

In some embodiments, the subject is a human.

In another aspect, the disclosure provides a composition containing a population of cells that express a transgene encoding GAA protein fused to a GILT tag (GILT.GAA protein), wherein the GILT tag includes an IGF-II mutein including an Ala amino acid substitution at a position corresponding to Arg37 of SEQ ID NO: 15.

In some embodiments of the foregoing aspect, the human IGF-II mutein has an amino acid sequence that is at least 70% (e.g., at least 75%, 80%, 85%, 90%, 95%, 96%, 97%, 98%, 99%, or more) identical to the amino acid sequence of mature human IGF-II (SEQ ID NO: 15). In some embodiments, the human IGF-II mutein has an amino acid sequence that is at least 80% (e.g., at least 85%, 90%, 91%, 92%, 93%, 94%, 95%, 96%, 97%, 98%, 99%, or more) identical to the amino acid sequence of mature human IGF-II (SEQ ID NO: 15). In some embodiments, the human IGF-II mutein has an amino acid sequence that is at least 90% (e.g., at least 91%, 92%, 93%, 94%, 95%, 96%, 97%, 98%, 99%, or more) identical to the amino acid sequence of mature human IGF-II (SEQ ID NO: 15).

In some embodiments of the foregoing aspect, the GILT tag has an amino acid sequence that is at least 70% (e.g., at least 75%, 80%, 85%, 90%, 95%, 96%, 97%, 98%, 99%, or more) identical to the amino acid sequence of SEQ ID NO: 16. In some embodiments of the foregoing aspect, the GILT tag has an amino acid sequence that is at least 80% (e.g., at least 85%, 90%, 95%, 96%, 97%, 98%, 99%, or more) identical to the amino acid sequence of SEQ ID NO: 16. In some embodiments of the foregoing aspect, the GILT tag has an amino acid sequence that is at least 90% (e.g., at least 95%, 96%, 97%, 98%, 99%, or more) identical to the amino acid sequence of SEQ ID NO: 16.

In some embodiments of the foregoing aspect, the GILT tag has an amino acid sequence that is at least 70% (e.g., at least 75%, 80%, 85%, 90%, 95%, 96%, 97%, 98%, 99%, or more) identical to the amino acid sequence of SEQ ID NO: 17. In some embodiments of the foregoing aspect, the GILT tag has an amino acid sequence that is at least 80% (e.g., at least 85%, 90%, 95%, 96%, 97%, 98%, 99%, or more) identical to the amino acid sequence of SEQ ID NO: 17. In some embodiments of the foregoing aspect, the GILT tag has an amino acid sequence that is at least 90% (e.g., at least 95%, 96%, 97%, 98%, 99%, or more) identical to the amino acid sequence of SEQ ID NO: 17.

In some embodiments of the foregoing aspect, the GILT tag has an amino acid sequence that is at least 70% (e.g., at least 75%, 80%, 85%, 90%, 95%, 96%, 97%, 98%, 99%, or more) identical to the amino acid sequence of SEQ ID NO: 18. In some embodiments of the foregoing aspect, the GILT tag has an amino acid sequence that is at least 80% (e.g., at least 85%, 90%, 95%, 96%, 97%, 98%, 99%, or more) identical to the amino acid sequence of SEQ ID NO: 18. In some embodiments of the foregoing aspect, the GILT tag has an amino acid sequence that is at least 90% (e.g., at least 95%, 96%, 97%, 98%, 99%, or more) identical to the amino acid sequence of SEQ ID NO: 18.

In some embodiments of any of the foregoing aspects, the GILT tag has an amino acid sequence that is at least 70% (e.g., at least 75%, 80%, 85%, 90%, 95%, 96%, 97%, 98%, 99%, or more) identical to the amino acid sequence of SEQ ID NO: 19. In some embodiments of any of the foregoing aspects, the GILT tag has an amino acid sequence that is at least 80% (e.g., at least 85%, 90%, 95%, 96%, 97%, 98%, 99%, or more) identical to the amino acid sequence of SEQ ID NO: 19. In some embodiments of any of the foregoing aspects, the GILT tag has an amino acid sequence that is at least 90% (e.g., at least 95%, 96%, 97%, 98%, 99%, or more) identical to the amino acid sequence of SEQ ID NO: 19.

In some embodiments of any of the foregoing aspects, the GILT tag has an amino acid sequence that is at least 70% (e.g., at least 75%, 80%, 85%, 90%, 95%, 96%, 97%, 98%, 99%, or more) identical to the amino acid sequence of SEQ ID NO: 20. In some embodiments of any of the foregoing aspects, the GILT tag has an amino acid sequence that is at least 80% (e.g., at least 85%, 90%, 95%, 96%, 97%, 98%, 99%, or more) identical to the amino acid sequence of SEQ ID NO: 20. In some embodiments of any of the foregoing aspects, the GILT tag has an amino acid sequence that is at least 90% (e.g., at least 95%, 96%, 97%, 98%, 99%, or more) identical to the amino acid sequence of SEQ ID NO: 20.

In some embodiments of the foregoing aspect, the GILT tag is encoded by a polynucleotide having a nucleic acid sequence that is at least 85% (e.g., at least 90%, 91%, 92%, 93%, 94%, 95%, 96%, 97%, 98%, 99%, or more) identical to the nucleic acid sequence of SEQ ID NO: 21. In some embodiments of the foregoing aspect, the GILT tag is encoded by a polynucleotide having a nucleic acid sequence that is at least 90% (e.g., at least 91%, 92%, 93%, 94%, 95%, 96%, 97%, 98%, 99%, or more) identical to the nucleic acid sequence of SEQ ID NO: 21. In some embodiments of the foregoing aspect, the GILT tag is encoded by a polynucleotide having a nucleic acid sequence that is at least 95% (e.g., at least 96%, 97%, 98%, 99%, or more) identical to the nucleic acid sequence of SEQ ID NO: 21. In some embodiments, the GILT tag is encoded by a polynucleotide having the nucleic acid sequence of SEQ ID NO: 21.

In some embodiments of the foregoing aspect, the GILT tag is encoded by a polynucleotide having a nucleic acid sequence that is at least 85% (e.g., at least 90%, 91%, 92%, 93%, 94%, 95%, 96%, 97%, 98%, 99%, or more) identical to the nucleic acid sequence of SEQ ID NO: 22. In some embodiments of the foregoing aspect, the GILT tag is encoded by a polynucleotide having a nucleic acid sequence that is at least 90% (e.g., at least 91%, 92%, 93%, 94%, 95%, 96%, 97%, 98%, 99%, or more) identical to the nucleic acid sequence of SEQ ID NO: 22. In some embodiments of the foregoing aspect, the GILT tag is encoded by a polynucleotide having a nucleic acid sequence that is at least 95% (e.g., at least 96%, 97%, 98%, 99%, or more) identical to the nucleic acid sequence of SEQ ID NO: 22. In some embodiments, the GILT tag is encoded by a polynucleotide having the nucleic acid sequence of SEQ ID NO: 22.

In some embodiments of the foregoing aspect, the GILT tag is encoded by a polynucleotide having a nucleic acid sequence that is at least 85% (e.g., at least 90%, 91%, 92%, 93%, 94%, 95%, 96%, 97%, 98%, 99%, or more) identical to the nucleic acid sequence of SEQ ID NO: 23. In some embodiments of the foregoing aspect, the GILT tag is encoded by a polynucleotide having a nucleic acid sequence that is at least 90% (e.g., at least 91%, 92%, 93%, 94%, 95%, 96%, 97%, 98%, 99%, or more) identical to the nucleic acid sequence of SEQ ID NO: 23. In some embodiments of the foregoing aspect, the GILT tag is encoded by a polynucleotide having a nucleic acid sequence that is at least 95% (e.g., at least 96%, 97%, 98%, 99%, or more) identical to the nucleic acid sequence of SEQ ID NO: 23. In some embodiments, the GILT tag is encoded by a polynucleotide having the nucleic acid sequence of SEQ ID NO: 23.

In some embodiments of the foregoing aspect, the human IGF-II mutein has diminished binding affinity for the insulin receptor relative to the affinity of naturally-occurring human IGF-II for the insulin receptor, wherein the IGF-II mutein is resistant to furin cleavage, wherein the IGF-II mutein binds to the human cation-independent mannose-6-phosphate receptor in a mannose-6-phosphate-independent manner.

In some embodiments of the foregoing aspect, the transgene is operably linked to a promoter. In some embodiments, the promoter is a ubiquitous promoter. In some embodiments, the ubiquitous promoter is an EF1a promoter, PGK promoter, or β-LCR(HS4,3,2)-EFS promoter. In some embodiments, the promoter is a cell lineage-specific promoter. In some embodiments, the cell lineage-specific promoter is a CD68 promoter, CD11 b promoter, CX3CR1 promoter, AIF1 promoter, P2Y12 promoter, TMEM119 promoter, or CSF1R promoter. In some embodiments, the promoter is a viral promoter. In some embodiments, the viral promoter is an adenovirus late promoter, vaccinia virus 7.5K promoter, SV40 promoter, CMV promoter, HSV promoter, MMTV promoter, LTR of HIV promoter, Moloney virus promoter, EBV, or RSV promoter. In some embodiments, promoter is a synthetic promoter. In some embodiments, the promoter is a synthetic promoter. In some embodiments, the synthetic promoter is an MND promoter. In some embodiments, the MND promoter includes a polynucleotide having at least 85% (e.g., at least 90%, 91%, 92%, 93%, 94%, 95%, 96%, 97%, 98%, 99%, or more) sequence identity to the nucleic acid sequence of SEQ ID NO: 10. In some embodiments, the MND promoter includes a polynucleotide having at least 90% (e.g., at least 91%, 92%, 93%, 94%, 95%, 96%, 97%, 98%, 99%, or more) sequence identity to the nucleic acid sequence of SEQ ID NO: 10. In some embodiments, the MND promoter includes a polynucleotide having at least 95% (e.g., at least 96%, 97%, 98%, 99%, or more) sequence identity to the nucleic acid sequence of SEQ ID NO: 10. In some embodiments, the MND promoter includes a polynucleotide having at least 98% (e.g., at least 99%, or more) sequence identity to the nucleic acid sequence of SEQ ID NO: 10. In some embodiments, the MND promoter includes a polynucleotide having at least 99% sequence identity to the nucleic acid sequence of SEQ ID NO: 10. In some embodiments, the MND promoter includes a polynucleotide having the nucleic acid sequence of SEQ ID NO: 10. In some embodiments, the MND promoter includes a polynucleotide having at least 85% (e.g., at least 90%, 91%, 92%, 93%, 94%, 95%, 96%, 97%, 98%, 99%, or more) sequence identity to the nucleic acid sequence of SEQ ID NO: 11. In some embodiments, the MND promoter includes a polynucleotide having at least 90% (e.g., at least 91%, 92%, 93%, 94%, 95%, 96%, 97%, 98%, 99%, or more) sequence identity to the nucleic acid sequence of SEQ ID NO: 11. In some embodiments, the MND promoter includes a polynucleotide having at least 95% (e.g., at least 96%, 97%, 98%, 99%, or more) sequence identity to the nucleic acid sequence of SEQ ID NO: 11. In some embodiments, the MND promoter includes a polynucleotide having at least 98% (e.g., at least 99%, or more) sequence identity to the nucleic acid sequence of SEQ ID NO: 11. In some embodiments, the MND promoter includes a polynucleotide having at least 99% sequence identity to the nucleic acid sequence of SEQ ID NO: 11. In some embodiments, the MND promoter includes a polynucleotide having the nucleic acid sequence of SEQ ID NO: 11.

In some embodiments, the GAA is full-length GAA, such as GAA having an amino acid sequence of any one of SEQ ID NOs: 1-4 or a variant thereof having at least 85% (e.g., 90%, 95%, 96%, 97%, 98%, 99%, or more) sequence identity thereto (e.g. at least 85%, 90%, 95%, 96%, 97%, 98%, 99%, or more) sequence identity to the amino acid sequence of any one of SEQ ID NOs: 1-4.

In some embodiments, the transgene encodes a GAA protein having an amino acid sequence of SEQ ID NO: 1 or a variant thereof having at least 85% (e.g., at least 90%, 91%, 92%, 93%, 94%, 95%, 96%, 97%, 98%, 99%, or more) sequence identity to the amino acid sequence of SEQ ID NO: 1. In some embodiments, the transgene encodes a GAA protein having an amino acid sequence of SEQ ID NO: 1 or a variant thereof having at least 90% (e.g., at least 91%, 92%, 93%, 94%, 95%, 96%, 97%, 98%, 99%, or more) sequence identity to the amino acid sequence of SEQ ID NO: 1. In some embodiments the transgene encodes a GAA protein having an amino acid sequence of SEQ ID NO: 1 or a variant thereof having at least 95% (e.g., at least 96%, 97%, 98%, 99%, or more) sequence identity to the amino acid sequence of SEQ ID NO: 1. In some embodiments, the transgene encodes a GAA protein having an amino acid sequence of SEQ ID NO: 1 or a variant thereof having at least 98% (e.g., at least 99%, or more) sequence identity to the amino acid sequence of SEQ ID NO: 1. In some embodiments, the transgene encodes a GAA protein having an amino acid sequence of SEQ ID NO: 1 or a variant thereof having at least 99% sequence identity to the amino acid sequence of SEQ ID NO: 1. In some embodiments, the transgene encodes a GAA protein having the amino acid sequence of SEQ ID NO: 1.

In some embodiments, the transgene encodes a GAA protein having an amino acid sequence of SEQ ID NO: 2 or a variant thereof having at least 85% (e.g., at least 90%, 91%, 92%, 93%, 94%, 95%, 96%, 97%, 98%, 99%, or more) sequence identity to the amino acid sequence of SEQ ID NO: 2. In some embodiments, the transgene encodes a GAA protein having an amino acid sequence of SEQ ID NO: 2 or a variant thereof having at least 90% (e.g., at least 91%, 92%, 93%, 94%, 95%, 96%, 97%, 98%, 99%, or more) sequence identity to the amino acid sequence of SEQ ID NO: 2. In some embodiments the transgene encodes a GAA protein having an amino acid sequence of SEQ ID NO: 2 or a variant thereof having at least 95% (e.g., at least 96%, 97%, 98%, 99%, or more) sequence identity to the amino acid sequence of SEQ ID NO: 2. In some embodiments, the transgene encodes a GAA protein having an amino acid sequence of SEQ ID NO: 2 or a variant thereof having at least 98% (e.g., at least 99%, or more) sequence identity to the amino acid sequence of SEQ ID NO: 2. In some embodiments, the transgene encodes a GAA protein having an amino acid sequence of SEQ ID NO: 2 or a variant thereof having at least 99% sequence identity to the amino acid sequence of SEQ ID NO: 2. In some embodiments, the transgene encodes a GAA protein having the amino acid sequence of SEQ ID NO: 2.

In some embodiments, the transgene encodes a GAA protein having an amino acid sequence of SEQ ID NO: 3 or a variant thereof having at least 85% (e.g., at least 90%, 91%, 92%, 93%, 94%, 95%, 96%, 97%, 98%, 99%, or more) sequence identity to the amino acid sequence of SEQ ID NO: 3. In some embodiments, the transgene encodes a GAA protein having an amino acid sequence of SEQ ID NO: 3 or a variant thereof having at least 90% (e.g., at least 91%, 92%, 93%, 94%, 95%, 96%, 97%, 98%, 99%, or more) sequence identity to the amino acid sequence of SEQ ID NO: 3. In some embodiments the transgene encodes a GAA protein having an amino acid sequence of SEQ ID NO: 3 or a variant thereof having at least 95% (e.g., at least 96%, 97%, 98%, 99%, or more) sequence identity to the amino acid sequence of SEQ ID NO: 3. In some embodiments, the transgene encodes a GAA protein having an amino acid sequence of SEQ ID NO: 3 or a variant thereof having at least 98% (e.g., at least 99%, or more) sequence identity to the amino acid sequence of SEQ ID NO: 3. In some embodiments, the transgene encodes a GAA protein having an amino acid sequence of SEQ ID NO: 3 or a variant thereof having at least 99% sequence identity to the amino acid sequence of SEQ ID NO: 3. In some embodiments, the transgene encodes a GAA protein having the amino acid sequence of SEQ ID NO: 3.

In some embodiments, the transgene encodes a GAA protein having an amino acid sequence of SEQ ID NO: 4 or a variant thereof having at least 85% (e.g., at least 90%, 91%, 92%, 93%, 94%, 95%, 96%, 97%, 98%, 99%, or more) sequence identity to the amino acid sequence of SEQ ID NO: 4. In some embodiments, the transgene encodes a GAA protein having an amino acid sequence having at least 90% (e.g., at least 91%, 92%, 93%, 94%, 95%, 96%, 97%, 98%, 99%, or more) sequence identity to the amino acid sequence of SEQ ID NO: 4. In some embodiments the transgene encodes a GAA protein having an amino acid sequence having at least 95% (e.g., at least 96%, 97%, 98%, 99%, or more) sequence identity to the amino acid sequence of SEQ ID NO: 4. In some embodiments, the transgene encodes a GAA protein having an amino acid sequence having at least 98% (e.g., at least 99%, or more) sequence identity to the amino acid sequence of SEQ ID NO: 4. In some embodiments, the transgene encodes a GAA protein having an amino acid sequence having at least 99% sequence identity to the amino acid sequence of SEQ ID NO: 4. In some embodiments, the transgene encodes a GAA protein having the amino acid sequence of SEQ ID NO: 4.

In some embodiments, the GAA protein is encoded by a polynucleotide having the nucleic acid sequence of SEQ ID NO: 5 or a variant thereof having at least 85% (e.g., at least 90%, 91%, 92%, 93%, 94%, 95%, 96%, 97%, 98%, 99%, or more) sequence identity to the nucleic acid sequence of SEQ ID NO: 5. In some embodiments, the GAA protein is encoded by a polynucleotide having at least 90% (e.g., at least 91%, 92%, 93%, 94%, 95%, 96%, 97%, 98%, 99%, or more) sequence identity to the nucleic acid sequence of SEQ ID NO: 5. In some embodiments, the GAA protein is encoded by a polynucleotide having at least 95% (e.g., at least 96%, 97%, 98%, 99%, or more) sequence identity to the nucleic acid sequence of SEQ ID NO: 5. In some embodiments, the GAA protein is encoded by a polynucleotide having at least 98% (e.g., at least 99%, or more) sequence identity to the nucleic acid sequence of SEQ ID NO: 5. In some embodiments, the GAA protein is encoded by a polynucleotide having at least 99% sequence identity to the nucleic acid sequence of SEQ ID NO: 5. In some embodiments, the GAA protein is encoded by a polynucleotide having the nucleic acid sequence of SEQ ID NO: 5.

In some embodiments, the GAA protein is encoded by a polynucleotide having the nucleic acid sequence of SEQ ID NO: 6 or a variant thereof having at least 85% (e.g., at least 90%, 91%, 92%, 93%, 94%, 95%, 96%, 97%, 98%, 99%, or more) sequence identity to the nucleic acid sequence of SEQ ID NO: 6. In some embodiments, the GAA protein is encoded by a polynucleotide having at least 90% (e.g., at least 91%, 92%, 93%, 94%, 95%, 96%, 97%, 98%, 99%, or more) sequence identity to the nucleic acid sequence of SEQ ID NO: 6. In some embodiments, the GAA protein is encoded by a polynucleotide having at least 95% (e.g., at least 96%, 97%, 98%, 99%, or more) sequence identity to the nucleic acid sequence of SEQ ID NO: 6. In some embodiments, the GAA protein is encoded by a polynucleotide having at least 98% (e.g., at least 99%, or more) sequence identity to the nucleic acid sequence of SEQ ID NO: 6. In some embodiments, the GAA protein is encoded by a polynucleotide having at least 99% sequence identity to the nucleic acid sequence of SEQ ID NO: 6. In some embodiments, the GAA protein is encoded by a polynucleotide having the nucleic acid sequence of SEQ ID NO: 6.

In some embodiments, the GAA protein is encoded by a polynucleotide having the nucleic acid sequence of SEQ ID NO: 7 or a variant thereof having at least 85% (e.g., at least 90%, 91%, 92%, 93%, 94%, 95%, 96%, 97%, 98%, 99%, or more) sequence identity to the nucleic acid sequence of SEQ ID NO: 7. In some embodiments, the GAA protein is encoded by a polynucleotide having at least 90% (e.g., at least 91%, 92%, 93%, 94%, 95%, 96%, 97%, 98%, 99%, or more) sequence identity to the nucleic acid sequence of SEQ ID NO: 7. In some embodiments, the GAA protein is encoded by a polynucleotide having at least 95% (e.g., at least 96%, 97%, 98%, 99%, or more) sequence identity to the nucleic acid sequence of SEQ ID NO: 7. In some embodiments, the GAA protein is encoded by a polynucleotide having at least 98% (e.g., at least 99%, or more) sequence identity to the nucleic acid sequence of SEQ ID NO: 7. In some embodiments, the GAA protein is encoded by a polynucleotide having at least 99% sequence identity to the nucleic acid sequence of SEQ ID NO: 7. In some embodiments, the GAA protein is encoded by a polynucleotide having the nucleic acid sequence of SEQ ID NO: 7.

In some embodiments, the GAA protein is encoded by a polynucleotide having the nucleic acid sequence of SEQ ID NO: 8 or a variant thereof having at least 85% (e.g., at least 90%, 91%, 92%, 93%, 94%, 95%, 96%, 97%, 98%, 99%, or more) sequence identity to the nucleic acid sequence of SEQ ID NO: 8. In some embodiments, the GAA protein is encoded by a polynucleotide having at least 90% (e.g., at least 91%, 92%, 93%, 94%, 95%, 96%, 97%, 98%, 99%, or more) sequence identity to the nucleic acid sequence of SEQ ID NO: 8. In some embodiments, the GAA protein is encoded by a polynucleotide having at least 95% (e.g., at least 96%, 97%, 98%, 99%, or more) sequence identity to the nucleic acid sequence of SEQ ID NO: 8. In some embodiments, the GAA protein is encoded by a polynucleotide having at least 98% (e.g., at least 99%, or more) sequence identity to the nucleic acid sequence of SEQ ID NO: 8. In some embodiments, the GAA protein is encoded by a polynucleotide having at least 99% sequence identity to the nucleic acid sequence of SEQ ID NO: 8. In some embodiments, the GAA protein is encoded by a polynucleotide having the nucleic acid sequence of SEQ ID NO: 8.

In some embodiments, the GAA protein is encoded by a polynucleotide having the nucleic acid sequence of SEQ ID NO: 9 or a variant thereof having at least 85% (e.g., at least 90%, 91%, 92%, 93%, 94%, 95%, 96%, 97%, 98%, 99%, or more) sequence identity to the nucleic acid sequence of SEQ ID NO: 9. In some embodiments, the GAA protein is encoded by a polynucleotide having at least 90% (e.g., at least 91%, 92%, 93%, 94%, 95%, 96%, 97%, 98%, 99%, or more) sequence identity to the nucleic acid sequence of SEQ ID NO: 9. In some embodiments, the GAA protein is encoded by a polynucleotide having at least 95% (e.g., at least 96%, 97%, 98%, 99%, or more) sequence identity to the nucleic acid sequence of SEQ ID NO: 9. In some embodiments, the GAA protein is encoded by a polynucleotide having at least 98% (e.g., at least 99%, or more) sequence identity to the nucleic acid sequence of SEQ ID NO: 9. In some embodiments, the GAA protein is encoded by a polynucleotide having at least 99% sequence identity to the nucleic acid sequence of SEQ ID NO: 9. In some embodiments, the GAA protein is encoded by a polynucleotide having the nucleic acid sequence of SEQ ID NO: 9.

In some embodiments of the foregoing aspect, the cells are pluripotent cells or multipotent cells. In some embodiments, the pluripotent cells are ESCs. In some embodiments, the pluripotent cells are iPSCs. In some embodiments, the cells are CD34+ cells. In some embodiments, the cells are multipotent cells. In some embodiments, the multipotent cells are CD34+ cells. In some embodiments, the CD34+ cells are hematopoietic stem cells. In some embodiments, the CD34+ cells are myeloid cells. In some embodiments, the myeloid cells are myeloid progenitor cells. In some embodiments, the myeloid cells are erythrocytes, mast cells, megakaryocytes, thrombocytes, myeloblasts, basophils, neutrophils, eosinophils, monocytes, or macrophages. In some embodiments, the cells are blood line progenitor cells (BLPCs). In some embodiments, the BLPCs are monocytes. In some embodiments the cells are macrophages. In some embodiments, the cells are microglial progenitor cells. In some embodiments, the cells are microglia.

In some embodiments of the foregoing aspect, the GAA is full-length GAA.

In some embodiments of any of the foregoing aspects, the GAA includes a signal peptide.

In some embodiments of any of the foregoing aspects, the signal peptide is a GAA signal peptide.

In some embodiments of any of the foregoing aspects, the signal peptide is an IGF-II signal peptide. In some embodiments, the IGF-II signal peptide includes an amino acid sequence of SEQ ID NO: 12

In some embodiments of the foregoing aspect, the transgene encodes two or more GAA proteins (e.g., at least 2, 3, 4, 5, 6, 7, 8, 9, 10 or more GAA proteins). In some embodiments, the transgene encodes from two to ten GAA proteins (e.g., 2, 3, 4, 5, 6, 7, 8, 9, or 10 GAA proteins). In some embodiments, the transgene encodes from two to five GAA proteins (e.g., 2, 3, 4, or 5 GAA proteins). In some embodiments, the transgene encodes two GAA proteins. In some embodiments, the GAA transgenes are expressed from a single, polycistronic expression cassette. In some embodiments, the GAA transgenes are separated from one another by way of one or more (e.g., 1, 2, 3, 4, 5, 6, 7, 8, 9, or more) IRES. In some embodiments, the GAA transgenes are expressed from one or more (e.g., 1, 2, 3, 4, 5, 6, 7, 8, 9, 10, or more) monocistronic expression cassettes.

In some embodiments of the foregoing aspect, the transgene is a codon-optimized transgene.

In some embodiments of the foregoing aspect, the GILT.GAA protein includes a LDLRf binding (Rb) domain of ApoE, or a fragment, variant, or oligomer thereof. In some embodiments, the Rb domain of ApoE, or a fragment, variant, or oligomer thereof, is operably linked to the N-terminus of the GAA. In some embodiments, the Rb domain of ApoE, or a fragment, variant, or oligomer thereof is operably linked to the C-terminus of the GAA. In some embodiments, the GAA fusion protein contains 1 or more (e.g., 1, 2, 3, 4, 5, 6, 7, 8, 9, 10, or more) oligomers of the Rb domain of ApoE. In some embodiments, the Rb domain contains a region of ApoE having at least 70% sequence identity (e.g., at least 70%, 75%, 80%, 85%, 90%, 95%, 96%, 97%, 98%, 99%, or greater, sequence identity) to residues 25-185 of SEQ ID NO: 24. In some embodiments, the Rb domain contains a region of ApoE having at least 70% sequence identity (e.g., at least 70%, 75%, 80%, 85%, 90%, 95%, 96%, 97%, 98%, 99%, or greater, sequence identity) to residues 50-180 of SEQ ID NO: 24. In some embodiments, the Rb domain contains a region of ApoE having at least 70% sequence identity (e.g., at least 70%, 75%, 80%, 85%, 90%, 95%, 96%, 97%, 98%, 99%, or greater, sequence identity) to residues 75-175 of SEQ ID NO: 24. In some embodiments, the Rb domain contains a region of ApoE having at least 70% sequence identity (e.g., at least 70%, 75%, 80%, 85%, 90%, 95%, 96%, 97%, 98%, 99%, or greater, sequence identity) to residues 100-170 of SEQ ID NO: 24. In some embodiments, the Rb domain contains a region of ApoE having at least 70% sequence identity (e.g., at least 70%, 75%, 80%, 85%, 90%, 95%, 96%, 97%, 98%, 99%, or greater, sequence identity) to residues 125-160 of SEQ ID NO: 24. In some embodiments, the Rb domain contains a region of ApoE having at least 70% sequence identity (e.g., at least 70%, 75%, 80%, 85%, 90%, 95%, 96%, 97%, 98%, 99%, or greater, sequence identity) to residues 130-150 of SEQ ID NO: 24. In some embodiments, the Rb domain contains a region of ApoE having at least 70% sequence identity (e.g., at least 70%, 75%, 80%, 85%, 90%, 95%, 96%, 97%, 98%, 99%, or greater, sequence identity) to residues 148-173 or a portion thereof containing residues 159-167 of SEQ ID NO: 24, or a variant having at least 70% sequence identity (e.g., at least 70%, 75%, 80%, 85%, 90%, 95%, 96%, 97%, 98%, 99%, or greater, sequence identity) to residues 159-167 of SEQ ID NO: 24. In some embodiments, the Rb domain contains a region having at least 70% sequence identity (e.g., at least 70%, 75%, 80%, 85%, 90%, 95%, 96%, 97%, 98%, 99%, or greater, sequence identity) to the amino acid sequence of residues 159-167 of SEQ ID NO: 24.

In some embodiments of any of the foregoing aspects, the GILT.GAA protein does not include a low-density lipoprotein receptor family (LDLRf) binding (Rb) domain of apolipoprotein E (ApoE), or a fragment, variant, or oligomer thereof.

In some embodiments of the foregoing aspect, the transgene encoding GAA further contains a miRNA targeting sequence (e.g., a miR-126 targeting sequence). In some embodiments, the miRNA targeting sequence (e.g., a miR-126 targeting sequence) is located within the 3′ UTR of the transgene.

In some embodiments of the foregoing aspect, the GAA penetrates the BBB in the subject.

In some embodiments of the foregoing aspect, the transgene encoding GAA includes a polynucleotide encoding polypeptide that contains one or more amino acid substitutions, such as one or more conservative amino acid substitutions (e.g., 1, 2, 3, 4, 5, 6, 7, 8, 9, or 10 or more amino acid substitutions, such as 1, 2, 3, 4, 5, 6, 7, 8, 9, or 10 or more conservative amino acid substitutions), relative to a polypeptide having the sequence of any one of SEQ ID NOs: 1-4.

In some embodiments of the preceding aspect, the cells are pluripotent cells. In some embodiments, the pluripotent cells are ESCs. In some embodiments, the pluripotent cells are iPSCs. In some embodiments, the cells are CD34+ cells. In some embodiments, the cells are multipotent cells. In some embodiments, the multipotent cells are CD34+ cells. In some embodiments, the CD34+ cells are hematopoietic stem cells. In some embodiments, the CD34+ cells are myeloid cells. In some embodiments, the myeloid cells are myeloid progenitor cells. In some embodiments, the myeloid cells are erythrocytes, mast cells, megakaryocytes, thrombocytes, myeloblasts, basophils, neutrophils, eosinophils, monocytes, or macrophages. In some embodiments, the cells are blood line progenitor cells (BLPCs). In some embodiments, the BLPCs are monocytes. In some embodiments the cells are macrophages. In some embodiments, the cells are microglia.

In some embodiments, the cells are transduced ex vivo to express the GAA. In some embodiments, the cells are transfected ex vivo to express the GAA.

In another aspect, the present disclosure provides pharmaceutical composition including the composition of any of the foregoing aspects and embodiments, wherein the pharmaceutical composition further includes a pharmaceutically acceptable carrier, diluent, or excipient.

In another aspect, the present disclosure provides a kit including the composition of any of the foregoing aspects and embodiments, or the pharmaceutical composition of the foregoing aspect, and a package insert, wherein the package insert instructs a user of the kit to perform the method of any one of the foregoing aspects and embodiments.

Additional embodiments of the present invention are listed in the enumerated paragraphs below.
E1. A method of treating Pompe disease in a subject, the method including administering to the subject a composition including a population of cells including a transgene encoding a GAA protein fused to a GILT tag (GILT.GAA protein).
E2. A method of improving muscle function in a subject diagnosed as having Pompe disease, the method including administering to the subject a composition including a population of cells including a transgene encoding a GILT.GAA protein.
E3. A method of reducing glycogen accumulation in a subject diagnosed as having Pompe disease, the method including administering to the subject a composition including a population of cells including a transgene encoding GILT.GAA protein.
E4. A method of improving pulmonary function in a subject diagnosed as having Pompe disease, the method including administering to the subject a composition including a population of cells including a transgene encoding a GILT.GAA protein.
E5. A method of increasing GAA expression in a subject diagnosed as having Pompe disease, the method including administering to the subject a composition including a population of cells including a transgene encoding a GILT.GAA protein.
E6. The method of any one of E1-E5, wherein the GILT tag includes a human IGF-II mutein including an Ala amino acid substitution at a position corresponding to Arg 37 of SEQ ID NO: 15.
E7. The method of any E6, wherein the human IGF-II mutein has an amino acid sequence that is at least 70% (e.g., at least 75%, 80%, 85%, 90%, 95%, 96%, 97%, 98%, 99%, or more) identical to the amino acid sequence of mature human IGF-II (SEQ ID NO: 15).
E8. The method of E7, wherein the human IGF-II mutein has an amino acid sequence that is at least 80% (e.g., at least 85%, 90%, 95%, 96%, 97%, 98%, 99%, or more) identical to the amino acid sequence of mature human IGF-II (SEQ ID NO: 15).
E9. The method of E8, wherein the human IGF-II mutein has an amino acid sequence that is at least 90% (e.g., at least 91%, 92%, 93%, 94%, 95%, 96%, 97%, 98%, 99%, or more) identical to the amino acid sequence of mature human IGF-II (SEQ ID NO: 15).
E10. The method of any one of E1-E9, wherein the GILT tag has an amino acid sequence that is at least 70% (e.g., at least 75%, 80%, 85%, 90%, 95%, 96%, 97%, 98%, 99%, or more) identical to the amino acid sequence of SEQ ID NO: 16.
E11. The method of E10, wherein the GILT tag has an amino acid sequence that is at least 80% (e.g., at least 85%, 90%, 95%, 96%, 97%, 98%, 99%, or more) identical to the amino acid sequence of SEQ ID NO: 16.
E12. The method of E11, wherein the GILT tag has an amino acid sequence that is at least 90% (e.g., at least 91%, 92%, 93%, 94%, 95%, 96%, 97%, 98%, 99%, or more) identical to the amino acid sequence of SEQ ID NO: 16.
E13. The method of any one of E1-E9, wherein the GILT tag has an amino acid sequence that is at least 70% (e.g., at least 75%, 80%, 85%, 90%, 95%, 96%, 97%, 98%, 99%, or more) identical to the amino acid sequence of SEQ ID NO: 17.
E14. The method of E13, wherein the GILT tag has an amino acid sequence that is at least 80% (e.g., at least 85%, 90%, 95%, 96%, 97%, 98%, 99%, or more) identical to the amino acid sequence of SEQ ID NO: 17.
E15. The method of E14, wherein the GILT tag has an amino acid sequence that is at least 90% (e.g., at least 91%, 92%, 93%, 94%, 95%, 96%, 97%, 98%, 99%, or more) identical to the amino acid sequence of SEQ ID NO: 17.
E16. The method of any one of E1-E9, wherein the GILT tag has an amino acid sequence that is at least 70% (e.g., at least 75%, 80%, 85%, 90%, 95%, 96%, 97%, 98%, 99%, or more) identical to the amino acid sequence of SEQ ID NO: 18.
E17. The method of E16, wherein the GILT tag has an amino acid sequence that is at least 80% (e.g., at least 85%, 90%, 95%, 96%, 97%, 98%, 99%, or more) identical to the amino acid sequence of SEQ ID NO: 18.
E18. The method of E17, wherein the GILT tag has an amino acid sequence that is at least 90% (e.g., at least 91%, 92%, 93%, 94%, 95%, 96%, 97%, 98%, 99%, or more) identical to the amino acid sequence of SEQ ID NO: 18.
E19. The method of any one of E1-E9, wherein the GILT tag has an amino acid sequence that is at least 70% (e.g., at least 75%, 80%, 85%, 90%, 95%, 96%, 97%, 98%, 99%, or more) identical to the amino acid sequence of SEQ ID NO: 19.
E20. The method of E19, wherein the GILT tag has an amino acid sequence that is at least 80% (e.g., at least 85%, 90%, 95%, 96%, 97%, 98%, 99%, or more) identical to the amino acid sequence of SEQ ID NO: 19.
E21. The method of E20, wherein the GILT tag has an amino acid sequence that is at least 90% (e.g., at least 91%, 92%, 93%, 94%, 95%, 96%, 97%, 98%, 99%, or more) identical to the amino acid sequence of SEQ ID NO: 19.
E22. The method of any one of E1-E9, wherein the GILT tag has an amino acid sequence that is at least 70% (e.g., at least 75%, 80%, 85%, 90%, 95%, 96%, 97%, 98%, 99%, or more) identical to the amino acid sequence of SEQ ID NO: 20.
E23. The method of E22, wherein the GILT tag has an amino acid sequence that is at least 80% (e.g., at least 85%, 90%, 95%, 96%, 97%, 98%, 99%, or more) identical to the amino acid sequence of SEQ ID NO: 20.
E24. The method of E23, wherein the GILT tag has an amino acid sequence that is at least 90% (e.g., at least 91%, 92%, 93%, 94%, 95%, 96%, 97%, 98%, 99%, or more) identical to the amino acid sequence of SEQ ID NO: 20.
E25. The method of any one of E1-E24, wherein the GILT tag is encoded by a polynucleotide having a nucleic acid sequence that is at least 85% (e.g., at least 90%, 91%, 92%, 93%, 94%, 95%, 96%, 97%, 98%, 99%, or more) identical to the nucleic acid sequence of SEQ ID NO: 21.
E26. The method of E25, wherein the GILT tag is encoded by a polynucleotide having a nucleic acid sequence that is at least 90% (e.g., at least 91%, 92%, 93%, 94%, 95%, 96%, 97%, 98%, 99%, or more) identical to the nucleic acid sequence of SEQ ID NO: 21.
E27. The method of E26, wherein the GILT tag is encoded by a polynucleotide having a nucleic acid sequence that is at least 95% (e.g., at least 96%, 97%, 98%, 99%, or more) identical to the nucleic acid sequence of SEQ ID NO: 21.
E28. The method of E27, wherein the GILT tag is encoded by a polynucleotide having a nucleic acid sequence of SEQ ID NO: 21.
E29. The method of any one of E1-E24, wherein the GILT tag is encoded by a polynucleotide having a nucleic acid sequence that is at least 85% (e.g., at least 90%, 91%, 92%, 93%, 94%, 95%, 96%, 97%, 98%, 99%, or more) identical to the nucleic acid sequence of SEQ ID NO: 22.
E30. The method of E29, wherein the GILT tag is encoded by a polynucleotide having a nucleic acid sequence that is at least 90% (e.g., at least 91%, 92%, 93%, 94%, 95%, 96%, 97%, 98%, 99%, or more) identical to the nucleic acid sequence of SEQ ID NO: 22.
E31. The method of E30, wherein the GILT tag is encoded by a polynucleotide having a nucleic acid sequence that is at least 95% (e.g., at least 96%, 97%, 98%, 99%, or more) identical to the nucleic acid sequence of SEQ ID NO: 22.
E32. The method of E31, wherein the GILT tag is encoded by a polynucleotide having a nucleic acid sequence of SEQ ID NO: 22.
E33. The method of any one of E1-E24, wherein the GILT tag is encoded by a polynucleotide having a nucleic acid sequence that is at least 85% (e.g., at least 90%, 91%, 92%, 93%, 94%, 95%, 96%, 97%, 98%, 99%, or more) identical to the nucleic acid sequence of SEQ ID NO: 23.
E34. The method of E33, wherein the GILT tag is encoded by a polynucleotide having a nucleic acid sequence that is at least 90% (e.g., at least 91%, 92%, 93%, 94%, 95%, 96%, 97%, 98%, 99%, or more) identical to the nucleic acid sequence of SEQ ID NO: 23.
E35. The method of E34, wherein the GILT tag is encoded by a polynucleotide having a nucleic acid sequence that is at least 95% (e.g., at least 96%, 97%, 98%, 99%, or more) identical to the nucleic acid sequence of SEQ ID NO: 23.
E36. The method of E35, wherein the GILT tag is encoded by a polynucleotide having a nucleic acid sequence of SEQ ID NO: 23.
E37. The method of any one of E1-E36, wherein the human IGF-II mutein has diminished binding affinity for the insulin receptor relative to the affinity of naturally-occurring human IGF-II for the insulin receptor, wherein the IGF-II mutein is resistant to furin cleavage, wherein the IGF-II mutein binds to the human cation-independent mannose-E6-phosphate receptor in a mannose-E6-phosphate-independent manner.
E38. The method of any one of E1-E37, wherein the transgene is operably linked to a promoter.
E39. The method of E38, wherein the promoter is a ubiquitous promoter.
E40. The method of E39, wherein the ubiquitous promoter is an EF1α promoter, PGK1 promoter, or β-LCR(HS4,3,2)-EFS promoter.
E41. The method of E38, wherein the promoter is a cell lineage-specific promoter.
E42. The method of E41, wherein the cell lineage-specific promoter is a CD68 promoter, a CD11 b promoter, a CX3CR1 1 promoter, an AIF1 promoter, a P2Y12 promoter, a TMEM119 promoter, or a CSF1R promoter.
E43. The method of E38, wherein the promoter is a viral promoter.
E44. The method of E43, wherein the viral promoter is an adenovirus late promoter, vaccinia virus 7.5K promoter, SV40 promoter, CMV promoter, tk HSV promoter, MMTV promoter, LTR of HIV promoter, Moloney virus promoter, EBV promoter, or RSV promoter.
E45. The method of E38, wherein the promoter is a synthetic promoter.
E46. The method of E45, wherein the synthetic promoter is an MND promoter.
E47. The method of E46, wherein the MND promoter includes a polynucleotide having at least 85% (e.g., at least 90%, 91%, 92%, 93%, 94%, 95%, 96%, 97%, 98%, 99%, or more) sequence identity to the nucleic acid sequence of SEQ ID NO: 10, optionally wherein the MND promoter includes a polynucleotide having at least 90% (e.g., at least 91%, 92%, 93%, 94%, 95%, 96%, 97%, 98%, 99%, or more) sequence identity to the nucleic acid sequence of SEQ ID NO: 10, optionally wherein the MND promoter includes a polynucleotide having at least 95% (e.g., at least 96%, 97%, 98%, 99%, or more) sequence identity to the nucleic acid sequence of SEQ ID NO: 10, optionally wherein the MND promoter includes a polynucleotide having the nucleic acid sequence of SEQ ID NO: 10.
E48. The method of E46, wherein the MND promoter includes a polynucleotide having at least 85% (e.g., at least 90%, 91%, 92%, 93%, 94%, 95%, 96%, 97%, 98%, 99%, or more) sequence identity to the nucleic acid sequence of SEQ ID NO: 11, optionally wherein the MND promoter includes a polynucleotide having at least 90% (e.g., at least 91%, 92%, 93%, 94%, 95%, 96%, 97%, 98%, 99%, or more) sequence identity to the nucleic acid sequence of SEQ ID NO: 11, optionally wherein the MND promoter includes a polynucleotide having at least 95% (e.g., at least 96%, 97%, 98%, 99%, or more) sequence identity to the nucleic acid sequence of SEQ ID NO: 11, optionally wherein the MND promoter includes a polynucleotide having the nucleic acid sequence of SEQ ID NO: 11.
E49. The method of any one of E1-E48, wherein the transgene encodes a GAA protein having an amino acid sequence that is at least 85% (e.g., at least 90%, 91%, 92%, 93%, 94%, 95%, 96%, 97%, 98%, 99%, or more) identical to the amino acid sequence of SEQ ID NO: 1, optionally wherein the GAA protein has an amino acid sequence that is at least 90% (e.g., at least 91%, 92%, 93%, 94%, 95%, 96%, 97%, 98%, 99%, or more) identical to the amino acid sequence of SEQ ID NO: 1, optionally wherein the GAA protein has an amino acid sequence that is at least 95% (e.g., at least 96%, 97%, 98%, 99%, or more) identical to the amino acid sequence of SEQ ID NO: 1, optionally wherein the GAA protein has an amino acid sequence of SEQ ID NO: 1.
E50. The method of any one of E1-E48, wherein the transgene encodes a GAA protein having an amino acid sequence that is at least 85% (e.g., at least 90%, 91%, 92%, 93%, 94%, 95%, 96%, 97%, 98%, 99%, or more) identical to the amino acid sequence of SEQ ID NO: 2, optionally wherein the GAA protein has an amino acid sequence that is at least 90% (e.g., at least 91%, 92%, 93%, 94%, 95%, 96%, 97%, 98%, 99%, or more) identical to the amino acid sequence of SEQ ID NO: 2, optionally wherein the GAA protein has an amino acid sequence that is at least 95% (e.g., at least 96%, 97%, 98%, 99%, or more) identical to the amino acid sequence of SEQ ID NO: 2, optionally wherein the GAA protein has an amino acid sequence of SEQ ID NO: 2.
E51. The method of any one of E1-E48, wherein the transgene encodes a GAA protein having an amino acid sequence that is at least 85% (e.g., at least 90%, 91%, 92%, 93%, 94%, 95%, 96%, 97%, 98%, 99%, or more) identical to the amino acid sequence of SEQ ID NO: 3, optionally wherein the GAA protein has an amino acid sequence that is at least 90% (e.g., at least 91%, 92%, 93%, 94%, 95%, 96%, 97%, 98%, 99%, or more) identical to the amino acid sequence of SEQ ID NO: 3, optionally wherein the GAA protein has an amino acid sequence that is at least 95% (e.g., at least 96%, 97%, 98%, 99%, or more) identical to the amino acid sequence of SEQ ID NO: 3, optionally wherein the GAA protein has an amino acid sequence of SEQ ID NO: 3.
E52. The method of any one of E1-E48, wherein the transgene encodes a GAA protein having an amino acid sequence that is at least 85% (e.g., at least 90%, 91%, 92%, 93%, 94%, 95%, 96%, 97%, 98%, 99%, or more) identical to the amino acid sequence of SEQ ID NO: 4, optionally wherein the GAA protein has an amino acid sequence that is at least 90% (e.g., at least 91%, 92%, 93%, 94%, 95%, 96%, 97%, 98%, 99%, or more) identical to the amino acid sequence of SEQ ID NO: 4, optionally wherein the GAA protein has an amino acid sequence that is at least 95% (e.g., at least 96%, 97%, 98%, 99%, or more) identical to the amino acid sequence of SEQ ID NO: 4, optionally wherein the GAA protein has an amino acid sequence of SEQ ID NO: 4.
E53. The method of any one of E1-E48, wherein the GAA protein is encoded by a polynucleotide having at least 85% (e.g., at least 90%, 91%, 92%, 93%, 94%, 95%, 96%, 97%, 98%, 99%, or more) sequence identity to the nucleic acid sequence of SEQ ID NO: 5, optionally wherein the GAA protein is encoded by a polynucleotide having at least 90% (e.g., at least 91%, 92%, 93%, 94%, 95%, 96%, 97%, 98%, 99%, or more) sequence identity to the nucleic acid sequence of SEQ ID NO: 5, optionally wherein the GAA protein is encoded by a polynucleotide having at least 95% (e.g., at least 96%, 97%, 98%, 99%, or more) sequence identity to the nucleic acid sequence of SEQ ID NO: 5, optionally wherein the GAA protein is encoded by a polynucleotide having the nucleic acid sequence of SEQ ID NO: 5.
E54. The method of any one of E1-E48, wherein the GAA protein is encoded by a polynucleotide having at least 85% (e.g., at least 90%, 91%, 92%, 93%, 94%, 95%, 96%, 97%, 98%, 99%, or more) sequence identity to the nucleic acid sequence of SEQ ID NO: 6, optionally wherein the GAA protein is encoded by a polynucleotide having at least 90% (e.g., at least 91%, 92%, 93%, 94%, 95%, 96%, 97%, 98%, 99%, or more) sequence identity to the nucleic acid sequence of SEQ ID NO: 6, optionally wherein the GAA protein is encoded by a polynucleotide having at least 95% (e.g., at least 96%, 97%, 98%, 99%, or more) sequence identity to the nucleic acid sequence of SEQ ID NO: 6, optionally wherein the GAA protein is encoded by a polynucleotide having the nucleic acid sequence of SEQ ID NO: 6.
E55. The method of any one of E1-E48, wherein the GAA protein is encoded by a polynucleotide having at least 85% (e.g., at least 90%, 91%, 92%, 93%, 94%, 95%, 96%, 97%, 98%, 99%, or more) sequence identity to the nucleic acid sequence of SEQ ID NO: 7, optionally wherein the GAA protein is encoded by a polynucleotide having at least 90% (e.g., at least 91%, 92%, 93%, 94%, 95%, 96%, 97%, 98%, 99%, or more) sequence identity to the nucleic acid sequence of SEQ ID NO: 7, optionally wherein the GAA protein is encoded by a polynucleotide having at least 95% (e.g., at least 96%, 97%, 98%, 99%, or more) sequence identity to the nucleic acid sequence of SEQ ID NO: 7, optionally wherein the GAA protein is encoded by a polynucleotide having the nucleic acid sequence of SEQ ID NO: 7.
E56. The method of any one of E1-E48, wherein the GAA protein is encoded by a polynucleotide having at least 85% (e.g., at least 90%, 91%, 92%, 93%, 94%, 95%, 96%, 97%, 98%, 99%, or more) sequence identity to the nucleic acid sequence of SEQ ID NO: 8, optionally wherein the GAA protein is encoded by a polynucleotide having at least 90% (e.g., at least 91%, 92%, 93%, 94%, 95%, 96%, 97%, 98%, 99%, or more) sequence identity to the nucleic acid sequence of SEQ ID NO: 8, optionally wherein the GAA protein is encoded by a polynucleotide having at least 95% (e.g., at least 96%, 97%, 98%, 99%, or more) sequence identity to the nucleic acid sequence of SEQ ID NO: 8, optionally wherein the GAA protein is encoded by a polynucleotide having the nucleic acid sequence of SEQ ID NO: 8.
E57. The method of any one of E1-E48, wherein the GAA protein is encoded by a polynucleotide having at least 85% (e.g., at least 90%, 91%, 92%, 93%, 94%, 95%, 96%, 97%, 98%, 99%, or more) sequence identity to the nucleic acid sequence of SEQ ID NO: 9, optionally wherein the GAA protein is encoded by a polynucleotide having at least 90% (e.g., at least 91%, 92%, 93%, 94%, 95%, 96%, 97%, 98%, 99%, or more) sequence identity to the nucleic acid sequence of SEQ ID NO: 9, optionally wherein the GAA protein is encoded by a polynucleotide having at least 95% (e.g., at least 96%, 97%, 98%, 99%, or more) sequence identity to the nucleic acid sequence of SEQ ID NO: 9, optionally wherein the GAA protein is encoded by a polynucleotide having the nucleic acid sequence of SEQ ID NO: 9.
E58. The method of any one of E1-E57, wherein the GAA is a full-length GAA.
E59. The method of any one of E1-E58, wherein the GAA includes a signal peptide.
E60. The method of E59, wherein the signal peptide is a GAA signal peptide.
E61. The method of E59, wherein the signal peptide is an IGF-II signal peptide.
E62. The method of E61, wherein the IGF-II signal peptide includes an amino acid sequence of SEQ ID NO: 12.
E63. The method of any one of E1-E62, wherein the transgene encodes two or more (e.g., 3, 4, 5, 6, 7, 8, 9, 10, or more) GILT.GAA proteins.
E64. The method of any one of E1-E63, wherein the transgene is a codon-optimized GILT.GAA transgene.
E65. The method of any one of E1-E64, wherein the GILT.GAA protein includes an Rb domain of ApoE.
E66. The method of E65, wherein the Rb domain includes a portion of ApoE having the amino acid sequence of residues 25-185, 50-E80, 75-175, 100-170, 125-160, or 130-150 of SEQ ID NO: 24.
E67. The method of E65 or E66, wherein the Rb domain includes a region having at least 70% (e.g., at least 75%, 80%, 85%, 90%, 95%, 96%, 97%, 98%, 99%, or more) sequence identity to the amino acid sequence of residues 159-167 of SEQ ID NO: 24.
E68. The method of any one of E1-E67, wherein the transgene further includes a miR-126 targeting sequence in the 3′-UTR.
E69. The method of any one of E1-E68, wherein the cells are pluripotent cells or multipotent cells.
E70. The method of E69, wherein the multipotent cells are CD34+ cells.
E71. The method of E70, wherein the CD34+ cells are HSCs or myeloid cells.
E72. The method of E71, wherein the myeloid cells are MPCs.
E73. The method of E69, wherein the pluripotent cells are ESCs or iPSCs.
E74. The method of any one of E1-E68, wherein the cells are BLPCs, microglial progenitor cells, monocytes, macrophages, or microglia.
E75. The method of E74, wherein the BLPCs are monocytes.
E76. The method of any one of E1-E75, wherein an expression level of the transgene is measured in one or more organs, tissues, or body fluids of the subject.
E77. The method of E76, wherein the one or more body fluids is peripheral blood.
E78. The method of E76, wherein the one or more tissues is muscle tissue or nervous system tissue.
E79. The method of E76, wherein the one or more organs is the liver and/or the heart.
E80. The method of any one of E1-E79, wherein a population of endogenous cells in the subject has been ablated prior to administration of the composition.
E81. The method of any one of E1-E79, wherein the method further includes ablating a population of endogenous cells in the subject prior to administering the composition to the subject.
E82. The method of E80 or E81 wherein the endogenous cells are ablated using an agent selected from the group consisting of busulfan, PLX3397, PLX647, PLX5622, treosulfan, and clodronate liposomes, by radiation therapy, or a combination thereof.
E83. The method of E82, wherein the endogenous cells are ablated using busulfan.
E84. The method of any one of E80-E83, wherein the endogenous cells are pluripotent cells or multipotent cells.
E85. The method of E84, wherein the multipotent cells are CD34+ cells.
E86. The method of E85, wherein the CD34+ cells are HSCs or myeloid cells.
E87. The method of E86, wherein the myeloid cells are MPCs.
E88. The method of E84, wherein the pluripotent cells are ESCs or iPSCs.
E89. The method of any one of E80-E83, wherein the endogenous cells are BLPCs, microglial progenitor cells, monocytes, macrophages, or microglial cells.
E90. The method of E89, wherein the BLPCs are monocytes.
E91. The method of any one of E1-E90, wherein the composition is administered to the subject by way of systemic administration, by way of direct administration to the central nervous system of the subject, by way of direct administration to the bone marrow of the subject, or by way of bone marrow transplant including the composition.
E92. The method of any one of E1-E91, wherein the cells are autologous cells or allogeneic cells.
E93. The method of any one of E1-E92, wherein the cells are transfected or transduced ex vivo to express the GAA.
E94. The method of E93, wherein the cells are transduced with a viral vector selected from the group consisting of an AAV, an adenovirus, a parvovirus, a coronavirus, a rhabdovirus, a paramyxovirus, a picornavirus, an alphavirus, a herpes virus, a poxvirus, and a Retroviridae family virus.
E95. The method of E94, wherein the viral vector is a Retroviridae family viral vector.
E96. The method of E95, wherein the Retroviridae family viral vector is a lentiviral vector, alpharetroviral vector, or gamma retroviral vector.
E97. The method of any one of E94-E96, wherein the Retroviridae family viral vector includes a central polypurine tract, a woodchuck hepatitis virus post-transcriptional regulatory element, a 5′-LTR, HIV signal sequence, HIV Psi signal 5′-splice site, delta-GAG element, 3′-splice site, and a 3′-self inactivating LTR.
E98. The method of E94, wherein the viral vector is an AAV selected from the group consisting of AAV1, AAV2, AAV3, AAV4, AAV5, AAV6, AAV7, AAV8, AAV9, AAV10, and AAVrh74.
E99. The method of any one of E94-E98, wherein the viral vector is a pseudotyped viral vector.
E100. The method of E99, wherein the pseudotyped viral vector selected from the group consisting of a pseudotyped AAV, a pseudotyped adenovirus, a pseudotyped parvovirus, a pseudotyped coronavirus, a pseudotyped rhabdovirus, a pseudotyped paramyxovirus, a pseudotyped picornavirus, a pseudotyped alphavirus, a pseudotyped herpes virus, a pseudotyped poxvirus, and a pseudotyped Retroviridae family virus.
E101. The method of any one of E1-E100, wherein the subject with Pompe disease is a CRIM-negative subject.
E102. The method of E101, wherein the method further includes administering an ITI agent to the CRIM-negative subject prior to, concurrently with, or after the administration of the composition.
E103. The method of E102, wherein the ITI agent includes rituximab, methotrexate, and IVIG.
E104. The method of any one of E1-E103, wherein the subject with Pompe disease is a CRIM-positive subject.
E105. The method of any one of E1-E104, wherein the Pompe disease is an infantile-onset Pompe disease.
E106. The method of E105, wherein the subject is from about one month to about one year of age.
E107. The method of E106, wherein the subject is from about one month to about six months of age.
E108. The method of any one of E105-E107, wherein prior to administration of the composition to the subject, the subject exhibits one or more symptoms selected from feeding difficulties, failure to thrive, hypotonia, progressive weakness, respiratory distress, macroglossia, and cardiac hypertrophy.
E109. The method of any one of E1-E104, wherein the Pompe disease is a late-onset Pompe disease.
E110. The method of E109, wherein the subject exhibits endogenous GAA activity from about 1% to about 40% of the endogenous GAA activity of a human of the same gender and similar body mass index that does not have Pompe disease.
E111. The method of any one of E1-E110, wherein following the administration of the composition to the subject, the subject exhibits endogenous GAA activity of from about 10% to about 2000% of the endogenous GAA activity of a human of the same gender and similar body mass index that does not have Pompe disease.
E112. The method of any one of E1-E111, wherein the composition is administered to the subject in a dosage of 1×10⁵ as cells/kg to about 30×10⁷ cells/kg.
E113. The method of any one of E1-E112, wherein the subject is female.
E114. The method of any one of E1-E112, wherein the subject is male.
E115. The method of any one of E1-E114, wherein the composition is administered in an amount sufficient to reduce one or more of cardiomegaly, hypotonia, cardiomyopathy, respiratory distress, muscle weakness, feeding difficulties, failure to thrive, floppy baby appearance, delay in motor development, hepatomegaly, macroglossia, wide open mouth, wide open eyes, nasal flaring, respiratory rate, engagement of accessory muscles for breathing, frequency of chest infections, arrhythmia, heart failure, impaired cough, muscle weakness, difficulty masticating and swallowing, or the composition is administered in an amount sufficient to increase one or more of facial muscle tone, air flow in the left lower zone, and vital capacity.
E116. The method of any one of E1-E115, wherein the composition is administered in an amount sufficient to reduce glycogen accumulation in muscle cells, neural cells, and/or liver cells.
E117. The method of any one of E1-E116, wherein the composition is administered in an amount sufficient to increase GAA expression level and/or enzymatic activity in muscle cells, neural cells, and/or liver cells of the subject.
E118. The method of E116 or E117, wherein the neural cells are neurons or glial cells.
E119. The method of any one of E116-E118, wherein the muscle cells are skeletal muscle cells and/or cardiac muscle cells.
E120. The method of any one of E1-E119, wherein the composition is administered in an amount sufficient to reduce glycogen accumulation in muscle tissue and/or nervous tissue.
E121. The method of any one of E1-E120, wherein the composition is administered in an amount sufficient to increase GAA expression level and/or enzymatic activity in muscle tissue or nervous tissue.
E122. The method of E120 or E121, wherein the muscle tissue is of the heart, diaphragm, gastrocnemius muscle, quadriceps femoris muscle, and/or tibialis anterior muscle.
E123. The method of E120 or E121, wherein the nervous tissue is of the cerebellum, cerebrum, thoracic or cervical spinal cord, and/or hippocampus.
E124. The method of any one of E1-E123, wherein the subject has not previously received GAA ERT.
E125. The method of any one of E1-E124, wherein the subject has previously received GAA ERT.
E126. The method of any one of E1-E125, wherein the subject has atrophy in one or more tissues selected from heart, diaphragm, gastrocnemius muscle, quadriceps femoris muscle, tibialis anterior muscle, cerebellum, cerebrum, thoracic spinal cord, cervical spinal cord, and hippocampus tissue.
E127. A composition including a population of cells that express a transgene encoding a GAA protein fused to a GILT tag (GILT.GAA protein).
E128. The composition of E127, wherein the GILT tag includes an IGF-II mutein including an Ala amino acid substitution at a position corresponding to Arg37 of SEQ ID NO: 15.
E129. The composition of E128, wherein the human IGF-II mutein has an amino acid sequence that is at least 70% (e.g., at least 75%, 80%, 85%, 90%, 95%, 96%, 97%, 98%, 99%, or more) identical to the amino acid sequence of mature human IGF-II (SEQ ID NO: 15).
E130. The composition of E129, wherein the human IGF-II mutein has an amino acid sequence that is at least 80% (e.g., at least 85%, 90%, 95%, 96%, 97%, 98%, 99%, or more) identical to the amino acid sequence of mature human IGF-II (SEQ ID NO: 15).
E131. The composition of E130, wherein the human IGF-II mutein has an amino acid sequence that is at least 90% (e.g., at least 91%, 92%, 93%, 94%, 95%, 96%, 97%, 98%, 99%, or more) identical to the amino acid sequence of mature human IGF-II (SEQ ID NO: 15).
E132. The composition of any one of E127-E131, wherein the GILT tag has an amino acid sequence that is at least 70% (e.g., at least 75%, 80%, 85%, 90%, 95%, 96%, 97%, 98%, 99%, or more) identical to the amino acid sequence of SEQ ID NO: 16.
E133. The composition of E132, wherein the GILT tag has an amino acid sequence that is at least 80% (e.g., at least 85%, 90%, 95%, 96%, 97%, 98%, 99%, or more) identical to the amino acid sequence of SEQ ID NO: 16.
E134. The composition of E133, wherein the GILT tag has an amino acid sequence that is at least 90% (e.g., at least 91%, 92%, 93%, 94%, 95%, 96%, 97%, 98%, 99%, or more) identical to the amino acid sequence of SEQ ID NO: 16.
E135. The composition of any one of E127-E131, wherein the GILT tag has an amino acid sequence that is at least 70% (e.g., at least 75%, 80%, 85%, 90%, 95%, 96%, 97%, 98%, 99%, or more) identical to the amino acid sequence of SEQ ID NO: 17.
E136. The composition of E135, wherein the GILT tag has an amino acid sequence that is at least 80% (e.g., at least 85%, 90%, 95%, 96%, 97%, 98%, 99%, or more) identical to the amino acid sequence of SEQ ID NO: 17.
E137. The composition of E136, wherein the GILT tag has an amino acid sequence that is at least 90% (e.g., at least 91%, 92%, 93%, 94%, 95%, 96%, 97%, 98%, 99%, or more) identical to the amino acid sequence of SEQ ID NO: 17.
E138. The composition of any one of E127-E131, wherein the GILT tag has an amino acid sequence that is at least 70% (e.g., at least 75%, 80%, 85%, 90%, 95%, 96%, 97%, 98%, 99%, or more) identical to the amino acid sequence of SEQ ID NO: 18.
E139. The composition of E138, wherein the GILT tag has an amino acid sequence that is at least 80% (e.g., at least 85%, 90%, 95%, 96%, 97%, 98%, 99%, or more) identical to the amino acid sequence of SEQ ID NO: 18.
E140. The composition of E139, wherein the GILT tag has an amino acid sequence that is at least 90% (e.g., at least 91%, 92%, 93%, 94%, 95%, 96%, 97%, 98%, 99%, or more) identical to the amino acid sequence of SEQ ID NO: 18.
E141. The composition of any one of E127-E131, wherein the GILT tag has an amino acid sequence that is at least 70% (e.g., at least 75%, 80%, 85%, 90%, 95%, 96%, 97%, 98%, 99%, or more) identical to the amino acid sequence of SEQ ID NO: 19.
E142. The composition of E141, wherein the GILT tag has an amino acid sequence that is at least 80% (e.g., at least 85%, 90%, 95%, 96%, 97%, 98%, 99%, or more) identical to the amino acid sequence of SEQ ID NO: 19.
E143. The composition of E142, wherein the GILT tag has an amino acid sequence that is at least 90% (e.g., at least 91%, 92%, 93%, 94%, 95%, 96%, 97%, 98%, 99%, or more) identical to the amino acid sequence of SEQ ID NO: 19.
E144. The composition of any one of E127-E131, wherein the GILT tag has an amino acid sequence that is at least 70% (e.g., at least 75%, 80%, 85%, 90%, 95%, 96%, 97%, 98%, 99%, or more) identical to the amino acid sequence of SEQ ID NO: 20.
E145. The composition of E144, wherein the GILT tag has an amino acid sequence that is at least 80% (e.g., at least 85%, 90%, 95%, 96%, 97%, 98%, 99%, or more) identical to the amino acid sequence of SEQ ID NO: 20.
E146. The composition of E145, wherein the GILT tag has an amino acid sequence that is at least 90% (e.g., at least 91%, 92%, 93%, 94%, 95%, 96%, 97%, 98%, 99%, or more) identical to the amino acid sequence of SEQ ID NO: 20.
E147. The composition of any one of E127-E146, wherein the GILT tag is encoded by a polynucleotide having a nucleic acid sequence that is at least 85% (e.g., at least 90%, 91%, 92%, 93%, 94%, 95%, 96%, 97%, 98%, 99%, or more) identical to the nucleic acid sequence of SEQ ID NO: 21.
E148. The composition of E147, wherein the GILT tag is encoded by a polynucleotide having a nucleic acid sequence that is at least 90% (e.g., at least 91%, 92%, 93%, 94%, 95%, 96%, 97%, 98%, 99%, or more) identical to the nucleic acid sequence of SEQ ID NO: 21.
E149. The composition of E148, wherein the GILT tag is encoded by a polynucleotide having a nucleic acid sequence that is at least 95% (e.g., at least 96%, 97%, 98%, 99%, or more) identical to the nucleic acid sequence of SEQ ID NO: 21.
E150. The composition of E149, wherein the GILT tag is encoded by a polynucleotide having a nucleic acid sequence of SEQ ID NO: 21.
E151. The composition of any one of E127-E146, wherein the GILT tag is encoded by a polynucleotide having a nucleic acid sequence that is at least 85% (e.g., at least 90%, 91%, 92%, 93%, 94%, 95%, 96%, 97%, 98%, 99%, or more) identical to the nucleic acid sequence of SEQ ID NO: 22.
E152. The composition of E151, wherein the GILT tag is encoded by a polynucleotide having a nucleic acid sequence that is at least 90% (e.g., at least 91%, 92%, 93%, 94%, 95%, 96%, 97%, 98%, 99%, or more) identical to the nucleic acid sequence of SEQ ID NO: 22.
E153. The composition of E152, wherein the GILT tag is encoded by a polynucleotide having a nucleic acid sequence that is at least 95% (e.g., at least 96%, 97%, 98%, 99%, or more) identical to the nucleic acid sequence of SEQ ID NO: 22.
E154. The composition of E153, wherein the GILT tag is encoded by a polynucleotide having a nucleic acid sequence of SEQ ID NO: 22.
E155. The composition of any one of E127-E146, wherein the GILT tag is encoded by a polynucleotide having a nucleic acid sequence that is at least 85% (e.g., at least 90%, 91%, 92%, 93%, 94%, 95%, 96%, 97%, 98%, 99%, or more) identical to the nucleic acid sequence of SEQ ID NO: 23.
E156. The composition of E155, wherein the GILT tag is encoded by a polynucleotide having a nucleic acid sequence that is at least 90% (e.g., at least 91%, 92%, 93%, 94%, 95%, 96%, 97%, 98%, 99%, or more) identical to the nucleic acid sequence of SEQ ID NO: 23.
E157. The composition of E156, wherein the GILT tag is encoded by a polynucleotide having a nucleic acid sequence that is at least 95% (e.g., at least 96%, 97%, 98%, 99%, or more) identical to the nucleic acid sequence of SEQ ID NO: 23.
E158. The composition of any one of E127-E157, wherein the human IGF-II mutein has diminished binding affinity for the insulin receptor relative to the affinity of naturally-occurring human IGF-II for the insulin receptor, wherein the IGF-II mutein is resistant to furin cleavage, wherein the IGF-II mutein binds to the human cation-independent mannose-E6-phosphate receptor in a mannose-E6-phosphate-independent manner.
E159. The composition of any one of E127-E158, wherein the transgene is operably linked to a promoter.
E160. The composition of E159, wherein the promoter is a ubiquitous promoter.
E161. The composition of E160, wherein the ubiquitous promoter is an EF1α promoter, PGK1 promoter, or β-LCR(HS4,3,2)-EFS promoter.
E162. The composition of E159, wherein the promoter is a cell lineage-specific promoter.
E163. The composition of E162, wherein the cell lineage-specific promoter is a CD68 promoter, a CD11 b promoter, a CX3CR1 1 promoter, an AIF1 promoter, a P2Y12 promoter, a TMEM119 promoter, or a CSF1R promoter.
E164. The composition of E159, wherein the promoter is a viral promoter.
E165. The composition of E164, wherein the viral promoter is an adenovirus late promoter, vaccinia virus 7.5K promoter, SV40 promoter, CMV promoter, tk HSV promoter, MMTV promoter, LTR of HIV promoter, Moloney virus promoter, EBV promoter, or RSV promoter.
E166. The composition of E159, wherein the promoter is a synthetic promoter.
E167. The composition of E166, wherein the synthetic promoter is an MND promoter.
E168. The composition of E167, wherein the MND promoter includes a polynucleotide having at least 85% (e.g., at least 90%, 91%, 92%, 93%, 94%, 95%, 96%, 97%, 98%, 99%, or more) sequence identity to the nucleic acid sequence of SEQ ID NO: 10, optionally wherein the MND promoter includes a polynucleotide having at least 90% (e.g., at least 91%, 92%, 93%, 94%, 95%, 96%, 97%, 98%, 99%, or more) sequence identity to the nucleic acid sequence of SEQ ID NO: 10, optionally wherein the MND promoter includes a polynucleotide having at least 95% (e.g., at least 96%, 97%, 98%, 99%, or more) sequence identity to the nucleic acid sequence of SEQ ID NO: 10, optionally wherein the MND promoter includes a polynucleotide having the nucleic acid sequence of SEQ ID NO: 10.
E169. The composition of E167, wherein the MND promoter includes a polynucleotide having at least 85% (e.g., at least 90%, 91%, 92%, 93%, 94%, 95%, 96%, 97%, 98%, 99%, or more) sequence identity to the nucleic acid sequence of SEQ ID NO: 11, optionally wherein the MND promoter includes a polynucleotide having at least 90% (e.g., at least 91%, 92%, 93%, 94%, 95%, 96%, 97%, 98%, 99%, or more) sequence identity to the nucleic acid sequence of SEQ ID NO: 11, optionally wherein the MND promoter includes a polynucleotide having at least 95% (e.g., at least 96%, 97%, 98%, 99%, or more) sequence identity to the nucleic acid sequence of SEQ ID NO: 11, optionally wherein the MND promoter includes a polynucleotide having the nucleic acid sequence of SEQ ID NO: 11.
E170. The composition of any one of E127-E169, wherein the transgene encodes a GAA protein having an amino acid sequence that is at least 85% (e.g., at least 90%, 91%, 92%, 93%, 94%, 95%, 96%, 97%, 98%, 99%, or more) identical to the amino acid sequence of SEQ ID NO: 1, optionally wherein the GAA protein has an amino acid sequence that is at least 90% (e.g., at least 91%, 92%, 93%, 94%, 95%, 96%, 97%, 98%, 99%, or more) identical to the amino acid sequence of SEQ ID NO: 1, optionally wherein the GAA protein has an amino acid sequence that is at least 95% (e.g., at least 96%, 97%, 98%, 99%, or more) identical to the amino acid sequence of SEQ ID NO: 1, optionally wherein the GAA protein has an amino acid sequence of SEQ ID NO: 1.
E171. The composition of any one of E127-E170, wherein the transgene encodes a GAA protein having an amino acid sequence that is at least 85% (e.g., at least 90%, 91%, 92%, 93%, 94%, 95%, 96%, 97%, 98%, 99%, or more) identical to the amino acid sequence of SEQ ID NO: 2, optionally wherein the GAA protein has an amino acid sequence that is at least 90% (e.g., at least 91%, 92%, 93%, 94%, 95%, 96%, 97%, 98%, 99%, or more) identical to the amino acid sequence of SEQ ID NO: 2, optionally wherein the GAA protein has an amino acid sequence that is at least 95% (e.g., at least 96%, 97%, 98%, 99%, or more) identical to the amino acid sequence of SEQ ID NO: 2, optionally wherein the GAA protein has an amino acid sequence of SEQ ID NO: 2.
E172. The composition of any one of E127-E171, wherein the transgene encodes a GAA protein having an amino acid sequence that is at least 85% (e.g., at least 90%, 91%, 92%, 93%, 94%, 95%, 96%, 97%, 98%, 99%, or more) identical to the amino acid sequence of SEQ ID NO: 3, optionally wherein the GAA protein has an amino acid sequence that is at least 90% (e.g., at least 91%, 92%, 93%, 94%, 95%, 96%, 97%, 98%, 99%, or more) identical to the amino acid sequence of SEQ ID NO: 3, optionally wherein the GAA protein has an amino acid sequence that is at least 95% (e.g., at least 96%, 97%, 98%, 99%, or more) identical to the amino acid sequence of SEQ ID NO: 3, optionally wherein the GAA protein has an amino acid sequence of SEQ ID NO: 3.
E173. The composition of any one of E127-E172, wherein the transgene encodes a GAA protein having an amino acid sequence that is at least 85% (e.g., at least 90%, 91%, 92%, 93%, 94%, 95%, 96%, 97%, 98%, 99%, or more) identical to the amino acid sequence of SEQ ID NO: 4, optionally wherein the GAA protein has an amino acid sequence that is at least 90% (e.g., at least 91%, 92%, 93%, 94%, 95%, 96%, 97%, 98%, 99%, or more) identical to the amino acid sequence of SEQ ID NO: 4, optionally wherein the GAA protein has an amino acid sequence that is at least 95% (e.g., at least 96%, 97%, 98%, 99%, or more) identical to the amino acid sequence of SEQ ID NO: 4, optionally wherein the GAA protein has an amino acid sequence of SEQ ID NO: 4.
E174. The composition of any one of E127-E173, wherein the GAA protein is encoded by a polynucleotide having at least 85% (e.g., at least 90%, 91%, 92%, 93%, 94%, 95%, 96%, 97%, 98%, 99%, or more) sequence identity to the nucleic acid sequence of SEQ ID NO: 5, optionally wherein the GAA protein is encoded by a polynucleotide having at least 90% (e.g., at least 91%, 92%, 93%, 94%, 95%, 96%, 97%, 98%, 99%, or more) sequence identity to the nucleic acid sequence of SEQ ID NO: 5, optionally wherein the GAA protein is encoded by a polynucleotide having at least 95% (e.g., at least 96%, 97%, 98%, 99%, or more) sequence identity to the nucleic acid sequence of SEQ ID NO: 5, optionally wherein the GAA protein is encoded by a polynucleotide having the nucleic acid sequence of SEQ ID NO: 5.
E175. The composition of any one of E127-E174, wherein the GAA protein is encoded by a polynucleotide having at least 85% (e.g., at least 90%, 91%, 92%, 93%, 94%, 95%, 96%, 97%, 98%, 99%, or more) sequence identity to the nucleic acid sequence of SEQ ID NO: 6, optionally wherein the GAA protein is encoded by a polynucleotide having at least 90% (e.g., at least 91%, 92%, 93%, 94%, 95%, 96%, 97%, 98%, 99%, or more) sequence identity to the nucleic acid sequence of SEQ ID NO: 6, optionally wherein the GAA protein is encoded by a polynucleotide having at least 95% (e.g., at least 96%, 97%, 98%, 99%, or more) sequence identity to the nucleic acid sequence of SEQ ID NO: 6, optionally wherein the GAA protein is encoded by a polynucleotide having the nucleic acid sequence of SEQ ID NO: 6.
E176. The composition of any one of E127-E175, wherein the GAA protein is encoded by a polynucleotide having at least 85% (e.g., at least 90%, 91%, 92%, 93%, 94%, 95%, 96%, 97%, 98%, 99%, or more) sequence identity to the nucleic acid sequence of SEQ ID NO: 7, optionally wherein the GAA protein is encoded by a polynucleotide having at least 90% (e.g., at least 91%, 92%, 93%, 94%, 95%, 96%, 97%, 98%, 99%, or more) sequence identity to the nucleic acid sequence of SEQ ID NO: 7, optionally wherein the GAA protein is encoded by a polynucleotide having at least 95% (e.g., at least 96%, 97%, 98%, 99%, or more) sequence identity to the nucleic acid sequence of SEQ ID NO: 7, optionally wherein the GAA protein is encoded by a polynucleotide having the nucleic acid sequence of SEQ ID NO: 7.
E177. The composition of any one of E127-E176, wherein the GAA protein is encoded by a polynucleotide having at least 85% (e.g., at least 90%, 91%, 92%, 93%, 94%, 95%, 96%, 97%, 98%, 99%, or more) sequence identity to the nucleic acid sequence of SEQ ID NO: 8, optionally wherein the GAA protein is encoded by a polynucleotide having at least 90% (e.g., at least 91%, 92%, 93%, 94%, 95%, 96%, 97%, 98%, 99%, or more) sequence identity to the nucleic acid sequence of SEQ ID NO: 8, optionally wherein the GAA protein is encoded by a polynucleotide having at least 95% (e.g., at least 96%, 97%, 98%, 99%, or more) sequence identity to the nucleic acid sequence of SEQ ID NO: 8, optionally wherein the GAA protein is encoded by a polynucleotide having the nucleic acid sequence of SEQ ID NO: 8.
E178. The composition of any one of E127-E177, wherein the GAA protein is encoded by a polynucleotide having at least 85% (e.g., at least 90%, 91%, 92%, 93%, 94%, 95%, 96%, 97%, 98%, 99%, or more) sequence identity to the nucleic acid sequence of SEQ ID NO: 9, optionally wherein the GAA protein is encoded by a polynucleotide having at least 90% (e.g., at least 91%, 92%, 93%, 94%, 95%, 96%, 97%, 98%, 99%, or more) sequence identity to the nucleic acid sequence of SEQ ID NO: 9, optionally wherein the GAA protein is encoded by a polynucleotide having at least 95% (e.g., at least 96%, 97%, 98%, 99%, or more) sequence identity to the nucleic acid sequence of SEQ ID NO: 9, optionally wherein the GAA protein is encoded by a polynucleotide having the nucleic acid sequence of SEQ ID NO: 9.
E179. The composition of any one of E127-E178, wherein the GAA is a full-length GAA.
E180. The composition of any one of E127-E179, wherein the GAA includes a signal peptide.
E181. The composition of E180, wherein the signal peptide is a GAA signal peptide.
E182. The composition of E180, wherein the signal peptide is an IGF-II signal peptide.
E183. The composition of E182, wherein the IGF-II signal peptide includes the amino acid sequence of SEQ ID NO: 12.
E184. The composition of any one of E127-E183, wherein the transgene encodes two or more GAA transgenes.
E185. The composition of any one of E127-E184, wherein the transgene is a codon-optimized GAA transgene.
E186. The composition of any one of E127-E185, wherein the GILT.GAA protein includes a Rb domain of ApoE.
E187. The composition of E186, wherein the Rb domain includes a portion of ApoE having the amino acid sequence of residues 25-185, 50-180, 75-175, 100-170, 125-160, or 130-150 of SEQ ID NO: 24.
E188. The composition of E186 or E187, wherein the Rb domain includes a region having at least 70% (e.g., at least 75%, 80%, 85%, 90%, 95%, 96%, 97%, 98%, 99%, or more) sequence identity to the amino acid sequence of residues 159-167 of SEQ ID NO: 24.
E189. The composition of any one of E127-E188, wherein the transgene encoding GAA further includes a miR-E126 targeting sequence in the 3′-UTR.
E190. The composition of any one of E121-E183, wherein the cells are pluripotent cells or multipotent cells.
E191. The composition of E190, wherein the multipotent cells are CD34+ cells.
E192. The composition of E191, wherein the CD34+ cells are HSCs or myeloid cells.
E193. The composition of E192, wherein the myeloid cells are MPCs.
E194. The composition of E190, wherein the pluripotent cells are ESCs or iPSCs.
E195. The composition of any one of E127-E188, wherein the cells are BLPCs, microglial progenitor cells, macrophages, or microglia.
E196. The composition of E195, wherein the BLPCs are monocytes.
E197. The composition of any one of E127-E196, wherein the cells are transfected or transduced ex vivo to express the GAA.
E198. A pharmaceutical composition including the composition of any one of E127-E197, wherein the pharmaceutical composition further includes a pharmaceutically acceptable carrier, diluent, or excipient.
E199. A kit including the composition of any one of E127-E197, or the pharmaceutical composition of
E198, and a package insert, wherein the package insert instructs a user of the kit to perform the method of any one of E1-E126.

### BRIEF DESCRIPTION OF THE DRAWINGS

FIGS. 1A and 1B show bar graphs illustrating alpha acid-glucosidase (GAA) activity (FIG. 1A) and glycogen accumulation (FIG. 1B) measured in the diaphragm of male (M) or female (F) GAA knock-out (Gaa−/−) mice (GILTco1-m) treated with lineage negative hematopoietic stem cells (HSCs) transduced with a codon-optimized transgene encoding GAA protein fused to a glycosylation-independent lysosomal targeting (GILT) tag (GILT.GAA protein) containing an Ala amino acid substitution at a position corresponding to Arg37 of a mature human insulin-like growth factor II (IGF) protein (SEQ ID NO: 15). For comparison, Gaa−/− and wild-type GAA+/+ mice receiving no HSCs, Gaa−/− mice receiving 7.5 Gy radiation and non-transduced HSCs (7.5 Gy NT), Gaa−/− mice receiving 7.5 Gy radiation and HSCs encoding a codon-optimized GAA transgene lacking a GILT tag were used as control groups. Other tested groups include Gaa−/− and wild-type GAA+/+ mice receiving 7.5 Gy radiation and HSCs transduced with a transgene encoding green fluorescent protein (GFP). Numerical values in parentheses at the end of each group name (e.g., 0.75, 1.5, and 3) correspond to multiplicity of infection (MOI) of transduced cells.

FIGS. 2A and 2B show bar graphs illustrating GAA activity (FIG. 2A) and glycogen accumulation (FIG. 2B) measured in the cerebrum of male (M) or female (F) Gaa−/− mice (GILTco1-m) treated with lineage negative HSCs transduced with a codon-optimized transgene encoding GAA protein fused to a GILT tag (GILT.GAA protein) containing an Ala amino acid substitution at a position corresponding to Arg37 of a mature human insulin-like growth factor II (IGF) protein (SEQ ID NO: 15). For comparison, Gaa−/− and wild-type GAA+/+ mice receiving no HSCs, Gaa−/− mice receiving 7.5 Gy radiation and non-transduced HSCs (7.5 Gy NT), Gaa−/− mice receiving 7.5 Gy radiation and HSCs encoding a codon-optimized GAA transgene lacking a GILT tag were used as control groups. Other tested groups include Gaa−/− and wild-type GAA+/+ mice receiving 7.5 Gy radiation and HSCs transduced with a transgene encoding GFP. Numerical values in parentheses at the end of each group name (e.g., 0.75, 1.5, and 3) correspond to MOI of transduced cells.

FIGS. 3A and 3B show bar graphs illustrating GAA activity (FIG. 3A) and glycogen accumulation (FIG. 3B) measured in the cerebellum of male (M) or female (F) Gaa−/− mice (GILTco1-m) treated with lineage negative HSCs transduced with a codon-optimized transgene encoding GAA protein fused to a GILT tag (GILT.GAA protein) containing an Ala amino acid substitution at a position corresponding to Arg37 of a mature human insulin-like growth factor II (IGF) protein (SEQ ID NO: 15 For comparison, Gaa−/− and wild-type GAA+/+ mice receiving no HSCs, Gaa−/− mice receiving 7.5 Gy radiation and non-transduced HSCs (7.5 Gy NT), Gaa−/− mice receiving 7.5 Gy radiation and HSCs encoding a codon-optimized GAA transgene lacking a GILT tag were used as control groups. Other tested groups include Gaa−/− and wild-type GAA+/+ mice receiving 7.5 Gy radiation and HSCs transduced with a transgene encoding GFP. Numerical values in parentheses at the end of each group name (e.g., 0.75, 1.5, and 3) correspond to MOI of transduced cells.

FIGS. 4A and 4B show bar graphs illustrating GAA activity (FIG. 4A) and glycogen accumulation (FIG. 4B) measured in the spinal cord of male (M) or female (F) Gaa−/− mice (GILTco1-m) treated with lineage negative HSCs transduced with a codon-optimized transgene encoding GAA protein fused to a GILT tag (GILT.GAA protein) containing an Ala amino acid substitution at a position corresponding to Arg37 of a mature human insulin-like growth factor II (IGF) protein (SEQ ID NO: 15). For comparison, Gaa−/− and wild-type GAA+/+ mice receiving no HSCs, Gaa−/− mice receiving 7.5 Gy radiation and non-transduced HSCs (7.5 Gy NT), Gaa−/− mice receiving 7.5 Gy radiation and HSCs encoding a codon-optimized GAA transgene lacking a GILT tag were used as control groups. Other tested groups include Gaa−/− and wild-type GAA+/+ mice receiving 7.5 Gy radiation and HSCs transduced with a transgene encoding GFP. Numerical values in parentheses at the end of each group name (e.g., 0.75, 1.5, and 3) correspond to MOI of transduced cells.

FIGS. 5A and 5B show bar graphs illustrating GAA activity (FIG. 5A) and glycogen accumulation (FIG. 5B) measured in the quadriceps femoris muscles of male (M) or female (F) Gaa−/− mice (GILTco1-m) treated with lineage negative HSCs transduced with a codon-optimized transgene encoding GAA protein fused to a GILT tag (GILT.GAA protein) containing an Ala amino acid substitution at a position corresponding to Arg37 of a mature human insulin-like growth factor II (IGF) protein (SEQ ID NO: 15). For comparison, Gaa−/− and wild-type GAA+/+ mice receiving no HSCs, Gaa−/− mice receiving 7.5 Gy radiation and non-transduced HSCs (7.5 Gy NT), Gaa−/− mice receiving 7.5 Gy radiation and HSCs encoding a codon-optimized GAA transgene lacking a GILT tag were used as control groups. Other tested groups include Gaa−/− and wild-type GAA+/+ mice receiving 7.5 Gy radiation and HSCs transduced with a transgene encoding GFP. Numerical values in parentheses at the end of each group name (e.g., 0.75, 1.5, and 3) correspond to MOI of transduced cells.

FIGS. 6A and 6B show bar graphs illustrating GAA activity (FIG. 6A) and glycogen accumulation (FIG. 6B) measured in the gastrocnemius muscles of male (M) or female (F) Gaa−/− mice (GILTco1-m) treated with lineage negative HSCs transduced with a codon-optimized transgene encoding GAA protein fused to a GILT tag (GILT.GAA protein) containing an Ala amino acid substitution at a position corresponding to Arg37 of a mature human insulin-like growth factor II (IGF) protein (SEQ ID NO: 15). For comparison, Gaa−/− and wild-type GAA+/+ mice receiving no HSCs, Gaa−/− mice receiving 7.5 Gy radiation and non-transduced HSCs (7.5 Gy NT), Gaa−/− mice receiving 7.5 Gy radiation and HSCs encoding a codon-optimized GAA transgene lacking a GILT tag were used as control groups. Other tested groups include Gaa−/− and wild-type GAA+/+ mice receiving 7.5 Gy radiation and HSCs transduced with a transgene encoding GFP. Numerical values in parentheses at the end of each group name (e.g., 0.75, 1.5, and 3) correspond to MOI of transduced cells.

FIGS. 7A and 7B show bar graphs illustrating GAA activity (FIG. 7A) and glycogen accumulation (FIG. 7B) measured in the tibialis anterior muscles of male (M) or female (F) Gaa−/− mice (GILTco1-m) treated with lineage negative HSCs transduced with a codon-optimized transgene encoding GAA protein fused to a GILT tag (GILT.GAA protein) containing an Ala amino acid substitution at a position corresponding to Arg37 of a mature human insulin-like growth factor II (IGF) protein (SEQ ID NO: 15). For comparison, Gaa−/− and wild-type GAA+/+ mice receiving no HSCs, Gaa−/− mice receiving 7.5 Gy radiation and non-transduced HSCs (7.5 Gy NT), Gaa−/− mice receiving 7.5 Gy radiation and HSCs encoding a codon-optimized GAA transgene lacking a GILT tag were used as control groups. Other tested groups include Gaa−/− and wild-type GAA+/+ mice receiving 7.5 Gy radiation and HSCs transduced with a transgene encoding GFP. Numerical values in parentheses at the end of each group name (e.g., 0.75, 1.5, and 3) correspond to MOI of transduced cells.

FIGS. 8A and 8B show diagrams of a lentiviral vector-mediated provirus (FIG. 8A) and GAA-encoding sequences (FIG. 8B). FIG. 8A shows the arrangement of elements in a lentiviral-mediated proviral vector, including a HIV 5′ long terminal repeat with inactivated (delta) Unique 3′, Repeat, Unique 5′(5′ LTR); packaging signal (ψ); truncated HIV GAG sequence (delta GAG); Rev-Response Element (RRE); central polypurine tract (cPPT); MND promoter (MND); Transgene, cDNA of interest listed in FIG. 8B; Woodchuck hepatitis virus posttranscriptional regulatory element (WPRE); and inactivated (delta) Unique 3′, Repeat, Unique 5′ (3′ LTR). FIG. 8B shows an overview of modified GAA sequences, where IGF2 is insulin-like growth factor 2 IGF2; SPS is signal peptide sequence (native GAA SPS in GAAco construct and IGF2 SPS in all other constructs); “co” is codon optimized; GILT is glycosylation independent lysosomal targeting; “L” is a Gly-Ala-Pro peptide linker; R>A represents an Arginine to Alanine mutation; and ApoE is Apolipoprotein E tag. cDNA sequences were codon optimized using different codon-optimization algorithms.

FIGS. 9A and 9B show bar graphs illustrating GAA activity normalized to vector copy number (VCN) in cell lysates (FIG. 9A) and in conditioned media (FIG. 9B) of HAP1 GAA−/− cells transduced with variants of a transgene encoding a GAA protein fused to a GILT tag (GILT.GAA protein) containing an Ala amino acid substitution at a position corresponding to Arg37 of a mature human insulin-like growth factor II (IGF) protein (SEQ ID NO: 15). Eight of these transgenes contained unique sequences encoding codon-optimized GAA. The codon-optimized GAA-encoding sequences included two sequences generated using two different codon optimization algorithms (GILTco1-m and GILTco2-m) and a GAA sequence translating into a consensus amino acid sequence (GILTco3-m). Two of the transgenes further contained an ApoE sequence (GILTco1-m-ApoE1 and GILTco1-m-ApoE2). One transgene further encoded a Gly-Ala-Pro peptide linker within the GAA sequence (GILTco1-m-L), and two transgenes further encoded ApoE and a Gly-Ala-Pro peptide linker (GILTco1-m-ApoE1-L and GILTco1-m-ApoE2-L). Another tested group included HAP1 GAA−/− cells encoding a wild-type GAA transgene and a GILT tag containing an R37A IGF-II mutein (GILTm). For comparison, wild-type HAP1 cells, HAP1 GAA−/− cells encoding a codon-optimized GAA transgene and a GILT tag that lacks an R37A IGF-II mutein (GILTco), and HAP1 GAA^{-/-} cells transduced with a codon-optimized GAA transgene lacking a GILT tag (GAAco) were used as control groups. Other tested groups included GAA−/− HAP1 cells transduced with a transgene encoding green fluorescent protein (GFP).

FIGS. 10A and 10B show bar graphs illustrating GAA activity (FIG. 10A) and glycogen accumulation (FIG. 10B) measured in the heart of female Gaa−/− mice treated with lineage negative HSCs transduced with a transgene encoding a GAA protein fused to a GILT tag (GILT.GAA protein) containing an Ala amino acid substitution at a position corresponding to Arg37 of a mature human insulin-like growth factor II (IGF) protein (SEQ ID NO: 15). Seven of these transgenes contained unique sequences encoding codon-optimized GAA. The codon-optimized GAA-encoding sequences included two sequences generated using two different codon optimization algorithms (GILTco1-m and GILTco2-m) and a GAA sequence translating into a consensus amino acid sequence (GILTco3-m). Two of the transgenes further contained an ApoE sequence (GILTco1-m-ApoE1 and GILTco1-m-ApoE2). One transgene further encoded a Gly-Ala-Pro peptide linker within the GAA sequence (GILTco1-m-L), and one transgene further encoded ApoE and a Gly-Ala-Pro peptide linker (GILTco1-m-ApoE2-L). An additional tested group included Gaa−/− mice receiving 7.5 Gy radiation and HSCs encoding a wild-type GAA transgene and a GILT tag containing an R37A IGF-II mutein (GILTm). For comparison, Gaa−/− and wild-type GAA+/+ mice receiving no HSCs (NT), Gaa−/− mice receiving 7.5 Gy radiation and HSCs encoding a codon-optimized GAA transgene and a GILT tag that lacks an R37A IGF-II mutein (GILTco), and Gaa−/− mice receiving 7.5 Gy radiation and HSCs encoding a codon-optimized GAA transgene lacking a GILT tag (GAAco) were used as control groups. Other tested groups include Gaa−/− mice receiving 7.5 Gy radiation or Busulfex® and HSCs transduced with a transgene encoding green fluorescent protein (GFP) as well as wild-type GAA+/+ mice receiving 7.5 Gy radiation and HSCs transduced with a transgene encoding GFP.

FIGS. 11A and 11B show bar graphs illustrating GAA activity (FIG. 11A) and glycogen accumulation (FIG. 11B) measured in the diaphragm of female Gaa−/− mice treated with lineage negative HSCs transduced with a transgene encoding a GAA protein fused to a GILT tag (GILT.GAA protein) containing an Ala amino acid substitution at a position corresponding to Arg37 of a mature human insulin-like growth factor II (IGF) protein (SEQ ID NO: 15). Seven of these transgenes contained unique sequences encoding codon-optimized GAA. The codon-optimized GAA-encoding sequences included two sequences generated using two different codon optimization algorithms (GILTco1-m and GILTco2-m) and a GAA sequence translating into a consensus amino acid sequence (GILTco3-m). Two of the transgenes further contained an ApoE sequence (GILTco1-m-ApoE1 and GILTco1-m-ApoE2). One transgene further encoded a Gly-Ala-Pro peptide linker within the GAA sequence (GILTco1-m-L), and one transgene further encoded ApoE and a Gly-Ala-Pro peptide linker (GILTco1-m-ApoE2-L). An additional tested group included Gaa−/− mice receiving 7.5 Gy radiation and HSCs encoding a wild-type GAA transgene and a GILT tag containing an R37A IGF-II mutein (GILTm). For comparison, Gaa−/− and wild-type GAA+/+ mice receiving no HSCs (NT), Gaa−/− mice receiving 7.5 Gy radiation and HSCs encoding a codon-optimized GAA transgene and a GILT tag that lacks an R37A IGF-II mutein (GILTco), and Gaa−/− mice receiving 7.5 Gy radiation and HSCs encoding a codon-optimized GAA transgene lacking a GILT tag (GAAco) were used as control groups. Other tested groups include Gaa−/− mice receiving 7.5 Gy radiation or Busulfex® and HSCs transduced with a transgene encoding green fluorescent protein (GFP) as well as wild-type GAA+/+ mice receiving 7.5 Gy radiation and HSCs transduced with a transgene encoding GFP.

FIGS. 12A and 12B show bar graphs illustrating GAA activity (FIG. 12A) and glycogen accumulation (FIG. 12B) measured in the gastrocnemius muscle of female Gaa−/− mice treated with lineage negative HSCs transduced with a transgene encoding a GAA protein fused to a GILT tag (GILT.GAA protein) containing an Ala amino acid substitution at a position corresponding to Arg37 of a mature human insulin-like growth factor II (IGF) protein (SEQ ID NO: 15). Seven of these transgenes contained unique sequences encoding codon-optimized GAA. The codon-optimized GAA-encoding sequences included two sequences generated using two different codon optimization algorithms (GILTco1-m and GILTco2-m) and a GAA sequence translating into a consensus amino acid sequence (GILTco3-m). Two of the transgenes further contained an ApoE sequence (GILTco1-m-ApoE1 and GILTco1-m-ApoE2). One transgene further encoded a Gly-Ala-Pro peptide linker within the GAA sequence (GILTco1-m-L), and one transgene further encoded ApoE and a Gly-Ala-Pro peptide linker (GILTco1-m-ApoE2-L). An additional tested group included Gaa−/− mice receiving 7.5 Gy radiation and HSCs encoding a wild-type GAA transgene and a GILT tag containing an R37A IGF-II mutein (GILTm). For comparison, Gaa−/− and wild-type GAA+/+ mice receiving no HSCs (NT), Gaa−/− mice receiving 7.5 Gy radiation and HSCs encoding a codon-optimized GAA transgene and a GILT tag that lacks an R37A IGF-II mutein (GILTco), and Gaa−/− mice receiving 7.5 Gy radiation and HSCs encoding a codon-optimized GAA transgene lacking a GILT tag (GAAco) were used as control groups. Other tested groups include Gaa−/− mice receiving 7.5 Gy radiation or Busulfex® and HSCs transduced with a transgene encoding green fluorescent protein (GFP) as well as wild-type GAA+/+ mice receiving 7.5 Gy radiation and HSCs transduced with a transgene encoding GFP.

FIGS. 13A and 13B show bar graphs illustrating GAA activity (FIG. 13A) and glycogen accumulation (FIG. 13B) measured in the quadriceps femoris muscle of female Gaa−/− mice treated with lineage negative HSCs transduced with a transgene encoding a GAA protein fused to a GILT tag (GILT.GAA protein) containing an Ala amino acid substitution at a position corresponding to Arg37 of a mature human insulin-like growth factor II (IGF) protein (SEQ ID NO: 15). Seven of these transgenes contained unique sequences encoding codon-optimized GAA. The codon-optimized GAA-encoding sequences included two sequences generated using two different codon optimization algorithms (GILTco1-m and GILTco2-m) and a GAA sequence translating into a consensus amino acid sequence (GILTco3-m). Two of the transgenes further contained an ApoE sequence (GILTco1-m-ApoE1 and GILTco1-m-ApoE2). One transgene further encoded a Gly-Ala-Pro peptide linker within the GAA sequence (GILTco1-m-L), and one transgene further encoded ApoE and a Gly-Ala-Pro peptide linker (GILTco1-m-ApoE2-L). An additional tested group included Gaa−/− mice receiving 7.5 Gy radiation and HSCs encoding a wild-type GAA transgene and a GILT tag containing an R37A IGF-II mutein (GILTm). For comparison, Gaa−/− and wild-type GAA+/+ mice receiving no HSCs (NT), Gaa−/− mice receiving 7.5 Gy radiation and HSCs encoding a codon-optimized GAA transgene and a GILT tag that lacks an R37A IGF-II mutein (GILTco), and Gaa−/− mice receiving 7.5 Gy radiation and HSCs encoding a codon-optimized GAA transgene lacking a GILT tag (GAAco) were used as control groups. Other tested groups include Gaa−/− mice receiving 7.5 Gy radiation or Busulfex® and HSCs transduced with a transgene encoding green fluorescent protein (GFP) as well as wild-type GAA+/+ mice receiving 7.5 Gy radiation and HSCs transduced with a transgene encoding GFP.

FIGS. 14A and 14B show bar graphs illustrating GAA activity (FIG. 14A) and glycogen accumulation (FIG. 14B) measured in the tibialis anterior muscle of female Gaa−/− mice treated with lineage negative HSCs transduced with a transgene encoding a GAA protein fused to a GILT tag (GILT.GAA protein) containing an Ala amino acid substitution at a position corresponding to Arg37 of a mature human insulin-like growth factor II (IGF) protein (SEQ ID NO: 15). Seven of these transgenes contained unique sequences encoding codon-optimized GAA. The codon-optimized GAA-encoding sequences included two sequences generated using two different codon optimization algorithms (GILTco1-m and GILTco2-m) and a GAA sequence translating into a consensus amino acid sequence (GILTco3-m). Two of the transgenes further contained an ApoE sequence (GILTco1-m-ApoE1 and GILTco1-m-ApoE2). One transgene further encoded a Gly-Ala-Pro peptide linker within the GAA sequence (GILTco1-m-L), and one transgene further encoded ApoE and a Gly-Ala-Pro peptide linker (GILTco1-m-ApoE2-L). An additional tested group included Gaa−/− mice receiving 7.5 Gy radiation and HSCs encoding a wild-type GAA transgene and a GILT tag containing an R37A IGF-II mutein (GILTm). For comparison, Gaa−/− and wild-type GAA+/+ mice receiving no HSCs (NT), Gaa−/− mice receiving 7.5 Gy radiation and HSCs encoding a codon-optimized GAA transgene and a GILT tag that lacks an R37A IGF-II mutein (GILTco), and Gaa−/− mice receiving 7.5 Gy radiation and HSCs encoding a codon-optimized GAA transgene lacking a GILT tag (GAAco) were used as control groups. Other tested groups include Gaa−/− mice receiving 7.5 Gy radiation or Busulfex® and HSCs transduced with a transgene encoding green fluorescent protein (GFP) as well as wild-type GAA+/+ mice receiving 7.5 Gy radiation and HSCs transduced with a transgene encoding GFP.

FIGS. 15A and 15B show bar graphs illustrating GAA activity (FIG. 15A) and glycogen accumulation (FIG. 15B) measured in the cerebellum of female Gaa−/− mice treated with lineage negative HSCs transduced with a transgene encoding a GAA protein fused to a GILT tag (GILT.GAA protein) containing an Ala amino acid substitution at a position corresponding to Arg37 of a mature human insulin-like growth factor II (IGF) protein (SEQ ID NO: 15). Seven of these transgenes contained unique sequences encoding codon-optimized GAA. The codon-optimized GAA-encoding sequences included two sequences generated using two different codon optimization algorithms (GILTco1-m and GILTco2-m) and a GAA sequence translating into a consensus amino acid sequence (GILTco3-m). Two of the transgenes further contained an ApoE sequence (GILTco1-m-ApoE1 and GILTco1-m-ApoE2). One transgene further encoded a Gly-Ala-Pro peptide linker within the GAA sequence (GILTco1-m-L), and one transgene further encoded ApoE and a Gly-Ala-Pro peptide linker (GILTco1-m-ApoE2-L). An additional tested group included Gaa−/− mice receiving 7.5 Gy radiation and HSCs encoding a wild-type GAA transgene and a GILT tag containing an R37A IGF-II mutein (GILTm). For comparison, Gaa−/− and wild-type GAA+/+ mice receiving no HSCs (NT), Gaa−/− mice receiving 7.5 Gy radiation and HSCs encoding a codon-optimized GAA transgene and a GILT tag that lacks an R37A IGF-II mutein (GILTco), and Gaa−/− mice receiving 7.5 Gy radiation and HSCs encoding a codon-optimized GAA transgene lacking a GILT tag (GAAco) were used as control groups. Other tested groups include Gaa−/− mice receiving 7.5 Gy radiation or Busulfex® and HSCs transduced with a transgene encoding green fluorescent protein (GFP) as well as wild-type GAA+/+ mice receiving 7.5 Gy radiation and HSCs transduced with a transgene encoding GFP.

FIGS. 16A and 16B show bar graphs illustrating GAA activity (FIG. 16A) and glycogen accumulation (FIG. 16B) measured in the cerebrum of female Gaa−/− mice treated with lineage negative HSCs transduced with a transgene encoding a GAA protein fused to a GILT tag (GILT.GAA protein) containing an Ala amino acid substitution at a position corresponding to Arg37 of a mature human insulin-like growth factor II (IGF) protein (SEQ ID NO: 15). Seven of these transgenes contained unique sequences encoding codon-optimized GAA. The codon-optimized GAA-encoding sequences included two sequences generated using two different codon optimization algorithms (GILTco1-m and GILTco2-m) and a GAA sequence translating into a consensus amino acid sequence (GILTco3-m). Two of the transgenes further contained an ApoE sequence (GILTco1-m-ApoE1 and GILTco1-m-ApoE2). One transgene further encoded a Gly-Ala-Pro peptide linker within the GAA sequence (GILTco1-m-L), and one transgene further encoded ApoE and a Gly-Ala-Pro peptide linker (GILTco1-m-ApoE2-L). An additional tested group included Gaa−/− mice receiving 7.5 Gy radiation and HSCs encoding a wild-type GAA transgene and a GILT tag containing an R37A IGF-II mutein (GILTm). For comparison, Gaa−/− and wild-type GAA+/+ mice receiving no HSCs (NT), Gaa−/− mice receiving 7.5 Gy radiation and HSCs encoding a codon-optimized GAA transgene and a GILT tag that lacks an R37A IGF-II mutein (GILTco), and Gaa−/− mice receiving 7.5 Gy radiation and HSCs encoding a codon-optimized GAA transgene lacking a GILT tag (GAAco) were used as control groups. Other tested groups include Gaa−/− mice receiving 7.5 Gy radiation or Busulfex® and HSCs transduced with a transgene encoding green fluorescent protein (GFP) as well as wild-type GAA+/+ mice receiving 7.5 Gy radiation and HSCs transduced with a transgene encoding GFP.

FIG. 17 shows a bar graph illustrating GAA protein concentration measured in the plasma of female Gaa−/− mice treated with lineage negative HSCs transduced with a transgene encoding a GAA protein fused to a GILT tag (GILT.GAA protein) containing an Ala amino acid substitution at a position corresponding to Arg37 of a mature human insulin-like growth factor II (IGF) protein (SEQ ID NO: 15). Seven of these transgenes contained unique sequences encoding codon-optimized GAA. The codon-optimized GAA-encoding sequences included two sequences generated using two different codon optimization algorithms (GILTco1-m and GILTco2-m) and a GAA sequence translating into a consensus amino acid sequence (GILTco3-m). Two of the transgenes further contained an ApoE sequence (GILTco1-m-ApoE1 and GILTco1-m-ApoE2). One transgene further encoded a Gly-Ala-Pro peptide linker within the GAA sequence (GILTco1-m-L), and one transgene further encoded ApoE and a Gly-Ala-Pro peptide linker (GILTco1-m-ApoE2-L). An additional tested group included Gaa−/− mice receiving 7.5 Gy radiation and HSCs encoding a wild-type GAA transgene and a GILT tag containing an R37A IGF-II mutein (GILTm). For comparison, Gaa−/− and wild-type GAA+/+ mice receiving no HSCs (NT), Gaa−/− mice receiving 7.5 Gy radiation and HSCs encoding a codon-optimized GAA transgene and a GILT tag that lacks an R37A IGF-II mutein (GILTco), and Gaa−/− mice receiving 7.5 Gy radiation and HSCs encoding a codon-optimized GAA transgene lacking a GILT tag (GAAco) were used as control groups. Other tested groups include Gaa−/− mice receiving 7.5 Gy radiation or Busulfex® and HSCs transduced with a transgene encoding green fluorescent protein (GFP) as well as wild-type GAA+/+ mice receiving 7.5 Gy radiation and HSCs transduced with a transgene encoding GFP.

FIGS. 18A and 18B show bar graphs illustrating GAA activity measured in bone marrow at week 32 post-treatment (FIG. 18A) and peripheral blood at weeks 4, 16, and 31 post-treatment (FIG. 18B) of male (M) or female (F) Gaa−/− mice (GILTco1-m) treated with lineage negative HSCs transduced with a codon-optimized transgene encoding GAA protein fused to a GILT tag (GILT.GAA protein) containing an Ala amino acid substitution at a position corresponding to Arg37 of a mature human insulin-like growth factor II (IGF) protein (SEQ ID NO: 15). For comparison, Gaa−/− and wild-type GAA+/+ mice receiving no HSCs (NT) were used as control groups. Numerical values in parentheses at the end of each group name (e.g., 0.75, 1.5, and 3) correspond to MOI of transduced cells.

FIGS. 19A and 19B show bar graphs illustrating vector copy number (VCN) measured in bone marrow at week 32 post-treatment (FIG. 19A) and peripheral blood at week 28 post-treatment (FIG. 19B) of male (M) or female (F) Gaa−/− mice (GILTco1-m) treated with lineage negative HSCs transduced with a codon-optimized transgene encoding GAA protein fused to a GILT tag (GILT.GAA protein) containing an Ala amino acid substitution at a position corresponding to Arg37 of a mature human insulin-like growth factor II (IGF) protein (SEQ ID NO: 15). For comparison, Gaa−/− mice receiving no HSCs were used as a control group (NT). Numerical values in parentheses at the end of each group name (e.g., 0.75, 1.5, and 3) correspond to MOI of transduced cells.

FIGS. 20A and 20B show bar graphs illustrating urinary HEX4 concentration measured pre-treatment and at week 30 post-treatment of female (FIG. 20A) or male (FIG. 20B) Gaa−/− mice (GILTco1-m) treated with lineage negative HSCs transduced with a codon-optimized transgene encoding GAA protein fused to a GILT tag (GILT.GAA protein) containing an Ala amino acid substitution at a position corresponding to Arg37 of a mature human insulin-like growth factor II (IGF) protein (SEQ ID NO: 15). For comparison, Gaa−/− mice receiving no HSCs were used as a control group (NT). Numerical values in parentheses at the end of each group name (e.g., 0.75, 1.5, and 3) correspond to MOI of transduced cells.

FIGS. 21A and 21B show representative images and mapping of changes in glycogen content in the heart, diaphragm, quadriceps femoris muscle, cerebellum, cerebral cortex, hippocampus, thoracic and cervical spinal cord, gastrocnemius muscle, and tibialis anterior muscle at week 32 post-treatment of male (M) or female (F) Gaa−/− mice (GILTco1-m) treated with lineage negative HSCs transduced with a codon-optimized transgene encoding GAA protein fused to a GILT tag (GILT.GAA protein) containing an Ala amino acid substitution at a position corresponding to Arg37 of a mature human insulin-like growth factor II (IGF) protein (SEQ ID NO: 15). For comparison, Gaa−/− and wild-type GAA+/+ mice receiving no HSCs were used as control groups. Numerical values in parentheses at the end of each group name (e.g., 0.75, 1.5, and 3) correspond to MOI of transduced cells.

FIG. 22 shows bar graphs illustrating changes in vacuolation in the quadriceps femoris muscle, heart, diaphragm and brain at week 32 post-treatment of male (M) or female (F) Gaa−/− mice (GILTco1-m) treated with lineage negative HSCs transduced with a codon-optimized transgene encoding GAA protein fused to a GILT tag (GILT.GAA protein) containing an Ala amino acid substitution at a position corresponding to Arg37 of a mature human insulin-like growth factor II (IGF) protein (SEQ ID NO: 15). For comparison, Gaa−/− and wild-type GAA+/+ mice receiving no HSCs were used as control groups (NT). Numerical values in parentheses at the end of each group name (e.g., 0.75, 1.5, and 3) correspond to MOI of transduced cells. For severity scoring of vacuolation, a score was assigned as minimally (score 1) affected tissues having <50% of cells within the section with small discrete centralized regions of cytoplasmic vacuolation involving <10% of the cytoplasmic volume; mildly (score 2) affected tissues having larger regions of vacuolation involving >10% of the cytoplasmic volume affecting <50% of cells within the section and none to rare myofibers that were diffusely enlarged with overall decreased cytoplasmic staining intensity; moderately (score 3) affected tissues having regions of cytoplasmic vacuolation involving >10% of cell with >50% of myofibers showing evidence of myofiber degeneration characterized by enlargement of myofibers and overall decreased staining intensity; and markedly (score 4) affected tissues having overall enlargement and decreased staining intensity of the majority of myofibers with both centralized regions of cytoplasmic vacuolation and evidence of myofiber degeneration.

FIG. 23 shows a graph illustrating left ventricle mass index in male (M) or female (F) Gaa−/− mice (GILTco1-m) treated with lineage negative HSCs transduced with a codon-optimized transgene encoding GAA protein fused to a GILT tag (GILT.GAA protein) containing an Ala amino acid substitution at a position corresponding to Arg37 of a mature human insulin-like growth factor II (IGF) protein (SEQ ID NO: 15). For comparison, Gaa−/− and wild-type GAA+/+ mice receiving no HSCs (NT) were used as control groups. Numerical values in parentheses at the end of each group name (e.g., 0.75, 1.5, and 3) correspond to MOI of transduced cells.

FIGS. 24A-24D show bar graphs illustrating rotarod assessment at constant rotating speed (FIG. 24A) or accelerating speed (FIG. 24B), wire hang assessment (FIG. 24C), and gait analysis (FIG. 24D) in male or female Gaa−/− mice (GILTco1-m) treated with lineage negative HSCs transduced with a codon-optimized transgene encoding GAA protein fused to a GILT tag (GILT.GAA protein) containing an Ala amino acid substitution at a position corresponding to Arg37 of a mature human insulin-like growth factor II (IGF) protein (SEQ ID NO: 15). For comparison, Gaa−/− and wild-type GAA+/+ mice receiving no HSCs (NT) were used as control groups. Numerical values in parentheses at the end of each group name (e.g., 0.75, 1.5, and 3) correspond to MOI of transduced cells.

### DEFINITIONS

As used herein, the terms “ablate,” “ablating,” “ablation,” and the like refer to the depletion of one or more cells in a population of cells in vivo or ex vivo. In some embodiments of the present disclosure, it may be desirable to ablate endogenous cells within a subject (e.g., a subject undergoing treatment for Pompe disease) before administering a therapeutic population of cells (e.g., pluripotent cells, embryonic stem cells (ESCs), induced pluripotent stem cells (iPSCs), multipotent cells, CD34+ cells, hematopoietic stem cells (HSCs), myeloid progenitor cells (MPCs), blood line progenitor cells (BLPCs), monocytes, macrophages, microglial progenitor cells, or microglia) to the subject. This can be beneficial, for example, in order to provide the newly-administered cells with an environment within which the cells may engraft. Ablation of a population of cells can be performed in a manner that selectively targets a specific cell type, for example, using antibody-drug conjugates that bind to an antigen expressed on the target cell and subsequently engender the killing of the target cell. Additionally or alternatively, ablation may be performed in a non-specific manner using cytotoxins that do not localize to a particular cell type, but are instead capable of exerting their cytotoxic effects on a variety of different cells. Exemplary agents that may be used to ablate a population of endogenous cells in a subject, such as a population of endogenous microglia or microglial precursor cells in a subject undergoing therapy, e.g., for the treatment of Pompe disease, are busulfan, PLX3397, PLX647, PLX5622, treosulfan, clodronate liposomes, and combinations thereof. Examples of ablation include depletion of at least 5% of cells (e.g., at least 5%, 10%, 15%, 20%, 25%, 30%, 35%, 40%, 45%, 50%, or more) in a population of cells in vivo or in vitro. Quantifying cell counts within a sample of cells can be performed using a variety of cell-counting techniques, such as through the use of a counting chamber, a Coulter counter, flow cytometry, or other cell-counting methods known in the art.

As used herein, “administration” refers to providing or giving a subject a therapeutic agent (e.g., cells described herein) that includes a transgene (e.g., a transgene capable of expression in macrophages or microglia) encoding one or more acid alpha-glucosidase (GAA) proteins, by any effective route. Exemplary routes of administration are described herein and below (e.g. intracerebroventricular (ICV) injection, intrathecal (IT) injection, intraparenchymal (IP) injection, intravenous (IV) injection, and stereotactic injection).

As used herein, “allogeneic” means cells, tissue, DNA, or factors taken or derived from a different subject of the same species. For example, in the context of transduced, GAA-expressing cells that are administered to a subject for the treatment of Pompe disease, allogeneic cells may be cells that are obtained from a subject that is not the subject and are then transduced or transfected with a vector that directs the expression of GAA. The phrase “directs expression” refers to the polynucleotide containing a sequence that encodes the molecule to be expressed. The polynucleotide may contain additional sequence that enhances expression of the molecule in question.

As used herein, “autologous” refers to cells, tissue, DNA, or factors taken or derived from an individual's own tissues, cells, or DNA. For example, in the context of transduced, GAA-expressing cells that are administered to a subject for the treatment of Pompe disease, the autologous cells may be cells obtained from the subject that are then transduced or transfected with a vector that directs the expression of GAA.

As used herein, the term “ApoE” refers to apolipoprotein E, a member of a class of proteins involved in lipid transport. Apolipoprotein E is a fat-binding protein (apolipoprotein) that is part of the chylomicron and intermediate-density lipoprotein (IDLs). These are essential for the normal processing (catabolism) of triglyceride-rich lipoproteins. ApoE is encoded by the APOE gene. The term “ApoE” also refers to variants of the wild type ApoE protein, such as proteins having at least 85% identity (e.g., at least 85%, 86%, 87%, 88%, 89%, 90%, 95%, 96%, 97%, 98%, 99%, or 99.9% identity, or more) to the amino acid sequence of wild type ApoE, which is set forth in SEQ ID NO: 24.

As used herein, the term “blood lineage progenitor cell” or “BLPC” refers to any cell (e.g., a mammalian cell) capable of differentiating into one or more (e.g., 2, 3, 4, 5 or more) types of hematopoietic (i.e., blood) cells. A BLPC may differentiate into erythrocytes, leukocytes (e.g., such as granulocytes (e.g., basophils, eosinophils, neutrophils, and mast cells) or agranulocytes (e.g., lymphocytes and monocytes)), or thrombocytes. A BLPC may also include a differentiated blood cell (e.g., a monocyte) that can further differentiate into another blood cell type (e.g., a macrophage).

As used herein, the term “cell type” refers to a group of cells sharing a phenotype that is statistically separable based on gene expression data. For example, cells of a common cell type may share similar structural and/or functional characteristics, such as similar gene activation patterns and antigen presentation profiles. Cells of a common cell type may include those that are isolated from a common tissue (e.g., epithelial tissue, neural tissue, connective tissue, or muscle tissue) and/or those that are isolated from a common organ, tissue system, blood vessel, or other structure and/or region in an organism.

As used herein, the term “cistron” refers to a segment of a DNA or RNA sequence encoding a single protein or polypeptide product.

As used herein, “codon optimization” refers a process of modifying a nucleic acid sequence in accordance with the principle that the frequency of occurrence of synonymous codons (e.g., codons that code for the same amino acid) in coding DNA is biased in different species. Such codon degeneracy allows an identical polypeptide to be encoded by a variety of nucleotide sequences. Sequences modified in this way are referred to herein as “codon-optimized.” This process may be performed on any of the sequences described in this specification to enhance expression or stability. Codon optimization may be performed in a manner such as that described in, e.g., U.S. Pat. Nos. 7,561,972, 7,561,973, and 7,888,112, each of which is incorporated herein by reference in its entirety. The sequence surrounding the translational start site can be converted to a consensus Kozak sequence according to known methods. See, e.g., Kozak et al, Nucleic Acids Res. 15:8125-8148, incorporated herein by reference in its entirety. Multiple stop codons can be incorporated.

As used herein, the terms “condition” and “conditioning” refer to processes by which a subject is prepared for receipt of a transplant containing cells (e.g., pluripotent cells, ESCs, iPSCs, multipotent cells, CD34+ cells, HSCs, MPCs, BLPCs, monocytes, macrophages, microglial progenitor cells, or microglia). Such procedures promote the engraftment of a cell transplant, for example, by selectively depleting endogenous microglia or HSCs, thereby creating a vacancy filled by an exogenous cell transplant. According to the methods described herein, a subject may be conditioned for cell transplant therapy by administration to the subject of one or more agents capable of ablating endogenous microglia and/or hematopoietic stem or progenitor cells (e.g., busulfan, treosulfan, PLX3397, PLX647, PLX5622, and clodronate liposomes), radiation therapy, or a combination thereof. Conditioning may be myeloablative or non-myeloablative. Other cell-ablating agents and methods well known in the art (e.g., antibody-drug conjugates) may also be used.

As used herein, the terms “conservative mutation,” “conservative substitution,” and “˜conservative amino acid substitution” refer to a substitution of one or more amino acids for one or more different amino acids that exhibit similar physicochemical properties, such as polarity, electrostatic charge, and steric volume. These properties are summarized for each of the twenty naturally-occurring amino acids in Table 1 below.

TABLE 1
Representative physicochemical properties
of naturally occurring amino acids

| | | | | Electrostatic | |
|---|---|---|---|---|---|
| | 3 | 1 | Side- | character at | |
| | Letter | Letter | chain | physiological | Steric |
| Amino Acid | Code | Code | Polarity | pH (7.4) | Volume^{†} |
| Alanine | Ala | A | nonpolar | neutral | small |
| Arginine | Arg | R | polar | cationic | large |
| Asparagine | Asn | N | polar | neutral | intermediate |
| Aspartic | Asp | D | polar | anionic | intermediate |
| acid | | | | | |
| Cysteine | Cys | C | nonpolar | neutral | intermediate |
| Glutamic | Glu | E | polar | anionic | intermediate |
| acid | | | | | |
| Glutamine | Gln | Q | polar | neutral | intermediate |
| Glycine | Gly | G | nonpolar | neutral | small |
| Histidine | His | H | polar | Both neutral | large |
| | | | | and cationic | |
| | | | | forms in | |
| | | | | equilibrium | |
| | | | | at pH 7.4 | |
| Isoleucine | Ile | I | nonpolar | neutral | large |
| Leucine | Leu | L | nonpolar | neutral | large |
| Lysine | Lys | K | polar | cationic | large |
| Methionine | Met | M | nonpolar | neutral | large |
| Phenylalanine | Phe | F | nonpolar | neutral | large |
| Proline | Pro | P | nonpolar | neutral | intermediate |
| Serine | Ser | S | polar | neutral | small |
| Threonine | Thr | T | polar | neutral | intermediate |
| Tryptophan | Trp | W | nonpolar | neutral | bulky |
| Tyrosine | Tyr | Y | polar | neutral | large |
| Valine | Val | V | nonpolar | neutral | intermediate |

^{†}based on volume in A³: 50-100 is small, 100-150 is intermediate, 150-200 is large, and >200 is bulky

From this table it is appreciated that the conservative amino acid families include (i) G, A, V, L and I; (ii) D and E; (iii) C, S and T; (iv) H, K and R; (v) N and Q; and (vi) F, Y and W. A conservative mutation or substitution is therefore one that substitutes one amino acid for a member of the same amino acid family (e.g., a substitution of Ser for Thr or Lys for Arg).

As used herein, the terms “effective amount,” “therapeutically effective amount,” and a “sufficient amount” of composition, vector construct, viral vector, or cell described herein refer to a quantity sufficient to, when administered to the subject, including a mammal, for example a human, effect beneficial or desired results, including clinical results. As such, an “effective amount” or synonym thereof depends upon the context in which it is being applied. For example, in the context of treating Pompe disease, it is an amount of the composition, vector construct, viral vector, or cell sufficient to achieve a treatment response as compared to the response obtained without administration of the composition, vector construct, viral vector, or cell. The amount of a given composition described herein that will correspond to such an amount will vary depending upon various factors, such as the given agent, the pharmaceutical formulation, the route of administration, the type of disease or disorder, the identity of the subject (e.g., age, sex, weight) or host being treated, and the like, but can nevertheless be determined by one skilled in the art. Also, as used herein, a “therapeutically effective amount” of a composition, vector construct, viral vector, or cell of the present disclosure is an amount which results in a beneficial or desired result in a subject as compared to a control. As defined herein, a therapeutically effective amount of a composition, vector construct, viral vector, or cell of the present disclosure may be readily determined by one of ordinary skill by methods known in the art. Dosage regime may be adjusted to provide the optimum therapeutic response.

As used herein, the terms “embryonic stem cell” and “ES cell” refer to an embryo-derived totipotent or pluripotent stem cell, derived from the inner cell mass of a blastocyst that can be maintained in an in vitro culture under suitable conditions. ES cells are capable of differentiating into cells of any of the three vertebrate germ layers, e.g., the endoderm, the ectoderm, or the mesoderm. ES cells are also characterized by their ability propagate indefinitely under suitable in vitro culture conditions. See, for example, Thomson et al., Science 282:1145 (1998).

As used herein, the term “endogenous” describes a molecule (e.g., a polypeptide, nucleic acid, or cofactor) that is found naturally in a particular organism (e.g., a human) or in a particular location within an organism (e.g., an organ, a tissue, or a cell, such as a human cell).

As used herein, the term “engraft” and “engraftment” refer to the process by which hematopoietic stem cells and progenitor cells, whether such cells are produced endogenously within the body or transplanted using any of the administration methods described herein (e.g. intravenous injection, intracerebroventricular injection, intraosseous injection, and/or bone marrow transplant), repopulate a tissue. The term encompasses all events surrounding or leading up to engraftment, such as tissue homing of cells and colonization of cells within the tissue of interest.

As used herein, the term “enzyme replacement therapy” refers to the administration to a subject (e.g., a mammalian subject, such as a human) suffering from a genetic loss-of-function disease of the protein that is naturally defective or deficient in the subject. For example, in the context of a subject having Pompe disease, enzyme replacement therapy refers to administration of GAA protein to such a subject. Typically, enzyme replacement therapy involves administration of the therapeutic protein to the subject chronically, over the course of multiple doses throughout the subject's life.

As used herein, the term “express” refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein. Expression of a gene of interest in a subject can manifest, for example, by detecting: an increase in the quantity or concentration of mRNA encoding a corresponding protein (as assessed, e.g., using RNA detection procedures described herein or known in the art, such as quantitative polymerase chain reaction (qPCR) and RNA seq techniques), an increase in the quantity or concentration of a corresponding protein (as assessed, e.g., using protein detection methods described herein or known in the art, such as enzyme-linked immunosorbent assays (ELISA), among others), and/or an increase in the activity of a corresponding protein (e.g., in the case of an enzyme, as assessed using an enzymatic activity assay described herein or known in the art) in a sample obtained from the subject.

As used herein, the term “exogenous” describes a molecule (e.g., a polypeptide, nucleic acid, or cofactor) that is not found naturally in a particular organism (e.g., a human) or in a particular location within an organism (e.g., an organ, a tissue, or a cell, such as a human cell). Exogenous materials include those that are provided from an external source to an organism or to cultured matter extracted there from.

As used herein, the term “functional potential” as it pertains to a stem cell, such as a hematopoietic stem cell, refers to the functional properties of stem cells which include: 1) multi-potency (which refers to the ability to differentiate into multiple different blood lineages including, but not limited to granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., natural killer (NK) cells, B-cells and T-cells); 2) self-renewal (which refers to the ability of stem cells to give rise to daughter cells that have equivalent potential as the mother cell, and further that this ability can repeatedly occur throughout the lifetime of an individual without exhaustion); and 3) the ability of stem cells or progeny thereof to be reintroduced into a transplant recipient whereupon they home to the stem cell niche and re-establish productive and sustained cell growth and differentiation.

As used herein, the terms “hematopoietic stem cells” and “HSCs” refer to immature blood cells having the capacity to self-renew and to differentiate into mature blood cells of diverse lineages including but not limited to granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). It is known in the art that such cells may or may not include CD34+ cells. CD34+ cells are immature cells that express the CD34 cell surface marker. In humans, CD34+ cells are believed to include a subpopulation of cells with the stem cell properties defined above, whereas in mice, HSCs are CD34−. In addition, HSCs also refer to long term repopulating HSC (LT-HSC) and short-term repopulating HSC (ST-HSC). LT-HSC and ST-HSC are differentiated, based on functional potential and on cell surface marker expression. For example, human HSC are a CD34+, CD38−, CD45RA−, CD90+, CD49F+, and lin− (negative for mature lineage markers including CO2, CD3, CD4, CD7, CD8, CD10, CD11B, CD19, CD20, CD56, CD235A). In mice, bone marrow LT-HSC are CD34−, SCA-1+, C-kit+, CD135−, Slamf1/CD150+, CD48−, and lin− (negative for mature lineage markers including Ter119, CD11 b, Gr1, CD3, CD4, CD8, B220, IL-7ra), whereas ST-HS Care CD34+, SCA-1+, C-kit+, CD135−, Slamf1/CD150+, and lin− (negative for mature lineage markers including Ter119, CD11 b, Gr1, CD3, CD4, CD8, B220, IL-7ra). In addition, ST-HSC are less quiescent (i.e., more active) and more proliferative than LT-HSC under homeostatic conditions. However, LT-HSC have greater self-renewal potential (i.e., they survive throughout adulthood, and can be serially transplanted through successive recipients), whereas ST-HSC have limited self-renewal (i.e., they survive for only a limited period of time, and do not possess serial transplantation potential). Any of these HSCs can be used in any of the methods described herein. Optionally, ST-HSCs are useful because they are highly proliferative and thus, can more quickly give rise to differentiated progeny.

As used herein, the term “HLA-matched” refers to a donor-recipient pair in which none of the HLA antigens are mismatched between the donor and recipient, such as a donor providing a hematopoietic stem cell graft to a recipient in need of hematopoietic stem cell transplant therapy. HLA-matched (i.e., where all of the 6 alleles are matched) donor-recipient pairs have a decreased risk of graft rejection, as endogenous T cells and NK cells are less likely to recognize the incoming graft as foreign, and are thus less likely to mount an immune response against the transplant.

As used herein, the term “HLA-mismatched” refers to a donor-recipient pair in which at least one HLA antigen, in particular with respect to HLA-A, HLA-B, HLA-C, and HLA-DR, is mismatched between the donor and recipient, such as a donor providing a hematopoietic stem cell graft to a recipient in need of hematopoietic stem cell transplant therapy. In some embodiments, one haplotype is matched and the other is mismatched. HLA-mismatched donor-recipient pairs may have an increased risk of graft rejection relative to HLA-matched donor-recipient pairs, as endogenous T cells and NK cells are more likely to recognize the incoming graft as foreign in the case of an HLA-mismatched donor-recipient pair, and such T cells and NK cells are thus more likely to mount an immune response against the transplant.

As used herein, the terms “induced pluripotent stem cell,” “iPS cell,” and “iPSC” refer to a pluripotent stem cell that can be derived directly from a differentiated somatic cell. Human iPS cells can be generated by introducing specific sets of reprogramming factors into a non-pluripotent cell that can include, for example, Oct3/4, Sox family transcription factors (e.g., Sox1, Sox2, Sox3, Soxl5), Myc family transcription factors (e.g., c-Myc, 1-Myc, n-Myc), Kruppel-like family (KLF) transcription factors (e.g., KLF1, KLF2, KLF4, KLF5), and/or related transcription factors, such as NANOG, LIN28, and/or Glis1. Human iPS cells can also be generated, for example, by the use of miRNAs, small molecules that mimic the actions of transcription factors, or lineage specifiers. Human iPS cells are characterized by their ability to differentiate into any cell of the three vertebrate germ layers, e.g., the endoderm, the ectoderm, or the mesoderm. Human iPS cells are also characterized by their ability propagate indefinitely under suitable in vitro culture conditions. See, for example, Takahashi and Yamanaka, Cell 126:663 (2006).

As used herein, the term “IRES” refers to an internal ribosome entry site. In general, an IRES sequence is a feature that allows eukaryotic ribosomes to bind an mRNA transcript and begin translation without binding to a 5′ capped end. An mRNA containing an IRES sequence produces two translation products, one initiating form the 5′ end of the mRNA and the other from an internal translation mechanism mediated by the IRES.

As used herein, the term “lymphoid cell” refers to white blood cells of the lymphoid lineage that are derived from a common lymphoid progenitor cell. Lymphoid cells are generally part of the adaptive immunity arm of the immune system and include cells such as NK cells, B-cells, and T-cells. As used herein, lymphoid cells refers to a population of cells that cannot differentiate into cells of the myeloid lineage (e.g., MPCs, erythrocytes, mast cells, megakaryocytes, thrombocytes, myeloblasts, basophils, neutrophils, eosinophils, monocytes, and macrophages).

As used herein, the term “macrophage” refers to a type of white blood cell that engulfs and digests cellular debris, foreign substances, microbes, cancer cells, and anything else that does not have the types of proteins specific to healthy body cells on its surface in a process called phagocytosis. Macrophages are found in essentially all tissues, where they patrol for potential pathogens by amoeboid movement. They take various forms (with various names) throughout the body (e.g., histiocytes, Kupffer cells, alveolar macrophages, microglia, and others), but all are part of the mononuclear phagocyte system. Besides phagocytosis, they play a critical role in non-specific defense (innate immunity) and also 20 help initiate specific defense mechanisms (adaptive immunity) by recruiting other immune cells such as lymphocytes. For example, they are important as antigen presenters to T cells. Beyond increasing inflammation and stimulating the immune system, macrophages also play an important anti-inflammatory role and can decrease immune reactions through the release of cytokines.

As used herein, the terms “microglia” or “microglial cell” refer to a type of neuroglial cell found in the brain and spinal cord that function as resident macrophage cells and the principal line of immune defense in the central nervous system. Primary functions of microglial cells include immune surveillance, phagocytosis, extracellular signaling (e.g., production and release of cytokines, chemokines, prostaglandins, and reactive oxygen species), antigen presentation, and promotion of tissue repair and regeneration.

As used herein, the term “microglial progenitor cell” refers to a precursor cell that gives rise to microglial cells. Microglial precursor cells originate in the yolk sac during a limited period of embryonic development, infiltrate the brain mesenchyme, and perpetually renew themselves throughout life.

As used herein, the term “miRNA targeting sequence” refers to a nucleotide sequence located in the 3′-UTR of a target mRNA molecule which is complementary to a specific miRNA molecule (e.g. miR-126) such that they may hybridize and promote RNA-induced silencing complex-dependent and Dicer-dependent mRNA destabilization and/or cleavage, thereby preventing the expression of an mRNA transcript.

As used herein, the term “MND promoter” refers to a synthetic, constitutively active promoter derived from a myeloproliferative sarcoma virus (MSV). An MND promoter contains an MSV enhancer, a U3 region of a Maloney Murine Leukemia Virus, a deletion of a negative control region, and a substitution of a dl587rev primer binding site. An MND promoter may have, for example, the nucleic acid sequence of SEQ ID NO: 10 or SEQ ID NO: 11 or may be a variant thereof having at least 85% (e.g., at least 85%, 90%, 95%, 96%, 97%, 98%, 99%, or more) sequence identity to the nucleic acid sequence of SEQ ID NO: 10 or SEQ ID NO: 11. According to the disclosed methods and compositions, the MND promoter is suitable for incorporation into a transgene expression construct (e.g., a plasmid or viral vector) for driving expression of a GAA protein fused to a glycosylation-independent lysosomal targeting (GILT) tag (GILT.GAA protein)-encoding transgene in one or more target cell types.

As used herein, the term “monocistronic” refers to an RNA or DNA construct that contains the coding sequence for a single protein or polypeptide product.

As used herein, the term “monocyte” refers to a type of white blood cell (i.e., a leukocyte) that is capable of differentiating into macrophages and myeloid lineage dendritic cells. Monocytes constitute an important component of the vertebrate adaptive immune response. Three different types of monocytes are known to exist, including classical monocytes characterized by strong expression of the CD14 cell surface receptor and no CD16 expression (i.e., CD14++CD16−), non-classical monocytes exhibiting low levels of CD14 expression and co-expression of C16 (CD14+CD16+), and intermediate monocytes exhibiting high levels of CD14 expression and low levels of C16 expression (CD14++CD16+). Monocytes perform a variety of functions that serve the immune system, including phagocytosis, antigen presentation, and cytokine secretion.

As used herein, the term “multipotent cell” refers to a cell that possesses the ability to develop into multiple (e.g., 2, 3, 4, 5, or more) but not all differentiated cell types. Non-limiting examples of multipotent cells include cells of the hematopoietic lineage (e.g., granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). Examples of multipotent cells are CD34+ cells.

As used herein, the term “mutation” refers to a change in the nucleotide sequence of a gene. Mutations in a gene may occur naturally as a result of, for example, errors in DNA replication, DNA repair, irradiation, and exposure to carcinogens or mutations may be induced as a result of administration of a transgene expressing a mutant gene. Mutations may result from a single nucleotide substitution or deletion. The nomenclature for describing mutations and sequence variations uses the format “reference sequence.code,” wherein the reference sequence may be “c,” designating a coding DNA and the code may contain symbols including “>,” designating a single nucleotide substitution, “del,” designating a deletion, or may contain “a+b” in reference to substitutions occurring within an intron, wherein x denotes a number corresponding to a nucleotide within the coding DNA sequence (e.g., a nucleotide within an exon of a coding DNA sequence) and y corresponds to the number of nucleotides 3′ relative to x. Mutations may also result in a substitution of a single amino acid within the peptide chain. The nomenclature for describing mutations resulting amino acid substitutions uses the format “p.AnB,” where “p” designates the variation at the level of the protein, “A” designates the amino acid found in the wild type variant of the protein, “n” designates the number of the amino acid within the peptide chain, and “B” designates the new amino acid that resulted from the substitution.

As used herein, the term “myeloablative” or “myeloablation” refers to a conditioning regiment that substantially impairs or destroys the hematopoietic system, typically by exposure to a cytotoxic agent (e.g., busulfan) or radiation. Myeloablation encompasses complete myeloablation brought on by high doses of cytotoxic agent or total body irradiation that destroys the hematopoietic system.

As used herein, the term “myeloid cells” refers to blood cells derived from the bone marrow that belong to the myeloid cell lineage and arise from common myeloid progenitor cells that gives rise to granulocytes, monocytes, erythrocytes, and platelets. Non-limiting examples of myeloid cells include MPCs, erythrocytes, mast cells, megakaryocytes, thrombocytes, myeloblasts, basophils, neutrophils, eosinophils, monocytes, and macrophages. As used herein, myeloid cells refer specifically to a group of myeloid lineage cells that are not capable if differentiating into cells of the lymphoid lineage (e.g., NK cells, T-cells, B-cells, or plasma cells).

As used herein, the term “non-myeloablative” or “myelosuppressive” refers to a conditioning regiment that does not eliminate substantially all hematopoietic cells of host origin.

As used herein, the phrase “penetrate(s) the blood brain barrier (BBB)” refers to the ability of a therapeutic agent (e.g., a GAA protein fused to a GILT tag disclosed herein) to cross the BBB—a semipermeable layer of endothelial cells lining vascular tissue that prevents non-selective access of solutes in the blood from into the central nervous system (CNS). The tight packing of endothelial cells at the BBB prevents molecules larger than 400 daltons from entering the CNS, thereby posing a significant barrier to therapeutic efficacy of an agent in the absence agents that promote passage through the BBB. Accordingly, the present disclosure features GILT.GAA fusion proteins further containing a receptor-binding peptide (Rb) derived from apolipoprotein E (ApoE), which binds to low density lipoprotein receptor superfamily (LDLRf) members expressed on endothelial cells and brain parenchyma and promotes translocation of proteins across the BBB into the brain.

As used herein, the term “pluripotent cell” refers to a cell that possesses the ability to develop into more than one differentiated cell type, such as a cell type of the hematopoietic lineage (e.g., granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). Examples of pluripotent cells are ESCs and iPSCs.

As used herein, the term “plasmid” refers to a to an extrachromosomal circular double stranded DNA molecule into which additional DNA segments may be ligated. A plasmid is a type of vector, a nucleic acid molecule capable of transporting another nucleic acid to which it has been linked. Certain plasmids are capable of autonomous replication in a host cell into which they are introduced (e.g., bacterial plasmids having a bacterial origin of replication and episomal mammalian plasmids). Other vectors (e.g., non-episomal mammalian vectors) can be integrated into the genome of a host cell upon introduction into the host cell, and thereby are replicated along with the host genome. Certain plasmids are capable of directing the expression of genes to which they are operably linked.

As used herein, the term “polycistronic” refers to an RNA or DNA construct that contains the coding sequence for at least two protein or polypeptide products.

As used herein, the term “promoter” refers to a recognition site on DNA that is bound by an RNA polymerase. The polymerase drives transcription of the transgene. Exemplary promoters suitable for use with the compositions and methods described herein include synthetic promoters, which are regulatory nucleic acids that do not occur naturally in biological systems. Synthetic promoters contain parts of naturally occurring promoters combined with nucleic acids that do not occur in nature and can be optimized to express recombinant DNA using a variety of transgenes, vectors, and target cell types.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

As used herein, the term “pharmaceutically acceptable” refers to those compounds, materials, compositions and/or dosage forms, which are suitable for contact with the tissues of a subject, such as a mammal (e.g., a human) without excessive toxicity, irritation, allergic response and other problem complications commensurate with a reasonable benefit/risk ratio.

As used herein, a potent “receptor-binding peptide (Rb) derived from ApoE” has the ability to translocate proteins across the BBB into the brain when engineered as fusion proteins. This method can therefore function to selectively open the BBB for therapeutic agents (e.g., soluble GAA) when engineered as a fusion protein. This peptide can be readily attached to diagnostic or therapeutic agents without jeopardizing their biological functions or interfering with the important biological functions of ApoE due to the utilization of the Rb domain of ApoE, rather than the entire ApoE protein. This pathway is also an alternative uptake pathway that can facilitate further/secondary distribution within the brain after the agents reach the CNS due to the widespread expression of LDLRf members in brain parenchyma. Exemplary Rb domains can be found in the N-terminus of ApoE. For example, Rb domains useful in conjunction with the compositions and methods described herein are polypeptides having the amino acid sequence of residues 1 to 191 of SEQ ID NO: 24, residues 25 to 185 of SEQ ID NO: 24, residues 50 to 180 of SEQ ID NO: 24, residues 75 to 175 of SEQ ID NO: 24, residues 100 to 170 of SEQ ID NO: 24, or residues 125 to 165 of SEQ ID NO: 24, as well as variants thereof, such as polypeptides having at least 85% (e.g., 90%, 95%, 96%, 97%, 98%, 99%, or more) sequence identity (e.g., at least 85%, 86%, 87%, 88%, 89%, 90%, 95%, 96%, 97%, 98%, 99%, or greater, sequence identity) with respect to any of these sequences. An exemplary Rb domain is the region of ApoE having the amino acid sequence of residues 159 to 167 of SEQ ID NO: 24.

As used herein, the term “regulatory sequence” includes promoters, enhancers and other expression control elements (e.g., polyadenylation signals) that control the transcription or translation of the antibody chain genes. Such regulatory sequences are described, for example, in Perdew et al., Regulation of Gene Expression (Humana Press, New York, NY, (2014)); incorporated herein by reference.

As used herein, the term “sample” refers to a specimen (e.g., blood, blood component (e.g., serum or plasma), urine, saliva, amniotic fluid, cerebrospinal fluid, tissue (e.g., muscle, nervous, placental, or dermal), pancreatic fluid, chorionic villus sample, and cells), isolated from a subject.

As used herein, the term “signal peptide” refers to a short (usually between 16-30 amino acids) peptide region that directs translocation of the translated protein from the cytoplasm of the host to the lipid membrane for anchoring. Such signal peptides are generally located at the amino terminus of the newly translated protein. In some embodiments, the signal peptide is linked to the amino terminus. Typically, signal peptides are cleaved during transit through the endoplasmic reticulum. Cleavage is not essential as long as the protein retains its desired activity. Exemplary signal peptide includes the GAA signal peptide.

As used herein, the term “splice variant” refers to a transcribed product (i.e. RNA) of a single gene that can be processed to produce different mRNA molecules as a result of alternative inclusion or exclusion of specific exons (e.g. exon skipping) within the precursor mRNA. Proteins produced from translation of specific splice variants may differ in their structure and biological activity.

As used herein, the terms “stem cell” and “undifferentiated cell” refer to a cell in an undifferentiated or partially differentiated state that has the developmental potential to differentiate into multiple cell types. A stem cell is capable of proliferation and giving rise to more such stem cells while maintaining its functional potential. Stem cells can divide asymmetrically, which is known as obligatory asymmetrical differentiation, with one daughter cell retaining the functional potential of the parent stem cell and the other daughter cell expressing some distinct other specific function, phenotype and/or developmental potential from the parent cell. The daughter cells themselves can be induced to proliferate and produce progeny that subsequently differentiate into one or more mature cell types, while also retaining one or more cells with parental developmental potential. A differentiated cell may derive from a multipotent cell, which itself is derived from a multipotent cell, and so on. Alternatively, some of the stem cells in a population can divide symmetrically into two stem cells. Accordingly, the term “stem cell” refers to any subset of cells that have the developmental potential, under particular circumstances, to differentiate to a more specialized or differentiated phenotype, and which retain the capacity, under certain circumstances, to proliferate without substantially differentiating. In some embodiments, the term stem cell refers generally to a naturally occurring parent cell whose descendants (progeny cells) specialize, often in different directions, by differentiation, e.g., by acquiring completely individual characters, as occurs in progressive diversification of embryonic cells and tissues. Some differentiated cells also have the capacity to give rise to cells of greater developmental potential. Such capacity may be natural or may be induced artificially upon treatment with various factors. Cells that begin as stem cells might proceed toward a differentiated phenotype, but then can be induced to “reverse” and re-express the stem cell phenotype, a term often referred to as “dedifferentiation” or “reprogramming” or “retrodifferentiation” by persons of ordinary skill in the art.

As used herein, the term “transfection” refers to any of a wide variety of techniques commonly used for the introduction of exogenous DNA into a prokaryotic or eukaryotic host cell, e.g., electroporation, lipofection, calcium-phosphate precipitation, DEAE-dextran transfection, Nucleofection, squeeze-poration, sonoporation, optical transfection, Magnetofection, impalefection, and the like.

As used herein, the term “transgene” refers to a recombinant nucleic acid (e.g., DNA or cDNA) encoding a gene product (e.g., GAA). The gene product may be an RNA, peptide, or protein. In addition to the coding region for the gene product, the transgene may include or be operably linked to one or more elements to facilitate or enhance expression, such as a promoter, enhancer(s), destabilizing domain(s), response element(s), reporter element(s), insulator element(s), polyadenylation signal(s) and/or other functional elements. Embodiments of the disclosure may utilize any known suitable promoter, enhancer(s), destabilizing domain(s), response element(s), reporter element(s), insulator element(s), polyadenylation signal(s), and/or other functional elements.

As used herein, the terms “subject” and “patient” refer to an animal (e.g., a mammal, such as a human). A subject to be treated according to the methods described herein may be one who has been diagnosed with Pompe disease, or one at risk of developing these conditions. Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition.

As used herein, the term “infantile-onset Pompe disease” refers to the newborn/infantile form of Pompe disease. The infantile form typically presents within the first few months after birth with characteristics such as cardiomegaly, hypotonia, cardiomyopathy, respiratory distress, muscle weakness, feeding difficulties, and failure to thrive. This results in symptoms such as floppy baby appearance, delayed motor development, feeding difficulties, moderate hepatomegaly, macroglossia, wide open mouth, wide open eyes, nasal flaring, poor facial muscle tone, increased respiratory rate, engagement of accessory muscles for breathing, frequent chest infections, reduced air flow in the left lower zone, arrhythmias, and heart failure. Infantile-onset Pompe disease is generally characterized by residual GAA activity of less than 2% of normal activity.

As used herein, the term “late-onset Pompe disease” refers to the late onset form of Pompe disease, which occurs later in life and is distinguished from the infantile form on the basis of lack of cardiac involvement, slower progression, and prominent skeletal involvement—particularly in the lower limbs. The late onset symptoms may include impaired cough, chest infections, hypotonia, progressive muscle weakness, delayed motor development, difficulty masticating and swallowing, and lower vital capacity. Clinical outcome is generally dependent on the age of onset with better outcomes associated with later symptom onset. Late-onset Pompe disease is generally characterized by residual GAA activity of 10-20% of normal activity.

As used herein, the terms “transduction” and “transduce” refer to a method of introducing a viral vector construct or a part thereof into a cell and subsequent expression of a transgene encoded by the vector construct or part thereof in the cell.

As used herein, “treatment” and “treating” refer to an approach for obtaining beneficial or desired results, e.g., clinical results. Beneficial or desired results can include, but are not limited to, alleviation or amelioration of one or more symptoms or conditions, diminishment of extent of disease or condition, stabilized (i.e., not worsening) state of disease, disorder, or condition, preventing spread of disease or condition, delay or slowing the progress of the disease or condition, amelioration or palliation of the disease or condition, and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. “Treatment” can also mean prolonging survival as compared to expected survival if not receiving treatment. Those in need of treatment include those already with the condition or disorder, as well as those prone to have the condition or disorder or those in which the condition or disorder is to be prevented. In the context of Pompe disease, clinically desirable outcomes for a patient may include an increase in the expression levels of GAA protein or polynucleotides (e.g., DNA or RNA, such as mRNA) encoding GAA, increased GAA enzymatic activity (e.g., by 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, or more). Methods known in the art can be used for the determination of GAA protein expression levels. Exemplary methods include, e.g., ELISA assays and immunohistochemistry. Expression levels of polynucleotides encoding GAA may be ascertained by way of nucleic acid detection assays (e.g., RNA Seq). Clinically desirable patient outcomes are described herein.

As used herein, the term “atrophy” refers to reduction or degeneration of body tissues, such as muscle tissue (e.g., heart, diaphragm, gastrocnemius muscle, quadriceps femoris muscle, or tibialis anterior muscle tissue) and/or nervous tissue (e.g., cerebellum, cerebrum, thoracic or cervical spinal cord, or hippocampus tissue).

As used herein, the term “vector” includes a nucleic acid vector, e.g., a DNA vector, such as a plasmid, an RNA vector, virus, or other suitable replicon (e.g., viral vector). A variety of vectors have been developed for the delivery of polynucleotides encoding exogenous proteins into a prokaryotic or eukaryotic cell. Examples of such expression vectors are disclosed in, e.g., WO 1994/011026; incorporated herein by reference as it pertains to vectors suitable for the expression of a gene of interest. Expression vectors suitable for use with the compositions and methods described herein contain a polynucleotide sequence as well as, e.g., additional sequence elements used for the expression of proteins and/or the integration of these polynucleotide sequences into the genome of a mammalian cell. Certain vectors that can be used for the expression of GAA as described herein include plasmids that contain regulatory sequences, such as promoter and enhancer regions, which direct gene transcription. Other useful vectors for expression of GAA contain polynucleotides that enhance the rate of translation of these genes or improve the stability or nuclear export of the mRNA that results from gene transcription. These sequence elements include, e.g., 5′ and 3′ untranslated regions, an IRES, and polyadenylation signal site in order to direct efficient transcription of the gene carried on the expression vector. The expression vectors suitable for use with the compositions and methods described herein may also contain a polynucleotide encoding a marker for selection of cells that contain such a vector. Examples of a suitable marker are genes that encode resistance to antibiotics, such as ampicillin, chloramphenicol, kanamycin, nourseothricin, or zeocin.

As used herein in the context of a therapeutic protein, such as GAA, the use of the protein name refers to the gene encoding the protein or the corresponding protein product, depending upon the context, as will be appreciated by one of skill in the art. The term “GAA” includes wild-type forms of the GAA gene or protein, as well as variants (e.g., splice variants, truncations, concatemers, and fusion constructs, among others) of wild-type GAA proteins that retain therapeutic activity of the wild-type GAA protein, as well as nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to an amino acid sequence of a wild-type GAA protein, such as any one of SEQ ID NOs: 1-4, below.

### DETAILED DESCRIPTION

The present disclosure provides compositions and methods that can be used for treating glycogen storage disorders, particularly, type II glycogen storage disorder, also known as Pompe disease in a subject (such as a mammalian subject, for example, a human). Using the compositions and methods described herein, one can treat Pompe disease in a subject (e.g., a mammalian subject, such as, e.g., a human subject) by administering a population of cells (e.g., pluripotent cells, embryonic stem cells (ESCs), induced pluripotent stem cells (iPSCs), multipotent cells, CD34+ cells, hematopoietic stem cells (HSCs), myeloid progenitor cells (MPCs), blood line progenitor cells (BLPCs), monocytes, macrophages, microglial progenitor cells, or microglia) containing a transgene encoding an acid alpha-glucosidase (GAA) protein fused to a glycosylation-independent lysosomal targeting (GILT) tag (GILT.GAA protein) containing an insulin-like growth factor II (IGF-II) mutein that contains an Ala amino acid substitution at a position corresponding to Arg37 of SEQ ID NO: 15 (i.e., an R37A substitution/mutation). For example, described herein are compositions containing cells that have been modified ex-vivo to express GAA. The sections that follow describe the compositions and methods useful for the treatment of Pompe disease in further detail.

### Pompe Disease

Pompe disease is an autosomal recessive lysosomal storage disorder affecting muscle and nerve tissue. The primary pathology of this disease is excessive accumulation of glycogen within the lysosome of the cell resulting from deficient expression of the lysosomal GAA enzyme. Pathological deposition of lysosomal glycogen in Pompe patients results in a clinical presentation characterized by systemic muscle weakness (myopathy), including in muscle tissue of the heart and skeletal muscles, as well as liver and neural tissues.

Pompe disease is expressed in one of two forms, namely the newborn/infantile form (infantile-onset Pompe disease) or the late onset form (late-onset Pompe disease). The infantile form typically presents within the first few months after birth with characteristics such as cardiomegaly, hypotonia, cardiomyopathy, respiratory distress, muscle weakness, feeding difficulties, and failure to thrive. This results in symptoms such as floppy baby appearance, delayed motor development, feeding difficulties, moderate hepatomegaly, macroglossia, wide open mouth, wide open eyes, nasal flaring, poor facial muscle tone, increased respiratory rate, engagement of accessory muscles for breathing, frequent chest infections, reduced air flow in the left lower zone, arrhythmias, and heart failure. The late onset form, by definition, occurs later in life and is distinguished from the infantile form on the basis of lack of cardiac involvement, slower progression, and prominent skeletal involvement—particularly in the lower limbs. The late onset symptoms may include impaired cough, chest infections, hypotonia, progressive muscle weakness, delayed motor development, difficulty masticating and swallowing, and lower vital capacity. Clinical outcome is generally dependent on the age of onset with better outcomes associated with later symptom onset.

### Acid Alpha-Glucosidase

Pompe disease is directly linked to mutations in the GAA gene (also known as acid maltase) with an autosomal recessive inheritance pattern. Disease presentation requires that the patient inherits a defective copy of the GAA gene from each parent. The GAA gene is located on the long arm of chromosome 17 at 17q25.2-q25.3 and has so far been associated with a total of 289 mutations, 197 of which are pathogenic. The molecular basis for Pompe disease is generally due to three mutations, with a T to G transversion being the most common. This transversion results in aberrant RNA splicing by interrupting an RNA splicing site. In some cases of Pompe disease, reduced levels of the 110-kDa GAA precursor protein are observed, while in other cases normal levels of 110-kDa precursor protein are synthesized but not processed into the mature, properly glycosylated 76- and 70-kDa GAA forms.

The GAA protein is a lysosomal hydrolase that is responsible for hydrolyzing the alpha-1,4 and alpha-1,6 linkages in glycogen, maltose, and isomaltose. Reduced amounts of GAA within the lysosome results in excessive deposition of glycogen within lysosomes and cytoplasm, which disrupts normal functioning of the cells. There is generally a correlation between the severity of the disease and the residual acid GAA, the activity being 10-20% of normal in late onset and less than 2% in early onset forms of the disease.

Clinical management of Pompe disease has largely employed physical and occupational therapeutic interventions and diet control in order to mitigate disease symptoms. More recent strategies have focused on supplementing the deficient GAA levels in a patient by way of enzyme replacement therapy (ERT), which delivers a recombinant form of human GAA (rhGAA) produced from CHO cells (Myozyme and Lumizyme; Genzyme Corporation) via intravenous injection. However, ERT suffers from multiple challenges such as toxic immunogenicity, difficulty in targeting the recombinant GAA to target tissues and/or subcellular compartments, and fast clearance from the body. Unlike these treatments, the compositions and methods described herein provide the benefit of delivering a composition that provides long-lasting efficacy, reduced immunogenicity, reduced frequency of administration, and targeted delivery to affected tissues and/or subcellular compartments. As such, the compositions and methods described herein represent a potential curative therapy.

The compositions and methods described herein can be used to treat Pompe disease by administering a population of cells (e.g., pluripotent cells, ESCs, iPSCs, multipotent cells, CD34+ cells, HSCs, MPCs, BLPCs, monocytes, macrophages, microglial progenitor cells, or microglia) containing a transgene encoding a GILT.GAA protein containing the R37A IGF-II mutein. The compositions and methods described herein can be used to treat a subject with normal GAA activity, reduced GAA activity, and a subject whose GAA mutational status and/or GAA activity level is unknown. The compositions and methods described herein may also be administered as a preventative treatment to a subject at risk of developing Pompe disease, e.g., a subject with a GAA mutation or a subject with reduced GAA activity.

GAA-encoding constructs that may be used in conjunction with the compositions and methods described herein include polynucleotides that encode wild-type GAA (any one of the amino acid sequences which are shown as SEQ ID NOS. 1-4) or a variant thereof, such as a polynucleotide that encodes a protein having at least 85% (e.g., 90%, 95%, 96%, 97%, 98%, 99%, or more) sequence identity (e.g., at least 85%, 90%, 95%, 96%, 97%, 98%, 99%, or more, sequence identity) to any of the amino acid sequences of SEQ ID NOS. 1-4.

In some embodiments, the GAA has an amino acid sequence of SEQ ID NO: 1 or is a variant thereof having at least 85% (e.g., 90%, 95%, 96%, 97%, 98%, 99%, or more) sequence identity to the amino acid sequence of SEQ ID NO: 1. In some embodiments, the GAA has an amino acid sequence having at least 90% (e.g., at least 90%, 91%, 92%, 93%, 94%, 95%, 96%, 97%, 98%, 99%, or more) sequence identity to the amino acid sequence of SEQ ID NO: 1. In some embodiments, the GAA has an amino acid sequence having at least 95% (e.g., at least 96%, 97%, 98%, 99%, or more) sequence identity to the amino acid sequence of SEQ ID NO: 1. In some embodiments, the GAA has an amino acid sequence having at least 98% (e.g., 99% or more) sequence identity to the amino acid sequence of SEQ ID NO: 1. In some embodiments, the GAA has an amino acid sequence having at least 99% sequence identity to the amino acid sequence of SEQ ID NO: 1. In some embodiments, the GAA has the amino acid sequence of SEQ ID NO: 1.

In some embodiments, the GAA has an amino acid sequence of SEQ ID NO: 2 or is a variant thereof having at least 85% (e.g., 90%, 95%, 96%, 97%, 98%, 99%, or more) sequence identity to the amino acid sequence of SEQ ID NO: 2. In some embodiments, the GAA has an amino acid sequence having at least 90% (e.g., at least 90%, 91%, 92%, 93%, 94%, 95%, 96%, 97%, 98%, 99%, or more) sequence identity to the amino acid sequence of SEQ ID NO: 2. In some embodiments, the GAA has an amino acid sequence having at least 95% (e.g., at least 96%, 97%, 98%, 99%, or more) sequence identity to the amino acid sequence of SEQ ID NO: 2. In some embodiments, the GAA has an amino acid sequence having at least 98% (e.g., 99% or more) sequence identity to the amino acid sequence of SEQ ID NO: 2. In some embodiments, the GAA has an amino acid sequence having at least 99% sequence identity to the amino acid sequence of SEQ ID NO: 2. In some embodiments, the GAA has the amino acid sequence of SEQ ID NO: 2.

In some embodiments, the GAA has an amino acid sequence of SEQ ID NO: 3 or is a variant thereof having at least 85% (e.g., 90%, 95%, 96%, 97%, 98%, 99%, or more) sequence identity to the amino acid sequence of SEQ ID NO: 3. In some embodiments, the GAA has an amino acid sequence having at least 90% (e.g., at least 90%, 91%, 92%, 93%, 94%, 95%, 96%, 97%, 98%, 99%, or more) sequence identity to the amino acid sequence of SEQ ID NO: 3. In some embodiments, the GAA has an amino acid sequence having at least 95% (e.g., at least 96%, 97%, 98%, 99%, or more) sequence identity to the amino acid sequence of SEQ ID NO: 3. In some embodiments, the GAA has an amino acid sequence having at least 98% (e.g., 99% or more) sequence identity to the amino acid sequence of SEQ ID NO: 3. In some embodiments, the GAA has an amino acid sequence having at least 99% sequence identity to the amino acid sequence of SEQ ID NO: 3. In some embodiments, the GAA has the amino acid sequence of SEQ ID NO: 3.

In some embodiments, the GAA has an amino acid sequence of SEQ ID NO: 3 or is a variant thereof having at least 85% (e.g., 90%, 95%, 96%, 97%, 98%, 99%, or more) sequence identity to the amino acid sequence of SEQ ID NO: 4. In some embodiments, the GAA has an amino acid sequence having at least 90% (e.g., at least 90%, 91%, 92%, 93%, 94%, 95%, 96%, 97%, 98%, 99%, or more) sequence identity to the amino acid sequence of SEQ ID NO: 4. In some embodiments, the GAA has an amino acid sequence having at least 95% (e.g., at least 96%, 97%, 98%, 99%, or more) sequence identity to the amino acid sequence of SEQ ID NO: 4. In some embodiments, the GAA has an amino acid sequence having at least 98% (e.g., 99% or more) sequence identity to the amino acid sequence of SEQ ID NO: 4. In some embodiments, the GAA has an amino acid sequence having at least 99% sequence identity to the amino acid sequence of SEQ ID NO: 4. In some embodiments, the GAA has the amino acid sequence of SEQ ID NO: 4.

In some embodiments, the GAA has an amino acid of any one of the full or partial GAA amino acid sequences disclosed in WO 2005/078077, which is incorporated by reference herein as it relates to GAA amino acid sequences.

In some embodiments, the transgene encoding GAA includes a GAA polynucleotide having the nucleic acid sequence of SEQ ID NO: 5 or a variant thereof having at least 85% (e.g., 90%, 95%, 96%, 97%, 98%, 99%, or more) sequence identity to the nucleic acid sequence of SEQ ID NO: 5. In some embodiments, the transgene encoding GAA includes a GAA polynucleotide having at least 90% (e.g., at least 90%, 91%, 92%, 93%, 94%, 95%, 96%, 97%, 98%, 99%, or more) sequence identity to the nucleic acid sequence of SEQ ID NO: 5. In some embodiments, the transgene encoding GAA includes a GAA polynucleotide having at least 95% (e.g., at least 96%, 97%, 98%, 99%, or more) sequence identity to the nucleic acid sequence of SEQ ID NO: 5. In some embodiments, the transgene encoding GAA includes a GAA polynucleotide having at least 98% (e.g., 99% or more) sequence identity to the nucleic acid sequence of SEQ ID NO: 5. In some embodiments, the transgene encoding GAA includes a GAA polynucleotide having at least 99% sequence identity to the nucleic acid sequence of SEQ ID NO: 5. In some embodiments, the transgene encoding GAA includes a GAA polynucleotide having the nucleic acid sequence of SEQ ID NO: 5.

In some embodiments, the transgene encoding GAA includes a GAA polynucleotide having the nucleic acid sequence of SEQ ID NO: 6 or a variant thereof having at least 85% (e.g., 90%, 95%, 96%, 97%, 98%, 99%, or more) sequence identity to the nucleic acid sequence of SEQ ID NO: 6. In some embodiments, the transgene encoding GAA includes a GAA polynucleotide having at least 90% (e.g., at least 90%, 91%, 92%, 93%, 94%, 95%, 96%, 97%, 98%, 99%, or more) sequence identity to the nucleic acid sequence of SEQ ID NO: 6. In some embodiments, the transgene encoding GAA includes a GAA polynucleotide having at least 95% (e.g., at least 96%, 97%, 98%, 99%, or more) sequence identity to the nucleic acid sequence of SEQ ID NO: 6. In some embodiments, the transgene encoding GAA includes a GAA polynucleotide having at least 98% (e.g., 99% or more) sequence identity to the nucleic acid sequence of SEQ ID NO: 6. In some embodiments, the transgene encoding GAA includes a GAA polynucleotide having at least 99% sequence identity to the nucleic acid sequence of SEQ ID NO: 6. In some embodiments, the transgene encoding GAA includes a GAA polynucleotide having the nucleic acid sequence of SEQ ID NO: 6.

In some embodiments, the transgene encoding GAA includes a GAA polynucleotide having the nucleic acid sequence of SEQ ID NO: 7 or a variant thereof having at least 85% (e.g., 90%, 95%, 96%, 97%, 98%, 99%, or more) sequence identity to the nucleic acid sequence of SEQ ID NO: 7. In some embodiments, the transgene encoding GAA includes a GAA polynucleotide having at least 90% (e.g., at least 90%, 91%, 92%, 93%, 94%, 95%, 96%, 97%, 98%, 99%, or more) sequence identity to the nucleic acid sequence of SEQ ID NO: 7. In some embodiments, the transgene encoding GAA includes a GAA polynucleotide having at least 95% (e.g., at least 96%, 97%, 98%, 99%, or more) sequence identity to the nucleic acid sequence of SEQ ID NO: 7. In some embodiments, the transgene encoding GAA includes a GAA polynucleotide having at least 98% (e.g., 99% or more) sequence identity to the nucleic acid sequence of SEQ ID NO: 7. In some embodiments, the transgene encoding GAA includes a GAA polynucleotide having at least 99% sequence identity to the nucleic acid sequence of SEQ ID NO: 7. In some embodiments, the transgene encoding GAA includes a GAA polynucleotide having the nucleic acid sequence of SEQ ID NO: 7.

In some embodiments, the transgene encoding GAA includes a GAA polynucleotide having the nucleic acid sequence of SEQ ID NO: 8 or a variant thereof having at least 85% (e.g., 90%, 95%, 96%, 97%, 98%, 99%, or more) sequence identity to the nucleic acid sequence of SEQ ID NO: 8. In some embodiments, the transgene encoding GAA includes a GAA polynucleotide having at least 90% (e.g., at least 90%, 91%, 92%, 93%, 94%, 95%, 96%, 97%, 98%, 99%, or more) sequence identity to the nucleic acid sequence of SEQ ID NO: 8. In some embodiments, the transgene encoding GAA includes a GAA polynucleotide having at least 95% (e.g., at least 96%, 97%, 98%, 99%, or more) sequence identity to the nucleic acid sequence of SEQ ID NO: 8. In some embodiments, the transgene encoding GAA includes a GAA polynucleotide having at least 98% (e.g., 99% or more) sequence identity to the nucleic acid sequence of SEQ ID NO: 8. In some embodiments, the transgene encoding GAA includes a GAA polynucleotide having at least 99% sequence identity to the nucleic acid sequence of SEQ ID NO: 8. In some embodiments, the transgene encoding GAA includes a GAA polynucleotide having the nucleic acid sequence of SEQ ID NO: 8.

In some embodiments, the transgene encoding GAA includes a GAA polynucleotide having the nucleic acid sequence of SEQ ID NO: 9 or a variant thereof having at least 85% (e.g., 90%, 95%, 96%, 97%, 98%, 99%, or more) sequence identity to the nucleic acid sequence of SEQ ID NO: 9. In some embodiments, the transgene encoding GAA includes a GAA polynucleotide having at least 90% (e.g., at least 90%, 91%, 92%, 93%, 94%, 95%, 96%, 97%, 98%, 99%, or more) sequence identity to the nucleic acid sequence of SEQ ID NO: 9. In some embodiments, the transgene encoding GAA includes a GAA polynucleotide having at least 95% (e.g., at least 96%, 97%, 98%, 99%, or more) sequence identity to the nucleic acid sequence of SEQ ID NO: 9. In some embodiments, the transgene encoding GAA includes a GAA polynucleotide having at least 98% (e.g., 99% or more) sequence identity to the nucleic acid sequence of SEQ ID NO: 9. In some embodiments, the transgene encoding GAA includes a GAA polynucleotide having at least 99% sequence identity to the nucleic acid sequence of SEQ ID NO: 9. In some embodiments, the transgene encoding GAA includes a GAA polynucleotide having the nucleic acid sequence of SEQ ID NO: 9.

In some embodiments, the transgene encodes two or more GAA (or GILT.GAA) transgenes (e.g., at least 2, 3, 4, 5, 6, 7, 8, 9, 10 or more GAA (or GILT.GAA) transgenes). In some embodiments, the transgene encodes from two to ten GAA (or GILT.GAA) transgenes (e.g., 2, 3, 4, 5, 6, 7, 8, 9, or 10 GAA (or GILT.GAA) transgenes). In some embodiments, the transgene encodes from two to five GAA (or GILT.GAA) transgenes (e.g., 2, 3, 4, or 5 GAA (or GILT.GAA) transgenes). In some embodiments, the transgene encodes two GAA (or GILT.GAA) transgenes. In some embodiments, the GAA (or GILT.GAA) transgenes are expressed from a single, polycistronic expression cassette. In some embodiments, the GAA (or GILT.GAA) transgenes are separated from one another by way of one or more (e.g., 1, 2, 3, 4, 5, 6, 7, 8, 9, or more) IRES. In some embodiments, the GAA (or GILT.GAA) transgenes are expressed from one or more (e.g., 1, 2, 3, 4, 5, 6, 7, 8, 9, 10, or more) monocistronic expression cassettes.

In some embodiments, the polynucleotide encoding wild type GAA may be a codon-optimized polynucleotide to confer resistance against degradation by nucleases and inhibitory RNAs directed to endogenous GAA, as described in detail below.

Wild-type human GAA may have the amino acid sequence of SEQ ID NO: 1 (GenBank reference number: CAA68763.1) or may be a variant thereof having at least 85% (e.g., at least 90%, 91%, 92%, 93%, 94%, 95%, 96%, 97%, 98%, 99%, or more) sequence identity to the amino acid sequence of SEQ ID NO: 1, as is shown below.

(SEQ ID NO: 1)
MGVRHPPCSHRLLAVCALVSLATAALLGHILLHDFLLVPRELSGSSPVLE
ETHPAHQQGASRPGPRDAQAHPGRPRAVPTQCDVPPNSRFDCAPDKAITQ
EQCEARGCCYIPAKQGLQGAQMGQPWCFFPPSYPSYKLENLSSSEMGYTA
TLTRTTPTFFPKDILTLRLDVMMETENRLHFTIKDPANRRYEVPLETPRV
HSRAPSPLYSVEFSEEPFGVIVHRQLDGRVLLNTTVAPLFFADQFLQLST
SLPSQYITGLAEHLSPLMLSTSWTRITLWNRDLAPTPGANLYGSHPFYLA
LEDGGSAHGVFLLNSNAMDVVLQPSPALSWRSTGGILDVYIFLGPEPKSV
VQQYLDVVGYPFMPPYWGLGFHLCRWGYSSTAITRQVVENMTRAHFPLDV
QWNDLDYMDSRRDFTFNKDGFRDFPAMVQELHQGGRRYMMIVDPAISSSG
PAGSYRPYDEGLRRGVFITNETGQPLIGKVWPGSTAFPDFTNPTALAWWE
DMVAEFHDQVPFDGMWIDMNEPSNFIRGSEDGCPNNELENPPYVPGVVGG
TLQAATICASSHQFLSTHYNLHNLYGLTEAIASHRALVKARGTRPFVISR
STFAGHGRYAGHWTGDVWSSWEQLASSVPEILQFNLLGVPLVGADVCGFL
GNTSEELCVRWTQLGAFYPFMRNHNSLLSLPQEPYSFSEPAQQAMRKALT
LRYALLPHLYTLFHQAHVAGETVARPLFLEFPKDSSTWTVDHQLLWGEAL
LITPVLQAGKAEVTGYFPLGTWYDLQTVPIEALGSLPPPPAAPREPAIHS
EGQWVTLPAPLDTINVHLRAGYIIPLQGPGLTTTESRQQPMALAVALTKG
GEARGELFWDDGESLEVLERGAYTQVIFLARNNTIVNELVRVTSEGAGLQ
LQKVTVLGVATAPQQVLSNGVPVSNFTYSPDTKVLDICVSLLMGEQFLVS
WC

Additionally or alternatively, wild-type human GAA may have the amino acid sequence of SEQ ID NO: 2 (UniProt identifier number: P10253-1; CCDS ID: 32760.1) or may be a variant thereof having at least 85% (e.g., at least 90%, 91%, 92%, 93%, 94%, 95%, 96%, 97%, 98%, 99%, or more) sequence identity to the amino acid sequence of SEQ ID NO: 2. The human GAA protein may also be any one of the natural variants of GAA described under UniProt identifier number P10253-1.

(SEQ ID NO: 2)
MGVRHPPCSHRLLAVCALVSLATAALLGHILLHDFLLVPRELSGSSPVLE
ETHPAHQQGASRPGPRDAQAHPGRPRAVPTQCDVPPNSRFDCAPDKAITQ
EQCEARGCCYIPAKQGLQGAQMGQPWCFFPPSYPSYKLENLSSSEMGYTA
TLTRTTPTFFPKDILTLRLDVMMETENRLHFTIKDPANRRYEVPLETPHV
HSRAPSPLYSVEFSEEPFGVIVRRQLDGRVLLNTTVAPLFFADQFLQLST
SLPSQYITGLAEHLSPLMLSTSWTRITLWNRDLAPTPGANLYGSHPFYLA
LEDGGSAHGVFLLNSNAMDVVLQPSPALSWRSTGGILDVYIFLGPEPKSV
VQQYLDVVGYPFMPPYWGLGFHLCRWGYSSTAITRQVVENMTRAHFPLDV
QWNDLDYMDSRRDFTFNKDGFRDFPAMVQELHQGGRRYMMIVDPAISSSG
PAGSYRPYDEGLRRGVFITNETGQPLIGKVWPGSTAFPDFTNPTALAWWE
DMVAEFHDQVPFDGMWIDMNEPSNFIRGSEDGCPNNELENPPYVPGVVGG
TLQAATICASSHQFLSTHYNLHNLYGLTEAIASHRALVKARGTRPFVISR
STFAGHGRYAGHWTGDVWSSWEQLASSVPEILQFNLLGVPLVGADVCGFL
GNTSEELCVRWTQLGAFYPFMRNHNSLLSLPQEPYSFSEPAQQAMRKALT
LRYALLPHLYTLFHQAHVAGETVARPLFLEFPKDSSTWTVDHQLLWGEAL
LITPVLQAGKAEVTGYFPLGTWYDLQTVPVEALGSLPPPPAAPREPAIHS
EGQWVTLPAPLDTINVHLRAGYIIPLQGPGLTTTESRQQPMALAVALTKG
GEARGELFWDDGESLEVLERGAYTQVIFLARNNTIVNELVRVTSEGAGLQ
LQKVTVLGVATAPQQVLSNGVPVSNFTYSPDTKVLDICVSLLMGEQFLVS
WC

Additionally or alternatively, human GAA may also have the amino acid sequence of SEQ ID NO: 3 (UniProt identifier number: I3L3L3-1) or may be a variant thereof having at least 85% (e.g., at least 90%, 91%, 92%, 93%, 94%, 95%, 96%, 97%, 98%, 99%, or more) sequence identity to the amino acid sequence of SEQ ID NO: 3.

(SEQ ID NO: 3)
MGVRHPPCSHRLLAVCALVSLATAALLGHILLHDFLLVPRELSGSSPVLE
ETHPAHQQGASRPGPRDAQAHPGRPRAVPTQCDVPPNSRFDCAPDKAITQ
EQCEARGCCYIPAKQGLQGAQMGQPWCFFPPSYPSYKLENLSSSEMGYTA
TLTRTTPTFFPKDILTLRLDVMMETENRLHFTIKDPANRRYEVPLETPHV
HSRAPSPLYSVEFSEEPFGVIVRRQLDGRVLLNTTVAPLFFADQFLQLST
SLPSQYITGLAEHLSPLMLSTSWTRITLWNRDLA

Additionally or alternatively, human GAA may also have the amino acid sequence of SEQ ID NO: 4 (UniProt identifier number: I3L0S5-1) a variant thereof having at least 85% (e.g., at least 90%, 91%, 92%, 93%, 94%, 95%, 96%, 97%, 98%, 99%, or more) sequence identity to the amino acid sequence of SEQ ID NO: 4.

(SEQ ID NO: 4)
MGVRHPPCSHRLLAVCALVSLATAALLGHILLHDFLLVPRELSGSSPVLE
ETHPAHQQGASRPGPRDAQAHPGRPRAVPTQCDVPPNSRFDCAPDKAITQ
EQCEARGCCYIPAKQGLQGAQMGQPWCFFPPSYPSYKLENLSSSEMGYTA
TLTRTTPTFFPKDILTLRLDVMMETENRLHFTIKDPANRRYEVPLETPHV
HSRAPSPL

The polynucleotide encoding GAA may have the nucleic acid sequence of SEQ ID NO: 5 (GenBank reference number: Y00839.1) or may be a variant thereof having at least 85% (e.g., at least 90%, 91%, 92%, 93%, 94%, 95%, 96%, 97%, 98%, 99%, or more) sequence identity to the nucleic acid sequence of SEQ ID NO: 5.

(SEQ ID NO: 5)
CAGTTGGGAAAGCTGAGGTTGTCGCCGGGGCCGCGGGTGGAGGTCGGGGATGAG
GCAGCAGGTAGGACAGTGACCTCGGTGACGCGAAGGACCCCGGCCACCTCTAGGT
TCTCCTCGTCCGCCCGTTGTTCAGCGAGGGAGGCTCTGGGCCTGCCGCAGCTGAC
GGGGAAACTGAGGCACGGAGCGGGCCTGTAGGAGCTGTCCAGGCCATCTCCAACC
ATGGGAGTGAGGCACCCGCCCTGCTCCCACCGGCTCCTGGCCGTCTGCGCCCTCG
TGTCCTTGGCAACCGCTGCACTCCTGGGGCACATCCTACTCCATGATTTCCTGCTG
GTTCCCCGAGAGCTGAGTGGCTCCTCCCCAGTCCTGGAGGAGACTCACCCAGCTC
ACCAGCAGGGAGCCAGCAGACCAGGGCCCCGGGATGCCCAGGCACACCCCGGCC
GTCCCAGAGCAGTGCCCACACAGTGCGACGTCCCCCCCAACAGCCGCTTCGATTG
CGCCCCTGACAAGGCCATCACCCAGGAACAGTGCGAGGCCCGCGGCTGCTGCTAC
ATCCCTGCAAAGCAGGGGCTGCAGGGAGCCCAGATGGGGCAGCCCTGGTGCTTCT
TCCCACCCAGCTACCCCAGCTACAAGCTGGAGAACCTGAGCTCCTCTGAAATGGGC
TACACGGCCACCCTGACCCGTACCACCCCCACCTTCTTCCCCAAGGACATCCTGAC
CCTGCGGCTGGACGTGATGATGGAGACTGAGAACCGCCTCCACTTCACGATCAAAG
ATCCAGCTAACAGGCGCTACGAGGTGCCCTTGGAGACCCCGCGTGTCCACAGCCG
GGCACCGTCCCCACTCTACAGCGTGGAGTTCTCCGAGGAGCCCTTCGGGGTGATC
GTGCACCGGCAGCTGGACGGCCGCGTGCTGCTGAACACGACGGTGGCGCCCCTG
TTCTTTGCGGACCAGTTCCTTCAGCTGTCCACCTCGCTGCCCTCGCAGTATATCACA
GGCCTCGCCGAGCACCTCAGTCCCCTGATGCTCAGCACCAGCTGGACCAGGATCA
CCCTGTGGAACCGGGACCTTGCGCCCACGCCCGGTGCGAACCTCTACGGGTCTCA
CCCTTTCTACCTGGCGCTGGAGGACGGGGGGTCGGCACACGGGGTGTTCCTGCTA
AACAGCAATGCCATGGATGTGGTCCTGCAGCCGAGCCCTGCCCTTAGCTGGAGGTC
GACAGGTGGGATCCTGGATGTCTACATCTTCCTGGGCCCAGAGCCCAAGAGCGTG
GTGCAGCAGTACCTGGACGTTGTGGGATACCCGTTCATGCCGCCATACTGGGGCCT
GGGCTTCCACCTGTGCCGCTGGGGCTACTCCTCCACCGCTATCACCCGCCAGGTG
GTGGAGAACATGACCAGGGCCCACTTCCCCCTGGACGTCCAATGGAACGACCTGG
ACTACATGGACTCCCGGAGGGACTTCACGTTCAACAAGGATGGCTTCCGGGACTTC
CCGGCCATGGTGCAGGAGCTGCACCAGGGGGGCCGGCGCTACATGATGATCGTGG
ATCCTGCCATCAGCAGCTCGGGCCCTGCCGGGAGCTACAGGCCCTACGACGAGGG
TCTGCGGAGGGGGGTTTTCATCACCAACGAGACCGGCCAGCCGCTGATTGGGAAG
GTATGGCCCGGGTCCACTGCCTTCCCCGACTTCACCAACCCCACAGCCCTGGCCTG
GTGGGAGGACATGGTGGCTGAGTTCCATGACCAGGTGCCCTTCGACGGCATGTGG
ATTGACATGAACGAGCCTTCCAACTTCATCAGAGGCTCTGAGGACGGCTGCCCCAA
CAATGAGCTGGAGAACCCACCCTACGTGCCTGGGGTGGTTGGGGGGACCCTCCAG
GCGGCCACCATCTGTGCCTCCAGCCACCAGTTTCTCTCCACACACTACAACCTGCA
CAACCTCTACGGCCTGACCGAAGCCATCGCCTCCCACAGGGCGCTGGTGAAGGCT
CGGGGGACACGCCCATTTGTGATCTCCCGCTCGACCTTTGCTGGCCACGGCCGATA
CGCCGGCCACTGGACGGGGGACGTGTGGAGCTCCTGGGAGCAGCTCGCCTCCTC
CGTGCCAGAAATCCTGCAGTTTAACCTGCTGGGGGTGCCTCTGGTCGGGGCCGAC
GTCTGCGGCTTCCTGGGCAACACCTCAGAGGAGCTGTGTGTGCGCTGGACCCAGC
TGGGGGCCTTCTACCCCTTCATGCGGAACCACAACAGCCTGCTCAGTCTGCCCCAG
GAGCCGTACAGCTTCAGCGAGCCGGCCCAGCAGGCCATGAGGAAGGCCCTCACCC
TGCGCTACGCACTCCTCCCCCACCTCTACACACTGTTCCACCAGGCCCACGTCGCG
GGGGAGACCGTGGCCCGGCCCCTCTTCCTGGAGTTCCCCAAGGACTCTAGCACCT
GGACTGTGGACCACCAGCTCCTGTGGGGGGAGGCCCTGCTCATCACCCCAGTGCT
CCAGGCCGGGAAGGCCGAAGTGACTGGCTACTTCCCCTTGGGCACATGGTACGAC
CTGCAGACGGTGCCAATAGAGGCCCTTGGCAGCCTCCCACCCCCACCTGCAGCTC
CCCGTGAGCCAGCCATCCACAGCGAGGGGCAGTGGGTGACGCTGCCGGCCCCCC
TGGACACCATCAACGTCCACCTCCGGGCTGGGTACATCATCCCCCTGCAGGGCCCT
GGCCTCACAACCACAGAGTCCCGCCAGCAGCCCATGGCCCTGGCTGTGGCCCTGA
CCAAGGGTGGAGAGGCCCGAGGGGAGCTGTTCTGGGACGATGGAGAGAGCCTGG
AAGTGCTGGAGCGAGGGGCCTACACACAGGTCATCTTCCTGGCCAGGAATAACAC
GATCGTGAATGAGCTGGTACGTGTGACCAGTGAGGGAGCTGGCCTGCAGCTGCAG
AAGGTGACTGTCCTGGGCGTGGCCACGGCGCCCCAGCAGGTCCTCTCCAACGGTG
TCCCTGTCTCCAACTTCACCTACAGCCCCGACACCAAGGTCCTGGACATCTGTGTCT
CGCTGTTGATGGGAGAGCAGTTTCTCGTCAGCTGGTGTTAGCCGGGCGGAGTGTGT
TAGTCTCTCCAGAGGGAGGCTGGTTCCCCAGGGAAGCAGAGCCTGTGTGCGGGCA
GCAGCTGTGTGCGGGCCTGGGGGTTGCATGTGTCACCTGGAGCTGGGCACTAACC
ATTCCAAGCCGCCGCATCGCTTGTTTCCACCTCCTGGGCCGGGGCTCTGGCCCCCA
ACGTGTCTAGGAGAGCTTTCTCCCTAGATCGCACTGTGGGCCGGGGCCTGGAGGG
CTGCTCTGTGTTAATAAGATTGTAAGGTTTGCCCTCCTCACCTGTTGCCGGCATGCG
GGTAGTATTAGCCACCCCCCTCCATCTGTTCCCAGCACCGGAGAAGGGGGTGCTCA
GGTGGAGGTGTGGGGTATGCACCTGAGCTCCTGCTTCGCGCCTGCTGCTCTGCCC
CAACGCGACCGCTTCCCGGCTGCCCAGAGGGCTGGATGCCTGCCGGTCCCCGAGC
AAGCCTGGGAACTCAGGAAAATTCACAGGACTTGGGAGATTCTAAATCTTAAGTGCA
ATTATTTTAATAAAAGGGGCATTTGGAATC

Additionally or alternatively, the polynucleotide encoding GAA may have the nucleic acid sequence of SEQ ID NO: 6 (NCBI Reference Sequence: NM_000152.4) or may be a variant thereof having at least 85% (e.g., at least 90%, 91%, 92%, 93%, 94%, 95%, 96%, 97%, 98%, 99%, or more) sequence identity to the nucleic acid sequence of SEQ ID NO: 6.

(SEQ ID NO: 6)
GCCCCGCGACGAGCTCCCGCCGGTCACGTGACCCGCCTCTGCGCGCCCCCGGG
CACGACCCCGGAGTCTCCGCGGGGGGCCAGGGCGCGCGTGCGCGGAGGTGAG
CCGGGCCGGGGCTGCGGGGCTTCCCTGAGCGCGGGCCGGGTCGGTGGGGCGG
TCGGCTGCCCGCGCGGCCTCTCAGTTGGGAAAGCTGAGGTTGTCGCCGGGGCC
GCGGGTGGAGGTCGGGGATGAGGCAGCAGGTAGGACAGTGACCTCGGTGACGC
GAAGGACCCCGGCCACCTCTAGGTTCTCCTCGTCCGCCCGTTGTTCAGCGAGGG
AGGCTCTGCGCGTGCCGCAGCTGACGGGGAAACTGAGGCACGGAGCGGGCCTG
TAGGAGCTGTCCAGGCCATCTCCAACCATGGGAGTGAGGCACCCGCCCTGCTCC
CACCGGCTCCTGGCCGTCTGCGCCCTCGTGTCCTTGGCAACCGCTGCACTCCTG
GGGCACATCCTACTCCATGATTTCCTGCTGGTTCCCCGAGAGCTGAGTGGCTCCT
CCCCAGTCCTGGAGGAGACTCACCCAGCTCACCAGCAGGGAGCCAGCAGACCAG
GGCCCCGGGATGCCCAGGCACACCCCGGCCGTCCCAGAGCAGTGCCCACACAG
TGCGACGTCCCCCCCAACAGCCGCTTCGATTGCGCCCCTGACAAGGCCATCACC
CAGGAACAGTGCGAGGCCCGCGGCTGTTGCTACATCCCTGCAAAGCAGGGGCTG
CAGGGAGCCCAGATGGGGCAGCCCTGGTGCTTCTTCCCACCCAGCTACCCCAGC
TACAAGCTGGAGAACCTGAGCTCCTCTGAAATGGGCTACACGGCCACCCTGACCC
GTACCACCCCCACCTTCTTCCCCAAGGACATCCTGACCCTGCGGCTGGACGTGAT
GATGGAGACTGAGAACCGCCTCCACTTCACGATCAAAGATCCAGCTAACAGGCGC
TACGAGGTGCCCTTGGAGACCCCGCATGTCCACAGCCGGGCACCGTCCCCACTC
TACAGCGTGGAGTTCTCCGAGGAGCCCTTCGGGGTGATCGTGCGCCGGCAGCTG
GACGGCCGCGTGCTGCTGAACACGACGGTGGCGCCCCTGTTCTTTGCGGACCAG
TTCCTTCAGCTGTCCACCTCGCTGCCCTCGCAGTATATCACAGGCCTCGCCGAGC
ACCTCAGTCCCCTGATGCTCAGCACCAGCTGGACCAGGATCACCCTGTGGAACC
GGGACCTTGCGCCCACGCCCGGTGCGAACCTCTACGGGTCTCACCCTTTCTACC
TGGCGCTGGAGGACGGGGGGTCGGCACACGGGGTGTTCCTGCTAAACAGCAATG
CCATGGATGTGGTCCTGCAGCCGAGCCCTGCCCTTAGCTGGAGGTCGACAGGTG
GGATCCTGGATGTCTACATCTTCCTGGGCCCAGAGCCCAAGAGCGTGGTGCAGC
AGTACCTGGACGTTGTGGGATACCCGTTCATGCCGCCATACTGGGGCCTGGGCT
TCCACCTGTGCCGCTGGGGCTACTCCTCCACCGCTATCACCCGCCAGGTGGTGG
AGAACATGACCAGGGCCCACTTCCCCCTGGACGTCCAGTGGAACGACCTGGACT
ACATGGACTCCCGGAGGGACTTCACGTTCAACAAGGATGGCTTCCGGGACTTCCC
GGCCATGGTGCAGGAGCTGCACCAGGGCGGCCGGCGCTACATGATGATCGTGG
ATCCTGCCATCAGCAGCTCGGGCCCTGCCGGGAGCTACAGGCCCTACGACGAGG
GTCTGCGGAGGGGGGTTTTCATCACCAACGAGACCGGCCAGCCGCTGATTGGGA
AGGTATGGCCCGGGTCCACTGCCTTCCCCGACTTCACCAACCCCACAGCCCTGG
CCTGGTGGGAGGACATGGTGGCTGAGTTCCATGACCAGGTGCCCTTCGACGGCA
TGTGGATTGACATGAACGAGCCTTCCAACTTCATCAGGGGCTCTGAGGACGGCTG
CCCCAACAATGAGCTGGAGAACCCACCCTACGTGCCTGGGGTGGTTGGGGGGAC
CCTCCAGGCGGCCACCATCTGTGCCTCCAGCCACCAGTTTCTCTCCACACACTAC
AACCTGCACAACCTCTACGGCCTGACCGAAGCCATCGCCTCCCACAGGGCGCTG
GTGAAGGCTCGGGGGACACGCCCATTTGTGATCTCCCGCTCGACCTTTGCTGGC
CACGGCCGATACGCCGGCCACTGGACGGGGGACGTGTGGAGCTCCTGGGAGCA
GCTCGCCTCCTCCGTGCCAGAAATCCTGCAGTTTAACCTGCTGGGGGTGCCTCTG
GTCGGGGCCGACGTCTGCGGCTTCCTGGGCAACACCTCAGAGGAGCTGTGTGTG
CGCTGGACCCAGCTGGGGGCCTTCTACCCCTTCATGCGGAACCACAACAGCCTG
CTCAGTCTGCCCCAGGAGCCGTACAGCTTCAGCGAGCCGGCCCAGCAGGCCATG
AGGAAGGCCCTCACCCTGCGCTACGCACTCCTCCCCCACCTCTACACACTGTTCC
ACCAGGCCCACGTCGCGGGGGAGACCGTGGCCCGGCCCCTCTTCCTGGAGTTC
CCCAAGGACTCTAGCACCTGGACTGTGGACCACCAGCTCCTGTGGGGGGAGGCC
CTGCTCATCACCCCAGTGCTCCAGGCCGGGAAGGCCGAAGTGACTGGCTACTTC
CCCTTGGGCACATGGTACGACCTGCAGACGGTGCCAGTAGAGGCCCTTGGCAGC
CTCCCACCCCCACCTGCAGCTCCCCGTGAGCCAGCCATCCACAGCGAGGGGCAG
TGGGTGACGCTGCCGGCCCCCCTGGACACCATCAACGTCCACCTCCGGGCTGGG
TACATCATCCCCCTGCAGGGCCCTGGCCTCACAACCACAGAGTCCCGCCAGCAG
CCCATGGCCCTGGCTGTGGCCCTGACCAAGGGTGGGGAGGCCCGAGGGGAGCT
GTTCTGGGACGATGGAGAGAGCCTGGAAGTGCTGGAGCGAGGGGCCTACACACA
GGTCATCTTCCTGGCCAGGAATAACACGATCGTGAATGAGCTGGTACGTGTGACC
AGTGAGGGAGCTGGCCTGCAGCTGCAGAAGGTGACTGTCCTGGGCGTGGCCAC
GGCGCCCCAGCAGGTCCTCTCCAACGGTGTCCCTGTCTCCAACTTCACCTACAGC
CCCGACACCAAGGTCCTGGACATCTGTGTCTCGCTGTTGATGGGAGAGCAGTTTC
TCGTCAGCTGGTGTTAGCCGGGCGGAGTGTGTTAGTCTCTCCAGAGGGAGGCTG
GTTCCCCAGGGAAGCAGAGCCTGTGTGCGGGCAGCAGCTGTGTGCGGGCCTGG
GGGTTGCATGTGTCACCTGGAGCTGGGCACTAACCATTCCAAGCCGCCGCATCG
CTTGTTTCCACCTCCTGGGCCGGGGCTCTGGCCCCCAACGTGTCTAGGAGAGCT
TTCTCCCTAGATCGCACTGTGGGCCGGGGCCCTGGAGGGCTGCTCTGTGTTAATA
AGATTGTAAGGTTTGCCCTCCTCACCTGTTGCCGGCATGCGGGTAGTATTAGCCA
CCCCCCTCCATCTGTTCCCAGCACCGGAGAAGGGGGTGCTCAGGTGGAGGTGTG
GGGTATGCACCTGAGCTCCTGCTTCGCGCCTGCTGCTCTGCCCCAACGCGACCG
CTGCCCGGCTGCCCAGAGGGCTGGATGCCTGCCGGTCCCCGAGCAAGCCTGGG
AACTCAGGAAAATTCACAGGACTTGGGAGATTCTAAATCTTAAGTGCAATTATTTTT
AATAAAAGGGGCATTTGGAATCAGCAAAAAAAAAAAAAAAAAAAAAAAAAAAAAA

Additionally or alternatively, human GAA may be encoded by a polynucleotide having the sequence of SEQ ID NO: 7 (NCBI Reference Number: NM_001079803.2) or may be a variant thereof having at least 85% (e.g., at least 90%, 91%, 92%, 93%, 94%, 95%, 96%, 97%, 98%, 99%, or more) sequence identity to the nucleic acid sequence of SEQ ID NO: 7.

(SEQ ID NO: 7)
GCCCCGCGACGAGCTCCCGCCGGTCACGTGACCCGCCTCTGCGCGCCCCCGGGC
ACGACCCCGGAGTCTCCGCGGGGGGCCAGGGCGCGCGTGCGCGGAGGTTCTCCT
CGTCCGCCCGTTGTTCAGCGAGGGAGGCTCTGCGCGTGCCGCAGCTGACGGGGAA
ACTGAGGCACGGAGCGGGCCTGTAGGAGCTGTCCAGGCCATCTCCAACCATGGGA
GTGAGGCACCCGCCCTGCTCCCACCGGCTCCTGGCCGTCTGCGCCCTCGTGTCCT
TGGCAACCGCTGCACTCCTGGGGCACATCCTACTCCATGATTTCCTGCTGGTTCCC
CGAGAGCTGAGTGGCTCCTCCCCAGTCCTGGAGGAGACTCACCCAGCTCACCAGC
AGGGAGCCAGCAGACCAGGGCCCCGGGATGCCCAGGCACACCCCGGCCGTCCCA
GAGCAGTGCCCACACAGTGCGACGTCCCCCCCAACAGCCGCTTCGATTGCGCCCC
TGACAAGGCCATCACCCAGGAACAGTGCGAGGCCCGCGGCTGTTGCTACATCCCT
GCAAAGCAGGGGCTGCAGGGAGCCCAGATGGGGCAGCCCTGGTGCTTCTTCCCAC
CCAGCTACCCCAGCTACAAGCTGGAGAACCTGAGCTCCTCTGAAATGGGCTACACG
GCCACCCTGACCCGTACCACCCCCACCTTCTTCCCCAAGGACATCCTGACCCTGCG
GCTGGACGTGATGATGGAGACTGAGAACCGCCTCCACTTCACGATCAAAGATCCAG
CTAACAGGCGCTACGAGGTGCCCTTGGAGACCCCGCATGTCCACAGCCGGGCACC
GTCCCCACTCTACAGCGTGGAGTTCTCCGAGGAGCCCTTCGGGGTGATCGTGCGC
CGGCAGCTGGACGGCCGCGTGCTGCTGAACACGACGGTGGCGCCCCTGTTCTTTG
CGGACCAGTTCCTTCAGCTGTCCACCTCGCTGCCCTCGCAGTATATCACAGGCCTC
GCCGAGCACCTCAGTCCCCTGATGCTCAGCACCAGCTGGACCAGGATCACCCTGT
GGAACCGGGACCTTGCGCCCACGCCCGGTGCGAACCTCTACGGGTCTCACCCTTT
CTACCTGGCGCTGGAGGACGGCGGGTCGGCACACGGGGTGTTCCTGCTAAACAGC
AATGCCATGGATGTGGTCCTGCAGCCGAGCCCTGCCCTTAGCTGGAGGTCGACAG
GTGGGATCCTGGATGTCTACATCTTCCTGGGCCCAGAGCCCAAGAGCGTGGTGCA
GCAGTACCTGGACGTTGTGGGATACCCGTTCATGCCGCCATACTGGGGCCTGGGC
TTCCACCTGTGCCGCTGGGGCTACTCCTCCACCGCTATCACCCGCCAGGTGGTGGA
GAACATGACCAGGGCCCACTTCCCCCTGGACGTCCAGTGGAACGACCTGGACTACA
TGGACTCCCGGAGGGACTTCACGTTCAACAAGGATGGCTTCCGGGACTTCCCGGC
CATGGTGCAGGAGCTGCACCAGGGCGGCCGGCGCTACATGATGATCGTGGATCCT
GCCATCAGCAGCTCGGGCCCTGCCGGGAGCTACAGGCCCTACGACGAGGGTCTGC
GGAGGGGGGTTTTCATCACCAACGAGACCGGCCAGCCGCTGATTGGGAAGGTATG
GCCCGGGTCCACTGCCTTCCCCGACTTCACCAACCCCACAGCCCTGGCCTGGTGG
GAGGACATGGTGGCTGAGTTCCATGACCAGGTGCCCTTCGACGGCATGTGGATTGA
CATGAACGAGCCTTCCAACTTCATCAGGGGCTCTGAGGACGGCTGCCCCAACAAT
GAGCTGGAGAACCCACCCTACGTGCCTGGGGTGGTTGGGGGGACCCTCCAGGCG
GCCACCATCTGTGCCTCCAGCCACCAGTTTCTCTCCACACACTACAACCTGCACAAC
CTCTACGGCCTGACCGAAGCCATCGCCTCCCACAGGGCGCTGGTGAAGGCTCGGG
GGACACGCCCATTTGTGATCTCCCGCTCGACCTTTGCTGGCCACGGCCGATACGCC
GGCCACTGGACGGGGGACGTGTGGAGCTCCTGGGAGCAGCTCGCCTCCTCCGTG
CCAGAAATCCTGCAGTTTAACCTGCTGGGGGTGCCTCTGGTCGGGGCCGACGTCT
GCGGCTTCCTGGGCAACACCTCAGAGGAGCTGTGTGTGCGCTGGACCCAGCTGGG
GGCCTTCTACCCCTTCATGCGGAACCACAACAGCCTGCTCAGTCTGCCCCAGGAGC
CGTACAGCTTCAGCGAGCCGGCCCAGCAGGCCATGAGGAAGGCCCTCACCCTGCG
CTACGCACTCCTCCCCCACCTCTACACACTGTTCCACCAGGCCCACGTCGCGGGGG
AGACCGTGGCCCGGCCCCTCTTCCTGGAGTTCCCCAAGGACTCTAGCACCTGGACT
GTGGACCACCAGCTCCTGTGGGGGGAGGCCCTGCTCATCACCCCAGTGCTCCAGG
CCGGGAAGGCCGAAGTGACTGGCTACTTCCCCTTGGGCACATGGTACGACCTGCA
GACGGTGCCAGTAGAGGCCCTTGGCAGCCTCCCACCCCCACCTGCAGCTCCCCGT
GAGCCAGCCATCCACAGCGAGGGGCAGTGGGTGACGCTGCCGGCCCCCCTGGAC
ACCATCAACGTCCACCTCCGGGCTGGGTACATCATCCCCCTGCAGGGCCCTGGCCT
CACAACCACAGAGTCCCGCCAGCAGCCCATGGCCCTGGCTGTGGCCCTGACCAAG
GGTGGGGAGGCCCGAGGGGAGCTGTTCTGGGACGATGGAGAGAGCCTGGAAGTG
CTGGAGCGAGGGGCCTACACACAGGTCATCTTCCTGGCCAGGAATAACACGATCGT
GAATGAGCTGGTACGTGTGACCAGTGAGGGAGCTGGCCTGCAGCTGCAGAAGGTG
ACTGTCCTGGGCGTGGCCACGGCGCCCCAGCAGGTCCTCTCCAACGGTGTCCCTG
TCTCCAACTTCACCTACAGCCCCGACACCAAGGTCCTGGACATCTGTGTCTCGCTGT
TGATGGGAGAGCAGTTTCTCGTCAGCTGGTGTTAGCCGGGGGGAGTGTGTTAGTCT
CTCCAGAGGGAGGCTGGTTCCCCAGGGAAGCAGAGCCTGTGTGCGGGCAGCAGCT
GTGTGCGGGCCTGGGGGTTGCATGTGTCACCTGGAGCTGGGCACTAACCATTCCA
AGCCGCCGCATCGCTTGTTTCCACCTCCTGGGCCGGGGCTCTGGCCCCCAACGTG
TCTAGGAGAGCTTTCTCCCTAGATCGCACTGTGGGCCGGGGCCCTGGAGGGCTGC
TCTGTGTTAATAAGATTGTAAGGTTTGCCCTCCTCACCTGTTGCCGGCATGCGGGTA
GTATTAGCCACCCCCCTCCATCTGTTCCCAGCACCGGAGAAGGGGGTGCTCAGGTG
GAGGTGTGGGGTATGCACCTGAGCTCCTGCTTCGCGCCTGCTGCTCTGCCCCAAC
GCGACCGCTGCCCGGCTGCCCAGAGGGCTGGATGCCTGCCGGTCCCCGAGCAAG
CCTGGGAACTCAGGAAAATTCACAGGACTTGGGAGATTCTAAATCTTAAGTGCAATT
ATTTTTAATAAAAGGGGCATTTGGAATCAGCAAAAAAAAAAAAAAAAAAAAAAAAAAA
AAA

Additionally or alternatively, human GAA may be encoded by a polynucleotide having the sequence of SEQ ID NO: 8 (NCBI Reference Sequence: NM_001079804.2) or may be a variant thereof having at least 85% (e.g., at least 90%, 91%, 92%, 93%, 94%, 95%, 96%, 97%, 98%, 99%, or more) sequence identity to the nucleic acid sequence of SEQ ID NO: 8.

(SEQ ID NO: 8)
GCCCCGCGACGAGCTCCCGCCGGTCACGTGACCCGCCTCTGCGCGCCCCCGGG
CACGACCCCGGAGTCTCCGCGGGGGGCCAGGGCGCGCGTGCGCGGAGGCCTG
TAGGAGCTGTCCAGGCCATCTCCAACCATGGGAGTGAGGCACCCGCCCTGCTCC
CACCGGCTCCTGGCCGTCTGCGCCCTCGTGTCCTTGGCAACCGCTGCACTCCTG
GGGCACATCCTACTCCATGATTTCCTGCTGGTTCCCCGAGAGCTGAGTGGCTCCT
CCCCAGTCCTGGAGGAGACTCACCCAGCTCACCAGCAGGGAGCCAGCAGACCAG
GGCCCCGGGATGCCCAGGCACACCCCGGCCGTCCCAGAGCAGTGCCCACACAG
TGCGACGTCCCCCCCAACAGCCGCTTCGATTGCGCCCCTGACAAGGCCATCACC
CAGGAACAGTGCGAGGCCCGCGGCTGTTGCTACATCCCTGCAAAGCAGGGGCTG
CAGGGAGCCCAGATGGGGCAGCCCTGGTGCTTCTTCCCACCCAGCTACCCCAGC
TACAAGCTGGAGAACCTGAGCTCCTCTGAAATGGGCTACACGGCCACCCTGACCC
GTACCACCCCCACCTTCTTCCCCAAGGACATCCTGACCCTGCGGCTGGACGTGAT
GATGGAGACTGAGAACCGCCTCCACTTCACGATCAAAGATCCAGCTAACAGGCGC
TACGAGGTGCCCTTGGAGACCCCGCATGTCCACAGCCGGGCACCGTCCCCACTC
TACAGCGTGGAGTTCTCCGAGGAGCCCTTCGGGGTGATCGTGCGCCGGCAGCTG
GACGGCCGCGTGCTGCTGAACACGACGGTGGCGCCCCTGTTCTTTGCGGACCAG
TTCCTTCAGCTGTCCACCTCGCTGCCCTCGCAGTATATCACAGGCCTCGCCGAGC
ACCTCAGTCCCCTGATGCTCAGCACCAGCTGGACCAGGATCACCCTGTGGAACC
GGGACCTTGCGCCCACGCCCGGTGCGAACCTCTACGGGTCTCACCCTTTCTACC
TGGCGCTGGAGGACGGGGGGTCGGCACACGGGGTGTTCCTGCTAAACAGCAATG
CCATGGATGTGGTCCTGCAGCCGAGCCCTGCCCTTAGCTGGAGGTCGACAGGTG
GGATCCTGGATGTCTACATCTTCCTGGGCCCAGAGCCCAAGAGCGTGGTGCAGC
AGTACCTGGACGTTGTGGGATACCCGTTCATGCCGCCATACTGGGGCCTGGGCT
TCCACCTGTGCCGCTGGGGCTACTCCTCCACCGCTATCACCCGCCAGGTGGTGG
AGAACATGACCAGGGCCCACTTCCCCCTGGACGTCCAGTGGAACGACCTGGACT
ACATGGACTCCCGGAGGGACTTCACGTTCAACAAGGATGGCTTCCGGGACTTCCC
GGCCATGGTGCAGGAGCTGCACCAGGGGGCCGGCGCTACATGATGATCGTGG
ATCCTGCCATCAGCAGCTCGGGCCCTGCCGGGAGCTACAGGCCCTACGACGAGG
GTCTGCGGAGGGGGGTTTTCATCACCAACGAGACCGGCCAGCCGCTGATTGGGA
AGGTATGGCCCGGGTCCACTGCCTTCCCCGACTTCACCAACCCCACAGCCCTGG
CCTGGTGGGAGGACATGGTGGCTGAGTTCCATGACCAGGTGCCCTTCGACGGCA
TGTGGATTGACATGAACGAGCCTTCCAACTTCATCAGGGGCTCTGAGGACGGCTG
CCCCAACAATGAGCTGGAGAACCCACCCTACGTGCCTGGGGTGGTTGGGGGGAC
CCTCCAGGCGGCCACCATCTGTGCCTCCAGCCACCAGTTTCTCTCCACACACTAC
AACCTGCACAACCTCTACGGCCTGACCGAAGCCATCGCCTCCCACAGGGCGCTG
GTGAAGGCTCGGGGGACACGCCCATTTGTGATCTCCCGCTCGACCTTTGCTGGC
CACGGCCGATACGCCGGCCACTGGACGGGGGACGTGTGGAGCTCCTGGGAGCA
GCTCGCCTCCTCCGTGCCAGAAATCCTGCAGTTTAACCTGCTGGGGGTGCCTCTG
GTCGGGGCCGACGTCTGCGGCTTCCTGGGCAACACCTCAGAGGAGCTGTGTGTG
CGCTGGACCCAGCTGGGGGCCTTCTACCCCTTCATGCGGAACCACAACAGCCTG
CTCAGTCTGCCCCAGGAGCCGTACAGCTTCAGCGAGCCGGCCCAGCAGGCCATG
AGGAAGGCCCTCACCCTGCGCTACGCACTCCTCCCCCACCTCTACACACTGTTCC
ACCAGGCCCACGTCGCGGGGGAGACCGTGGCCCGGCCCCTCTTCCTGGAGTTC
CCCAAGGACTCTAGCACCTGGACTGTGGACCACCAGCTCCTGTGGGGGGAGGCC
CTGCTCATCACCCCAGTGCTCCAGGCCGGGAAGGCCGAAGTGACTGGCTACTTC
CCCTTGGGCACATGGTACGACCTGCAGACGGTGCCAGTAGAGGCCCTTGGCAGC
CTCCCACCCCCACCTGCAGCTCCCCGTGAGCCAGCCATCCACAGCGAGGGGCAG
TGGGTGACGCTGCCGGCCCCCCTGGACACCATCAACGTCCACCTCCGGGCTGGG
TACATCATCCCCCTGCAGGGCCCTGGCCTCACAACCACAGAGTCCCGCCAGCAG
CCCATGGCCCTGGCTGTGGCCCTGACCAAGGGTGGGGAGGCCCGAGGGGAGCT
GTTCTGGGACGATGGAGAGAGCCTGGAAGTGCTGGAGCGAGGGGCCTACACACA
GGTCATCTTCCTGGCCAGGAATAACACGATCGTGAATGAGCTGGTACGTGTGACC
AGTGAGGGAGCTGGCCTGCAGCTGCAGAAGGTGACTGTCCTGGGCGTGGCCAC
GGCGCCCCAGCAGGTCCTCTCCAACGGTGTCCCTGTCTCCAACTTCACCTACAGC
CCCGACACCAAGGTCCTGGACATCTGTGTCTCGCTGTTGATGGGAGAGCAGTTTC
TCGTCAGCTGGTGTTAGCCGGGCGGAGTGTGTTAGTCTCTCCAGAGGGAGGCTG
GTTCCCCAGGGAAGCAGAGCCTGTGTGCGGGCAGCAGCTGTGTGCGGGCCTGG
GGGTTGCATGTGTCACCTGGAGCTGGGCACTAACCATTCCAAGCCGCCGCATCG
CTTGTTTCCACCTCCTGGGCCGGGGCTCTGGCCCCCAACGTGTCTAGGAGAGCT
TTCTCCCTAGATCGCACTGTGGGCCGGGGCCCTGGAGGGCTGCTCTGTGTTAATA
AGATTGTAAGGTTTGCCCTCCTCACCTGTTGCCGGCATGCGGGTAGTATTAGCCA
CCCCCCTCCATCTGTTCCCAGCACCGGAGAAGGGGGTGCTCAGGTGGAGGTGTG
GGGTATGCACCTGAGCTCCTGCTTCGCGCCTGCTGCTCTGCCCCAACGCGACCG
CTGCCCGGCTGCCCAGAGGGCTGGATGCCTGCCGGTCCCCGAGCAAGCCTGGG
AACTCAGGAAAATTCACAGGACTTGGGAGATTCTAAATCTTAAGTGCAATTATTTTT
AATAAAAGGGGCATTTGGAATCAGCAAAAAAAAAAAAAAAAAAAAAAAAAAAAAA

Additionally or alternatively, human GAA may be encoded by a polynucleotide having the sequence of SEQ ID NO: 9 (CCDS ID: 32760.1) or may be a variant thereof having at least 85% (e.g., at least 90%, 91%, 92%, 93%, 94%, 95%, 96%, 97%, 98%, 99%, or more) sequence identity to the nucleic acid sequence of SEQ ID NO: 9.

(SEQ ID NO: 9)
ATGGGAGTGAGGCACCCGCCCTGCTCCCACCGGCTCCTGGCCGTCTGCG
CCCTCGTGTCCTTGGCAACCGCTGCACTCCTGGGGCACATCCTACTCCA
TGATTTCCTGCTGGTTCCCCGAGAGCTGAGTGGCTCCTCCCCAGTCCTG
GAGGAGACTCACCCAGCTCACCAGCAGGGAGCCAGCAGACCAGGGCCCC
GGGATGCCCAGGCACACCCCGGCCGTCCCAGAGCAGTGCCCACACAGTG
CGACGTCCCCCCCAACAGCCGCTTCGATTGCGCCCCTGACAAGGCCATC
ACCCAGGAACAGTGCGAGGCCCGCGGCTGTTGCTACATCCCTGCAAAGC
AGGGGCTGCAGGGAGCCCAGATGGGGCAGCCCTGGTGCTTCTTCCCACC
CAGCTACCCCAGCTACAAGCTGGAGAACCTGAGCTCCTCTGAAATGGGC
TACACGGCCACCCTGACCCGTACCACCCCCACCTTCTTCCCCAAGGACA
TCCTGACCCTGCGGCTGGACGTGATGATGGAGACTGAGAACCGCCTCCA
CTTCACGATCAAAGATCCAGCTAACAGGCGCTACGAGGTGCCCTTGGAG
ACCCCGCATGTCCACAGCCGGGCACCGTCCCCACTCTACAGCGTGGAGT
TCTCCGAGGAGCCCTTCGGGGTGATCGTGCGCCGGCAGCTGGACGGCCG
CGTGCTGCTGAACACGACGGTGGCGCCCCTGTTCTTTGCGGACCAGTTC
CTTCAGCTGTCCACCTCGCTGCCCTCGCAGTATATCACAGGCCTCGCCG
AGCACCTCAGTCCCCTGATGCTCAGCACCAGCTGGACCAGGATCACCCT
GTGGAACCGGGACCTTGCGCCCACGCCCGGTGCGAACCTCTACGGGTCT
CACCCTTTCTACCTGGCGCTGGAGGACGGGGGGTCGGCACACGGGGTGT
TCCTGCTAAACAGCAATGCCATGGATGTGGTCCTGCAGCCGAGCCCTGC
CCTTAGCTGGAGGTCGACAGGTGGGATCCTGGATGTCTACATCTTCCTG
GGCCCAGAGCCCAAGAGCGTGGTGCAGCAGTACCTGGACGTTGTGGGAT
ACCCGTTCATGCCGCCATACTGGGGCCTGGGCTTCCACCTGTGCCGCTG
GGGCTACTCCTCCACCGCTATCACCCGCCAGGTGGTGGAGAACATGACC
AGGGCCCACTTCCCCCTGGACGTCCAGTGGAACGACCTGGACTACATGG
ACTCCCGGAGGGACTTCACGTTCAACAAGGATGGCTTCCGGGACTTCCC
GGCCATGGTGCAGGAGCTGCACCAGGGGGGCCGGCGCTACATGATGATC
GTGGATCCTGCCATCAGCAGCTCGGGCCCTGCCGGGAGCTACAGGCCCT
ACGACGAGGGTCTGCGGAGGGGGGTTTTCATCACCAACGAGACCGGCCA
GCCGCTGATTGGGAAGGTATGGCCCGGGTCCACTGCCTTCCCCGACTTC
ACCAACCCCACAGCCCTGGCCTGGTGGGAGGACATGGTGGCTGAGTTCC
ATGACCAGGTGCCCTTCGACGGCATGTGGATTGACATGAACGAGCCTTC
CAACTTCATCAGGGGCTCTGAGGACGGCTGCCCCAACAATGAGCTGGAG
AACCCACCCTACGTGCCTGGGGTGGTTGGGGGGACCCTCCAGGCGGCCA
CCATCTGTGCCTCCAGCCACCAGTTTCTCTCCACACACTACAACCTGCA
CAACCTCTACGGCCTGACCGAAGCCATCGCCTCCCACAGGGCGCTGGTG
AAGGCTCGGGGGACACGCCCATTTGTGATCTCCCGCTCGACCTTTGCTG
GCCACGGCCGATACGCCGGCCACTGGACGGGGGACGTGTGGAGCTCCTG
GGAGCAGCTCGCCTCCTCCGTGCCAGAAATCCTGCAGTTTAACCTGCTG
GGGGTGCCTCTGGTCGGGGCCGACGTCTGCGGCTTCCTGGGCAACACCT
CAGAGGAGCTGTGTGTGCGCTGGACCCAGCTGGGGGCCTTCTACCCCTT
CATGCGGAACCACAACAGCCTGCTCAGTCTGCCCCAGGAGCCGTACAGC
TTCAGCGAGCCGGCCCAGCAGGCCATGAGGAAGGCCCTCACCCTGCGCT
ACGCACTCCTCCCCCACCTCTACACACTGTTCCACCAGGCCCACGTCGC
GGGGGAGACCGTGGCCCGGCCCCTCTTCCTGGAGTTCCCCAAGGACTCT
AGCACCTGGACTGTGGACCACCAGCTCCTGTGGGGGGAGGCCCTGCTCA
TCACCCCAGTGCTCCAGGCCGGGAAGGCCGAAGTGACTGGCTACTTCCC
CTTGGGCACATGGTACGACCTGCAGACGGTGCCAGTAGAGGCCCTTGGC
AGCCTCCCACCCCCACCTGCAGCTCCCCGTGAGCCAGCCATCCACAGCG
AGGGGCAGTGGGTGACGCTGCCGGCCCCCCTGGACACCATCAACGTCCA
CCTCCGGGCTGGGTACATCATCCCCCTGCAGGGCCCTGGCCTCACAACC
ACAGAGTCCCGCCAGCAGCCCATGGCCCTGGCTGTGGCCCTGACCAAGG
GTGGGGAGGCCCGAGGGGAGCTGTTCTGGGACGATGGAGAGAGCCTGGA
AGTGCTGGAGCGAGGGGCCTACACACAGGTCATCTTCCTGGCCAGGAAT
AACACGATCGTGAATGAGCTGGTACGTGTGACCAGTGAGGGAGCTGGCC
TGCAGCTGCAGAAGGTGACTGTCCTGGGCGTGGCCACGGCGCCCCAGCA
GGTCCTCTCCAACGGTGTCCCTGTCTCCAACTTCACCTACAGCCCCGAC
ACCAAGGTCCTGGACATCTGTGTCTCGCTGTTGATGGGAGAGCAGTTTC
TCGTCAGCTGGTGTTAG

According to the methods described herein, a subject (such as a subject having or at risk of developing Pompe disease) can be administered a cell containing a transgene that includes a polynucleotide encoding a polypeptide having any one of amino acid sequences of SEQ ID NOS. 1-4, or a polynucleotide encoding a polypeptide having at least 85% (e.g., 90%, 95%, 96%, 97%, 98%, 99%, or more) sequence identity (e.g., 85%, 90%, 95%, 96%, 97%, 98%, 99%, or more, sequence identity) to any one of the amino acid sequences of SEQ ID NOS. 1-4, or a polynucleotide encoding a polypeptide that contains one or more conservative amino acid substitutions (e.g., 1, 2, 3, 4, 5, 6, 7, 8, 9, or 10 or more conservative amino acid substitutions) relative to any one of SEQ ID NOS. 1-4, provided that the GAA analog encoded retains the therapeutic function of wild type GAA. The activity of GAA is important for normal microglial phagocytic competency and regulation of inflammatory cytokine production. Loss of GAA leads to altered neuro-immune responses and neurodegeneration.

In some embodiments, the expression level of the GAA transgene is measured in one or more organs, tissues, or body fluids of a subject. In some embodiments, the one or more body fluids is peripheral blood. In some embodiments, the one or more tissues is muscle tissue or nervous system tissue. In some embodiments, the muscle tissue is skeletal muscle or cardiac muscle. In some embodiments, the one or more organs is the heart, the brain or spinal cord, or the liver.

### Host Cells

Cells that may be used in conjunction with the compositions and methods described herein include cells that are capable of undergoing further differentiation (e.g., pluripotent cells, ESCs, iPSCs, CD34+ cells, HSCs, MPCs, BLPCs, monocytes, or microglial progenitor cells) or differentiated cells (e.g., macrophages or microglia). For example, one type of cell that can be used in conjunction with the compositions and methods described herein is a pluripotent cell. A pluripotent cell is a cell that possesses the ability to develop into more than one differentiated cell type. Examples of pluripotent cells are ESCs and iPSCs. ESCs and iPSCs have the ability to differentiate into cells of the ectoderm, which gives rise to the skin and nervous system, endoderm, which forms the gastrointestinal and respiratory tracts, endocrine glands, liver, and pancreas, and mesoderm, which forms bone, cartilage, muscles, connective tissue, and most of the circulatory system. Another type of cell that can be used in conjunction with the compositions and methods described herein is a multipotent cell. A multipotent cell is a cell that possesses the ability to differentiate into multiple, but not all cell types. A non-limiting example of a multipotent cell is a CD34+ cell (e.g., HSCs or MPC).

Cells that may be used in conjunction with the compositions and methods described herein include HSCs and MPCs. HSCs are immature blood cells that have the capacity to self-renew and to differentiate into mature blood cells including diverse lineages including but not limited to granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). Human HSCs are CD34+. In addition, HSCs also refer to long term repopulating HSC (LT-HSC) and short-term repopulating HSC (ST-HSC). Any of these HSCs can be used in conjunction with the compositions and methods described herein.

HSCs can differentiate into myeloid progenitor cells, which are also CD34+. Myeloid progenitors can further differentiate into granulocytes (e.g., promyelocytes, neutrophils, eosinophils, and basophils), erythrocytes (e.g., reticulocytes, erythrocytes), thrombocytes (e.g., megakaryoblasts, platelet producing megakaryocytes, and platelets), monocytes (e.g., monocytes and macrophages), dendritic cells, and microglia. Common myeloid progenitors can be characterized by cell surface molecules and are known to be lin−, SCA1−, c-kit+, CD34+, and CD16/32^{mid}.

HSCs and myeloid progenitors can be obtained from blood products. A blood product is a product obtained from the body or an organ of the body containing cells of hematopoietic origin. Such sources include unfractionated bone marrow, umbilical cord, placenta, peripheral blood, or mobilized peripheral blood. All of the aforementioned crude or unfractionated blood products can be enriched for cells having HSC or myeloid progenitor cell characteristics in a number of ways. For example, the more mature, differentiated cells can be selected against based on cell surface molecules they express. The blood product may be fractionated by positively selecting for CD34+ cells, which include a subpopulation of hematopoietic stem cells capable of self-renewal, multi-potency, and that can be re-introduced into a transplant recipient whereupon they home to the hematopoietic stem cell niche and reestablish productive and sustained hematopoiesis. Such selection is accomplished using, for example, commercially available magnetic anti-CD34 beads (Dynal, Lake Success, NY). Myeloid progenitor cells can also be isolated based on the markers they express. Unfractionated blood products can be obtained directly from a donor or retrieved from cryopreservative storage. HSCs and myeloid progenitor cells can also be obtained from by differentiation of ES cells, iPS cells or other reprogrammed mature cell types.

Cells that may be used in conjunction with the compositions and methods described herein include allogeneic cells and autologous cells. All of the aforementioned cell types are capable of differentiating into microglia. Cells described herein may also differentiate into microglial progenitors or microglial stem cells. Differentiation may occur ex vivo or in vivo. Methods for ex vivo differentiation of human ESCs and iPSCs are known by those of skill in the art and are described in Muffat et al., Nature Medicine 22:1358-1367 (2016) and Pandya et al., Nature Neuroscience (2017) epub ahead of print, the disclosures of which are incorporated herein by reference as they pertain to methods of differentiating pluripotent cells into microglia.

### Microglia

Cells that may be used in conjunction with the compositions and methods described herein include those that are capable of differentiating into microglial cells or cells that are differentiated microglial cells. Microglia are myeloid-derived cells that serve as the immune cells, or resident macrophages, of the central nervous system. Microglia are highly similar to macrophages, both genetically and functionally, and share the ability dynamically exhibit pro-inflammatory and anti-inflammatory states. Microglia with pro-inflammatory phenotypes have been observed in mouse models of Pompe disease, such as the double transgenic GAA knock-out (Gaa−/−) mouse (Turner et al. Respir Physiol Neurobiol 227:48-55, 2016; Korlimarla et al. Ann Trans Med 7(13):289, 2019). It is unclear whether pro-inflammatory microglia are a cause or consequence of neuroinflammation, but once microglia are classically activated, they can secrete pro-inflammatory cytokines, e.g., TNF-α, IL-1β, and IL-6, chemokines, and nitric oxide, which can lead to sustained inflammation, neuronal damage, and further activation of microglia. This positive feedback loop can be harmful to brain tissue; therefore, methods of reducing microglial pro-inflammatory signaling and/or anti-inflammatory signaling in microglia may help Pompe disease patients presenting with neuroinflammation.

### Expression of GAA in Mammalian Cells

GAA activity is reduced in patients with Pompe disease. The compositions and methods described herein target this dysfunction by administering cells (e.g., pluripotent cells, ESCs, iPSCs, multipotent cells, CD34+ cells, HSCs, MPCs, BLPCs, monocytes, macrophages, microglial progenitor cells, or microglia) containing a transgene encoding a GILT.GAA protein containing the R37A IGF-II mutein. In order to utilize these agents for therapeutic application in the treatment of Pompe disease, these agents can be directed to the interior of the cell, and in particular examples, to particular organelles or the plasma membrane, such as, e.g., the lysosome. A wide array of methods has been established for the delivery of such proteins to mammalian cells and for the stable expression of genes encoding such proteins in mammalian cells.

### Polynucleotides Encoding GAA

One platform that can be used to achieve therapeutically effective intracellular concentrations of GAA in mammalian cells (e.g., pluripotent cells, ESCs, iPSCs, multipotent cells, CD34+ cells, HSCs, MPCs, BLPCs, monocytes, macrophages, microglial progenitor cells, or microglia) is via the stable expression of genes encoding these agents (e.g., by integration into the nuclear or mitochondrial genome of a mammalian cell). These genes are polynucleotides that encode the primary amino acid sequence of the corresponding protein. In order to introduce such exogenous genes into a mammalian cell, these genes can be incorporated into a vector. Vectors can be introduced into a cell by a variety of methods, including transformation, transfection, direct uptake, projectile bombardment, and by encapsulation of the vector in a liposome. Examples of suitable methods of transfecting or transforming cells are calcium phosphate precipitation, electroporation, microinjection, infection, lipofection, and direct uptake. Such methods are described in more detail, for example, in Green et al., Molecular Cloning: A Laboratory Manual, Fourth Edition (Cold Spring Harbor University Press, New York (2014)); and Ausubel et al., Current Protocols in Molecular Biology (John Wiley & Sons, New York (2015)), the disclosures of each of which are incorporated herein by reference.

GAA can also be introduced into a mammalian cell by targeting a vector containing a gene encoding such an agent to cell membrane phospholipids. For example, vectors can be targeted to the phospholipids on the extracellular surface of the cell membrane by linking the vector molecule to a VSV-G protein, a viral protein with affinity for all cell membrane phospholipids. Such, a construct can be produced using methods well known to those of skill in the field.

### Promoter Sequences

Recognition and binding of the polynucleotide encoding GAA by mammalian RNA polymerase is important for gene expression. As such, one may include sequence elements within the polynucleotide that exhibit a high affinity for transcription factors that recruit RNA polymerase and promote the assembly of the transcription complex at the transcription initiation site. As such, one may include sequence elements within the polynucleotide that exhibit a high affinity for transcription factors that recruit RNA polymerase and promote the assembly of the transcription complex at the transcription initiation site. Such sequence elements include, e.g., a mammalian promoter, the sequence of which can be recognized and bound by specific transcription initiation factors and ultimately RNA polymerase. Examples of mammalian promoters have been described in Smith et al., Mol. Sys. Biol., 3:73, online publication, the disclosure of which is incorporated herein by reference.

In some embodiments of the disclosure, the GAA transgene may be expressed at sufficiently high levels so as to elicit a therapeutic benefit. Accordingly, transgene expression may be mediated by a promoter sequence capable of driving robust expression of the disclosed GAA constructs in target cells (e.g., pluripotent or multipotent cells). The present disclosure features heterologous promoters suitable for use with the methods and compositions disclosed herein. The term “heterologous promoter,” as used herein, refers to a promoter that is not found to be operatively linked to a given encoding sequence in nature. Useful heterologous control sequences generally include those derived from sequences encoding mammalian or viral genes.

Accordingly, polynucleotides suitable for use with the compositions and methods described herein also include those that encode GAA downstream of a mammalian promoter. Promoters that are useful for the expression of GAA in mammalian cells include, e.g., elongation factor 1-alpha (EF1α) promoter, phosphoglycerate kinase 1 (PGK) promoter, EF1a promoter containing elements of locus control region of the β-globin gene containing regions of erythroid-specific DNase I hypersensitivity (HS) regions 2, 3, and 4 (β-LCR(HS4,3,2)-EFS) promoter (see Montiel-Equihua et al. Mol Ther 20(7), 2012; Piras et al. Mol Ther 18:558-70, 2020; incorporated by reference herein as it pertains to the β-LCR(HS4,3,2)-EFS promoter), CD68 molecule (CD68) promoter (see Dahl et al., Molecular Therapy 23:835 (2015), incorporated herein by reference as it pertains to the use of PGK and CD68 promoters to express GAA), C-X3-C motif chemokine receptor 1 (CX3CR1) promoter, CD11 b promoter, allograft inflammatory factor 1 (AIF1) promoter, purinergic receptor P2Y12 (P2Y12) promoter, transmembrane protein 119 (TMEM119) promoter, and colony stimulating factor 1 receptor (CSF1R) promoter. Alternatively, promoters derived from viral genomes can also be used for the stable expression of these agents in mammalian cells. Examples of functional viral promoters that can be used to promote mammalian expression of these agents are adenovirus late promoter, vaccinia virus 7.5K promoter, simian virus 40 (SV40) promoter, cytomegalovirus promoter, tk promoter of herpes simplex virus (HSV), mouse mammary tumor virus (MMTV) promoter, long terminal repeat (LTR) promoter of human immunodeficiency virus (HIV), promoter of Moloney virus, Epstein Barr virus (EBV), Rous sarcoma virus (RSV), and the cytomegalovirus (CMV) promoter. Alternatively, synthetic promoters optimized for use in mammalian cells can be employed for stable expression of GAA. Such synthetic promoter sequence elements include, e.g., an MND promoter (such as, e.g., an MND promoter having a nucleic acid sequence of SEQ ID NO: 10 or SEQ ID NO: 11 or a variant thereof having at least 85% (e.g., at least 85%, 90%, 95%, 96%, 97%, 98%, 99%, or more) sequence identity to the nucleic acid sequence of SEQ ID NO: 10 or SEQ ID NO: 11) the sequence of which can be recognized and bound by specific transcription initiation factors and ultimately RNA polymerase. Thus, the present disclosure features a myeloproliferative sarcoma virus enhancer, negative control region deleted, dl587rev primer-binding site substituted (MND) promoter that can be incorporated into an expression cassette encoding a GAA transgene of the disclosure to drive robust transgene expression specifically in target cells. The MND promoter is a synthetic promoter sequence derived from a myeloproliferative sarcoma virus (MSV) and contains an MSV enhancer, a U3 region of a Maloney Murine Leukemia Virus, a deletion of a negative control region, and a substitution of a dl587rev primer binding site. The MND promoter may have a sequence of SEQ ID NO: 10 or may be a variant thereof having at least 85% (e.g., at least 85%, 90%, 95%, 96%, 97%, 98%, 99%, or more) sequence identity to the nucleic acid sequence of SEQ ID NO: 10, as is shown below.

(SEQ ID NO: 10)
GATCAAGGTTAGGAACAGAGAGACAGGAGAATATGGGCCAAACAGGATA
TCTGTGGTAAGCAGTTCCTGCCCCGGCTCAGGGCCAAGAACAGTTGGAA
CAGCAGAATATGGGCCAAACAGGATATCTGTGGTAAGCAGTTCCTGCCC
CGGCTCAGGGCCAAGAACAGATGGTCCCCAGATGCGGTCCCGCCCTCAG
CAGTTTCTAGAGAACCATCAGATGTTTCCAGGGTGCCCCAAGGACCTGA
AATGACCCTGTGCCTTATTTGAACTAACCAATCAGTTCGCTTCTCGCTT
CTGTTCGCGCGCTTCTGCTCCCCGAGCTCAATAAAAGAGCCCACAACCC
CTCACTCGGCGCGCCAGTCCTCCGATAGACTGCGTCGCCCGG

Additionally or alternatively, the MND promoter may have a sequence of SEQ ID NO: 11 or may be a variant thereof having at least 85% (e.g., at least 85%, 90%, 95%, 96%, 97%, 98%, 99%, or more) sequence identity to the nucleic acid sequence of SEQ ID NO: 11, as is shown below.

(SEQ ID NO: 11)
TTTATTTAGTCTCCAGAAAAAGGGGGGAATGAAAGACCCCACCTGTAGG
TTTGGCAAGCTAGGATCAAGGTTAGGAACAGAGAGACAGCAGAATATGG
GCCAAACAGGATATCTGTGGTAAGCAGTTCCTGCCCCGGCTCAGGGCCA
AGAACAGTTGGAACAGCAGAATATGGGCCAAACAGGATATCTGTGGTAA
GCAGTTCCTGCCCCGGCTCAGGGCCAAGAACAGATGGTCCCCAGATGCG
GTCCCGCCCTCAGCAGTTTCTAGAGAACCATCAGATGTTTCCAGGGTGC
CCCAAGGACCTGAAATGACCCTGTGCCTTATTTGAACTAACCAATCAGT
TCGCTTCTCGCTTCTGTTCGCGCGCTTCTGCTCCCCGAGCTCAATAAAA
GAGCCCA

Accordingly, the present disclosure contemplates expression constructs encoding a GAA protein (e.g., a GAA protein having any one of the amino acid sequences of SEQ ID NOs. 1-4 or a variant thereof having at least 85% (e.g., at least 85%, 90%, 95%, 96%, 97%, 98%, 99%, or more) sequence identity to the nucleic acid sequence of SEQ ID NOs. 1-4 fused to a GILT tag containing an R37A IGF-II mutein, wherein the transgene is operably linked to an MND promoter (e.g., having the nucleic acid sequence of SEQ ID NO: 10 or SEQ ID NO: 11 or a variant thereof having at least 85% (e.g., 90%, 95%, 96%, 97%, 98%, 99%, or more) sequence identity to the nucleic acid sequence of SEQ ID NO: 10 or SEQ ID NO: 11. In some embodiments, the MND promoter has at least 90% (e.g., at least 90%, 91%, 92%, 93%, 94%, 95%, 96%, 97%, 98%, 99%, or more) sequence identity to the nucleic acid sequence of SEQ ID NO: 10 or SEQ ID NO: 11. In some embodiments, the MND promoter has at least 95% (e.g., at least 96%, 97%, 98%, 99%, or more) sequence identity to the nucleic acid sequence of SEQ ID NO: 10 or SEQ ID NO: 11. In some embodiments, the MND promoter has at least 99% sequence identity to the nucleic acid sequence of SEQ ID NO: 10 or SEQ ID NO: 11. In some embodiments, the MND promoter has the nucleic acid sequence of SEQ ID NO: 10 or SEQ ID NO: 11.

Once a polynucleotide encoding GAA has been incorporated into the nuclear DNA of a mammalian cell, the transcription of this polynucleotide can be induced by methods known in the art. For example, expression can be induced by exposing the mammalian cell to an external chemical reagent, such as an agent that modulates the binding of a transcription factor and/or RNA polymerase to the promoter and thus regulates gene expression. The chemical reagent can serve to facilitate the binding of RNA polymerase and/or transcription factors to the promoter, e.g., by removing a repressor protein that has bound the promoter. Alternatively, the chemical reagent can serve to enhance the affinity of the promoter for RNA polymerase and/or transcription factors such that the rate of transcription of the gene located downstream of the promoter is increased in the presence of the chemical reagent. Examples of chemical reagents that potentiate polynucleotide transcription by the above mechanisms are tetracycline and doxycycline. These reagents are commercially available (Life Technologies, Carlsbad, CA) and can be administered to a mammalian cell in order to promote gene expression according to established protocols.

Other DNA sequence elements that may be included in polynucleotides for use in the compositions and methods described herein are enhancer sequences. Enhancers represent another class of regulatory elements that induce a conformational change in the polynucleotide containing the gene of interest such that the DNA adopts a three-dimensional orientation that is favorable for binding of transcription factors and RNA polymerase at the transcription initiation site. Thus, polynucleotides for use in the compositions and methods described herein include those that encode GAA and additionally include a mammalian enhancer sequence. Many enhancer sequences are now known from mammalian genes, and examples are enhancers from the genes that encode mammalian globin, elastase, albumin, α-fetoprotein, and insulin. Enhancers for use in the compositions and methods described herein also include those that are derived from the genetic material of a virus capable of infecting a eukaryotic cell. Examples are the SV40 enhancer on the late side of the replication origin (bp 100-270), the cytomegalovirus early promoter enhancer, the polyoma enhancer on the late side of the replication origin, and adenovirus enhancers. Additional enhancer sequences that induce activation of eukaryotic gene transcription are disclosed in Yaniv et al., Nature 297:17 (1982). An enhancer may be spliced into a vector containing a polynucleotide encoding a water-forming NADH oxidase, for example, at a position 5′ or 3′ to this gene. In a preferred orientation, the enhancer is positioned at the 5′ side of the promoter, which in turn is located 5′ relative to the polynucleotide encoding GAA.

### Cell-Specific Gene Expression

Interfering RNA (RNAi) are widely used to knock down the expression of endogenous genes by delivering small interfering RNA (siRNA) into cells triggering the degradation of complementary mRNA. An additional application is to utilize the diversity of endogenous micro RNAs (miRNA) to negatively regulate the expression of exogenously introduced transgenes tagged with artificial miRNA target sequences. These miRNA target tagged transgenes can be negatively regulated according to the activity of a given miRNA which can be tissue, lineage, activation, or differentiation stage specific. These artificial miRNA target sequences (miRTs) can be recognized as targets by a specific miRNA thus inducing post-transcriptional gene silencing. While robust transgene expression in targeted cells can have beneficial therapeutic results, off target expression, such as the ectopic or non-regulated transgene expression in HSPCs or other progenitor cells, can have cytotoxic effects, which can result in counter-selection of transgene-containing cells leading to altered cellular behavior and reduced therapeutic efficacy. The incorporation of miRTs for miRNAs widely expressed in HSPCs and progenitors, but absent in cells of the myeloid lineage can allow for repressed transgene expression in HSPCs and other progenitor cells allowing for silent, long-term reservoir transgene-containing hematopoietic progeny, while allowing for robust transgene expression in differentiated, mature target cells. miR-126 is highly expressed in HSPCs, other progenitor cells, and cells of the erythroid lineage, but absent from those of the myeloid lineage (e.g., macrophages and microglia) (Gentner et al., Science Translational Medicine. 2:58ra34 (2010)). A miR-126 targeting sequence, for example, incorporated within a transgene would allow for targeted expression of the transgene in cells of the myeloid lineage and repressed expression in HSPCs and other progenitor cells, thus minimizing off-target cytotoxic effects. In some embodiments, the transgene encoding GAA agent may include a miR-126 targeting sequence.

### Signal Peptides

Polynucleotides encoding GAA may include one or more polynucleotides encoding a signal peptide. Signal peptides may have amino acid sequences of 16-30 residues in length, and may be located upstream of (i.e., 5′ to) a polynucleotide encoding GAA. These signal peptides allow for the recognition of the nascent polypeptides during synthesis by signal recognition particles resulting in translocation to the ER, packaging into transport vesicles, and translocation to a target cellular compartment, to the lipid membrane, or to the extracellular space. Exemplary signal peptides for protein translocation are those from GAA, IGF-II, alpha-1 antitrypsin, IL-2, IL-6, CD5, immunoglobulins, trypsinogen, serum albumin, prolactin, elastin, tissue plasminogen activator signal peptide (tPA-SP), and insulin. In some embodiments, the IGF-II signal peptide sequence has an amino acid sequence of SEQ ID NO: 12 or is a variant thereof having at least 70% (e.g., at least 75%, 80%, 85%, 90%, 95%, 96%, 97%, 98%, 99%, or more) sequence identity to the amino acid sequence of SEQ ID NO: 12.

(SEQ ID NO: 12)
MGIPMGKSMLVLLTFLAFASCCIA

In some embodiments, cells (e.g., pluripotent cells, ESCs, iPSCs, multipotent cells, CD34+ cells, HSCs, MPCs, BLPCs, monocytes, macrophages, microglial progenitor cells, or microglia) containing a transgene encoding GILT.GAA may be utilized as a therapeutic strategy to correct a protein deficiency (e.g., GAA) by infusing the missing protein into the bloodstream. As the blood perfuses patient tissues, GAA is taken up by cells and transported to its site of action.

### Glycosylation Independent Lysosomal Targeting

GILT technology can be utilized to target therapeutic enzymes (e.g., GAA) to lysosomes. Specifically, the GILT technology uses a peptide tag instead of M6P to engage the CI-MPR for lysosomal targeting. Typically, a GILT tag is a protein, peptide, or other moiety that binds the CI-MPR in a mannose-6-phosphateindependent manner. Advantageously, this technology mimics the normal biological mechanism for uptake of lysosomal enzymes, yet does so in a manner independent of mannose-6-phosphate. In some embodiments, the GAA is secreted as a GAA fusion protein containing GAA and a GILT tag. In some embodiments, a GILT tag is derived from the mature human IGF-II protein. Human IGF-II is a high affinity ligand for the CI-MPR; also referred to as IGF-II receptor. Binding of GILT-tagged therapeutic enzymes to the M6P/IGF-II receptor targets the protein to the lysosome via the endocytic pathway. A detailed description of GILT technology and the GILT tag can be found in U.S. Publication Nos. 20030082176, 20040006008, 20040005309, 20050281805, and 2009043207 the teachings of all of which are hereby incorporated by references in their entireties. In some embodiments, the GILT tag contains a linker. In some embodiments the linker is on the N-terminus of the GILT tag. In some embodiments, the linker is on the C-terminus of the GILT tag. In some embodiments, the linker contains the amino acid sequence Gly-Ala-Pro (SEQ ID NO: 13). In some embodiments, the linker is encoded by a nucleic acid sequence of GGCGCGCCG (SEQ ID NO: 14). In some embodiments, the GAP linker is covalently linked to the N-terminus of a GILT tag of the disclosure. In some embodiments, the GAP linker is covalently linked to the C-terminus of a GILT tag of the disclosure. In some embodiments, the GAP linker is inserted at an amino acid position between the N-terminus and the C-terminus of the GILT tag. In some embodiments, the GAP linker is covalently linked to the N-terminus of GAA of the disclosure. In some embodiments, the GAP linker is covalently linked to the C-terminus of GAA of the disclosure. In some embodiments, the GAP linker is inserted at an amino acid position between the N-terminus and the C-terminus of GAA of the disclosure. In some embodiments, the GAP linker is inserted at amino acid position 791 of a GAA protein having the amino acid sequence of SEQ ID NO: 1 or a variant thereof having at least 85% (e.g., at least 90%, 91%, 92%, 93%, 94%, 95%, 96%, 97%, 98%, 99%, or more) sequence identity to the amino acid sequence of SEQ ID NO: 1.

### Furin-Resistant GILT Tag

The IGF-II derived GILT tag may be subjected to proteolytic cleavage by furin during production in mammalian cells. Furin protease typically recognizes and cleaves a cleavage site having a consensus sequence Arg-X-X-Arg, where X is any amino acid. The cleavage site is positioned after the carboxy-terminal arginine (Arg) residue in the sequence. In some embodiments, a furin cleavage site has a consensus sequence Lys/Arg-X-X-X-Lys/Arg-Arg, where X is any amino acid. The cleavage site is positioned after the carboxy terminal arginine (Arg) residue in the sequence. The mature human IGF-II peptide sequence is shown below.

(SEQ ID NO: 15)
AYRPSETLCGGELVDTLQFVCGDRGFYFSRPASRVSRRSRGIVEECCFR
SCDLALLETYCATPAKSE

The mature human IGF-II contains two potential overlapping furin cleavage sites between residues 34-40 (bolded). Modified GILT tags that are resistant to cleavage by furin still retain ability to bind to the CI-MPR in a mannose-6-phosphate-independent manner. Specifically, furin-resistant GILT tags can be designed by mutating the amino acid sequence at one or more furin cleavage sites such that the mutation abolishes at least one furin cleavage site. Thus, in some embodiments, a furin-resistant GILT tag is a furin-resistant IGF-II mutein containing a mutation that abolishes at least one furin protease cleavage site or changes a sequence adjacent to the furin protease cleavage site such that the furin cleavage is prevented, inhibited, reduced or slowed down as compared to a wild-type IGF-II peptide (e.g., wild-type human mature IGF-II). A suitable mutation does not impact the ability of the furin-resistant GILT tag to bind to the human cation-independent mannose-6-phosphate receptor. In some embodiments, a furin-resistant IGF-II mutein suitable for use in conjunction with the compositions and methods described herein binds to the human cation-independent mannose-6-phosphate receptor in a mannose-6-phosphate-independent manner with a dissociation constant of 10⁻⁷ M or less (e.g., 10⁻⁸, 10⁻⁹, 10⁻¹⁰, 10⁻¹¹, or less) at pH 7.4. In some embodiments, a furin-resistant IGF-II mutein contains a mutation within a region corresponding to amino acids 30-40 (e.g., 31-40, 32-40, 33-40, 34-40, 30-39, 31-39, 32-39, 34-37, 32-39, 33-39, 34-39, 35-39, 36-39, 37-40, 34-40) of SEQ ID NO: 15. In some embodiments, a suitable mutation abolishes at least one furin protease cleavage site. A mutation can be amino acid substitutions, deletions, or insertions. For example, any one amino acid within the region corresponding to residues 30-40 (e.g., 31-40, 32-40, 33-40, 34-40, 30-39, 31-39, 32-39, 34-37, 32-39, 33-39, 34-39, 35-39, 36-39, 37-40, 34-40) of SEQ ID NO: 15 can be substituted with any other amino acid or deleted. For example, substitutions at position 34 may affect furin recognition of the first cleavage site. Insertion of one or more additional amino acids within each recognition site may abolish one or both furin cleavage sites. Deletion of one or more of the residues in the degenerate positions may also abolish both furin cleavage sites.

In some embodiments, a furin-resistant IGF-II mutein contains amino acid substitutions at positions corresponding to Arg37 or Arg40 of SEQ ID NO: 15. In some embodiments, a furin-resistant IGF-II mutein contains a Lys or Ala substitution at positions Arg37 or Arg40. Other substitutions are possible, including combinations of Lys and/or Ala mutations at both positions 37 and 40, or substitutions of amino acids other than Lys or Ala. In some embodiments, the furin-resistant IGF-II mutein suitable for use in conjunction with the compositions and methods described herein may contain additional mutations. For example, up to 30% or more of the residues of SEQ ID NO: 15 may be changed (e.g., up to 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30% or more residues may be changed). Thus, a furin-resistant IGF-II mutein suitable for use in conjunction with the compositions and methods described herein may have an amino acid sequence at least 70%, including at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99%, identical to SEQ ID NO: 15. In some embodiments, a furin-resistant IGF-II mutein suitable for use in conjunction with the compositions and methods described herein is targeted specifically to the CI-MPR. Particularly useful are mutations in the IGF-II polypeptide that result in a protein that binds the CI-MPR with high affinity (e.g., with a dissociation constant of 10⁻⁷ M or less at pH 7.4) while binding other receptors known to be bound by IGF-II with reduced affinity relative to native IGF-II. For example, a furin-resistant IGF-II mutein suitable for use in conjunction with the compositions and methods described herein can be modified to have diminished binding affinity for the IGF-I receptor relative to the affinity of naturally-occurring human IGF-II for the IGF-I receptor. Additional mutational strategies have been utilized and are discussed at length in the U.S. Publication No. 2009043207, which is hereby incorporated by reference. For example, substitution of IGF-II residues Tyr 27 with Leu, Leu 43 with Val, or Ser 26 with Phe diminishes the affinity of IGF-II for the IGF-I receptor by 94-, 56-, and 4-fold respectively (Torres et al., J. Mol. Biol. 248(2):385-401 (1995)). Deletion of residues 1-7 of human IGF-II resulted in a 30-fold decrease in affinity for the human IGF-I receptor and a concomitant 12-fold increase in affinity for the rat IGF-II receptor (Hashimoto et al., J. Biol. Chem. 270(30):18013-8 (1995)). The NMR structure of IGF-II shows that Thr 7 is located near residues 48 Phe and 50 Ser, as well as near the 9 Cys-4 7 Cys disulfide bridge. It is thought that interaction of Thr 7 with these residues can stabilize the flexible N-terminal hexapeptide required for IGF-I receptor binding (Terasawa et al., EMBO J. 13(23)5590-7 (1994)). At the same time, this interaction can modulate binding to the IGF-II receptor. Truncation of the C-terminus of IGF-II (residues 62-67) also appears to lower the affinity of IGF-II for the IGF-I receptor by 5-old (Roth et al., Biochem. Biophys. Res. Commun. 181(2):907-14 (1991)). The binding surfaces for the IGF-I and cation-independent M6P receptors are on separate faces of IGF-II. Based on structural and mutational data, functional cation-independent M6P binding domains can be constructed that are substantially smaller than human IGF-II. For example, the amino terminal amino acids (e.g., 1-7 or 2-7) and/or the carboxy terminal residues 62-67 can be deleted or replaced. Additionally, amino acids 29-40 can likely be eliminated or replaced without altering the folding of the remainder of the polypeptide or binding to the cation-independent M6P receptor. Thus, a targeting moiety including amino acids 8-28 and 41-61 can be constructed. These stretches of amino acids could perhaps be joined directly or separated by a linker. Alternatively, amino acids 8-28 and 41-61 can be provided on separate polypeptide chains. Comparable domains of insulin, which are homologous to IGF-II and have a tertiary structure closely related to the structure of IGF-II, have sufficient structural information to permit proper refolding into the appropriate tertiary structure, even when present in separate polypeptide chains (Wang et al., Trends Biochem. Sci. 16(8):279-281 (1991)). Thus, for example, amino acids 8-28, or a conservative substitution variant thereof, could be fused to a lysosomal enzyme; the resulting fusion protein could be admixed with amino acids 41-61, or a conservative substitution variant thereof, and administered to a patient. IGF-II can also be modified to minimize binding to serum IGF-binding proteins (Baxter, Am. J. Physiol Endocrinol Metab. 278(6):967-76(2000)) to avoid sequestration of IGF-II/GILT constructs. A number of studies have localized residues in IGF-II necessary for binding to IGF-binding proteins. Constructs with mutations at these residues can be screened for retention of high affinity binding to the M6P/IGF-II receptor and for reduced affinity for IGF binding proteins. For example, replacing Phe 26 of IGF-II with Ser is reported to reduce affinity of IGF-II for IGFBP-1 and -6, with no effect on binding to the M6P/IGF-II receptor (Bach et al., J. Biol. Chem. 268(13):9246-54 (1993)). Other substitutions, such as Lys for Glu 9, can also be advantageous. The analogous mutations, separately or in combination, in a region of IGF-I that is highly conserved with IGF-II result in large decreases in IGF-BP binding (Magee et al., Biochemistry 38(48):15863-70 (1999)).

An alternate approach is to identify minimal regions of IGF-II that can bind with high affinity to the M6P/IGF-II receptor. The residues that have been implicated in IGF-II binding to the M6P/IGF-II receptor mostly cluster on one face of IGF-II (Terasawa et al., EMBO J. 13(23):5590-7 (1994)). Although IGF-II tertiary structure is normally maintained by three intramolecular disulfide bonds, a peptide incorporating the amino acid sequence on the M6P/IGF-II receptor binding surface of IGF-II can be designed to fold properly and have binding activity. Such a minimal binding peptide is a highly preferred lysosomal targeting domain. For example, a preferred lysosomal targeting domain is amino acids 8-67 of human IGF-II. Designed peptides, based on the region around amino acids 48-55, which bind to the M6P/IGF-II receptor, are also desirable lysosomal targeting domains. Alternatively, a random library of peptides can be screened for the ability to bind the M6P/IGF-II receptor either via a yeast two hybrid assay, or via a phage display type assay.

Many furin-resistant IGF-II muteins described herein have reduced or diminished binding affinity for the insulin receptor. Thus, in some embodiments, a peptide tag suitable for use in conjunction with the compositions and methods described herein has reduced or diminished binding affinity for the insulin receptor relative to the affinity of naturally occurring human IGF-II for the insulin receptor. In some embodiments, peptide tags with reduced or diminished binding affinity for the insulin receptor suitable for use in conjunction with the compositions and methods described herein include peptide tags having a binding affinity for the insulin receptor that is more than 1.5-fold, 2-fold, 3-fold, 4-fold, 5-fold, 6-fold, 7-fold, 8-fold, 9-fold, 10-fold, 12-fold, 14-fold, 16-fold, 18-fold, 20-fold, 50-fold, 100-fold less than that of the wild-type mature human IGF-II. The binding affinity for the insulin receptor can be measured using various in vitro and in vivo assays known in the art.

In some embodiments, the GILT tag has an amino acid sequence having at least 70% sequence identity (e.g., 75%, 80%, 85%, 90%, 95%, 96%, 97%, 98%, 99%, or greater, sequence identity) to the amino acid sequence of SEQ NO. 16, as shown below.

(SEQ ID NO: 16)
GGGGAGGGGAGGGGAGGGGAGGGPSLCGGELVDTLQFVCGDRGFYFSRP
ASRVSARSRGIVEECCFRSCDLALLETYCATPAKSE

In some embodiments, the GILT tag has an amino acid sequence having at least 70% sequence identity (e.g., 75%, 80%, 85%, 90%, 95%, 96%, 97%, 98%, 99%, or greater, sequence identity) to the amino acid sequence of SEQ NO. 17, as shown below.

(SEQ ID NO: 17)
GAPGGGSPAPAPTPAPAPTPAPAGGGPSGAPLCGGELVDTLQFVCGDRG
FYFSRPASRVSARSRGIVEECCFRSCDLALLETYCATPAKSE

In some embodiments, the GILT tag has an amino acid sequence having at least 70% sequence identity (e.g., 75%, 80%, 85%, 90%, 95%, 96%, 97%, 98%, 99%, or greater, sequence identity) to the amino acid sequence of SEQ NO. 18, as shown below.

(SEQ ID NO: 18)
GAPGGSPAGSPTSTEEGTSESATPESGPGTSTEPSEGSAPGSPAGSPTS
TGPSGAPLCGGELVDTLQFVCGDRGFYFSRPASRVSARSRGIVEECCFR
SCDLALLETYCATPAKSE

In some embodiments, the GILT tag has an amino acid sequence having at least 70% sequence identity (e.g., 75%, 80%, 85%, 90%, 95%, 96%, 97%, 98%, 99%, or greater, sequence identity) to the amino acid sequence of SEQ NO. 19, as shown below.

(SEQ ID NO: 19)
MGIPMGKSMLVLLTFLAFASCCIAAYRPSETLCGGELVDTLQFVCGDRG
FYFSRPASRVSRRSRGIVEECCFRSCDLALLETYCATPAKSEGAP

In some embodiments, the GILT tag has an amino acid sequence having at least 70% sequence identity (e.g., 75%, 80%, 85%, 90%, 95%, 96%, 97%, 98%, 99%, or greater, sequence identity) to the amino acid sequence of SEQ NO. 20, as shown below

(SEQ ID NO: 20)
MGIPMGKSMLVLLTFLAFASCCIAALCGGELVDTLQFVCGDRGFYFSRP
ASRVSARSRGIVEECCFRSCDLALLETYCATPAKSEGAP

In some embodiments, the GILT tag is encoded by a nucleic acid sequence having at least 85% sequence identity (e.g., 85%, 90%, 95%, 96%, 97%, 98%, 99%, or greater, sequence identity) to the nucleic acid sequence of SEQ ID NO: 21, as shown below.

(SEQ ID NO: 21)
GGCGGAGGCGGAGCTGGTGGCGGCGGAGCAGGCGGTGGTGGTGCAGGCG
GCGGAGGTGCTGGCGGAGGACCATCTCTTTGTGGCGGAGAACTGGTGGA
CACCCTGCAGTTCGTGTGTGGCGACAGAGGCTTCTACTTTAGCAGACCC
GCCAGCAGAGTGTCCGCCAGATCTAGAGGAATCGTGGAAGAGTGCTGCT
TCAGAAGCTGCGACCTGGCACTGCTGGAAACCTACTGTGCCACACCAGC
CAAGAGCGAGTGATG

In some embodiments, the GILT tag is encoded by a nucleic acid sequence having at least 85% sequence identity (e.g., 85%, 90%, 95%, 96%, 97%, 98%, 99%, or greater, sequence identity) to the nucleic acid sequence of SEQ ID NO: 22, as shown below.

(SEQ ID NO: 22)
GGAGCACCAGGCGGAGGATCTCCAGCTCCTGCTCCTACACCAGCTCCAG
CACCGACGCCTGCTCCAGCTGGCGGAGGACCTTCTGGTGCACCTCTTTG
TGGCGGAGAGCTGGTGGATACCCTGCAGTTCGTGTGTGGCGACCGGGGC
TTCTACTTTAGCAGACCTGCCAGCAGAGTGTCCGCCAGATCTAGAGGCA
TCGTGGAAGAGTGCTGCTTCAGAAGCTGCGACCTGGCACTGCTGGAAAC
CTACTGTGCCACACCAGCCAAGAGCGAGTGATGA

In some embodiments, the GILT tag is encoded by a nucleic acid sequence having at least 85% sequence identity (e.g., 85%, 90%, 95%, 96%, 97%, 98%, 99%, or greater, sequence identity) to the nucleic acid sequence of SEQ ID NO: 23, as shown below.

(SEQ ID NO: 23)
GGAGCACCAGGCGGATCTCCAGCAGGATCTCCAACCTCTACCGAGGAAG
GCACAAGCGAGTCTGCCACACCTGAGTCTGGACCTGGCACAAGCACAGA
GCCTAGCGAAGGATCTGCCCCAGGTTCTCCTGCCGGCTCTCCTACAAGT
ACAGGACCTTCTGGCGCTCCACTGTGTGGCGGAGAACTGGTGGATACCC
TGCAGTTCGTGTGCGGCGACAGAGGCTTCTACTTTAGCAGACCCGCCAG
CAGAGTGTCCGCCAGATCTAGAGGAATCGTGGAAGAGTGCTGCTTCAGA
AGCTGCGATCTGGCACTGCTGGAAACCTACTGTGCCACACCAGCCAAGA
GCGAGTGATGA

In some embodiments, the GILT tag is fused to or near the N-terminus (e.g., less than 20, such as, e.g., less than 19, 18, 17, 16, 15, 14, 13, 12, 11, 10, 9, 8, 7, 6, 5, 4, 3, 2, or 1, amino acids upstream of the N-terminus) of the GAA protein. In some embodiments, the GILT tag is fused to or near the C-terminus (e.g., less than 20, such as, e.g., less than 19, 18, 17, 16, 15, 14, 13, 12, 11, 10, 9, 8, 7, 6, 5, 4, 3, 2, or 1, amino acids downstream of the C-terminus) of the GAA protein.

### ApoE Tag for Blood-Brain Barrier Penetrance of GAA

In some embodiments, the GAA (e.g., GILT.GAA fusion protein) is modified to penetrate the blood-brain barrier (BBB). Modifications for mediating BBB penetrance are well known in the art. Exemplary modifications are the use of tags containing the Rb domain (amino acid residues 148-173 of SEQ ID NO: 24) of ApoE. The complete ApoE peptide sequence is shown below.

(SEQ ID NO: 24)
MKVLWAALLVTFLAGCQAKVEQAVETEPEPELRQQTEWQSGQRWELALG
RFWDYLRWVQTLSEQVQEELLSSQVTQELRALMDETMKELKAYKSELEE
QLTPVAEETRARLSKELQAAQARLGADMEDVCGRLVQYRGEVQAMLGQS
TEELRVRLASHLRKLRKRLLRDADDLQKRLAVYQAGAREGAERGLSAIR
ERLGPLVEQGRVRAATVGSLAGQPLQERAQAWGERLRARMEEMGSRTRD
RLDEVKEQVAEVRAKLEEQAQQIRLQAEAFQARLKSWFEPLVEDMQRQW
AGLVEKVQAAVGTSAAPVPSDNH

ApoE is an important protein involved in lipid transport, and its cellular internalization is mediated by several members of the low-density lipoprotein (LDL) receptor gene family, including the LDL receptor, very low-density lipoprotein receptor (VLDLR), and LDL receptor-related proteins (LRPs, including LRP1, LRP2, and LRP8). The LDL receptor is found to be highly expressed in brain capillary endothelial cells (BCECs), with down-regulated expression observed in peripheral vessels. Restricted expressions of LRPs and VLDLR have also been shown prominently in the liver and brain when they have been detected in BCECs, neurons, and glial cells. Several members of the low-density lipoprotein receptor family (LDLRf) proteins, including LRP1 and VLDLR, but not LDLR, are highly expressed in BBB-forming BCECs. These proteins can bind ApoE to facilitate their transcytosis into the abluminal side of the BBB.

In addition, receptor-associated protein (RAP), an antagonist as well as a ligand for both LRP1 and VLDLR, has been shown to have higher permeability across the BBB than transferrin in vivo and in vitro (Pan et al., J. Cell Sci. 117:5071-8 (2004)), indicating that these lipoprotein receptors (LDLRf) can represent efficient BBB delivery targets despite their lower expression than the transferrin receptor. As described herein, a potent Rb peptide derived from ApoE, has the ability to translocate protein across the BBB into the brain when engineered as fusion proteins. This method can therefore function to selectively open the BBB for therapeutic agents (e.g., GAA) when engineered as a fusion protein. This peptide can be readily attached to diagnostic or therapeutic agents without jeopardizing their biological functions or interfering with the important biological functions of ApoE due to the utilization of the Rb domain of ApoE, rather than the entire ApoE protein. This pathway is also an alternative uptake pathway that can facilitate further/secondary distribution within the brain after the agents reach the CNS due to the widespread expression of LDLRf members in brain parenchyma. Regardless of application strategies, e.g., enzyme replacement therapy or cell-based, gene-based therapy, both the quantity and distribution of therapeutics within the brain parenchyma will have a significant impact on the clinical outcome of disease treatment. The development of and a detailed description of the use of the Rb domain of ApoE in targeted delivery of proteins across the BBB can be found in U.S. Publication No. 20140219974, which is hereby incorporated by reference in its entirety.

In some embodiments, the GILT.GAA fusion protein has a peptide sequence containing the LDLRf Rb domain of SEQ ID NO: 24, or a fragment, variant, or oligomer thereof. An exemplary receptor-binding domain can be found in the N-terminus of ApoE, for example, between amino acid residues 1 to 191 of SEQ ID NO: 24, between amino acid residues 25 to 185 of SEQ ID NO: 24, between amino acid residues 50 to 180 of SEQ ID NO: 24, between amino acid residues 75 to 175 of SEQ ID NO: 24, between amino acid residues 100 to 170 of SEQ ID NO: 24, or between amino acid residues 125 to 165 of SEQ ID NO: 24. An exemplary receptor-binding domain has the amino acid sequence of residues 159 to 167 of SEQ ID NO: 24.

In some embodiments, the ApoE tag contains an amino acid of SEQ ID NO: 25 or is a variant thereof having at least 70% (e.g., at least 75%, 80%, 85%, 90%, 95%, 96%, 97%, 98%, 99%, or more) sequence identity to the amino acid sequence of SEQ ID NO: 25 (LRKLRKRLLLRKLRKRLL).

In some embodiments the ApoE tag contains a GAP linker on the N-terminus of the ApoE tag. In some embodiments the ApoE tag contains a GAP linker on the C-terminus of the ApoE tag. In some embodiments the ApoE tag contains a GAP linker on the N-terminus and C-terminus of the ApoE tag. In some embodiments, the ApoE tag is inserted into one or more regions of the of a GILT tag. In some embodiments, the ApoE tag (e.g., SEQ ID NO: 25) is inserted between amino acid positions 25 and 26 of the GILT tag of SEQ ID NO: 20. In some embodiments, the ApoE tag (e.g., SEQ ID NO: 25) is covalently linked to the N-terminus of the GILT tag. In some embodiments, the ApoE tag (e.g., SEQ ID NO: 25) is covalently linked to C-terminus of the GILT tag. In some embodiments, the combined GILT tag and ApoE tag sequence has an amino acid of SEQ ID NO: 26 or is a variant thereof having at least 70% (e.g., at least 75%, 80%, 85%, 90%, 95%, 96%, 97%, 98%, 99%, or more) sequence identity to the amino acid sequence of SEQ ID NO: 26.

(SEQ ID NO: 26)
MGIPMGKSMLVLLTFLAFASCCIAAGAPLRKLRKRLLLRKLRKRLLGAP
LCGGELVDTLQFVCGDRGFYFSRPASRVSARSRGIVEECCFRSCDLALL
ETYCATPAKSE

In some embodiments, the peptide sequence containing the receptor-binding domain of ApoE can include at least one amino acid mutation, deletion, addition, or substitution. In some embodiments, the amino acid substitutions can be a combination of two or more mutations, deletions, additions, or substitutions. In some embodiments, the at least one substation is a conservative substitution. In some embodiments, the at least one amino acid addition includes addition of a selected sequence already found in the Rb domain of ApoE. A person of ordinary skill in the art will recognize suitable modifications that can be made to the sequence while retaining some degree of the biochemical activity for transport across the BBB.

### Vectors for the Expression of GAA

In addition to achieving high rates of transcription and translation, stable expression of an exogenous gene in a mammalian cell (e.g., pluripotent cell, ESC, iPSC, multipotent cell, CD34+ cell, HSC, MPC, BLPC, monocyte, macrophage, microglial progenitor cell, or microglial cell) can be achieved by integration of the polynucleotide containing the gene into the nuclear genome of the mammalian cell. A variety of vectors for the delivery and integration of polynucleotides encoding exogenous proteins into the nuclear DNA of a mammalian cell have been developed. Examples of expression vectors are disclosed in, e.g., WO 1994/011026 and are incorporated herein by reference. Expression vectors for use in the compositions and methods described herein contain a polynucleotide that encodes GILT.GAA, as well as, e.g., additional sequence elements used for the expression of these agents and/or the integration of these polynucleotides into the genome of a mammalian cell. Certain vectors that can be used for the expression of GAA include plasmids that contain regulatory sequences, such as promoter and enhancer regions, which direct gene transcription. Other useful vectors for expression of GAA contain polynucleotides that enhance the rate of translation of these genes or improve the stability or nuclear export of the mRNA that results from gene transcription. These sequence elements include, e.g., 5′ and 3′ untranslated regions, an IRES, and polyadenylation signal site in order to direct efficient transcription of the gene carried on the expression vector. The expression vectors suitable for use with the compositions and methods described herein may also contain a polynucleotide encoding a marker for selection of cells that contain such a vector. Examples of a suitable marker are genes that encode resistance to antibiotics, such as ampicillin, chloramphenicol, kanamycin, nourseothricin.

### Viral Vectors for Expression of GAA

Viral genomes provide a rich source of vectors that can be used for the efficient delivery of exogenous genes into a mammalian cell (e.g., pluripotent cell, ESC, iPSC, multipotent cell, CD34+ cell, HSC, MPC, BLPC, monocyte, macrophage, microglial progenitor cell, or microglial cell). Viral genomes are particularly useful vectors for gene delivery as the polynucleotides contained within such genomes are typically incorporated into the nuclear genome of a mammalian cell by generalized or specialized transduction. These processes occur as part of the natural viral replication cycle, and do not require added proteins or reagents in order to induce gene integration. Examples of viral vectors are a retrovirus (e.g., Retroviridae family viral vector), adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses are: avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus (Coffin, J. M., Retroviridae: The viruses and their replication, Virology, Third Edition (Lippincott-Raven, Philadelphia, (1996))). Other examples are murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in McVey et al., (U.S. Pat. No. 5,801,030), the teachings of which are incorporated herein by reference.

### Retroviral Vectors

The delivery vector used in the methods and compositions described herein may be a retroviral vector. One type of retroviral vector that may be used in the methods and compositions described herein is a lentiviral vector. Lentiviral vectors (LVs), a subset of retroviruses, transduce a wide range of dividing and non-dividing cell types with high efficiency, conferring stable, long-term expression of the transgene. An overview of optimization strategies for packaging and transducing LVs is provided in Delenda, The Journal of Gene Medicine 6: S125 (2004), the disclosure of which is incorporated herein by reference.

The use of lentivirus-based gene transfer techniques relies on the in vitro production of recombinant lentiviral particles carrying a highly deleted viral genome in which the transgene of interest is accommodated. In particular, the recombinant lentivirus are recovered through the in trans co-expression in a permissive cell line of (1) the packaging constructs, i.e., a vector expressing the Gag-Pol precursors together with Rev (alternatively expressed in trans); (2) a vector expressing an envelope receptor, generally of an heterologous nature; and (3) the transfer vector, consisting in the viral cDNA deprived of all open reading frames, but maintaining the sequences required for replication, incapsidation, and expression, in which the sequences to be expressed are inserted.

A LV used in the methods and compositions described herein may include one or more of a 5′-Long terminal repeat (LTR), HIV signal sequence, HIV Psi signal 5′-splice site (SD), delta-GAG element, Rev Responsive Element (RRE), 3′-splice site (SA), and 3′-self inactivating LTR (SIN-LTR). The lentiviral vector optionally includes a central polypurine tract (cPPT) and a woodchuck hepatitis virus post-transcriptional regulatory element (WPRE), as described in U.S. Pat. No. 6,136,597, the disclosure of which is incorporated herein by reference as it pertains to WPRE. The lentiviral vector may further include a pHR′ backbone, which may include for example as provided below.

The Lentigen LV described in Lu et al., Journal of Gene Medicine 6:963 (2004) may be used to express the DNA molecules and/or transduce cells. A LV used in the methods and compositions described herein may a 5′-Long terminal repeat (LTR), HIV signal sequence, HIV Psi signal 5′-splice site (SD), delta-GAG element, Rev Responsive Element (RRE), 3′-splice site (SA), and 3′-self inactivating L TR (SIN-LTR). It will be readily apparent to one skilled in the art that optionally one or more of these regions is substituted with another region performing a similar function.

Enhancer elements can be used to increase expression of modified DNA molecules or increase the lentiviral integration efficiency. The LV used in the methods and compositions described herein may include a nef sequence. The LV used in the methods and compositions described herein may include a cPPT sequence which enhances vector integration. The cPPT acts as a second origin of the (+)-strand DNA synthesis and introduces a partial strand overlap in the middle of its native HIV genome. The introduction of the cPPT sequence in the transfer vector backbone strongly increased the nuclear transport and the total amount of genome integrated into the DNA of target cells. The LV used in the methods and compositions described herein may include a Woodchuck Posttranscriptional Regulatory Element (WPRE). The WPRE acts at the transcriptional level, by promoting nuclear export of transcripts and/or by increasing the efficiency of polyadenylation of the nascent transcript, thus increasing the total amount of mRNA in the cells. The addition of the WPRE to LV results in a substantial improvement in the level of transgene expression from several different promoters, both in vitro and in vivo. The LV used in the methods and compositions described herein may include both a cPPT sequence and WPRE sequence. The vector may also include an IRES sequence that permits the expression of multiple polypeptides from a single promoter.

In addition to IRES sequences, other elements which permit expression of multiple polypeptides are useful. The vector used in the methods and compositions described herein may include a protein cleavage site that allows expression of more than one polypeptide. Examples of protein cleavage sites that allow expression of more than one polypeptide are described in Klump et al., Gene Ther.; 8:811 (2001), Osborn et al., Molecular Therapy 12:569 (2005), Szymczak and Vignali, Expert Opin Biol Ther. 5:627 (2005), and Szymczak et al., Nat Biotechnol. 22:589 (2004), the disclosures of which are incorporated herein by reference as they pertain to protein cleavage sites that allow expression of more than one polypeptide. It will be readily apparent to one skilled in the art that other elements that permit expression of multiple polypeptides identified in the future are useful and may be utilized in the vectors suitable for use with the compositions and methods described herein.

The vector used in the methods and compositions described herein may, be a clinical grade vector.

### Viral Regulatory Elements

The viral regulatory elements are components of delivery vehicles used to introduce nucleic acid molecules into a host cell (e.g., pluripotent cells, ESC, iPSC, multipotent cell, CD34+ cell, HSC, MPC, BLPC, monocyte, macrophage, microglial progenitor cell, or microglial cell). The viral regulatory elements are optionally retroviral regulatory elements. For example, the viral regulatory elements may be the LTR and gag sequences from HSC1 or MSCV. The retroviral regulatory elements may be from lentiviruses or they may be heterologous sequences identified from other genomic regions. One skilled in the art would also appreciate that as other viral regulatory elements are identified, these may be used with the nucleic acid molecules described herein.

### Adeno-Associated Viral Vectors for Nucleic Acid Delivery

Nucleic acids of the compositions and methods described herein may be incorporated into rAAV vectors and/or virions in order to facilitate their introduction into a cell (e.g., pluripotent cells, ESC, iPSC, multipotent cell, CD34+ cell, HSC, MPC, BLPC, monocyte, macrophage, microglial progenitor cell, or microglial cell). AAV vectors can be used in the central nervous system, and appropriate serotypes are discussed in Pignataro et al., J Neural Transm. (2017), epub ahead of print, the disclosure of which is incorporated herein by reference as it pertains to AAV serotypes useful in CNS gene therapy. rAAV vectors useful in the compositions and methods described herein are recombinant nucleic acid constructs that include (1) a heterologous sequence to be expressed (e.g., a polynucleotide encoding GAA) and (2) viral sequences that facilitate integration and expression of the heterologous genes. The viral sequences may include those sequences of AAV that are required in cis for replication and packaging (e.g., functional ITRs) of the DNA into a virion. Such rAAV vectors may also contain marker or reporter genes. Useful rAAV vectors have one or more of the AAV WT genes deleted in whole or in part but retain functional flanking ITR sequences. The AAV ITRs may be of any serotype suitable for a particular application. Methods for using rAAV vectors are described, for example, in Tai et al., J. Biomed. Sci. 7:279 (2000), and Monahan and Samulski, Gene Delivery 7:24 (2000), the disclosures of each of which are incorporated herein by reference as they pertain to AAV vectors for gene delivery.

The nucleic acids and vectors described herein can be incorporated into a rAAV virion in order to facilitate introduction of the nucleic acid or vector into a cell. The capsid proteins of AAV compose the exterior, non-nucleic acid portion of the virion and are encoded by the AAV cap gene. The cap gene encodes three viral coat proteins, VP1, VP2, and VP3, which are required for virion assembly. The construction of rAAV virions has been described, for example, in U.S. Pat. Nos. 5,173,414; 5,139,941; 5,863,541; 5,869,305; 6,057,152; and 6,376,237; as well as in Rabinowitz et al., J. Virol. 76:791 (2002) and Bowles et al., J. Virol. 77:423 (2003), the disclosures of each of which are incorporated herein by reference as they pertain to AAV vectors for gene delivery.

rAAV virions useful in conjunction with the compositions and methods described herein include those derived from a variety of AAV serotypes including AAV 1, 2, 3, 4, 5, 6, 7, 8, 9, 10 and rh74. For targeting cells located in or delivered to the central nervous system, AAV2, AAV9, and AAV10 may be particularly useful. Construction and use of AAV vectors and AAV proteins of different serotypes are described, for example, in Chao et al., Mol. Ther. 2:619 (2000); Davidson et al., Proc. Natl. Acad. Sci. USA 97:3428 (2000); Xiao et al., J. Virol. 72:2224 (1998); Halbert et al., J. Virol. 74:1524 (2000); Halbert et al., J. Virol. 75:6615 (2001); and Auricchio et al., Hum. Molec. Genet. 10:3075 (2001), the disclosures of each of which are incorporated herein by reference as they pertain to AAV vectors for gene delivery.

Also useful in conjunction with the compositions and methods described herein are pseudotyped rAAV vectors. Pseudotyped vectors include AAV vectors of a given serotype pseudotyped with a capsid gene derived from a serotype other than the given serotype (e.g., AAV1, AAV2, AAV3, AAV4, AAV5, AAV6, AAV7, AAV8, AAV9, and AAV10, among others). Techniques involving the construction and use of pseudotyped rAAV virions are known in the art and are described, for example, in Duan et al., J. Virol. 75:7662 (2001); Halbert et al., J. Virol. 74:1524 (2000); Zolotukhin et al., Methods, 28:158 (2002); and Auricchio et al., Hum. Molec. Genet. 10:3075 (2001).

AAV virions that have mutations within the virion capsid may be used to infect particular cell types more effectively than non-mutated capsid virions. For example, suitable AAV mutants may have ligand insertion mutations for the facilitation of targeting AAV to specific cell types. The construction and characterization of AAV capsid mutants including insertion mutants, alanine screening mutants, and epitope tag mutants is described in Wu et al., J. Virol. 74:8635 (2000). Other rAAV virions that can be used in methods described herein include those capsid hybrids that are generated by molecular breeding of viruses as well as by exon shuffling. See, e.g., Soong et al., Nat. Genet. 25:436 (2000) and Kolman and Stemmer, Nat. Biotechnol. 19:423 (2001).

### Methods for the Delivery of Exogenous Nucleic Acids to Target Cells

Techniques that can be used to introduce a polynucleotide, such as codon-optimized DNA or RNA (e.g., mRNA, tRNA, siRNA, miRNA, shRNA, chemically modified RNA) into a mammalian cell e.g., pluripotent cells, ESC, iPSC, multipotent cell, CD34+ cell, HSC, MPC, BLPC, monocyte, macrophage, microglial progenitor cell, or microglial cell) are well known in the art. For example, electroporation can be used to permeabilize mammalian cells (e.g., human target cells) by the application of an electrostatic potential to the cell of interest. Mammalian cells, such as human cells, subjected to an external electric field in this manner are subsequently predisposed to the uptake of exogenous nucleic acids. Electroporation of mammalian cells is described in detail, e.g., in Chu et al., Nucleic Acids Research 15:1311 (1987), the disclosure of which is incorporated herein by reference. A similar technique, Nucleofection™, utilizes an applied electric field in order to stimulate the uptake of exogenous polynucleotides into the nucleus of a eukaryotic cell. Nucleofection™ and protocols useful for performing this technique are described in detail, e.g., in Distler et al., Experimental Dermatology 14:315 (2005), as well as in US 2010/0317114, the disclosures of each of which are incorporated herein by reference.

Additional techniques useful for the transfection of target cells are the squeeze-poration methodology. This technique induces the rapid mechanical deformation of cells in order to stimulate the uptake of exogenous DNA through membranous pores that form in response to the applied stress. This technology is advantageous in that a vector is not required for delivery of nucleic acids into a cell, such as a human target cell. Squeeze-poration is described in detail, e.g., in Sharei et al., Journal of Visualized Experiments 81:e50980 (2013), the disclosure of which is incorporated herein by reference.

Lipofection represents another technique useful for transfection of target cells. This method involves the loading of nucleic acids into a liposome, which often presents cationic functional groups, such as quaternary or protonated amines, towards the liposome exterior. This promotes electrostatic interactions between the liposome and a cell due to the anionic nature of the cell membrane, which ultimately leads to uptake of the exogenous nucleic acids, for example, by direct fusion of the liposome with the cell membrane or by endocytosis of the complex. Lipofection is described in detail, for example, in U.S. Pat. No. 7,442,386, the disclosure of which is incorporated herein by reference. Similar techniques that exploit ionic interactions with the cell membrane to provoke the uptake of foreign nucleic acids are contacting a cell with a cationic polymer-nucleic acid complex. Exemplary cationic molecules that associate with polynucleotides so as to impart a positive charge favorable for interaction with the cell membrane are activated dendrimers (described, e.g., in Dennig, Topics in Current Chemistry 228:227 (2003), the disclosure of which is incorporated herein by reference) polyethylenimine, and diethylaminoethyl (DEAE)-dextran, the use of which as a transfection agent is described in detail, for example, in Gulick et al., Current Protocols in Molecular Biology 40:1:9.2:9.2.1 (1997), the disclosure of which is incorporated herein by reference. Magnetic beads are another tool that can be used to transfect target cells in a mild and efficient manner, as this methodology utilizes an applied magnetic field in order to direct the uptake of nucleic acids. This technology is described in detail, for example, in US 2010/0227406, the disclosure of which is incorporated herein by reference.

Another useful tool for inducing the uptake of exogenous nucleic acids by target cells is laserfection, also called optical transfection, a technique that involves exposing a cell to electromagnetic radiation of a particular wavelength in order to gently permeabilize the cells and allow polynucleotides to penetrate the cell membrane. The bioactivity of this technique is similar to, and in some cases found superior to, electroporation.

Impalefection is another technique that can be used to deliver genetic material to target cells. It relies on the use of nanomaterials, such as carbon nanofibers, carbon nanotubes, and nanowires. Needle-like nanostructures are synthesized perpendicular to the surface of a substrate. DNA containing the gene, intended for intracellular delivery, is attached to the nanostructure surface. A chip with arrays of these needles is then pressed against cells or tissue. Cells that are impaled by nanostructures can express the delivered gene(s). An example of this technique is described in Shalek et al., PNAS 107:25 1870 (2010), the disclosure of which is incorporated herein by reference.

Magnetofection can also be used to deliver nucleic acids to target cells. The magnetofection principle is to associate nucleic acids with cationic magnetic nanoparticles. The magnetic nanoparticles are made of iron oxide, which is fully biodegradable, and coated with specific cationic proprietary molecules varying upon the applications. Their association with the gene vectors (DNA, siRNA, viral vector, etc.) is achieved by salt-induced colloidal aggregation and electrostatic interaction. The magnetic particles are then concentrated on the target cells by the influence of an external magnetic field generated by magnets. This technique is described in detail in Scherer et al., Gene Therapy 9:102 (2002), the disclosure of which is incorporated herein by reference.

Another useful tool for inducing the uptake of exogenous nucleic acids by target cells is sonoporation, a technique that involves the use of sound (typically ultrasonic frequencies) for modifying the permeability of the cell plasma membrane permeabilize the cells and allow polynucleotides to penetrate the cell membrane. This technique is described in detail, e.g., in Rhodes et al., Methods in Cell Biology 82:309 (2007), the disclosure of which is incorporated herein by reference.

Microvesicles represent another potential vehicle that can be used to modify the genome of a target cell according to the methods described herein. For example, microvesicles that have been induced by the co-overexpression of the glycoprotein VSV-G with, e.g., a genome-modifying protein, such as a nuclease, can be used to efficiently deliver proteins into a cell that subsequently catalyze the site-specific cleavage of an endogenous polynucleotide so as to prepare the genome of the cell for the covalent incorporation of a polynucleotide of interest, such as a gene or regulatory sequence. The use of such vesicles, also referred to as Gesicles, for the genetic modification of eukaryotic cells is described in detail, e.g., in Quinn et al., Genetic Modification of Target Cells by Direct Delivery of Active Protein [abstract]. In: Methylation changes in early embryonic genes in cancer [abstract], in: Proceedings of the 18th Annual Meeting of the American Society of Gene and Cell Therapy; 2015 May 13, Abstract No. 122.

### Methods of Diagnosis

Subjects may be diagnosed as having Pompe disease using methods well-known in the art, such as, e.g., the methods described in herein. Because infantile-onset and late-onset Pompe disease may exhibit differential symptoms, it is appropriate to distinguish diagnostic criteria suitable for each of these forms of Pompe disease. For example, diagnosis of infantile-onset Pompe diseases in a subject may be guided by identification of cardiovascular symptoms, including but not limited to cardiomegaly, cardiomyopathy (hypertrophic with or without left ventricular outflow tract obstruction (LVOTO)), heart murmurs or gallops, pulsatile precordium, cardiac-related excessive sweating, congestive heart failure, arrhythmias (e.g., supraventricular tachycardia), cardiac arrest during surgery. Infantile-onset Pompe disease may also be guided by diagnosing the patient with pulmonary symptoms, including frequent respiratory infections, respiratory distress or insufficiency, nasal flaring, use of accessory muscles to breathe, and decreased and/or coarse breath sounds. A patient with infantile-Pompe disease may also be identified by a diagnosis confirming one or more neurological symptoms, such as, e.g., hypotonia, head lag, floppy baby appearance, frog leg position, developmental delay, gross motor delay, and loss of early motor milestones. Gastrointestinal symptoms, such as, e.g., failure to thrive and feeding difficulties, which may result from macroglossia, open mouth, low facial tone, decreased gag reflex, poor suck and swallow control, and hepatomegaly, may also be indicative of a confirmatory diagnosis of Pompe disease in a subject.

Late-onset Pompe disease may be diagnosed in a subject using a number of clinical criteria including the absence of cardiac symptoms (discussed above), but the presence of pulmonary or pulmonary-related symptoms (e.g., frequent respiratory infections, respiratory insufficiency/distress, sleep apnea, orthopnea, exertional dyspnea, weak cough, somnolence, and morning headaches), musculoskeletal symptoms (e.g., limb-girdle weakness (e.g., progressive proximal limb-girdle muscle weakness that is greater in the lower extremities as compared to upper extremities), back pain, exercise intolerance, rigid spine syndrome, gait abnormalities, lordosis/scoliosis, hypotonia, and Gower sign), or gastrointestinal (or related) symptoms (e.g., feeding and swallowing difficulties, difficulty maintaining weight, difficulty chewing or jaw muscle fatigue, decreased gag reflex, and hepatomegaly).

Methods well-known in the art may be employed for the diagnosis of a subject as having Pompe disease. Such methods may include genetic testing to ascertain GAA mutational status, biochemical testing (e.g., enzymology) of GAA activity in fibroblasts or muscle tissue, and histological/histochemical analysis (e.g., of muscle) to detect abnormal glycogen accumulation within the lysosome or cytoplasm or to detect the presence of vacuolated cells. Laboratory testing of blood or urine may be performed to detect increased levels of creatine kinase (CK), aspartate aminotransferase (AST), alanine aminotransferase (ALT), or lactic dehydrogenase (LDH) in blood or glucose tetrasaccharide (Glc4) in urine, measure GAA activity in dried blood spots, lymphocytes, or leukocytes, which may be indicative of a positive GAA diagnosis in a subject. Clinical studies may also be carried out using chest X-rays (to detect cardiomegaly), electrocardiograms (to detect murmurs, gallops, tachycardia, etc.), echocardiography (to detect cardiomyopathy), electrophysiology, such as, e.g., electromyogram or neurocardiogenic syncope testing (to detect myopathy), lung function testing while lying and sitting, and muscle strength testing to identify a subject as having or at risk of developing Pompe disease.

The subject's cognitive function may be assessed by performing cognitive tests that evaluate performance across one or more cognitive domains including but not limited to complex attention, executive function, learning and memory, language, perceptual-motor function, and social cognition. Comparison of cognitive function in the subject relative to a norm appropriate for the subjects age, medical history, education, socioeconomic status, and lifestyle (e.g., a reference population, such as, e.g., a general population) may be done to determine the diagnosis with respect to Pompe disease in the subject. Non-limiting examples of cognitive tests include Eight-item Informant Interview to Differentiate Aging and Dementia (AD8), Annual Wellness Visit (AWV), General Practitioner Assessment of Cognition (GPCOG), Health Risk Assessment (HRA), Memory Impairment Screen (MIS), Mini Mental Status Exam (MMSE), Montreal Cognitive Assessment (MoCA), St. Louis University Mental Status Exam (SLUMS), and Short Informant Questionnaire on Cognitive Decline in the Elderly (Short IQCODE). Additionally or alternatively, the use of F18-fluorodeoxyglucose PET scans or MRI scans may be used to determine the presence of neurodegeneration in a subject with Pompe disease.

Furthermore, the subject may be tested for the presence of biomarkers specific to Pompe disease. For example, a subject may be tested for the presence of biomarkers that indicate that the subject has Pompe disease, such as the presence of elevated CK, AST, ALT, or LDH levels in blood or elevated levels of Glc4 in urine of the subject, and/or presence of mutations in the GAA gene in the subject.

Further still, the subject diagnosed with Pompe disease can be further stratified as a cross-reactive immunological material (CRIM)-positive or CRIM-negative Pompe disease patient. Pompe patients having a complete absence of endogenous GAA may be considered as CRIM-negative, which may result in production of IgG antibodies that can target exogenously delivered GAA for clearance by the immune system. High amounts of exogenous GAA (e.g., rhGAA) IgG antibodies are generally associated with a rapid clinical decline and death in patients with infantile Pompe disease. Thus, upon a confirmatory diagnosis of a Pompe disease patient as being CRIM-negative, a skilled physician may recommend concomitant administration of an immune tolerance inducing (ITI) agent with the disclosed composition. An exemplary ITI agent that may be administered prior to, concomitantly with, or following administration of the disclosed composition may include a combination of rituximab, methotrexate, and/or intravenous immunoglobulin (IVIG). Different combinations of rituximab, methotrexate, and/or IVIG may also include cyclophosphamide, plasmapheresis, and/or bortezomib.

### Methods of Treatment

### Selection of Subjects

Subjects that may be treated as described herein are subjects having or at risk of developing Pompe disease. The compositions and methods described herein can be used to treat subjects with normal GAA activity, reduced GAA activity, and subjects whose GAA mutational status and/or GAA activity level is unknown. The compositions and methods described herein may also be administered as a preventative treatment to subjects at risk of developing Pompe disease, e.g., subjects with a GAA mutation and/or subjects with reduced GAA activity. Subjects at risk for Pompe disease may show early symptoms of Pompe disease or may not yet be symptomatic when treatment is administered.

In some embodiments, the methods and compositions described herein may be administered to subjects with GAA mutations that include, for example, any one of the GAA mutations disclosed in Peruzzo et al. (Ann Trans Med 7(13):278, 2019), which is incorporated by reference herein as it relates to Pompe disease-associated GAA mutations. In some embodiments, the methods and compositions described herein may be administered to subjects carrying any other pathogenic mutation in the GAA gene.

### Routes of Administration

The cells and compositions described herein may be administered to a subject with Pompe disease by a variety of routes, such as intracerebroventricularly, intrathecally, intraparenchymally, stereotactically, intravenously, intraosseously, or by means of a bone marrow transplant. In some embodiments, the cells and compositions described herein may be administered to a subject systemically (e.g., intravenously), directly to the central nervous system (CNS) (e.g., intracerebroventricularly, intrathecally, intraparenchymally, or stereotactically), or directly into the bone marrow (e.g., intraosseously). In some embodiments, the cells and compositions described herein are administered to a subject intracerebroventricularly into the cerebral lateral ventricles (a description of this method can be found in Capotondo et al., Science Advances 3:e1701211 (2017), incorporated herein by reference as it pertains to intracerebroventricular injection of hematopoietic stem and progenitor cells into the cerebral lateral ventricles of mouse models). The most suitable route for administration in any given case will depend on the particular cell or composition administered, the subject, pharmaceutical formulation methods, administration methods (e.g., administration time and administration route), the subject's age, body weight, sex, severity of the diseases being treated, the subject's diet, and the subject's excretion rate. Multiple routes of administration may be used to treat a single subject, e.g., intracerebroventricular or stereotactic injection and intravenous injection, intracerebroventricular or stereotactic injection and intraosseous injection, intracerebroventricular or stereotactic injection and bone marrow transplant, intracerebroventricular or stereotactic injection and intraparenchymal injection, intrathecal injection and intravenous injection, intrathecal injection and intraosseous injection, intrathecal injection and bone marrow transplant, intrathecal injection and intraparenchymal injection, intraparenchymal injection and intravenous injection, intraparenchymal injection and intraosseous injection, or intraparenchymal injection and bone marrow transplant. Multiple routes of administration may be used to treat a single subject at one time, or the subject may receive treatment via one route of administration first, and receive treatment via another route of administration during a second appointment, e.g., 1 week later, 2 weeks later, 1 month later, 6 months later, or 1 year later. Cells may be administered to a subject once, or cells may be administered one or more times (e.g., 2-10 times) per week, month, or year to a subject for treatment of Pompe disease.

### Conditioning

Prior to administration of cells (e.g., pluripotent cells, ESCs, iPSCs, multipotent cells, CD34+ cells, HSCs, MPCs, BLPCs, monocytes, macrophages, microglial progenitor cells, or microglia) or compositions, it may be advantageous to deplete or ablate endogenous cells, such as endogenous microglia and/or hematopoietic stem and progenitor cells. Microglia and/or hematopoietic stem and progenitor cells can be ablated through the use of chemical agents (e.g., busulfan, treosulfan, PLX3397, PLX647, PLX5622, or clodronate liposomes), irradiation, or a combination thereof. The agents used for cell ablation may be BBB penetrating (e.g., busulfan) or may lack the ability to cross the BBB (e.g., treosulfan). Exemplary microglia and/or hematopoietic stem and progenitor cells ablating agents are busulfan (Capotondo et al., PNAS 109:15018 (2012), the disclosure of which is incorporated by reference as it pertains to the use of busulfan to ablate microglia), treosulfan, PLX3397, PLX647, PLX5622, or clodronate liposomes. Other agents for the depletion of endogenous microglia and/or hematopoietic stem and progenitor cells include cytotoxins covalently conjugated to antibodies or antigen binding fragments thereof capable of binding antigens expressed by hematopoietic stem cells so as to form an antibody-drug conjugate. Cytotoxins suitable for antibody drug conjugates include DNA-intercalating agents, (e.g., anthracyclines), agents capable of disrupting the mitotic spindle apparatus (e.g., vinca alkaloids, maytansine, maytansinoids, and derivatives thereof), RNA polymerase inhibitors (e.g., an amatoxin, such as a-amanitin and derivatives thereof), agents capable of disrupting protein biosynthesis (e.g., agents that exhibit rRNA N-glycosidase activity, such as saporin and ricin A-chain), among others known in the art. Ablation may eliminate all microglia and/or hematopoietic stem and progenitor cells, or it may reduce microglia and/or hematopoietic stem and progenitor cells numbers by at least 5% (e.g., at least 5%, 10%, 20%, 30%, 40%, 50%, 60%, 70%, 80%, 90%, 95%, or more). One or more agents to ablate microglia and/or hematopoietic stem and progenitor cells may be administered at least one week (e.g., 1, 2, 3, 4, 5, or 6 weeks or more) before administration of the cells or compositions described herein. Cells administered in the methods described herein may replace the ablated microglia and/or hematopoietic stem and progenitor cells, and may repopulate the brain following intracerebroventricular, stereotactic, intravenous, or intraosseous injection, or following bone marrow transplant. Cells administered intravenously, intraosseously, or by bone marrow transplant may cross the blood brain barrier to enter the brain and differentiate into microglia. Cells administered to the brain, e.g., cells administered intracerebroventricularly or stereotactically, can differentiate into microglia in vivo or can be differentiated into microglia ex vivo.

### Stem Cell Rescue

The methods described herein may include administering to a subject a population of cells (e.g., pluripotent cells, ESCs, iPSCs, multipotent cells, CD34+ cells, HSCs, MPCs, BLPCs, monocytes, macrophages, microglial progenitor cells, or microglia). These cells may be cells that have not been modified to contain the transgene encoding GILT.GAA. The cells may be administered systemically (e.g., intravenously), or by bone marrow transplantation to reconstitute the bone marrow compartment following conditioning as described herein. For example, these cells may migrate to a stem cell niche and increase the quantity of cells of the hematopoietic lineage at such a site by, for example, 1%, 2%, 3%, 4%, 5%, 6%, 7%, 8%, 9%, 10%, 11%, 12%, 13%, 14%, 15%, 16%, 3517%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or more. Administration may occur prior to or following administration of the composition of the described herein.

### Selection of Donor Cells

In some embodiments, the subject is the donor. In such cases, withdrawn cells (e.g., pluripotent cells, ESCs, iPSCs, multipotent cells, CD34+ cells, HSCs, MPCs, BLPCs, monocytes, macrophages, microglial progenitor cells, or microglia) may be re-infused into the subject following modification (e.g., incorporation of the transgene encoding GILT.GAA), such that the cells may subsequently home to hematopoietic tissue and establish productive hematopoiesis, thereby populating or repopulating a line of cells that is defective or deficient in the subject (e.g., a population of microglia). In this scenario, the transplanted cells are least likely to undergo graft rejection, as the infused cells are derived from the subject and express the same HLA class me and class II antigens as expressed by the subject. Alternatively, the subject and the donor may be distinct. In some embodiments, the subject and the donor are related, and may, for example, be HLA-matched. As described herein, HLA-matched donor-recipient pairs have a decreased risk of graft rejection, as endogenous T cells and NK cells within the transplant recipient are less likely to recognize the incoming hematopoietic stem or progenitor cell graft as foreign and are thus less likely to mount an immune response against the transplant. Exemplary HLA-matched donor-recipient pairs are donors and recipients that are genetically related, such as familial donor-recipient pairs (e.g., sibling donor-recipient pairs). In some embodiments, the subject and the donor are HLA-mismatched, which occurs when at least one HLA antigen, in particular with respect to HLA-A, HLA-B and HLA-DR, is mismatched between the donor and recipient. To reduce the likelihood of graft rejection, for example, one haplotype may be matched between the donor and recipient, and the other may be mismatched.

### Pharmaceutical Compositions and Dosing

The number of cells administered to a subject for the treatment of Pompe disease as described herein may depend, for example, on the expression level of GAA, the subject, pharmaceutical formulation methods, administration methods (e.g., administration time and administration route), the subject's age, body weight, sex, severity of the disease being treated, and whether or not the subject has been treated with agents to ablate endogenous microglia. The number of cells administered may be, for example, from 1×10⁶ cells/kg to 1×10¹² cells/kg, or more (e.g., 1×10⁷ cells/kg, 1×10⁸ cells/kg, 1×10⁹ cells/kg, 1×10¹⁰ cells/kg, 1×10¹¹ cells/kg, 1×10¹² cells/kg, or more). Cells may be administered in an undifferentiated state, or after partial or complete differentiation into microglia. The number of cells may be administered in any suitable dosage following conditioning. Non-limiting examples of dosages are about 1×10⁵ as cells/kg of recipient to about 30×10⁷ cells/kg (e.g., from about 2×10⁵ as cells/kg to about 29×10⁷ cells/kg, from about 3×10⁵ as cells/kg to about 28×10⁷ cells/kg, from about 4×10⁵ as cells/kg to about 27×10⁷ cells/kg, from about 5×10⁵ as cells/kg to about 26×10⁷ cells/kg, from about 5×10⁵ as cells/kg to about 25×10⁷ cells/kg, from about 6×10⁵ as cells/kg to about 24×10⁷ cells/kg, from about 7×10⁵ as cells/kg to about 23×10⁷ cells/kg, from about 8×10⁵ as cells/kg to about 22×10⁷ cells/kg, from about 9×10⁵ as cells/kg to about 21×10⁷ cells/kg, from about 1×10⁶ cells/kg to about 20×10⁷ cells/kg, from about 2×10⁶ cells/kg to about 19×10⁷ cells/kg, from about 3×10⁶ cells/kg to about 19×10⁷ cells/kg, from about 4×10⁶ cells/kg to about 18×10⁷ cells/kg, from about 5×10⁶ cells/kg to about 17×10⁷ cells/kg, from about 6×10⁶ cells/kg to about 16×10⁷ cells/kg, from about 7×10⁶ cells/kg to about 15×10⁷ cells/kg, from about 8×10⁶ cells/kg to about 10×10⁷ cells/kg, and from about 9×10⁶ cells/kg to about 5×10⁷ cells/kg). Additional exemplary dosages are from about 1×10¹⁰ cells/kg of recipient to about 1×10¹² cells/kg (e.g., from about 2×10¹⁰ cells/kg to about 9×10¹¹ cells/kg, from about 3×10¹⁰ cells/kg to about 8×10¹¹ cells/kg, from about 4×10¹⁰ cells/kg to about 7×10¹¹ cells/kg, from about 5×10¹⁰ cells/kg to about 6×10¹¹ cells/kg, from about 5×10¹⁰ cells/kg to about 1×10¹² cells/kg, from about 6×10¹⁰ cells/kg to about 1×10¹² cells/kg, from about 7×10¹⁰ cells/kg to about 1×10¹² cells/kg, from about 8×10¹⁰ cells/kg to about 1×10¹² cells/kg, from about 9×10¹⁰ cells/kg to about 1×10¹² cells/kg, and from about 1×10¹¹ cells/kg to about 1×10¹² cells/kg), among others.

The cells and compositions described herein can be administered in an amount sufficient to improve one or more pathological features in the Pompe disease. For example, administration of the cells or compositions described herein may reduce the occurrence or severity of cardiomegaly, hypotonia, cardiomyopathy, respiratory distress, muscle weakness, feeding difficulties, failure to thrive, floppy baby appearance, delay in motor development, hepatomegaly, macroglossia, wide open mouth, wide open eyes, nasal flaring, respiratory rate, engagement of accessory muscles for breathing, frequency of chest infections, arrhythmia, heart failure, impaired cough, muscle weakness, difficulty masticating and swallowing, and/or loss of brain tissue in the subject. or the composition may increase one or more of facial muscle tone, air flow in the left lower zone, and vital capacity, improve the cognitive performance of the subject and/or motor function of the subject.

Cognition and motor function can be assessed using standard neurological tests before and after treatment and proteins can be detected in plasma and CSF using ELISA. Neurodegeneration can be assessed using F18-fluorodeoxyglucose PET scans or MRI scans. The subject may be evaluated 1 month, 2 months, 3 months, 4 months, 5 months, 6 months or more following administration of the population of cells depending on the route of administration used for treatment. Depending on the outcome of the evaluation, the subject may receive additional treatments.

### Combination Therapy

As is discussed herein, CRIM-negative Pompe disease patients may require administration of one or more ITI agents in order to improve the efficacy of exogenously-delivered GAA transgenes. Accordingly, the present disclosure contemplates a combination therapy comprising the compositions disclosed here together with an ITI. An exemplary ITI agent that may be administered prior to, concomitantly with, or following administration of the disclosed composition may include a combination of rituximab, methotrexate, and/or intravenous immunoglobulin (IVIG). Different combinations of rituximab, methotrexate, and/or IVIG may also include cyclophosphamide, plasmapheresis, and/or bortezomib.

The ITI agent can be administered at the same time (e.g., administration of all agents occurs within 15 minutes, 10 minutes, 5 minutes, 2 minutes or less) as the composition of the disclosure. The agents can also be administered simultaneously via co-formulation. The disclosed composition and the ITI agent can also be administered sequentially, such that the action of the two overlaps and their combined effect is such that the reduction in a symptom, or other parameter related to the disorder is greater than what would be observed with one agent or treatment delivered alone or in the absence of the other. The effect of the composition and the ITI agent supplement can be partially additive, wholly additive, or greater than additive (e.g., synergistic). Sequential or substantially simultaneous administration of each of the composition and the ITI agent can be performed by any appropriate route including, but not limited to, oral routes, intravenous routes, intramuscular routes, local routes, and direct absorption through mucous membrane tissues. The disclosed composition and the ITI agent can be administered by the same route or by different routes. For example, an composition may be administered by intravenous injection while the ITI agent can be orally. The disclosed composition may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours up to 24 hours or up to 1-7, 1-14, 1-21 or 1-30 days before or after the ITI agent.

Surgical intervention may also be performed in conjunction with the disclosed methods.

### Kits

The compositions described herein can be provided in a kit for use in treating Pompe disease. Compositions may include host cells described herein (e.g., pluripotent cells, ESCs, iPSCs, multipotent cells, CD34+ cells, HSCs, MPCs, BLPCs, monocytes, macrophages, microglial progenitor cells, or microglia) that contain a transgene encoding GILT.GAA. Cells may be cryopreserved, e.g., in dimethyl sulfoxide (DMSO), glycerol, or another cryoprotectant. The kit can include a package insert that instructs a user of the kit, such as a physician, to perform the methods described herein. The kit may optionally include a syringe or other device for administering the composition.

### EXAMPLES

The following examples are put forth so as to provide those of ordinary skill in the art with a description of how the compositions and methods described herein may be used, made, and evaluated, and are intended to be purely exemplary of the disclosure and are not intended to limit the scope of what the inventors regard as their disclosure.

### Example 1. Generation of a Cell Containing a Transgene Encoding Alpha Acid-Glucosidase Fused to a Glycosylation-Independent Lysosomal Targeting Tag Containing an Insulin-Like Growth Factor II Mutein

An exemplary method for making cells (e.g., pluripotent cells, ESCs, iPSCs, multipotent cells, CD34+ cells, HSCs, MPCs, BLPCs, monocytes, macrophages, microglial progenitor cells, or microglia) that contain an acid alpha-glucosidase (GAA) protein fused to a glycosylation-independent lysosomal targeting (GILT) tag (GILT.GAA protein) containing an insulin-like growth factor II (IGF-II) mutein harboring an Ala amino acid substitution at a position corresponding to Arg37 of SEQ ID NO: 15 (R37A substitution/mutation) for use in the compositions and methods described herein is by way of transduction. Retroviral vectors (e.g., a lentiviral vector, alpharetroviral vector, or gammaretroviral vector) containing promoter, such as an elongation factor 1-alpha (EF1α) promoter, phosphoglycerate kinase 1 (PGK) promoter, β-LCR(HS4,3,2)-EFS promoter, CD68 molecule (CD68) promoter, C-X3-C motif chemokine receptor 1 (CX3CR1) promoter, CD11 b promoter, allograft inflammatory factor 1 (AIF1) promoter, purinergic receptor P2Y12 (P2Y12) promoter, transmembrane protein 119 (TMEM119) promoter, colony stimulating factor 1 receptor (CSF1R) promoter, adenovirus late promoter, vaccinia virus 7.5K promoter, simian virus 40 (SV40) promoter, cytomegalovirus promoter, tk promoter of herpes simplex virus (HSV), mouse mammary tumor virus (MMTV) promoter, long terminal repeat (LTR) promoter of human immunodeficiency virus (HIV), promoter of moloney virus, Epstein barr virus (EBV), Rous sarcoma virus (RSV), cytomegalovirus (CMV) promoter, or a myeloproliferative sarcoma virus enhancer, negative control region deleted, dl587rev primer-binding site substituted (MND) promoter (SEQ ID NO: 10 or SEQ ID NO: 11), and the polynucleotide encoding GILT.GAA protein can be engineered using standard techniques known in the art. After the retroviral vector is engineered, the retrovirus can be used to transduce cells to generate a population of cells that express GAA.

Additional exemplary methods for making cells that contain a transgene encoding GILT.GAA for use in the compositions and methods described herein is transfection. Using molecular biology techniques known in the art, plasmid DNA containing a promoter and the polynucleotide encoding GILT.GAA can be produced. For example, the GAA gene fused to a polynucleotide encoding the GILT tag may be amplified from a human cell line using PCR-based techniques known in the art, or the gene may be synthesized, for example, using solid-phase polynucleotide synthesis procedures. The polynucleotide containing the GILT.GAA transgene and promoter can then be ligated into a plasmid of interest, for example, using suitable restriction endonuclease-mediated cleavage and ligation protocols. After the plasmid DNA is engineered, the plasmid can be used to transfect the cells using, for example, electroporation or another transfection technique described herein to generate a population of cells that express GILT.GAA. In both exemplary methods described herein, GILT.GAA fusion protein may further contain a peptide sequence containing the low-density lipoprotein receptor superfamily (LDLRf) receptor-binding (Rb) domain of apolipoprotein E (ApoE) to allow for the penetrance of the GAA fusion protein across the blood-brain barrier.

### Example 2. Administration of a Population of Cells Containing a Transgene Encoding GAA to a Subject Suffering from Pompe Disease

According to the methods disclosed herein, a physician of skill in the art can treat a subject, such as a mammalian subject (e.g., a human subject), so as to reduce or alleviate symptoms of Pompe disease. To this end, a physician of skill in the art can administer to the human subject a population of cells (e.g., pluripotent cells, ESCs, iPSCs, multipotent cells, CD34+ cells, HSCs, MPCs, BLPCs, monocytes, macrophages, microglial progenitor cells, or microglia) containing a transgene encoding GILT.GAA protein containing an IGF-II mutein harboring an Ala amino acid substitution at a position corresponding to Arg37 of SEQ ID NO: 15. The cells can be transduced or transfected ex vivo to express GILT.GAA using techniques described herein or known in the art. The population of cells containing the transgene encoding GILT.GAA operably linked to a promoter may be administered to the subject, for example, systemically (e.g., intravenously), directly to the CNS (e.g., intracerebroventricularly or stereotactically), or directly into the bone marrow (e.g., intraosseously), to treat Pompe disease. The cells can also be administered to the subject by multiple routes of administration, for example, intravenously and intracerebroventricularly. The cells are administered in a therapeutically effective amount, such as from 1×10⁵ as cells/kg of recipient to about 30×10⁷ cells/kg (e.g., from about 2×10⁵ as cells/kg to about 29×10⁷ cells/kg, from about 3×10⁵ as cells/kg to about 28×10⁷ cells/kg, from about 4×10⁵ as cells/kg to about 27×10⁷ cells/kg, from about 5×10⁵ as cells/kg to about 26×10⁷ cells/kg, from about 5×10⁵ as cells/kg to about 25×10⁷ cells/kg, from about 6×10⁵ as cells/kg to about 24×10⁷ cells/kg, from about 7×10⁵ as cells/kg to about 23×10⁷ cells/kg, from about 8×10⁵ as cells/kg to about 22×10⁷ cells/kg, from about 9×10⁵ as cells/kg to about 21×10⁷ cells/kg, from about 1×10⁶ cells/kg to about 20×10⁷ cells/kg, from about 2×10⁶ cells/kg to about 19×10⁷ cells/kg, from about 3×10⁶ cells/kg to about 19×10⁷ cells/kg, from about 4×10⁶ cells/kg to about 18×10⁷ cells/kg, from about 5×10⁶ cells/kg to about 17×10⁷ cells/kg, from about 6×10⁶ cells/kg to about 16×10⁷ cells/kg, from about 7×10⁶ cells/kg to about 15×10⁷ cells/kg, from about 8×10⁶ cells/kg to about 10×10⁷ cells/kg, and from about 9×10⁶ cells/kg to about 5×10⁷ cells/kg).

Before the population of cells is administered to the subject, one or more agents may be administered to the subject to ablate the subject's endogenous microglia and/or hematopoietic stem and progenitor cells, for example, busulfan, treosulfan, PLX3397, PLX647, PLX5622, and/or clodronate liposomes. Other methods of cell ablation well known in the art, such as irradiation, may be used alone or in combination with one or more of the aforementioned agents to ablate the subject's microglia and/or hematopoietic stem and progenitor cells. These agents and/or treatments may ablate endogenous microglia and/or hematopoietic stem and progenitor cells by at least 5% (e.g., at least 5%, 10%, 15%, 20%, 25%, 30%, 35%, 40%, 45%, 50%, 60%, 70%, 80%, 90%, 99%, or more), as assessed by PET imaging techniques known in the art. If the population of cells is administered to the subject after microglial ablation, the cells can repopulate the brain, differentiating into microglia. The population of cells can be administered to the subject from, for example, 1 week to 1 month (e.g., 1 week, 2 weeks, 3 weeks, 4, weeks) or more after microglial ablation.

Following ablation of the subject's endogenous microglia and/or hematopoietic stem and progenitor cells, a population of cells may be administered to the subject systemically (e.g., intravenously), or by bone marrow transplantation to reconstitute the bone marrow compartment. The number of cells may be administered in any suitable dosage following conditioning. Non-limiting examples of dosages are about 1×10⁵ as cells/kg of recipient to about 30×10⁷ cells/kg (e.g., from about 2×10⁵ as cells/kg to about 29×10⁷ cells/kg, from about 3×10⁵ as cells/kg to about 28×10⁷ cells/kg, from about 4×10⁵ as cells/kg to about 27×10⁷ cells/kg, from about 5×10⁵ as cells/kg to about 26×10⁷ cells/kg, from about 5×10⁵ as cells/kg to about 25×10⁷ cells/kg, from about 6×10⁵ as cells/kg to about 24×10⁷ cells/kg, from about 7×10⁵ as cells/kg to about 23×10⁷ cells/kg, from about 8×10⁵ as cells/kg to about 22×10⁷ cells/kg, from about 9×10⁵ as cells/kg to about 21×10⁷ cells/kg, from about 1×10⁶ cells/kg to about 20×10⁷ cells/kg, from about 2×10⁶ cells/kg to about 19×10⁷ cells/kg, from about 3×10⁶ cells/kg to about 19×10⁷ cells/kg, from about 4×10⁶ cells/kg to about 18×10⁷ cells/kg, from about 5×10⁶ cells/kg to about 17×10⁷ cells/kg, from about 6×10⁶ cells/kg to about 16×10⁷ cells/kg, from about 7×10⁶ cells/kg to about 15×10⁷ cells/kg, from about 8×10⁶ cells/kg to about 10×10⁷ cells/kg, and from about 9×10⁶ cells/kg to about 5×10⁷ cells/kg). Administration may occur prior to or following administration of the cells containing a transgene encoding GAA. The population of cells can be administered to the subject in an amount sufficient to treat one or more of the pathological features of Pompe disease. For example, the population of cells can be administered in an amount sufficient to reduce one or more of cardiomegaly, hypotonia, cardiomyopathy, respiratory distress, muscle weakness, feeding difficulties, failure to thrive, floppy baby appearance, delay in motor development, hepatomegaly, macroglossia, wide open mouth, wide open eyes, nasal flaring, respiratory rate, engagement of accessory muscles for breathing, frequency of chest infections, arrhythmia, heart failure, impaired cough, muscle weakness, difficulty masticating and swallowing, or the composition can be administered in an amount sufficient to increase one or more of facial muscle tone, air flow in the left lower zone, and vital capacity. A standard neurological examination can also be performed by the physician before and after treatment to evaluate changes in cognitive performance and motor function. The subject may be evaluated, for example, 1 month, 2 months, 3 months, 4 months, 5 months, 6 months or more following administration of the population of cells depending on the route of administration used for treatment. A finding of reduced cardiomegaly, hypotonia, cardiomyopathy, respiratory distress, muscle weakness, feeding difficulties, failure to thrive, floppy baby appearance, delay in motor development, hepatomegaly, macroglossia, wide open mouth, wide open eyes, nasal flaring, respiratory rate, engagement of accessory muscles for breathing, frequency of chest infections, arrhythmia, heart failure, impaired cough, muscle weakness, difficulty masticating and swallowing or increased facial muscle tone, air flow in the left lower zone, and vital capacity following administration of a population of cells containing a transgene encoding GILT.GAA provides an indication that the treatment has successfully treated the Pompe disease.

### Example 3. Establishing Therapeutic Expression of GAA Activity in Mouse Models of Pompe Disease Treated with Lineage Negative Hematopoietic Stem Cells Transduced with a Transgene Encoding GILT.GAA

The objective of this study was to ascertain the therapeutic efficacy of lineage negative HSCs transduced with a transgene encoding a GILT.GAA fusion protein under transcriptional control of an MND promoter.

To this end, 176 GAA knock-out mice (Gaa−/−) and 52 wild-type mice (Gaa+/+) were employed as part of an experimental study. Multiple groups of thirteen Gaa−/− mice, each male and female, were first conditioned with myeloablative 7.5 Gy radiation to ablate endogenous microglial cells, and subsequently treated (by way of intravenous injection) with 5×10⁵ lineage negative HSCs from donor Gaa−/− mice, the cells having been previously transduced with a lentiviral vector containing a codon-optimized transgene encoding a GILT.GAA protein containing an R37A IGF-II mutein (MOI: 0.75, 1.5, and 3) (see Table 2 below). For comparison, Gaa−/− and wild-type GAA+/+ mice receiving no HSCs, Gaa−/− mice receiving 7.5 Gy radiation and non-transduced HSCs (7.5 Gy NT), Gaa−/− mice receiving 7.5 Gy radiation and HSCs encoding a codon-optimized GAA transgene lacking a GILT tag were used as control groups. Other tested groups include Gaa−/− and wild-type GAA+/+ mice receiving 7.5 Gy radiation and HSCs transduced with a transgene encoding green fluorescent protein (GFP) (see Table 2 below).

TABLE 2
Description of experimental groups

| | | | Number of | | Target Vector | Number | |
|---|---|---|---|---|---|---|---|
| | Donor | Recipient | Recipient | | Copy Number | of Donor | |
| Group | Phenotype | Phenotype | Mice | Lentiviral Identity | (VCN) | Cells | Conditioning |
| 1 | Gaa−/− | Gaa−/− | 13 M/13 F | GILT.R37A.GAAco | 1.5 | 5 × 10⁵ | 7.5 Gy |
| 2 | Gaa−/− | Gaa−/− | 13 M/13 F | GILT.R37A.GAAco | 0.75 | 5 × 10⁵ | 7.5 Gy |
| 3 | Gaa−/− | Gaa−/− | 13 M/13 F | GILT.R37A.GAAco | 3 | 5 × 10⁵ | 7.5 Gy |
| 4 | Gaa−/− | Gaa−/− | 13 M/13 F | GFP | 1.5 | 5 × 10⁵ | 7.5 Gy |
| 5 | Gaa+/+ | Gaa+/+ | 13 M/13 F | GFP | 1.5 | 5 × 10⁵ | 7.5 Gy |
| 6 | Gaa−/− | Gaa−/− | 13 M/13 F | GAAco (control) | 1.5 | 5 × 10⁵ | 7.5 Gy |
| 7 | N/A | Gaa−/− | 13 M/13 F | N/A | N/A | N/A | N/A |
| 8 | N/A | Gaa+/+ | 13 M/13 F | N/A | N/A | N/A | N/A |
| 9 | Gaa−/− | Gaa−/− | 13 M/7 F | Non-transduced | N/A | 5 × 10⁵ | 7.5 Gy |

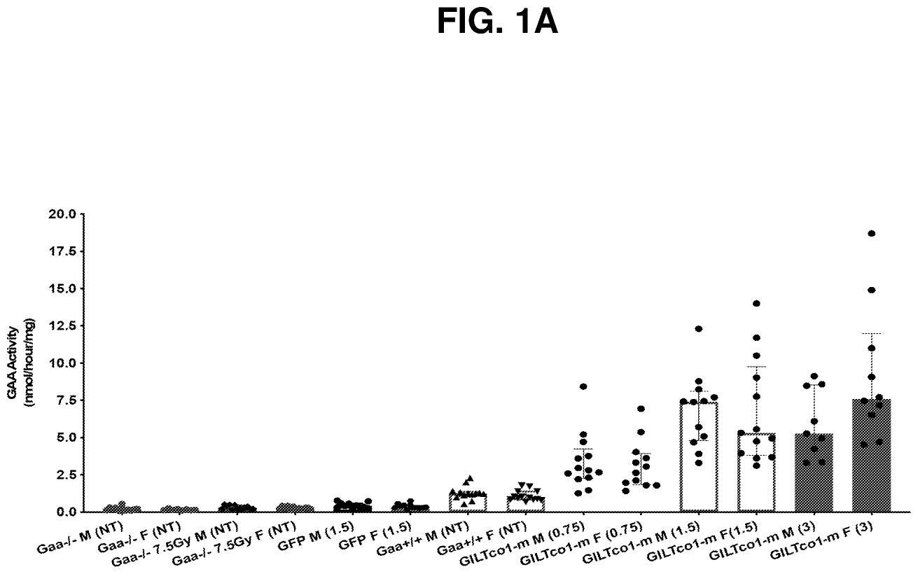

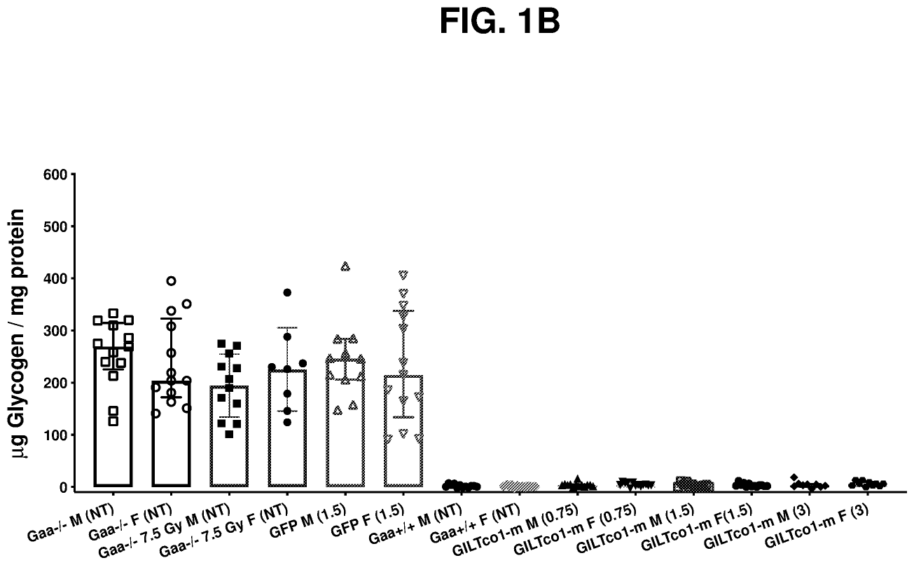

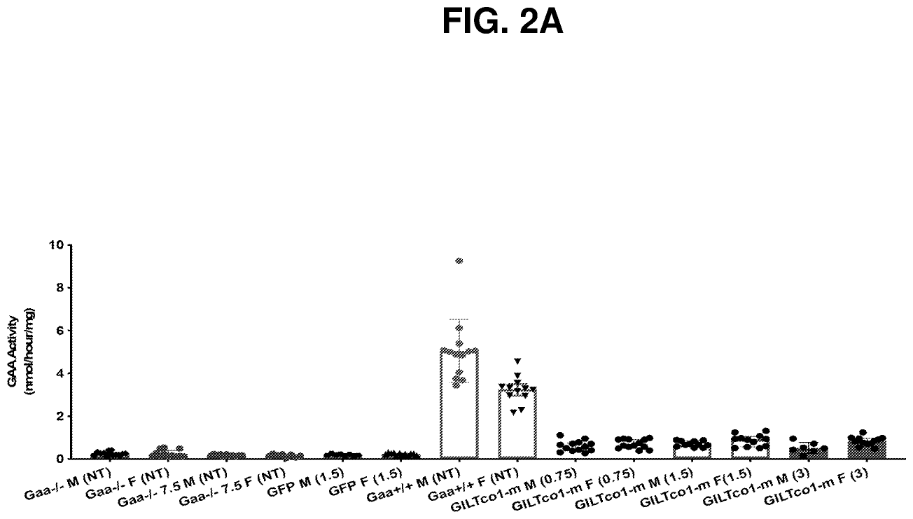

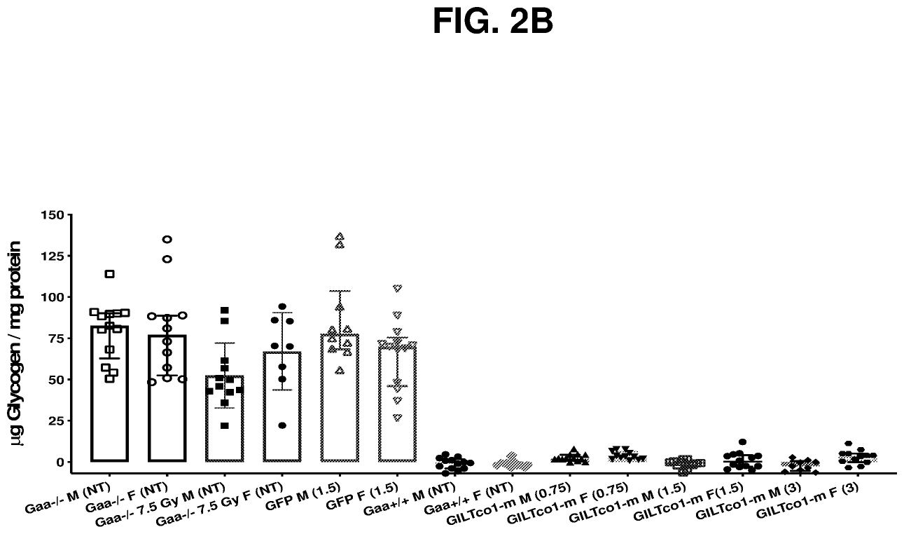

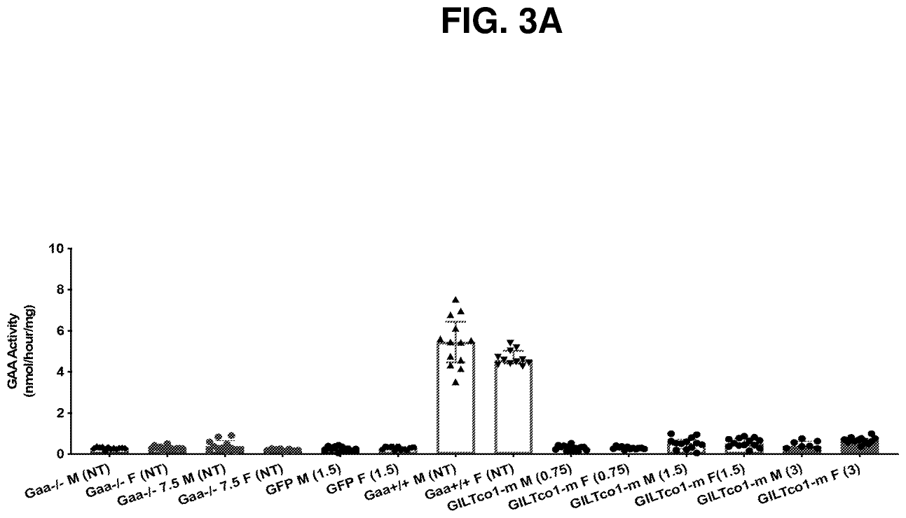

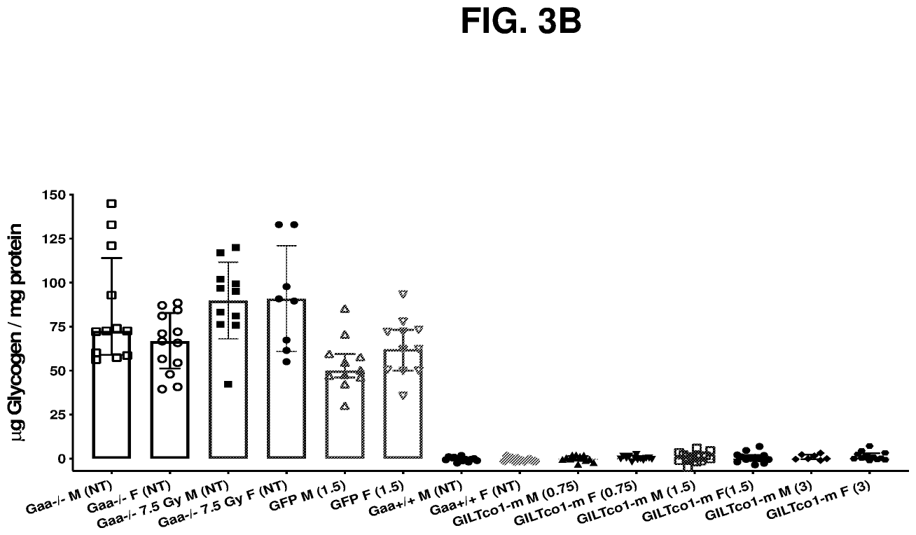

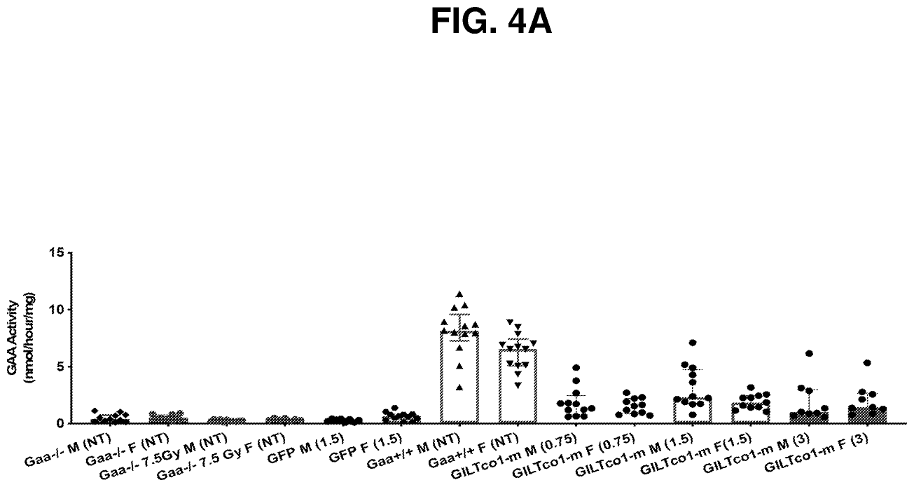

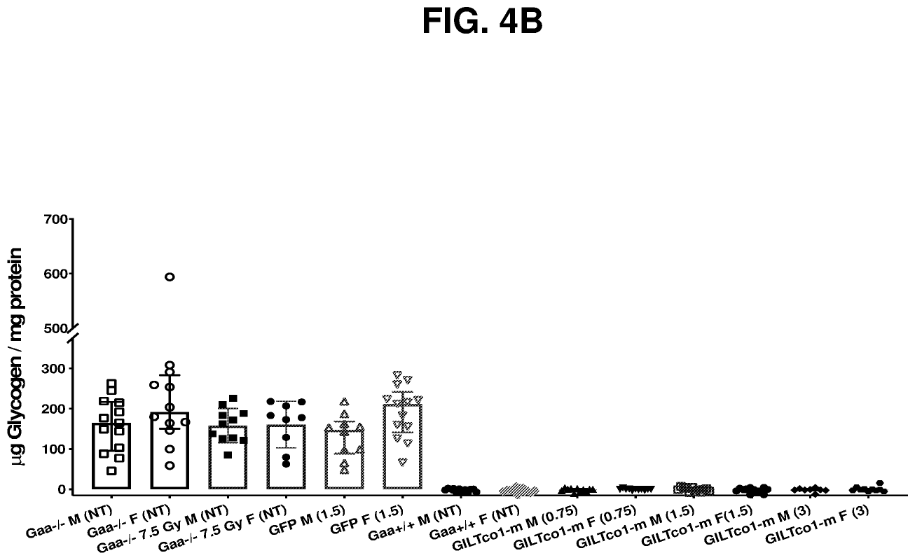

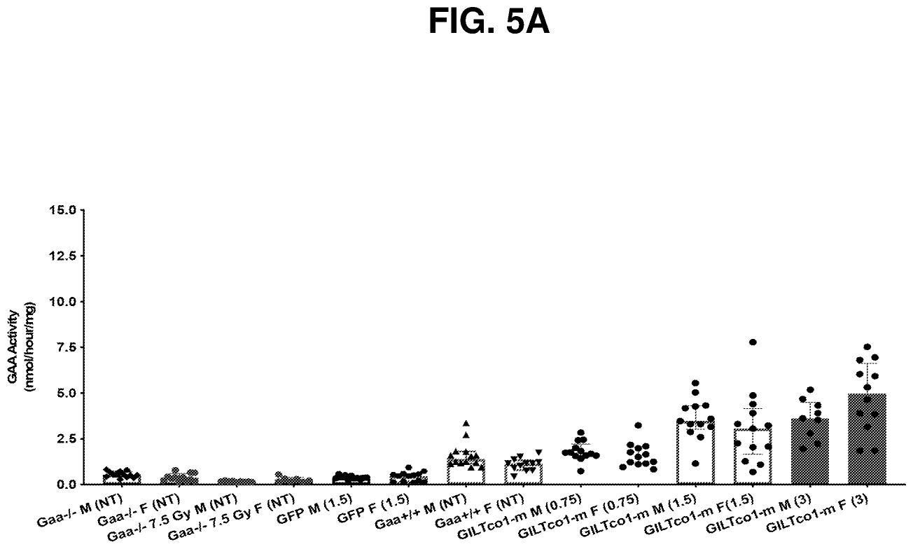

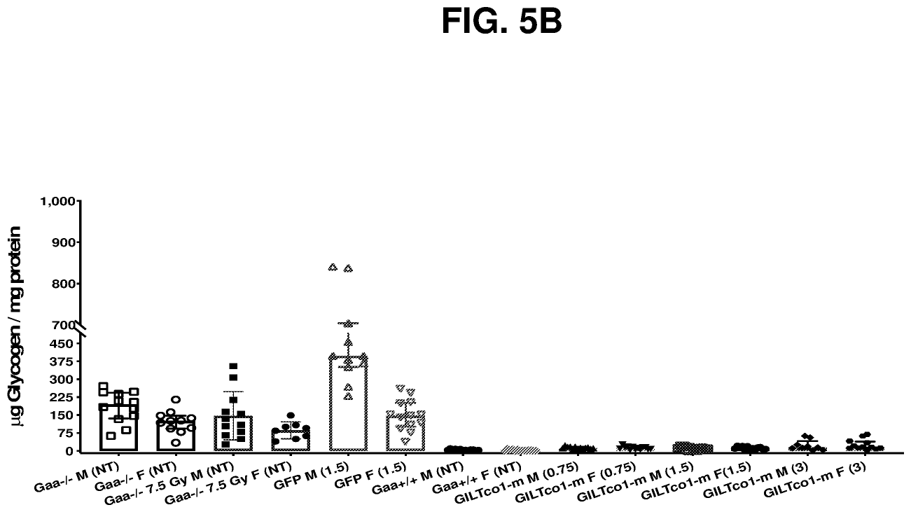

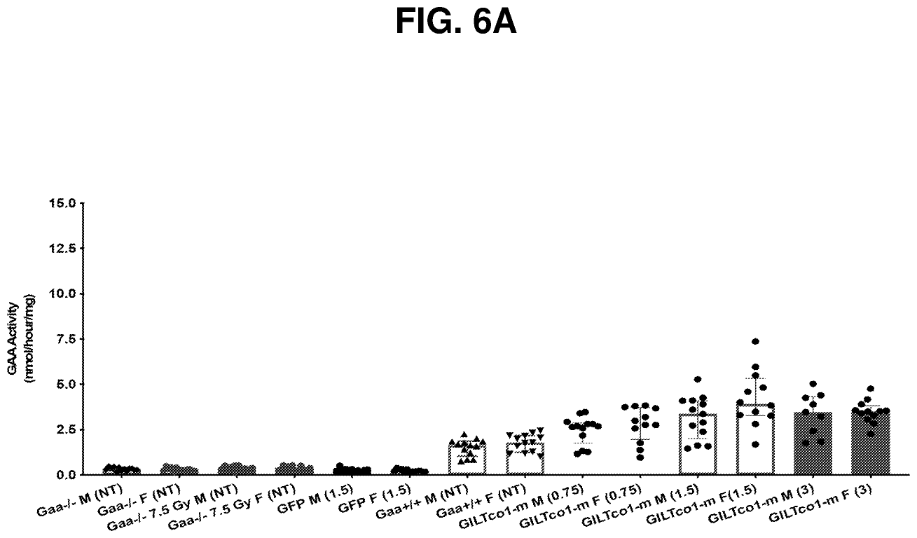

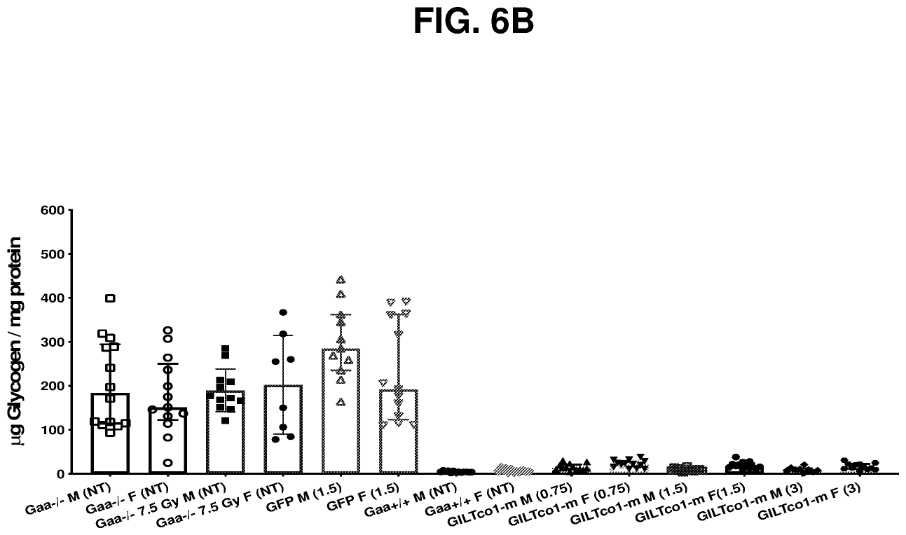

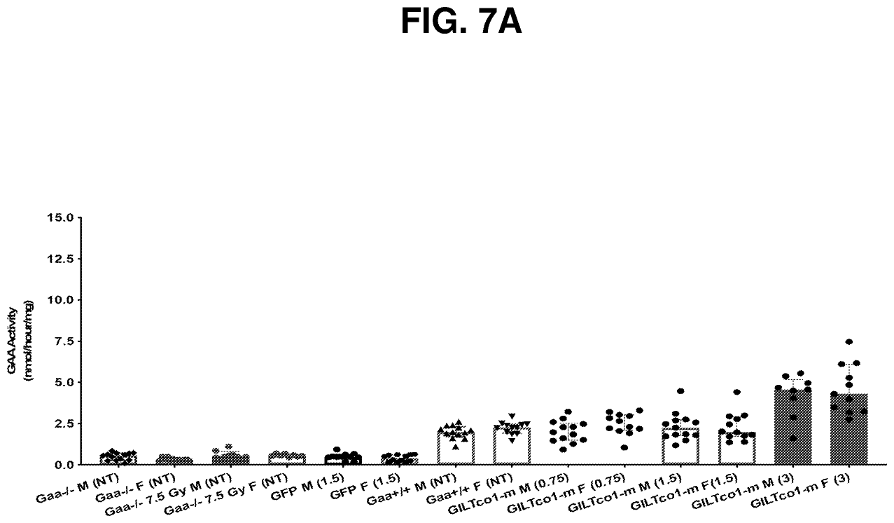

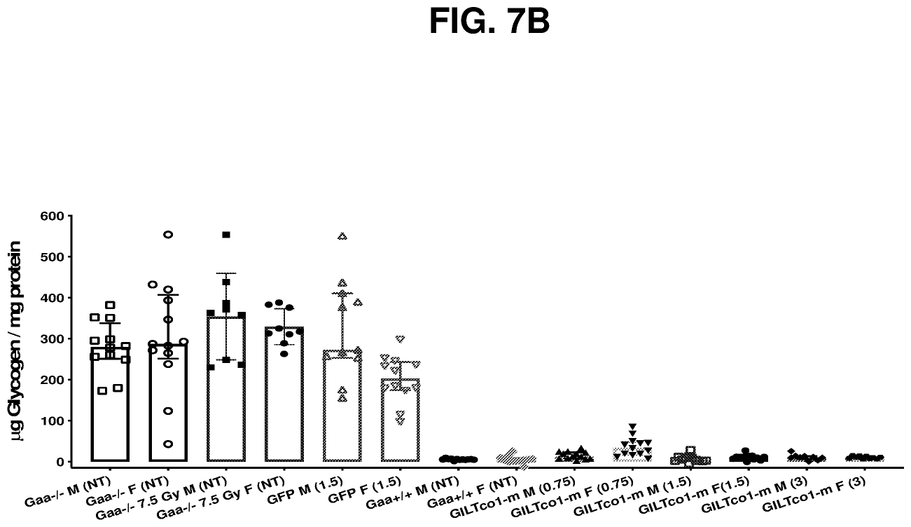

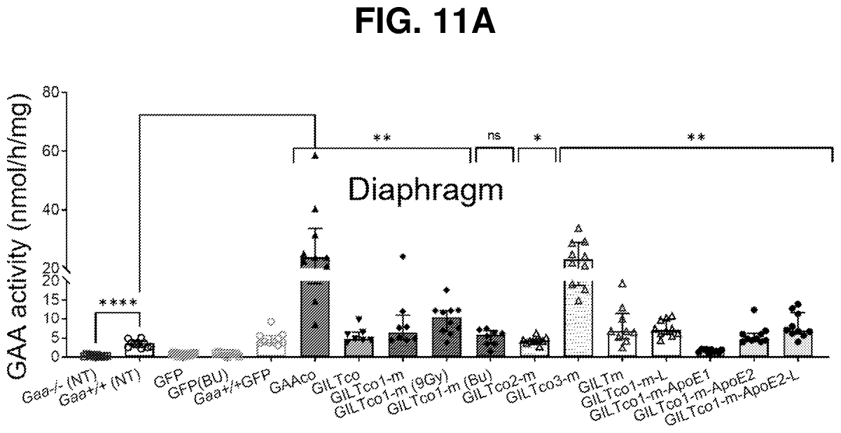

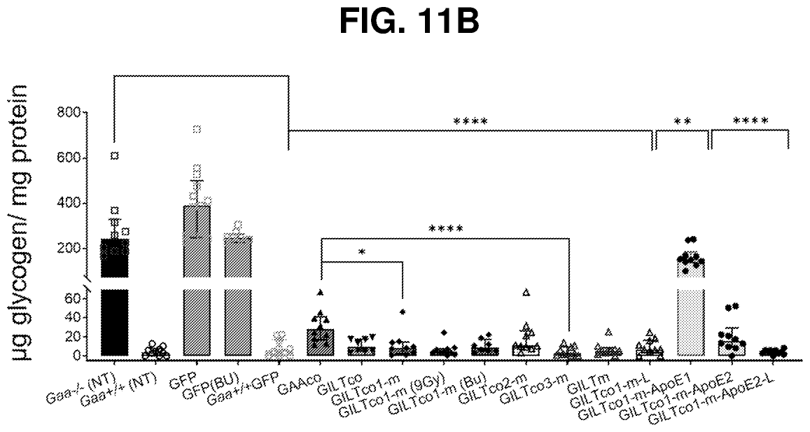

Subsequent to injection (or lack thereof), GAA activity was measured using an enzymatic assay with 4-methylumbelliferyl alpha-D-glucopyronoside as fluorogenic substrate across different experimental groups (see van Til et al. Blood 115:5329-37, 2010; Bijvoet et al. Hum Mol Genet 8:2145-53, 1999; and Bijvoet et al. Hum Mol Genet 7:53-62, 1998). The level of glycogen accumulation was also measured using a method which measures degradation of glycogen into glucose. The amount of glucose was determined by a glucose oxidase method described in Til et al. Blood 115:5329-37 (see also van der Ploeg et al. J Neurol Sci 79:327-36, 1987; Reuser et al. Am J Hum Genet 30:132-43, 1978; and van Hove et al. PNAS 93:65, 1996), and reported as percent values relative to treatment naïve Gaa−/− mice. GAA activity and glycogen accumulation were measured across various muscle tissue and nervous system tissue, including the diaphragm (FIGS. 1A and 11B), cerebrum (FIGS. 2A and 2B), cerebellum (FIGS. 3A and 3B), spinal cord (FIGS. 4A and 4B), quadriceps femoris muscle (FIGS. 5A and 5B), gastrocnemius muscle (FIGS. 6A and 6B), and tibialis anterior muscle (FIGS. 7A and 7B). All Gaa−/− treatment groups receiving HSCs transduced the GILT.R37A.GAA lentiviral vector exhibited a dose-dependent increase in GAA activity across a majority of the tested tissues. Glycogen reduction in target tissues appeared to be dose-independent as near-complete reduction of glycogen accumulation was observed at even the lowest viral vector dose (MOI 0.75).

Thus, the above findings demonstrate the therapeutic efficacy of administering pluripotent stem cells (such as HSCs) transduced with a lentiviral vector containing a transgene encoding a GILT.GAA fusion protein containing an R37A IGF-II mutein in a murine model of Pompe disease. This presents a potentially curative strategy for the treatment of human Pompe disease patients.

### Example 4. Secretion of GAA Protein from HAP1 GAA−/− Cells Transduced with Variants of a Transgene Encoding a GILT.GAA Protein

GAA is poorly secreted by cells. This study was conducted to assess the secretion of GAA protein from cells transduced with variants of a transgene encoding a GILT.GAA fusion protein under transcriptional control of an MND promoter.

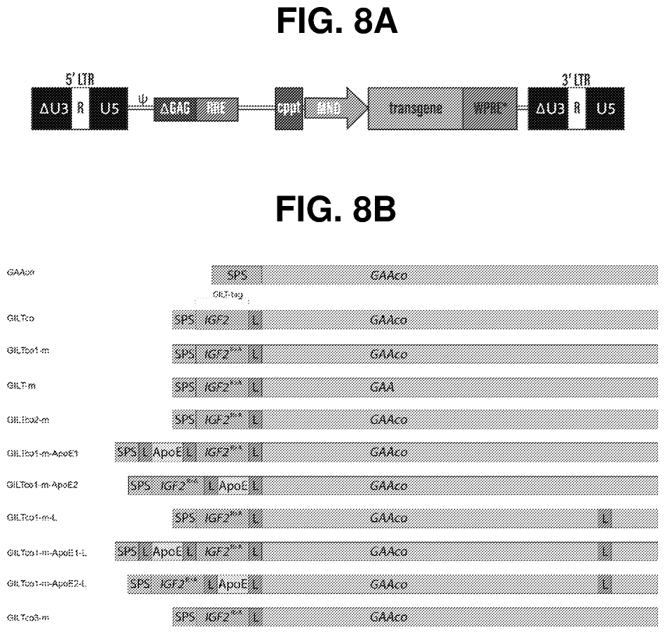

HAP1 GAA−/− cells were transduced with nine lentiviral vectors encoding variants of a GILT.GAA protein containing an R37A IGF-II mutein (MOI: 3) (FIGS. 8A and 8B). Eight of these vectors contained unique sequences encoding codon-optimized GAA. Two codon-optimized GAA-encoding sequences in the lentiviral vectors, GILTco1-m and GILTco2-m, were generated through two different codon optimization algorithms. A GAA sequence translating into a consensus amino acid sequence was also used in the lentiviral vectors to encode codon-optimized GAA (GILT-co3-m). Two lentiviral vectors further contained a sequence encoding ApoE upstream of the GILT-tag (GILTco1-m-ApoE1) or downstream of the GILT-tag (GILTco1-m-ApoE2). One lentiviral vector further encoded a Gly-Ala-Pro peptide linker sequence located within a codon-optimized GAA amino acid sequence (GILTco1-m-L), and two lentiviral vectors further encoded ApoE and a Gly-Ala-Pro linker (GILTco1-m-ApoE1-L and GILTco1-m-ApoE2-L). Another tested group included HAP1 GAA−/− cells encoding a wild-type GAA transgene and a GILT tag containing an R37A IGF-II mutein (GILTm). For comparison, wild-type HAP1 cells, HAP1 GAA−/− cells encoding a codon-optimized GAA transgene and a GILT tag that lacks an R37A IGF-II mutein (GILTco), and HAP1 GAA^{-/-} cells transduced with a codon-optimized GAA transgene lacking a GILT tag (GAAco) were used as control groups. Other tested groups included GAA−/− HAP1 cells transduced with a transgene encoding green fluorescent protein (GFP).

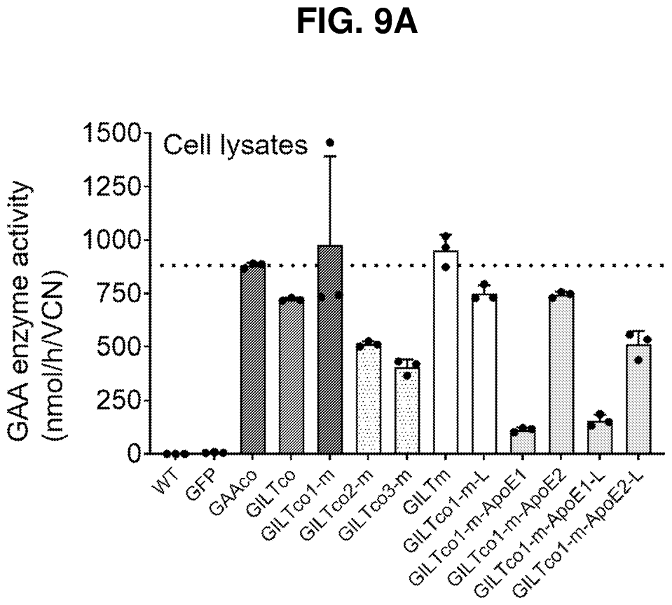

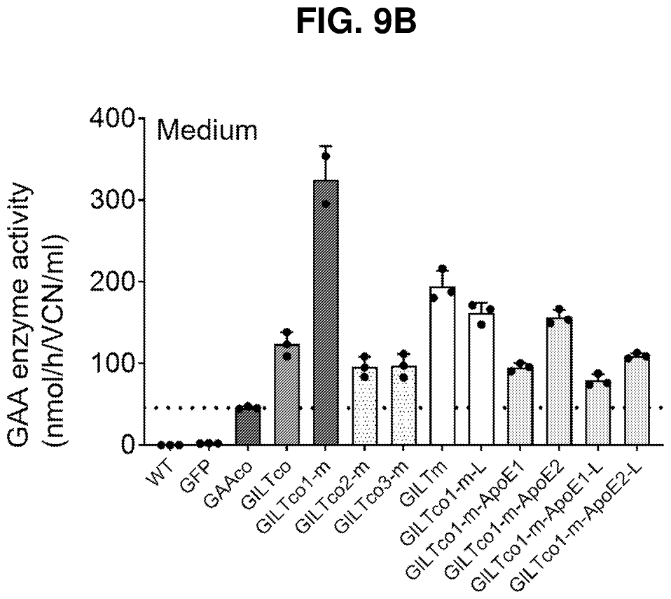

Conditioned media of vector transduced cells and cell pellets were collected on day 11 for VCN-normalized GAA activity measurement. GAA activity was measured as described Jack et al. Genet Med 8(5):307-12, 2006. The results are shown in FIGS. 9A and 9B.

### Example 5. Comparing Therapeutic Expression of GAA Activity in Mouse Models of Pompe Disease Treated with Lineage Negative Hematopoietic Stem Cells Transduced with Variants of a Transgene Encoding a GILT.GAA Protein

The objective of this study was to compare the therapeutic efficacy of lineage negative HSCs transduced with variants of a transgene encoding a GILT.GAA fusion protein under transcriptional control of an MND promoter. The transgenes contained distinct features, such as different codon-optimized sequences, ApoE moieties, and linker sequences.

To this end, 159 GAA knock-out mice (Gaa−/−) and 26 wild-type mice (Gaa+/+) were employed as part of an experimental study. Multiple groups of ten Gaa−/− female mice were first conditioned with myeloablative 7.5 Gy radiation, 9 Gy radiation, or Busulfex® (4×25/mg/kg) to ablate endogenous microglial cells, and subsequently treated (by way of intravenous injection) with 5×10⁵ lineage negative HSCs from donor male Gaa−/− mice, the cells having been previously transduced with a lentiviral vector containing a transgene encoding a GILT.GAA protein containing an R37A IGF-II mutein (see Table 3 below). Seven of these lentiviral vectors selected for testing in this study contained unique sequences encoding codon-optimized GAA (FIGS. 8A and 8B). Two codon-optimized GAA-encoding sequences in the lentiviral vectors, GILTco1-m and GILTco2-m, were generated through two different codon optimization algorithms. A GAA sequence translating into a consensus amino acid sequence was also used in the lentiviral constructs to encode codon-optimized GAA (GILT-co3-m). Two lentiviral vectors further contained a sequence encoding ApoE upstream of the GILT-tag (GILTco1-m-ApoE1) or downstream of the GILT-tag (GILTco1-m-ApoE2). One lentiviral vector further encoded a Gly-Ala-Pro peptide linker sequence within a codon-optimized GAA amino acid sequence (GILTco1-m-L), and another lentiviral vector encoded ApoE and a Gly-Ala-Pro peptide linker (GILTco1-m-ApoE2-L). An additional tested group included Gaa−/− mice receiving 7.5 Gy radiation and HSCs encoding a wild-type GAA transgene and a GILT tag containing an R37A IGF-II mutein (GILTm).

For comparison, Gaa−/− and wild-type GAA+/+ mice receiving no HSCs (NT), Gaa−/− mice receiving 7.5 Gy radiation and HSCs encoding a codon-optimized GAA transgene and a GILT tag that lacks an R37A IGF-II mutein (GILTco), and Gaa−/− mice receiving 7.5 Gy radiation and HSCs encoding a codon-optimized GAA transgene lacking a GILT tag (GAAco) were used as control groups. Other tested groups include Gaa−/− mice receiving 7.5 Gy radiation or Busulfex® and HSCs transduced with a transgene encoding green fluorescent protein (GFP) as well as wild-type GAA+/+ mice receiving 7.5 Gy radiation and HSCs transduced with a transgene encoding GFP (see Table 3 below).

TABLE 3
Description of experimental groups

| | | | Number of | | Number | | |
|---|---|---|---|---|---|---|---|
| | Donor | Recipient | Recipient | | of Donor | | |
| Group | Phenotype | Phenotype | Mice | Lentiviral Identity | Cells | Conditioning | |
| 1 | Gaa−/− | Gaa−/− | 10 | GILTco1-m | 5 × 10⁵ | 7.5 | Gy |
| 2 | Gaa−/− | Gaa−/− | 10 | GILTco1-m | 5 × 10⁵ | 9 | Gy |
| 3 | Gaa−/− | Gaa−/− | 10 | GILTco1-m | 5 × 10⁵ | Busulfex ® | |
| | | | | | | (4 × 25/mg/kg) | |
| 4 | Gaa−/− | Gaa−/− | 10 | GILTco1-m-L | 5 × 10⁵ | 7.5 | Gy |
| 5 | Gaa−/− | Gaa−/− | 10 | GILTco1-m-ApoE1 | 5 × 10⁵ | 7.5 | Gy |
| 6 | Gaa−/− | Gaa−/− | 10 | GILTco1-m-ApoE2 | 5 × 10⁵ | 7.5 | Gy |
| 7 | Gaa−/− | Gaa−/− | 10 | GILTco1-m-ApoE2-L | 5 × 10⁵ | 7.5 | Gy |
| 8 | Gaa−/− | Gaa−/− | 10 | GILTco2-m | 5 × 10⁵ | 7.5 | Gy |
| 9 | Gaa−/− | Gaa−/− | 10 | GILTco3-m | 5 × 10⁵ | 7.5 | Gy |
| 10 | Gaa−/− | Gaa−/− | 10 | GILTco | 5 × 10⁵ | 7.5 | Gy |
| 11 | Gaa−/− | Gaa−/− | 10 | GILTm | 5 × 10⁵ | 7.5 | Gy |
| 12 | Gaa−/− | Gaa−/− | 13 | GFP | 5 × 10⁵ | 7.5 | Gy |
| 13 | Gaa−/− | Gaa−/− | 10 | GFP | 5 × 10⁵ | Busulfex ® | |
| | | | | | | (4 × 25/mg/kg) | |
| 14 | Gaa+/+ | Gaa+/+ | 10 | GFP | 5 × 10⁵ | 7.5 | Gy |
| 15 | Gaa−/− | Gaa−/− | 10 | GAAco (control) | 5 × 10⁵ | 7.5 | Gy |

| | | | | | | |
|---|---|---|---|---|---|---|
| 16 | N/A | Gaa+/+ | 16 | NT | N/A | N/A |
| 17 | N/A | Gaa−/− | 16 | NT | N/A | N/A |

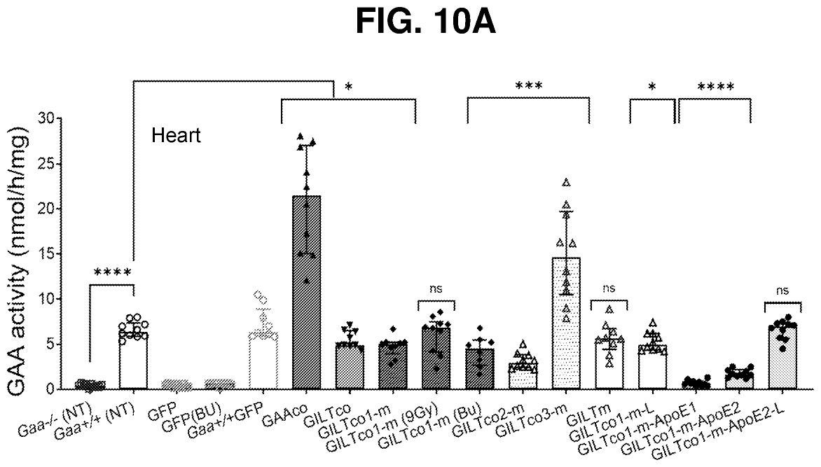

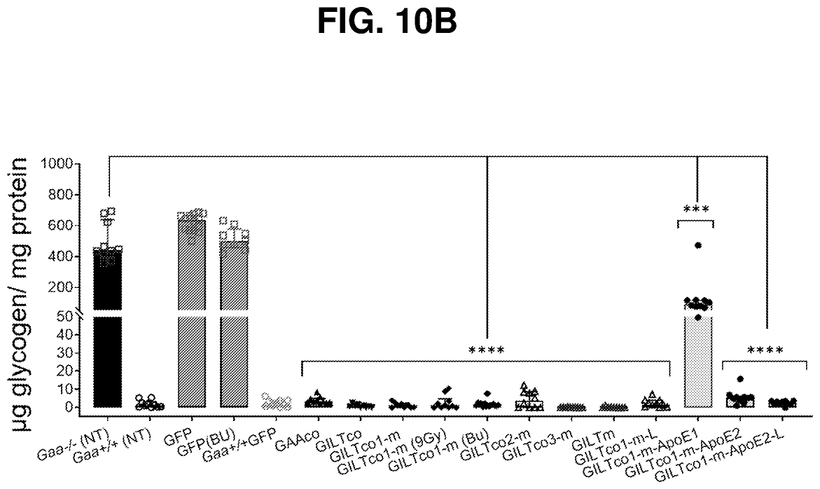

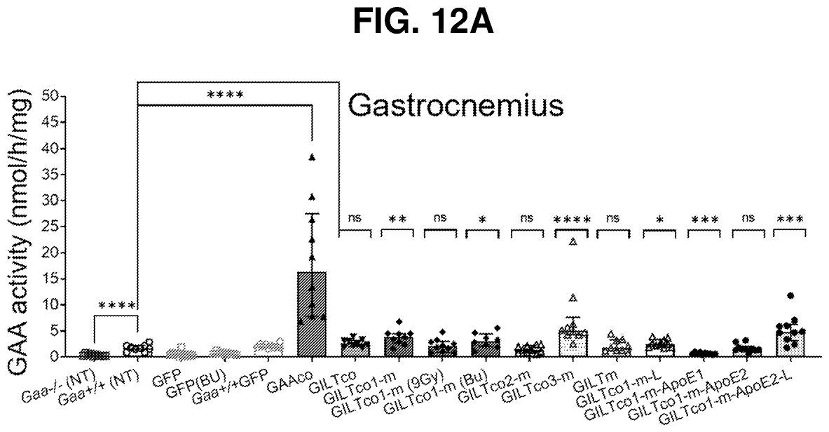

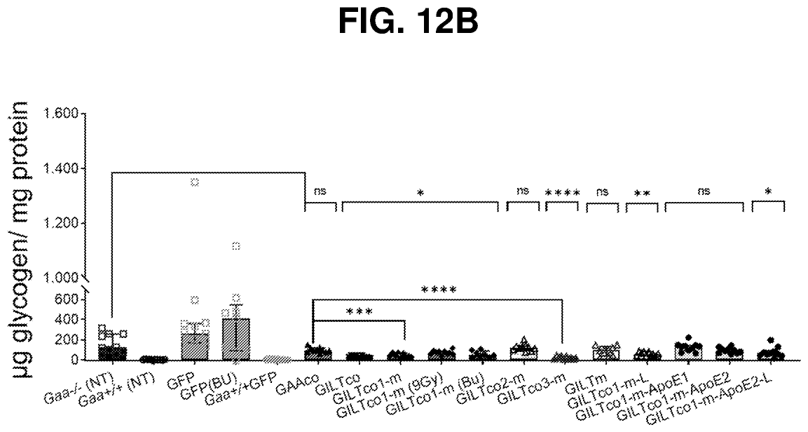

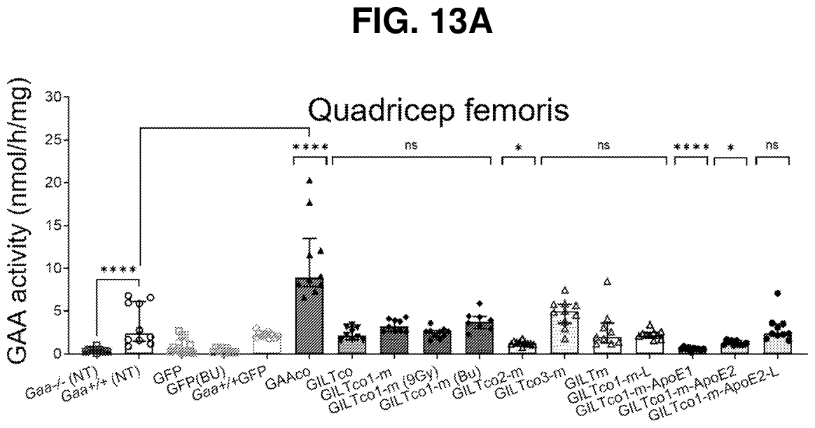

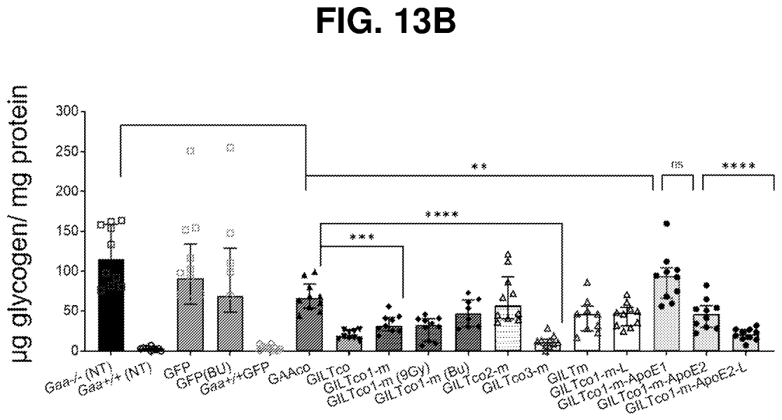

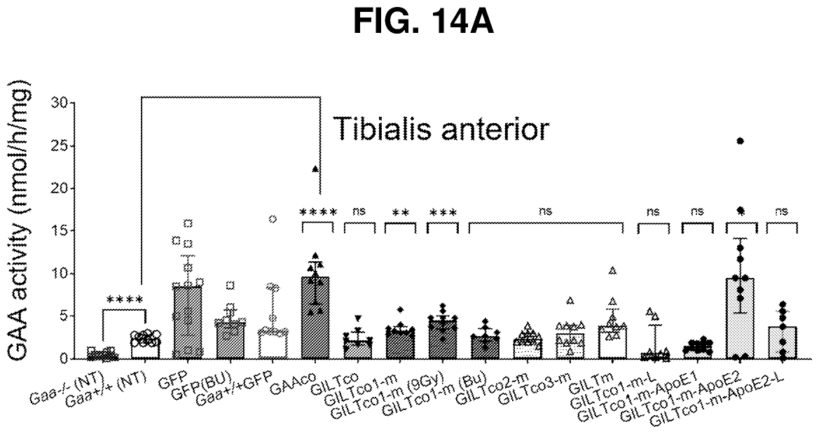

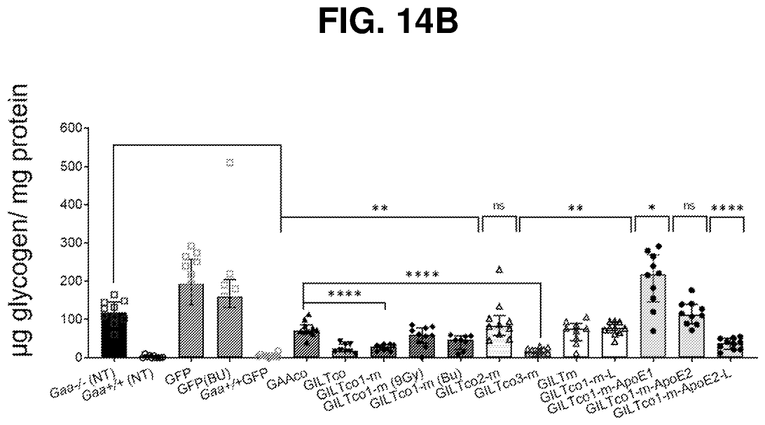

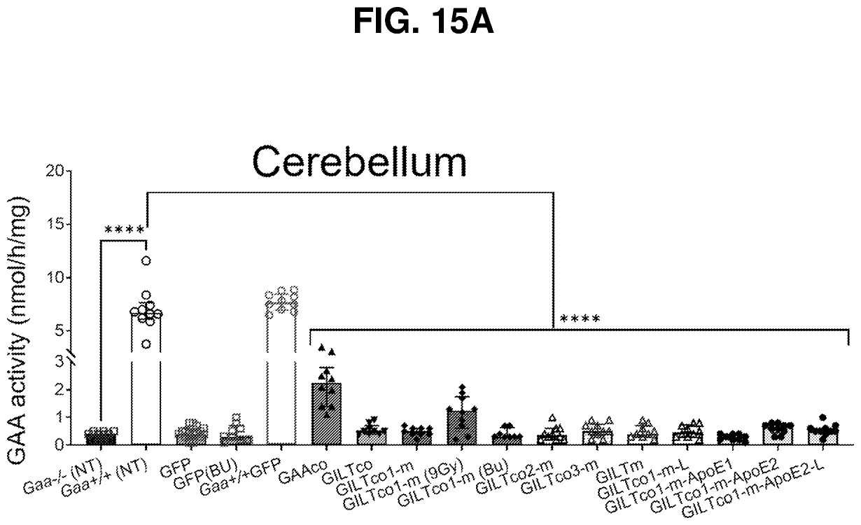

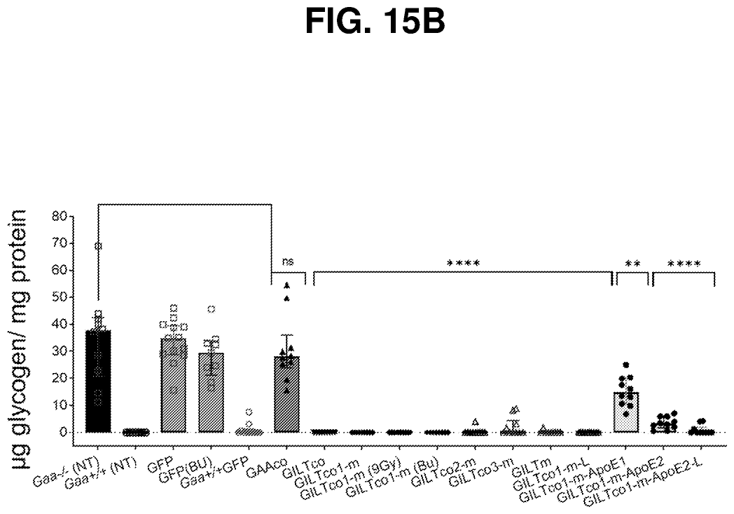

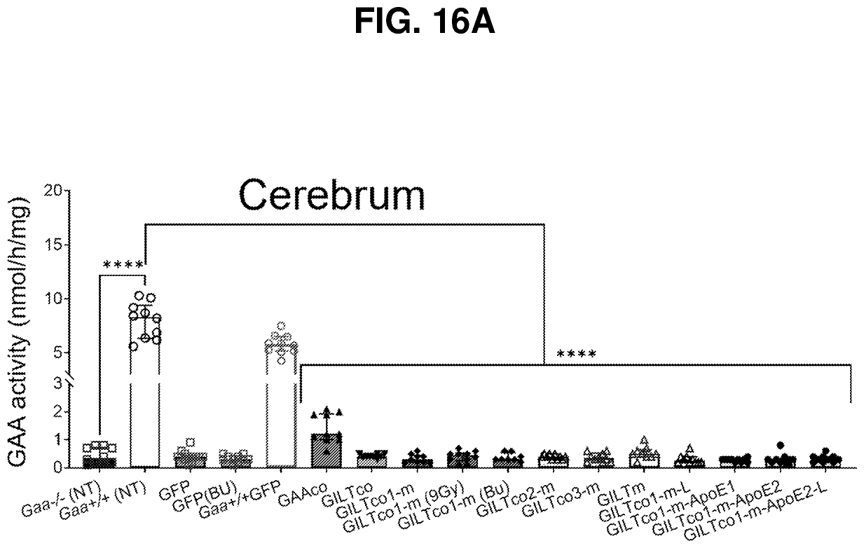

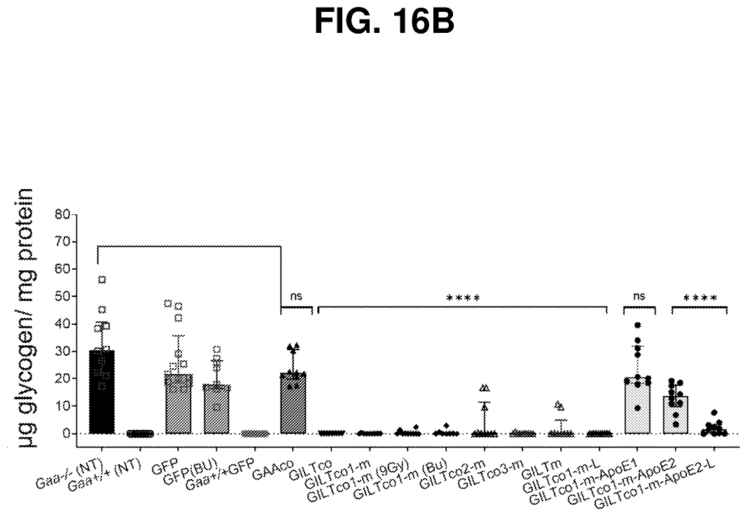

Subsequent to injection (or lack thereof), GAA activity was measured across different experimental groups using an enzymatic assay described in Jack et al. Genet Med 8(5):307-12, 2006. The level of glycogen accumulation was also measured using a method described in Okumiya et al. Mol Genet Metab 88(1):22-8, 2006 and reported as percent values relative to treatment naïve Gaa−/− mice. GAA activity and glycogen accumulation were measured across various muscle tissue and nervous system tissue, including the heart (FIGS. 10A and 10B), diaphragm (FIGS. 11A and 11B), gastrocnemius muscle (FIGS. 12A and 12B), quadriceps femoris muscle (FIGS. 13A and 13B), tibialis anterior muscle (FIGS. 14A and 14B), cerebellum (FIGS. 15A and 15B), and cerebrum (FIGS. 16A and 16B). The results of these experiments are shown in FIGS. 10A-16B.

The results of this study revealed that, surprisingly, attempts to enhance BBB-crossing delivery and CNS pharmacodynamics through incorporation of an additional CNS-specific tag, ApoE, into a transgene encoding a GILT.GAA fusion protein were unsuccessful. The findings of these experiments also demonstrated high therapeutic efficacy of HSCs transduced with several of the tested lentiviral constructs encoding a codon-optimized GAA protein fused to a GILT tag containing an R37A IGF-II mutein, such as GILTco1-m, in Gaa−/− mice.

### Example 6. Safety of Lineage Negative Hematopoietic Stem Cells Transduced with a Transgene Encoding GILT.GAA for the Treatment of Pompe Disease

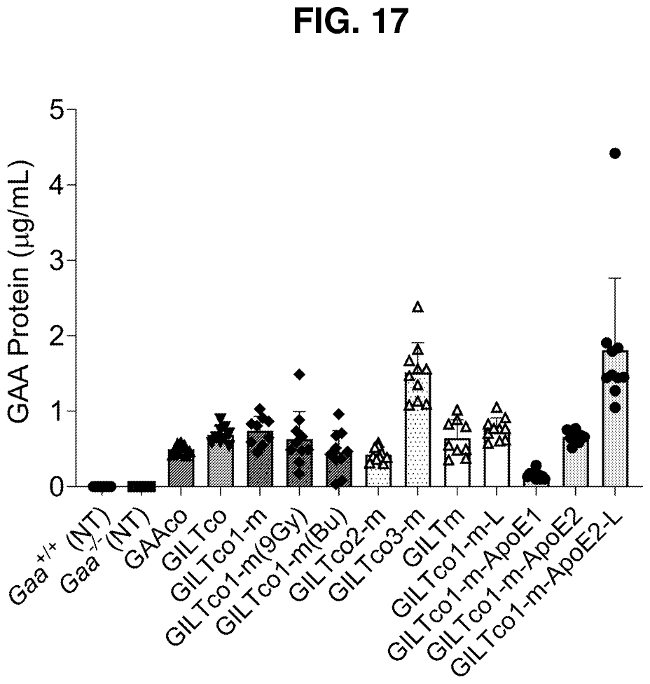

In a clinical trial, the use of reveglucosidase, an IGF2-tagged GAA analog, induced transient hypoglycemia at high dose infusions (20 mg/kg) in 88% of late-onset Pompe patients (Byrne et al., Orphanet J Rare Dis. 2017; 12(1):144). To assess the safety of lineage negative hematopoietic stem cells encoding a GAA protein fused to a GILT tag, plasma GAA protein concentrations in the experimental groups outlined in Example 5 were quantified using Western Blot analysis (FIG. 17). The range of GAA protein was 0.03 to 4.4 μg/mL in all tested groups. Based on an adult blood volume of 5 liters and an ERT dose of 20 mg/kg, the levels detected in the plasma of Gaa−/− mice administered hematopoietic stem cells encoding GILT.GAA was approximately 45- to 131-fold lower than what would theoretically be infused into late-onset Pompe patients through a bolus injection as described in Byrne et al., 2017. The factors included in these calculations are presented in Table 4 below.

TABLE 4
Comparison of GAA plasma concentration in Gaa−/− mice administered
hematopoietic stem cells encoding GILT.GAA and GAA plasma concentration
in Pompe patients administered a bolus injection of reveglucosidase

| | | | | | Data from | |
|---|---|---|---|---|---|---|
| | | | | | present | |
| | | | | | preclinical | |
| Data from clinical trial (Byrne et al., 2017) | | | | | mouse study | |
| | High | Total | Blood | Theoretical | Highest mouse | |
| Weight | Dose | max dose | volume | blood conc. | plasma GAA | Approximate |
| (kg) | (mg/kg) | (mg) | (L) | (mg/mL) | conc. (μg/mL) | fold reduction |
| 49.2 | 20 | 984 | 5 | 0.1968 | 4.4 | 45 |
| 144.5 | 20 | 2890 | 5 | 0.578 | 4.4 | 131 |

### Example 7. Establishing Therapeutic Efficacy of GAA Activity in Mouse Models of Pompe Disease Treated with Lineage Negative Hematopoietic Stem Cells Transduced with a Transgene Encoding GILTco1-m

A 32-week experimental study was conducted to further assess the therapeutic efficacy of HSCs transduced with the GILTco1-m construct described in Example 5.

To this end, 104 GAA knock-out mice (Gaa−/−) and 26 wild-type mice (Gaa+/+) were employed as part of the study. Multiple groups of thirteen Gaa−/− mice, each male and female, were first conditioned with myeloablative 7.5 Gy radiation to ablate endogenous microglial cells, and subsequently treated (by way of intravenous injection) with 5×10 lineage negative HSCs from donor Gaa−/− mice, the cells having been previously transduced with a lentiviral vector containing GILTco1-m (MOI: 0.75, 1.5, and 3) (see Table 5 below). For comparison, Gaa−/− and wild-type GAA+/+ mice receiving no HSCs were used as control groups.

TABLE 5
Description of experimental groups

| | | | Number of | | Target Vector | Number | |
|---|---|---|---|---|---|---|---|
| | Donor | Recipient | Recipient | Lentiviral | Copy Number | of Donor | |
| Group | Phenotype | Phenotype | Mice | Identity | (VCN) | Cells | Conditioning |
| 1 | Gaa−/− | Gaa−/− | 13 M/13 F | GILTco1-m | 0.75 | 5 × 10⁵ | 7.5 Gy |
| 2 | Gaa−/− | Gaa−/− | 13 M/13 F | GILTco1-m | 1.5 | 5 × 10⁵ | 7.5 Gy |
| 3 | Gaa−/− | Gaa−/− | 13 M/13 F | GILTco1-m | 3 | 5 × 10⁵ | 7.5 Gy |
| 4 | N/A | Gaa−/− | 13 M/13 F | N/A | N/A | N/A | N/A |
| 5 | N/A | Gaa+/+ | 13 M/13 F | N/A | N/A | N/A | N/A |

### GAA Activity

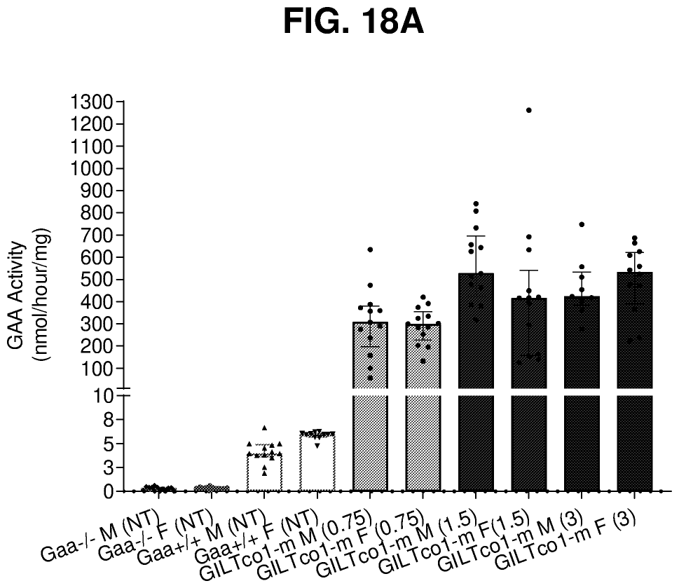

Subsequent to injection (or lack thereof), GAA activity was measured using an enzymatic assay described in Jack et al. Genet Med 8(5):307-12, 2006 across different experimental groups. GAA activity was measured in bone marrow at termination (week 32 post-treatment) and in plasma at weeks 4, 16, and 31 post-treatment. The results are shown in FIGS. 18A and 18B, respectively.

### Vector Copy Number Analysis

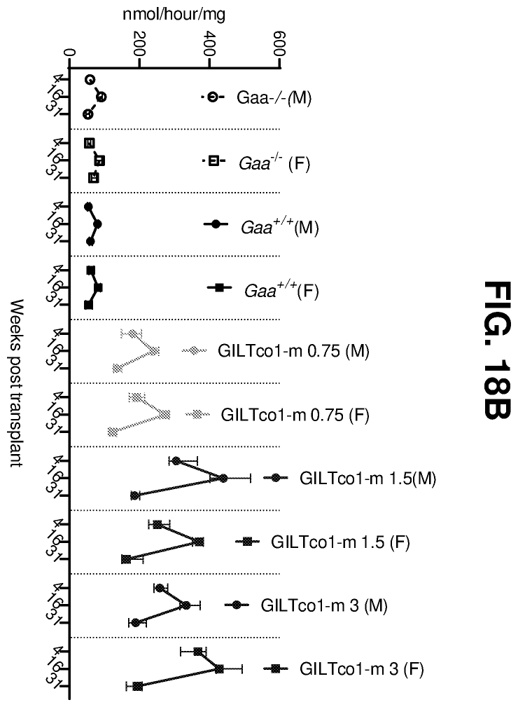

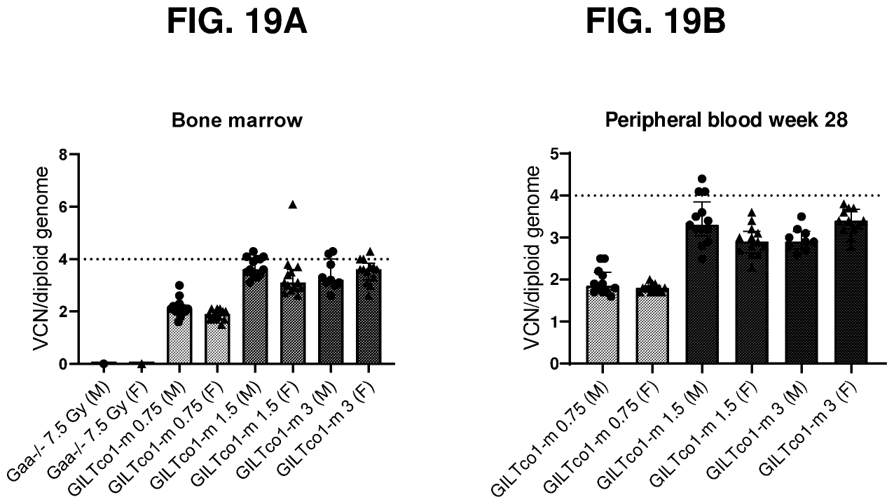

Vector copy number was measured subsequent to injection (or lack thereof) in bone marrow and peripheral blood across the experimental groups. Bone marrow samples collected at termination (32 weeks post-treatment) were processed for gDNA and quantified with Quant-iT™ assay kit or Nanodrop One. This qPCR assay consisted of oligonucleotide primers and probe mix containing either a TaqMan 6-carboxyfluorescenin (FAM) or VICTM fluorescent probe designed to amplify HIV Psi vector and Gtdc1 housekeeping gene sequences. A plasmid containing both sequences was used as a reference standard in a range of 50 to 5e10⁷ copies. Data was reported as VCN/diploid genome. The results are shown in FIGS. 19A and 19B.

### Urinary Analysis

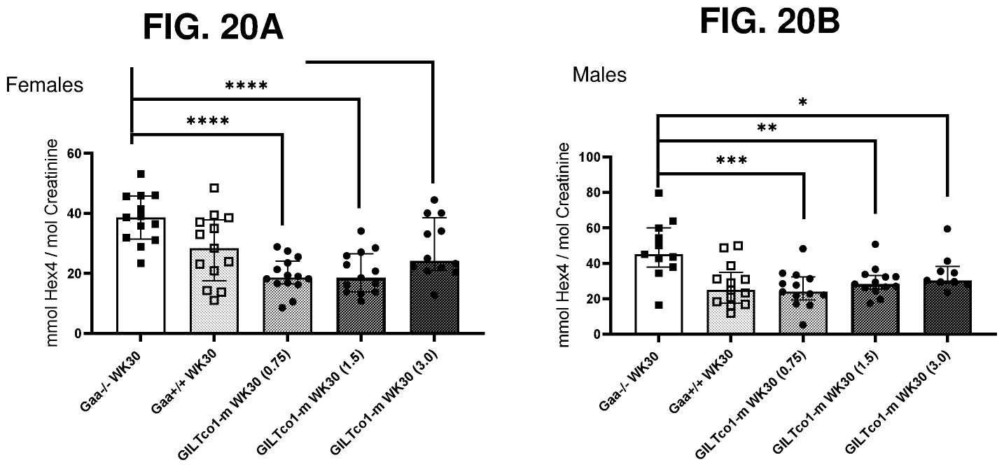

Urinary 6-α-D-glucopyranosyl maltotriose (HEX4) glucotetrasaccharide has been used as a biomarker to measure efficacy of ERT in Pompe patients and efficacy of AAV gene therapy in Gaa−/− mice. Accordingly, urinary HEX4 glucotetrasaccharide concentration was measured pre-treatment and at week 30 post-treatment in the experimental groups. Mice were fasted before urine collection for HEX4 glucotetrasaccharide. Quantification of HEX4 was conducted using a Shimadzu instrument (Columbia, MD) via protein precipitation using acetonitrile and 4 mM uric acid with 0.2% NH4OH as surrogate matrix. Calibration standards and quality controls were run in surrogate matrix and mouse urine matrix in duplicate. Results were normalized by measurement of creatinine in the urine sample via a commercial kit. The results are shown in FIGS. 20A and 20B.

### Histological Analysis of Glycogen Accumulation and Vacuolation

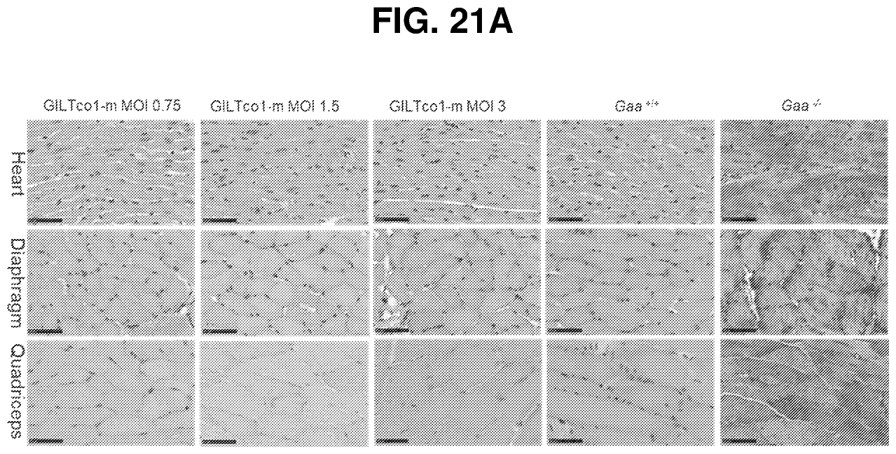

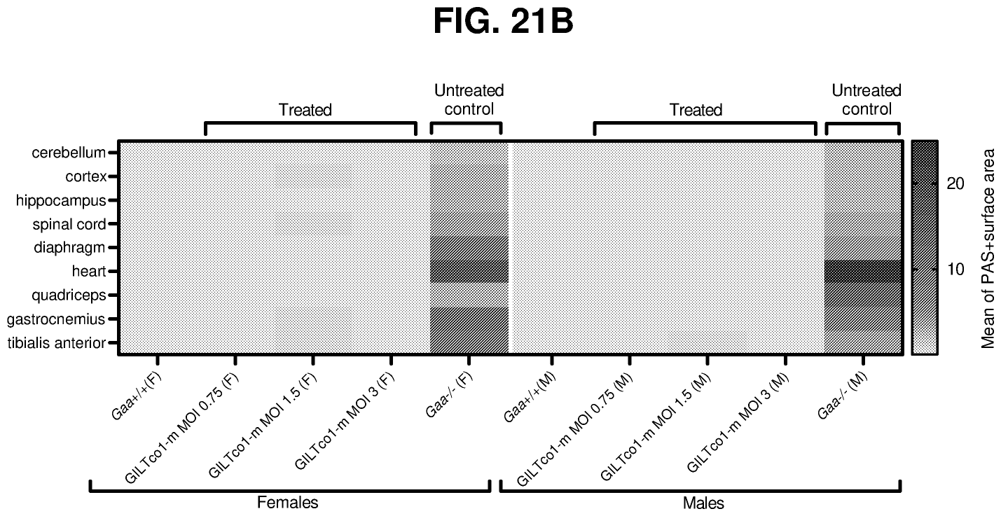

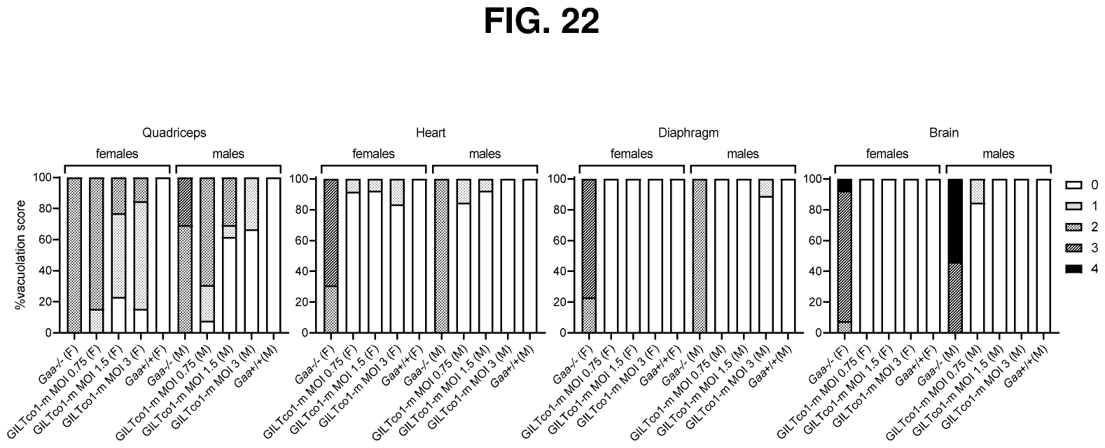

To confirm the results of the glycogen accumulation biochemical assays described in Examples 3 and 5 and to quantify myofiber and CNS vacuolation, histological analysis was performed. Following scheduled necropsy, sections of the cerebral cortex, cerebellum, hippocampus, and/or brainstem, thoracic and cervical spinal cord, heart, quadriceps femoris muscle, diaphragm, gastrocnemius muscle, and tibialis anterior muscle were processed for Periodic Acid-Schiff staining (FIGS. 21A and 21B) and hematoxylin and eosin staining (FIG. 22). The sections were evaluated for glycogen accumulation and vacuolation by light microscopy. For severity scoring of vacuolation, a score was assigned as minimally (score 1) affected tissues having <50% of cells within the section with small discrete centralized regions of cytoplasmic vacuolation involving <10% of the cytoplasmic volume; mildly (score 2) affected tissues having larger regions of vacuolation involving >10% of the cytoplasmic volume affecting <50% of cells within the section and none to rare myofibers that were diffusely enlarged with overall decreased cytoplasmic staining intensity; moderately (score 3) affected tissues having regions of cytoplasmic vacuolation involving >10% of cell with >50% of myofibers showing evidence of myofiber degeneration characterized by enlargement of myofibers and overall decreased staining intensity; and markedly (score 4) affected tissues having overall enlargement and decreased staining intensity of the majority of myofibers with both centralized regions of cytoplasmic vacuolation and evidence of myofiber degeneration.

### Echocardiography Evaluation

Cardiac hypertrophy is a hallmark of infantile-onset Pompe patients and Gaa−/− mice. To assess the therapeutic efficacy of HSCs transduced with the GILTco1-m construct on cardiac hypertrophy, left ventricle mass index was calculated across the experimental groups using echocardiography as follows:

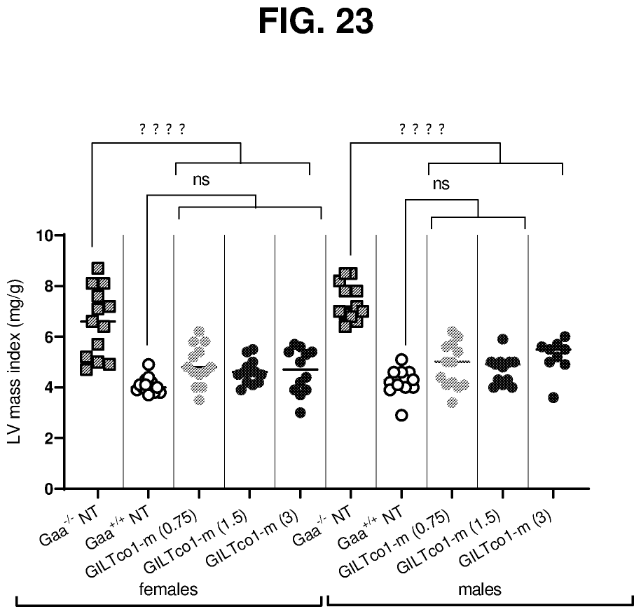

Mass index=0.8(1.04([LVIDd+PWTd+IVSTd]³−[LVIDd]³))+0.6 g/body surface area

where LVIDd is Left Ventricular Internal Diameter in Diastole, PWTd is Posterior Wall Thickness in Diastole, and IVSTd is Interventricular Septum Thickness in Diastole. The results are shown in FIG. 23.

### Motor Function Assessments

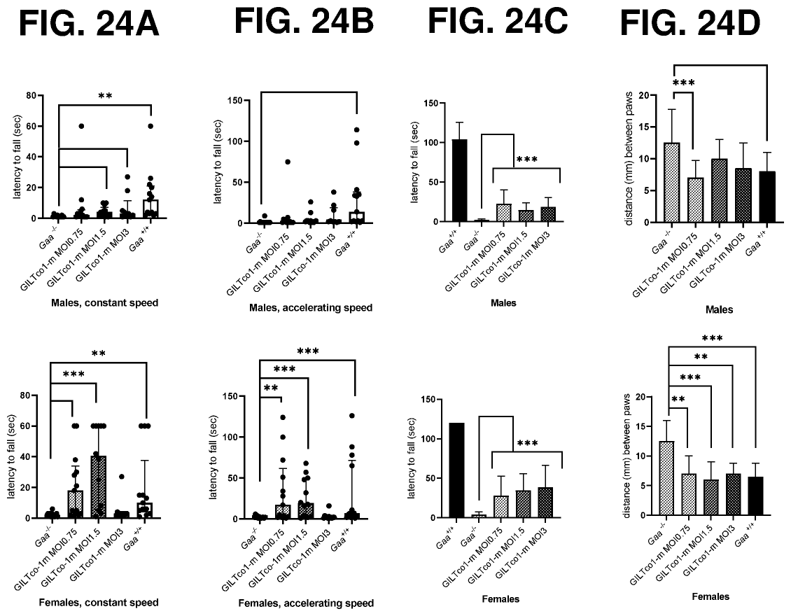

Rotarod assessment, wire hang assessment, and gait analysis were conducted to evaluate motor function across the experimental groups. In the rotarod assessment, animal latency to fall was measured at constant rotating speed (5 rpm for 60 seconds) and accelerating rotating speed (4 to 15 rpm over a 3.05-minute period). The results of the rotarod assessment are shown in FIGS. 24A and 24B, respectively. In the wire hang assessment, animal latency to fall was measured in three trials. The results of the wire hang assessment are shown in FIG. 24C. Gait analysis was evaluated as distance between two paw prints, the results of which are shown in FIG. 24D. Significant differences were observed in the motor function assessments in males and females treated with HSCs transduced with a lentiviral vector containing GILTco1-m compared to GAA−/− mice. Sex differences were also observed.

### Other Embodiments

Various modifications and variations of the described disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure that are obvious to those skilled in the art are intended to be within the scope of the disclosure.

Other embodiments are in the claims.

1. A method of treating Pompe disease in a subject, the method comprising administering to the subject a composition comprising a population of cells comprising a transgene encoding an acid alpha-glucosidase (GAA) protein fused to a glycosylation-independent lysosomal targeting (GILT) tag (GILT.GAA protein), wherein the GILT tag comprises a human insulin-like growth factor II (IGF-II) mutein comprising an Ala amino acid substitution at a position corresponding to Arg 37 of SEQ ID NO: 15.

2. A method of improving muscle function in a subject diagnosed as having Pompe disease, the method comprising administering to the subject a composition comprising a population of cells comprising a transgene encoding a GILT.GAA protein, wherein the GILT tag comprises an IGF-II mutein comprising an Ala amino acid substitution at a position corresponding to Arg 37 of SEQ ID NO: 15.

3. A method of reducing glycogen accumulation in a subject diagnosed as having Pompe disease, the method comprising administering to the subject a composition comprising a population of cells comprising a transgene encoding a GILT.GAA protein, wherein the GILT tag comprises an IGF-II mutein comprising an Ala amino acid substitution at a position corresponding to Arg 37 of SEQ ID NO: 15.

4. A method of improving pulmonary function in a subject diagnosed as having Pompe disease, the method comprising administering to the subject a composition comprising a population of cells comprising a transgene encoding GILT.GAA protein, wherein the GILT tag comprises an IGF-II mutein comprising an Ala amino acid substitution at a position corresponding to Arg 37 of SEQ ID NO: 15.

5. A method of increasing GAA expression in a subject diagnosed as having Pompe disease, the method comprising administering to the subject a composition comprising a population of cells comprising a transgene encoding a GILT.GAA protein, wherein the GILT tag comprises an IGF-II mutein comprising an Ala amino acid substitution at a position corresponding to Arg 37 of SEQ ID NO: 15.

6. The method of any one of claims 1-5, wherein the human IGF-II mutein has an amino acid sequence that is at least 70% identical to the amino acid sequence of mature human IGF-II (SEQ ID NO: 15).

7. The method of any one of claims 1-6, wherein the GILT tag has an amino acid sequence that is at least 70% identical to the amino acid sequence of SEQ ID NO: 16.

8. The method of any one of claims 1-6, wherein the GILT tag has an amino acid sequence that is at least 70% identical to the amino acid sequence of SEQ ID NO: 17.

9. The method of any one of claims 1-6, wherein the GILT tag has an amino acid sequence that is at least 70% identical to the amino acid sequence of SEQ ID NO: 18.

10. The method of any one of claims 1-6, wherein the GILT tag has an amino acid sequence that is at least 70% identical to the amino acid sequence of SEQ ID NO: 19.

11. The method of any one of claims 1-6, wherein the GILT tag has an amino acid sequence that is at least 70% identical to the amino acid sequence of SEQ ID NO: 20.

12. The method of any one of claims 1-11, wherein the GILT tag is encoded by a polynucleotide having a nucleic acid sequence that is at least 85% identical to the nucleic acid sequence of SEQ ID NO: 21.

13. The method of any one of claims 1-11, wherein the GILT tag is encoded by a polynucleotide having a nucleic acid sequence that is at least 85% identical to the nucleic acid sequence of SEQ ID NO: 22.

14. The method of any one of claims 1-11, wherein the GILT tag is encoded by a polynucleotide having a nucleic acid sequence that is at least 85% identical to the nucleic acid sequence of SEQ ID NO: 23.

15. The method of any one of claims 1-14, wherein the human IGF-II mutein has diminished binding affinity for the insulin receptor relative to the affinity of naturally-occurring human IGF-II for the insulin receptor, wherein the IGF-II mutein is resistant to furin cleavage, wherein the IGF-II mutein binds to the human cation-independent mannose-6-phosphate receptor in a mannose-6-phosphate-independent manner.

16. The method of any one of claims 1-15, wherein the transgene is operably linked to a promoter.

17. The method of claim 16, wherein the promoter is a ubiquitous promoter.

18. The method of claim 16, wherein the promoter is a cell lineage-specific promoter.

19. The method of claim 16, wherein the promoter is a viral promoter.

20. The method of claim 16, wherein the promoter is a synthetic promoter.

21. The method of claim 20, wherein the synthetic promoter is a Myeloproliferative Sarcoma Virus Enhancer, Negative Control Region Deleted, dl587rev Primer-Binding Site Substituted (MND) promoter.

22. The method of claim 21, wherein the MND promoter comprises a polynucleotide having at least 85% sequence identity to the nucleic acid sequence of SEQ ID NO: 10.

23. The method of claim 21, wherein the MND promoter comprises a polynucleotide having at least 85% sequence identity to the nucleic acid sequence of SEQ ID NO: 11.

24. The method of any one of claims 1-23, wherein the transgene encodes a GAA protein having an amino acid sequence that is at least 85% identical to the amino acid sequence of SEQ ID NO: 1.

25. The method of any one of claims 1-24, wherein the transgene encodes a GAA protein having an amino acid sequence that is at least 85% identical to the amino acid sequence of SEQ ID NO: 2.

26. The method of any one of claims 1-25, wherein the transgene encodes a GAA protein having an amino acid sequence that is at least 85% identical to the amino acid sequence of SEQ ID NO: 3.

27. The method of any one of claims 1-26, wherein the transgene encodes a GAA protein having an amino acid sequence that is at least 85% identical to the amino acid sequence of SEQ ID NO: 4.

28. The method of any one of claims 1-27, wherein the GAA protein is encoded by a polynucleotide having at least 85% sequence identity to the nucleic acid sequence of SEQ ID NO: 5.

29. The method of any one of claims 1-28, wherein the GAA protein is encoded by a polynucleotide having at least 85% sequence identity to the nucleic acid sequence of SEQ ID NO: 6.

30. The method of any one of claims 1-29, wherein the GAA protein is encoded by a polynucleotide having at least 85% sequence identity to the nucleic acid sequence of SEQ ID NO: 7.

31. The method of any one of claims 1-30, wherein the GAA protein is encoded by a polynucleotide having at least 85% sequence identity to the nucleic acid sequence of SEQ ID NO: 8.

32. The method of any one of claims 1-31, wherein the GAA protein is encoded by a polynucleotide having at least 85% sequence identity to the nucleic acid sequence of SEQ ID NO: 9.

33. The method of any one of claims 1-32, wherein the GAA is a full-length GAA.

34. The method of any one of claims 1-33, wherein the GAA comprises a signal peptide.

35. The method of claim 34, wherein the signal peptide is a GAA signal peptide.

36. The method of claim 34, wherein the signal peptide is an IGF-II signal peptide.

37. The method of claim 36, wherein the IGF-II signal peptide comprises an amino acid sequence of SEQ ID NO: 12.

38. The method of any one of claims 1-37, wherein the transgene encodes two or more GILT.GAA proteins.

39. The method of any one of claims 1-38, wherein the transgene is a codon-optimized GILT.GAA transgene.

40. The method of any one of claims 1-39, wherein the GILT.GAA protein comprises a receptor-binding (Rb) domain of apolipoprotein E (ApoE).

41. The method of claim 40, wherein the Rb domain comprises a portion of ApoE having the amino acid sequence of residues 25-185, 50-180, 75-175, 100-170, 125-160, or 130-150 of SEQ ID NO: 24.

42. The method of claim 40 or 41, wherein the Rb domain comprises a region having at least 70% sequence identity to the amino acid sequence of residues 159-167 of SEQ ID NO: 24.

43. The method of any one of claims 1-42, wherein the transgene further comprises a microRNA (miRNA)-126 (miR-126) targeting sequence in the 3′-UTR.

44. The method of any one of claims 1-43, wherein the cells are pluripotent cells or multipotent cells.

45. The method of any one of claims 1-44, wherein the composition is administered to the subject by way of systemic administration, by way of direct administration to the central nervous system of the subject, by way of direct administration to the bone marrow of the subject, or by way of bone marrow transplant comprising the composition.

46. The method of any one of claims 1-45, wherein the cells are autologous cells or allogeneic cells.

47. The method of any one of claims 1-46, wherein the cells are transfected or transduced ex vivo to express the GAA.

48. The method of claim 47, wherein the cells are transduced with a viral vector selected from the group consisting of an adeno-associated virus (AAV), an adenovirus, a parvovirus, a coronavirus, a rhabdovirus, a paramyxovirus, a picornavirus, an alphavirus, a herpes virus, a poxvirus, and a Retroviridae family virus.

49. The method of claim 48, wherein the viral vector is a Retroviridae family viral vector.

50. The method of claim 49, wherein the Retroviridae family viral vector is a lentiviral vector, alpharetroviral vector, or gamma retroviral vector.

51. The method of any one of claims 48-50, wherein the Retroviridae family viral vector comprises a central polypurine tract, a woodchuck hepatitis virus post-transcriptional regulatory element, a 5′-LTR, HIV signal sequence, HIV Psi signal 5′-splice site, delta-GAG element, 3′-splice site, and a 3′-self inactivating LTR.

52. The method of any one of claims 1-51, wherein the subject with Pompe disease is a cross-reactive immunological material (CRIM)-negative subject.

53. The method of claim 52, wherein the method further comprises administering an immune tolerance induction (ITI) agent to the CRIM-negative subject prior to, concurrently with, or after the administration of the composition.

54. The method of claim 53, wherein the ITI agent comprises rituximab, methotrexate, and intravenous immunoglobulin (IVIG).

55. The method of any one of claims 1-51, wherein the subject with Pompe disease is a CRIM-positive subject.

56. The method of any one of claims 1-55, wherein the Pompe disease is an infantile-onset Pompe disease.

57. The method of claim 56, wherein the subject is from about one month to about one year of age.

58. The method of claim 57, wherein the subject is from about one month to about six months of age.

59. The method of any one of claims 56-58, wherein prior to administration of the composition to the subject, the subject exhibits one or more symptoms selected from feeding difficulties, failure to thrive, hypotonia, progressive weakness, respiratory distress, macroglossia, and cardiac hypertrophy.

60. The method of any one of claims 1-55, wherein the Pompe disease is a late-onset Pompe disease.

61. The method of any one of claims 1-60, wherein the composition is administered to the subject in a dosage of 1×10⁵ as cells/kg to about 30×10⁷ cells/kg.

62. The method of any one of claims 1-61, wherein the subject is female.

63. The method of any one of claims 1-61, wherein the subject is male.

64. The method of any one of claims 1-63, wherein the composition is administered in an amount sufficient to reduce one or more of cardiomegaly, hypotonia, cardiomyopathy, respiratory distress, muscle weakness, feeding difficulties, failure to thrive, floppy baby appearance, delay in motor development, hepatomegaly, macroglossia, wide open mouth, wide open eyes, nasal flaring, respiratory rate, engagement of accessory muscles for breathing, frequency of chest infections, arrhythmia, heart failure, impaired cough, muscle weakness, difficulty masticating and swallowing, or the composition is administered in an amount sufficient to increase one or more of facial muscle tone, air flow in the left lower zone, and vital capacity.

65. The method of any one of claims 1-64, wherein the composition is administered in an amount sufficient to reduce glycogen accumulation in muscle cells, neural cells, and/or liver cells.

66. The method of any one of claims 1-65, wherein the composition is administered in an amount sufficient to increase GAA expression level and/or enzymatic activity in muscle cells, neural cells, and/or liver cells of the subject.

67. The method of claim 65 or 66, wherein the neural cells are neurons or glial cells.

68. The method of claim 65 or 66, wherein the muscle cells are skeletal muscle cells and/or cardiac muscle cells.

69. The method of any one of claims 1-68, wherein the composition is administered in an amount sufficient to reduce glycogen accumulation in muscle tissue and/or nervous tissue.

70. The method of any one of claims 1-69, wherein the composition is administered in an amount sufficient to increase GAA expression level and/or enzymatic activity in muscle tissue or nervous tissue.

71. The method of claim 69 or 70, wherein the muscle tissue is of the heart, diaphragm, gastrocnemius muscle, quadriceps femoris muscle, and/or tibialis anterior muscle.

72. The method of claim 69 or 70, wherein the nervous tissue is of the cerebellum, cerebrum, thoracic or cervical spinal cord, and/or hippocampus.

73. The method of any one of claims 1-72, wherein the subject has not previously received GAA enzyme replacement therapy (ERT).

74. The method of any one of claims 1-72, wherein the subject has previously received GAA ERT.

75. The method of any one of claims 1-74, wherein the subject has atrophy in one or more tissues selected from heart, diaphragm, gastrocnemius muscle, quadriceps femoris muscle, tibialis anterior muscle, cerebellum, cerebrum, thoracic spinal cord, cervical spinal cord, and hippocampus tissue.

76. A composition comprising a population of cells that express a transgene encoding a GAA protein fused to a GILT tag (GILT.GAA protein), wherein the GILT tag comprises an IGF-II mutein comprising an Ala amino acid substitution at a position corresponding to Arg37 of SEQ ID NO: 15.

77. The composition of claim 76, wherein the human IGF-II mutein has an amino acid sequence that is at least 70% identical to the amino acid sequence of mature human IGF-II (SEQ ID NO: 15).

78. The composition of claim 76 or 77, wherein the GILT tag has an amino acid sequence that is at least 70% identical to the amino acid sequence of SEQ ID NO: 16.

79. The composition of claim 76 or 77, wherein the GILT tag has an amino acid sequence that is at least 70% identical to the amino acid sequence of SEQ ID NO: 17.

80. The composition of claim 76 or 77, wherein the GILT tag has an amino acid sequence that is at least 70% identical to the amino acid sequence of SEQ ID NO: 18.

81. The composition of claim 76 or 77, wherein the GILT tag has an amino acid sequence that is at least 70% identical to the amino acid sequence of SEQ ID NO: 19.

82. The composition of claim 76 or 77, wherein the GILT tag has an amino acid sequence that is at least 70% identical to the amino acid sequence of SEQ ID NO: 20.

83. The composition of any one of claims 76-82, wherein the GILT tag is encoded by a polynucleotide having a nucleic acid sequence that is at least 85% identical to the nucleic acid sequence of SEQ ID NO: 21.

84. The composition of any one of claims 76-82, wherein the GILT tag is encoded by a polynucleotide having a nucleic acid sequence that is at least 85% identical to the nucleic acid sequence of SEQ ID NO: 22.

85. The composition of any one of claims 76-82, wherein the GILT tag is encoded by a polynucleotide having a nucleic acid sequence that is at least 85% identical to the nucleic acid sequence of SEQ ID NO: 23.

86. The composition of any one of claims 76-82, wherein the human IGF-II mutein has diminished binding affinity for the insulin receptor relative to the affinity of naturally-occurring human IGF-II for the insulin receptor, wherein the IGF-II mutein is resistant to furin cleavage, wherein the IGF-II mutein binds to the human cation-independent mannose-6-phosphate receptor in a mannose-6-phosphate-independent manner.

87. The composition of any one of claims 76-86, wherein the transgene is operably linked to a promoter.

88. The composition of claim 87, wherein the promoter is a ubiquitous promoter.

89. The composition of claim 87, wherein the promoter is a cell lineage-specific promoter.

90. The composition of claim 87, wherein the promoter is a viral promoter.

91. The composition of claim 87, wherein the promoter is a synthetic promoter.

92. The composition of claim 91, wherein the synthetic promoter is an MND promoter.

93. The composition of claim 92, wherein the MND promoter comprises a polynucleotide having at least 85% sequence identity to the nucleic acid sequence of SEQ ID NO: 10.

94. The composition of claim 93, wherein the MND promoter comprises a polynucleotide having at least 85% sequence identity to the nucleic acid sequence of SEQ ID NO: 11.

95. The composition of any one of claims 76-94, wherein the transgene encodes a GAA protein having an amino acid sequence that is at least 85% identical to the amino acid sequence of SEQ ID NO: 1.

96. The composition of any one of claims 76-94, wherein the transgene encodes a GAA protein having an amino acid sequence that is at least 85% identical to the amino acid sequence of SEQ ID NO: 2.

97. The composition of any one of claims 76-94, wherein the transgene encodes a GAA protein having an amino acid sequence that is at least 85% identical to the amino acid sequence of SEQ ID NO: 3.

98. The composition of any one of claims 76-94, wherein the transgene encodes a GAA protein having an amino acid sequence that is at least 85% identical to the amino acid sequence of SEQ ID NO: 4.

99. The composition of any one of claims 76-98, wherein the GAA protein is encoded by a polynucleotide having at least 85% sequence identity to the nucleic acid sequence of SEQ ID NO: 5.

100. The composition of any one of claims 76-99, wherein the GAA protein is encoded by a polynucleotide having at least 85% sequence identity to the nucleic acid sequence of SEQ ID NO: 6.

101. The composition of any one of claims 76-100, wherein the GAA protein is encoded by a polynucleotide having at least 85% sequence identity to the nucleic acid sequence of SEQ ID NO: 7.

102. The composition of any one of claims 76-101, wherein the GAA protein is encoded by a polynucleotide having at least 85% sequence identity to the nucleic acid sequence of SEQ ID NO: 8.

103. The composition of any one of claims 76-102, wherein the GAA protein is encoded by a polynucleotide having at least 85% sequence identity to the nucleic acid sequence of SEQ ID NO: 9.

104. The composition of any one of claims 76-103, wherein the GAA is a full-length GAA.

105. The composition of any one of claims 76-104, wherein the GAA comprises a signal peptide.

106. The composition of claim 105, wherein the signal peptide is a GAA signal peptide.

107. The composition of claim 105, wherein the signal peptide is an IGF-II signal peptide.

108. The composition of claim 107, wherein the IGF-II signal peptide comprises an amino acid sequence of SEQ ID NO: 12.

109. The composition of any one of claims 76-108, wherein the transgene encodes two or more GAA transgenes.

110. The composition of any one of claims 76-109, wherein the transgene is a codon-optimized GAA transgene.

111. The composition of any one of claims 76-110, wherein the GILT.GAA protein comprises a Rb domain of ApoE.

112. The composition of claim 111, wherein the Rb domain comprises a portion of ApoE having the amino acid sequence of residues 25-185, 50-180, 75-175, 100-170, 125-160, or 130-150 of SEQ ID NO: 24.

113. The composition of claim 111 or claim 112, wherein the Rb domain comprises a region having at least 70% sequence identity to the amino acid sequence of residues 159-167 of SEQ ID NO: 24.

114. The composition of any one of claims 76-113, wherein the transgene encoding GAA further comprises a miR-126 targeting sequence in the 3′-UTR.

115. The composition of any one of claims 76-114, wherein the cells are pluripotent cells or multipotent cells.

116. A pharmaceutical composition comprising the composition of any one of claims 76-115, wherein the pharmaceutical composition further comprises a pharmaceutically acceptable carrier, diluent, or excipient.

117. A kit comprising the composition of any one of claims 76-115, or the pharmaceutical composition of claim 116, and a package insert, wherein the package insert instructs a user of the kit to perform the method of any one of claims 1-75.


SEQUENCE LISTING (26 sequences)

SEQ ID NO: 1 - protein, 952 aa, Homo sapiens
MGVRHPPCSHRLLAVCALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPRVHSRAPSPLYSVEFSEEPFGVIVHRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPIEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 2 - protein, 952 aa, Homo sapiens
MGVRHPPCSHRLLAVCALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 3 - protein, 284 aa, Homo sapiens
MGVRHPPCSHRLLAVCALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLA

SEQ ID NO: 4 - protein, 208 aa, Homo sapiens
MGVRHPPCSHRLLAVCALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPL

SEQ ID NO: 5 - DNA, 3624 bp, Homo sapiens
cagttgggaaagctgaggttgtcgccggggccgcgggtggaggtcggggatgaggcagcaggtaggacagtgacctcggtgacgcgaaggaccccggccacctctaggttctcctcgtccgcccgttgttcagcgagggaggctctgggcctgccgcagctgacggggaaactgaggcacggagcgggcctgtaggagctgtccaggccatctccaaccatgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgctgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcgtgtccacagccgggcaccgtccccactctacagcgtggagttctccgaggagcccttcggggtgatcgtgcaccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccaatggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcagaggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccaatagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggagaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttagccgggcggagtgtgttagtctctccagagggaggctggttccccagggaagcagagcctgtgtgcgggcagcagctgtgtgcgggcctgggggttgcatgtgtcacctggagctgggcactaaccattccaagccgccgcatcgcttgtttccacctcctgggccggggctctggcccccaacgtgtctaggagagctttctccctagatcgcactgtgggccggggcctggagggctgctctgtgttaataagattgtaaggtttgccctcctcacctgttgccggcatgcgggtagtattagccacccccctccatctgttcccagcaccggagaagggggtgctcaggtggaggtgtggggtatgcacctgagctcctgcttcgcgcctgctgctctgccccaacgcgaccgcttcccggctgcccagagggctggatgcctgccggtccccgagcaagcctgggaactcaggaaaattcacaggacttgggagattctaaatcttaagtgcaattattttaataaaaggggcatttggaatc

SEQ ID NO: 6 - DNA, 3837 bp, Homo sapiens
gccccgcgacgagctcccgccggtcacgtgacccgcctctgcgcgcccccgggcacgaccccggagtctccgcgggcggccagggcgcgcgtgcgcggaggtgagccgggccggggctgcggggcttccctgagcgcgggccgggtcggtggggcggtcggctgcccgcgcggcctctcagttgggaaagctgaggttgtcgccggggccgcgggtggaggtcggggatgaggcagcaggtaggacagtgacctcggtgacgcgaaggaccccggccacctctaggttctcctcgtccgcccgttgttcagcgagggaggctctgcgcgtgccgcagctgacggggaaactgaggcacggagcgggcctgtaggagctgtccaggccatctccaaccatgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccagtagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttagccgggcggagtgtgttagtctctccagagggaggctggttccccagggaagcagagcctgtgtgcgggcagcagctgtgtgcgggcctgggggttgcatgtgtcacctggagctgggcactaaccattccaagccgccgcatcgcttgtttccacctcctgggccggggctctggcccccaacgtgtctaggagagctttctccctagatcgcactgtgggccggggccctggagggctgctctgtgttaataagattgtaaggtttgccctcctcacctgttgccggcatgcgggtagtattagccacccccctccatctgttcccagcaccggagaagggggtgctcaggtggaggtgtggggtatgcacctgagctcctgcttcgcgcctgctgctctgccccaacgcgaccgctgcccggctgcccagagggctggatgcctgccggtccccgagcaagcctgggaactcaggaaaattcacaggacttgggagattctaaatcttaagtgcaattatttttaataaaaggggcatttggaatcagcaaaaaaaaaaaaaaaaaaaaaaaaaaaaaa

SEQ ID NO: 7 - DNA, 3652 bp, Homo sapiens
gccccgcgacgagctcccgccggtcacgtgacccgcctctgcgcgcccccgggcacgaccccggagtctccgcgggcggccagggcgcgcgtgcgcggaggttctcctcgtccgcccgttgttcagcgagggaggctctgcgcgtgccgcagctgacggggaaactgaggcacggagcgggcctgtaggagctgtccaggccatctccaaccatgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccagtagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttagccgggcggagtgtgttagtctctccagagggaggctggttccccagggaagcagagcctgtgtgcgggcagcagctgtgtgcgggcctgggggttgcatgtgtcacctggagctgggcactaaccattccaagccgccgcatcgcttgtttccacctcctgggccggggctctggcccccaacgtgtctaggagagctttctccctagatcgcactgtgggccggggccctggagggctgctctgtgttaataagattgtaaggtttgccctcctcacctgttgccggcatgcgggtagtattagccacccccctccatctgttcccagcaccggagaagggggtgctcaggtggaggtgtggggtatgcacctgagctcctgcttcgcgcctgctgctctgccccaacgcgaccgctgcccggctgcccagagggctggatgcctgccggtccccgagcaagcctgggaactcaggaaaattcacaggacttgggagattctaaatcttaagtgcaattatttttaataaaaggggcatttggaatcagcaaaaaaaaaaaaaaaaaaaaaaaaaaaaaa

SEQ ID NO: 8 - DNA, 3572 bp, Homo sapiens
gccccgcgacgagctcccgccggtcacgtgacccgcctctgcgcgcccccgggcacgaccccggagtctccgcgggcggccagggcgcgcgtgcgcggaggcctgtaggagctgtccaggccatctccaaccatgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccagtagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttagccgggcggagtgtgttagtctctccagagggaggctggttccccagggaagcagagcctgtgtgcgggcagcagctgtgtgcgggcctgggggttgcatgtgtcacctggagctgggcactaaccattccaagccgccgcatcgcttgtttccacctcctgggccggggctctggcccccaacgtgtctaggagagctttctccctagatcgcactgtgggccggggccctggagggctgctctgtgttaataagattgtaaggtttgccctcctcacctgttgccggcatgcgggtagtattagccacccccctccatctgttcccagcaccggagaagggggtgctcaggtggaggtgtggggtatgcacctgagctcctgcttcgcgcctgctgctctgccccaacgcgaccgctgcccggctgcccagagggctggatgcctgccggtccccgagcaagcctgggaactcaggaaaattcacaggacttgggagattctaaatcttaagtgcaattatttttaataaaaggggcatttggaatcagcaaaaaaaaaaaaaaaaaaaaaaaaaaaaaa

SEQ ID NO: 9 - DNA, 2859 bp, Homo sapiens
atgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccagtagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttag

SEQ ID NO: 10 - DNA, 385 bp, Artificial Sequence
  Synthetic Construct
gatcaaggttaggaacagagagacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgg

SEQ ID NO: 11 - DNA, 399 bp, Artificial Sequence
  Synthetic Construct
tttatttagtctccagaaaaaggggggaatgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagccca

SEQ ID NO: 12 - protein, 24 aa, Homo sapiens
MGIPMGKSMLVLLTFLAFASCCIA

SEQ ID NO: 13 - protein, 3 aa, Artificial Sequence
  Synthetic Construct
GAP

SEQ ID NO: 14 - DNA, 9 bp, Artificial Sequence
  Synthetic Construct
ggcgcgccg

SEQ ID NO: 15 - protein, 67 aa, Artificial Sequence
  Synthetic Construct
AYRPSETLCGGELVDTLQFVCGDRGFYFSRPASRVSRRSRGIVEECCFRSCDLALLETYCATPAKSE

SEQ ID NO: 16 - protein, 85 aa, Artificial Sequence
  Synthetic Construct
GGGGAGGGGAGGGGAGGGGAGGGPSLCGGELVDTLQFVCGDRGFYFSRPASRVSARSRGIVEECCFRSCDLALLETYCATPAKSE

SEQ ID NO: 17 - protein, 91 aa, Artificial Sequence
  Synthetic Construct
GAPGGGSPAPAPTPAPAPTPAPAGGGPSGAPLCGGELVDTLQFVCGDRGFYFSRPASRVSARSRGIVEECCFRSCDLALLETYCATPAKSE

SEQ ID NO: 18 - protein, 116 aa, Artificial Sequence
  Synthetic Construct
GAPGGSPAGSPTSTEEGTSESATPESGPGTSTEPSEGSAPGSPAGSPTSTGPSGAPLCGGELVDTLQFVCGDRGFYFSRPASRVSARSRGIVEECCFRSCDLALLETYCATPAKSE

SEQ ID NO: 19 - protein, 94 aa, Artificial Sequence
  Synthetic Construct
MGIPMGKSMLVLLTFLAFASCCIAAYRPSETLCGGELVDTLQFVCGDRGFYFSRPASRVSRRSRGIVEECCFRSCDLALLETYCATPAKSEGAP

SEQ ID NO: 20 - protein, 88 aa, Artificial Sequence
  Synthetic Construct
MGIPMGKSMLVLLTFLAFASCCIAALCGGELVDTLQFVCGDRGFYFSRPASRVSARSRGIVEECCFRSCDLALLETYCATPAKSEGAP

SEQ ID NO: 21 - DNA, 260 bp, Artificial Sequence
  Synthetic Construct
ggcggaggcggagctggtggcggcggagcaggcggtggtggtgcaggcggcggaggtgctggcggaggaccatctctttgtggcggagaactggtggacaccctgcagttcgtgtgtggcgacagaggcttctactttagcagacccgccagcagagtgtccgccagatctagaggaatcgtggaagagtgctgcttcagaagctgcgacctggcactgctggaaacctactgtgccacaccagccaagagcgagtgatg

SEQ ID NO: 22 - DNA, 279 bp, Artificial Sequence
  Synthetic Construct
ggagcaccaggcggaggatctccagctcctgctcctacaccagctccagcaccgacgcctgctccagctggcggaggaccttctggtgcacctctttgtggcggagagctggtggataccctgcagttcgtgtgtggcgaccggggcttctactttagcagacctgccagcagagtgtccgccagatctagaggcatcgtggaagagtgctgcttcagaagctgcgacctggcactgctggaaacctactgtgccacaccagccaagagcgagtgatga

SEQ ID NO: 23 - DNA, 354 bp, Artificial Sequence
  Synthetic Construct
ggagcaccaggcggatctccagcaggatctccaacctctaccgaggaaggcacaagcgagtctgccacacctgagtctggacctggcacaagcacagagcctagcgaaggatctgccccaggttctcctgccggctctcctacaagtacaggaccttctggcgctccactgtgtggcggagaactggtggataccctgcagttcgtgtgcggcgacagaggcttctactttagcagacccgccagcagagtgtccgccagatctagaggaatcgtggaagagtgctgcttcagaagctgcgatctggcactgctggaaacctactgtgccacaccagccaagagcgagtgatga

SEQ ID NO: 24 - protein, 317 aa, Artificial Sequence [sequence omitted]

SEQ ID NO: 25 - protein, 18 aa, Artificial Sequence
  Synthetic Construct
LRKLRKRLLLRKLRKRLL

SEQ ID NO: 26 - protein, 109 aa, Artificial Sequence
  Synthetic Construct
MGIPMGKSMLVLLTFLAFASCCIAAGAPLRKLRKRLLLRKLRKRLLGAPLCGGELVDTLQFVCGDRGFYFSRPASRVSARSRGIVEECCFRSCDLALLETYCATPAKSE